(19) United States
(12) Patent Application Publication
(10) Pub. No.: US 2007/0299092 A1
(43) Pub. Date: Dec. 27, 2007

(54) Quinone Substituted Quinazoline and Quinoline Kinase Inhibitors

(75) Inventors: Middleton Brawner Floyd Jr, Suffern, NY (US); Thomas Nittoli, Nyack, NY (US); Allan Wissner, Ardsley, NY (US); Russell George Dushin, Garrison, NY (US); Ramaswamy Nilakantan, Closter, NJ (US); Charles Ingalls, Valley Cottage, NY (US); Heidi Leigh Fraser, Yorktown Heights, NY (US); Bernard Dean Johnson, Stony Point, NY (US)

Correspondence Address: Wyeth c/o Darby & Darby, P.C., P.O. BOX 770, Church Street Station, NEW YORK, NY, 10008-0770, US

(73) Assignee: Wyeth, Madison, NJ (US)

(21) Appl. No.: 11/569,306

(22) PCT Filed: May 11, 2005
(86) PCT No.: PCT/US05/16800

§ 371 Date: Dec. 8, 2006

Related U.S. Application Data
(60) Provisional application No. 60/573,251, filed on May 20, 2004.

(51) Int. Cl. C07D 239/72; A61K 31/517; A61P 19/02; A61P 27/00; C12N 9/99; A61P 3/00; A61P 35/00; A61P 9/00

(52) U.S. Cl. 514/266.1; 435/184; 544/283

(57) ABSTRACT

The present invention provides for compounds with the general formula: A compound of formula (1) having the structure (1) wherein Z is a radical selected from the group (a), (b), or (c) as well as methods and compositions containing these compounds useful for treatment of diseases that are characterized, at least in part, by excessive, abnormal, or inappropriate angiogenesis. These disease states, include but are not limited to, cancer, diabetic retinopathy, macular degeneration and rheumatoid arthritis. These compounds inhibit angiogenesis by inhibiting a tyrosine kinase receptor enzyme, specifically KDR, and binding to the KDR in an irreversible manner.

This application claims priority from U.S. Provisional Application Ser. No. 60/573,251, filed May 20, 2004, the disclosure of which is incorporated herein by reference in its entirety.

### 1. FIELD OF THE INVENTION

This invention relates to certain substituted quinazoline and quinoline compounds as well as the pharmaceutically acceptable salts thereof. The compounds of the present invention inhibit the action of certain growth factor receptor protein tyrosine kinases (PTK) that regulate blood vessel growth and function as anti-angiogenic agents.

### 2. BACKGROUND OF THE INVENTION

Growth of most solid tumors is dependent on the angiogenesis involving activation, proliferation and migration of vascular endothelial cells and their subsequent differentiation into capillary tubes. Angiogenesis of tumors allows them access to blood-derived oxygen and nutrients, and also provides them adequate perfusion. Hence inhibiting angiogenesis is an important therapeutic strategy for treating cancer as well as a number of chronic diseases, such as rheumatoid arthritis, psoriasis, diabetic retinopathy and age-related macular degeneration.

Tumor cells produce a number of angiogenic molecules. Vascular Endothelial Growth Factor (VEGF) is one such angiogenic factor. VEGF, a homodimeric disulfide-linked member of the Platelet-Derived Growth Factor (PDGF) family, is an endothelial cell-specific mitogen and is known to cause a profound increase in the vascular endothelial permeability in the affected tissues. VEGF is also a senescence-preventing survival factor for endothelial cells. Almost all nucleated tissues in the body possess the capability to express VEGF in response to various stimuli including hypoxia, glucose deprivation, advanced glycation products and inflammatory cytokines.

Growth-promoting angiogenic effects of VEGF are mediated predominantly via its signaling receptor Kinase insert Domain containing Receptor (KDR). This receptor is sometimes also referred to as Flk-1 or VEGFR-2. The effects of VEGF are also mediated by the Fms-Like Tyrosine kinase (Flt-1, also known as VEGFR-1).

KDR is a receptor protein tyrosine kinase with an extracellular VEGF-binding domain consisting of seven immunoglobulin-like domains and a cytoplasmic domain containing the catalytic tyrosine kinase domain split by a kinase-insert region. Binding to VEGF causes dimerization of KDR resulting in its autophosphorylation and initiation of signaling cascade. The expression of KDR is low on most endothelial cells. However, activation with angiogenic agents results in a significant upregulation of KDR on endothelial cells. Most angiogenized blood vessels express high levels of KDR. Therefore, compounds that inhibit the tyrosine kinase activity of KDR will also function as anti-angiogenic agents and are useful for the treatment of cancer and other diseases.

There are several benefits to the use of anti-angiogenic therapy for the treatment of cancer. Genetically unstable cancer cells often develop resistance to standard therapy. By targeting untransformed endothelial cells, resistance is less likely to develop. Additionally, slow growing tumors that are resistant to standard cytotoxic cancer therapy may be responsive to a continuous low to moderate dose of anti-angiogenic drugs. Moreover, since the therapeutic target is not the tumor cells itself, the anti-angiogenic drug therapy is effective against tumors from different tissue origins. The growth of solid tumors, such as lung, colorectal, breast and prostate, have been inhibited by targeting KDR in animal models as well as patients.

Neutralizing antibodies to VEGF and KDR have been developed that inhibit primary tumor growth, as well as metastases, in vivo. When these neutralizing antibodies are used in combination with standard cytotoxics, such as paclitaxel, efficacy of the cytotoxics is improved. Antisense RNA, ribozymes and DNAzyme technology that specifically diminish VEGR or KDR expression have been demonstrated to be effective in both cellular and animal models.

Some small molecule inhibitors of KDR kinase are also in development. Unlike RNA and antibody strategies, most of the small molecule inhibitors are non-selective and inhibit other related kinases, which may be of benefit since some of these kinases also may be involved in angiogenesis. These agents appear to be most effective when administered orally on a daily basis.

However, despite these benefits, the clinical results of the inhibitor therapy has been mixed. Phase I safety trials of small molecules and antibody monotherapy has shown minimal adverse side effects. However, combination trials with established cytotoxic therapy have resulted in more adverse events, such as vascular effects. In phase II and III clinical trials of solid tumors, some partial regressions have been observed. Some complete regressions, increased time to progression and increased survival time have been reported with the anti-VEGF antibody, alone or in combination therapy.

It is unknown why there is limited success with these agents. However, an alternative method of targeting KDR is to use irreversibly binding inhibitors. A tyrosine kinase, such as KDR, catalyses the transfer of a phosphate group from a molecule of ATP to a tyrosine residue located on a protein substrate. The reversible inhibitors of KDR so far known in the art are usually competitive with either the ATP or the protein substrate of the kinase. Some of these inhibitors can be competitive with both ATP and substrate simultaneously. The 4-anilinoquinazoline and 4-anilinoquinoline inhibitors of KDR known in the art and described below are reversible binding inhibitors that are competitive with ATP. Since the concentration of ATP in a cell is normally very high (millimolar), compounds that are competitive with ATP may show diminished efficacy and duration of action since it would be difficult for such compounds to reach the concentrations within the cell that are necessary to displace the ATP from its binding site for the extended time needed to inhibit tumor growth effectively.

The KDR inhibitors known to date are believed to reversibly bind to the target receptor, but compounds that irreversibly bind to certain other target receptors have been shown to be superior tumor suppressors. For example, Frey et al. (Proc. Natl. Acad. Sci. U.S.A. 95:12022-12027 (1998)) have reported small molecules purported to irreversibly inhibit epidermal growth factor receptor (EGFR) bind irreversibly to the receptor and alkylate a cysteine residue in the ATP binding pocket of the molecule. These compounds are said to be more potent suppressors of tumor growth in animal models. Others have reported that irreversible EGFR kinase inhibitors effectively suppress growth in human tumor cell models (Discafani et al., Biochem. Biopharmacol. 57:917-925 (1999)). Hence, the identification of compounds that irreversibly bind KDR offers the ability to identify new therapeutic compounds which are likely to be superior tumor suppressors compared to the reversible KDR inhibitors that are currently available.

As demonstrated below, many of the quinazoline and quinoline inhibitors of this invention have the unique ability of inhibiting KDR kinase in an irreversible manner or behave as if they are inhibiting in an irreversible manner and are therefore non-competitive with ATP or protein substrate. Thus, the compounds of the present invention would function as superior anti-angiogenic agents that are useful for the treatment of the aforementioned disease states.

For recent reviews on this subject see F. J. Giles, “The Emerging role of Angiogenesis Inhibitor in Hematologic Malignancies” Oncology Supplement 16:23-29 (2002); S. J. Boyer, “Small Molecule Inhibitors of KDR (VEGFR-2) Kinase: An Overview of Structure Activity Relationships”, Curr. Top. Med. Chem. 2:973-1000 (2002); J. Folkman, “Role of Angiogensis in Tumor Growth and Metastasis”, Seminars in Oncology 29:15-18 (2002); and R. K. Jain, “Tumor Angiogenesis and Accessibility: Role of Vascular Endothelial Growth Factor”, Seminars in Oncology 29:3-9 (2002).

This invention also relates to the manufacture of said quinazoline and quinolines. In addition to the above utilities, some of the compounds of the present invention are useful for the preparation of other compounds of this invention.

The compounds of this invention are certain substituted quinazoline and quinoline derivatives. Throughout this patent application, these ring systems will be numbered as indicated below:

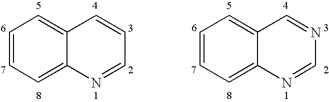

Unlike many of the quinoline compounds described in the prior art, the quinoline compounds of the present invention are substituted at the 4-position with a quinone moiety. There are reports of quinolines, unsubstituted at the 4-position, that are inhibitors of protein tyrosine kinases (Gazit A. et al., J. Med. Chem. 39(11):2170 (1996)). International patent applications WO 96/09294, WO 98/13350, WO 01/55116 and WO 02/12226 describe inhibitors of protein tyrosine kinases that include 4-anilino quinolines with a large variety of substituents on positions 5-8, but no quinone ring in the 4-position. U.S. Pat. No. 5,480,883 describes quinoline derivatives that are inhibitors of protein tyrosine kinases, but do not have an attached quinone ring. International patent applications WO 98/02434 and WO 98/02438 also describe quinoline derivatives that do not have an attached quinone ring.

3-Cyanoquinolines are also present in the literature. The compounds of the present invention differ from these compounds because of the quinone substitutent at the 4-position. Several patents and patent applications disclose compounds with an expanded anilino moiety at the 4-position. In U.S. Pat. No. 6,297,258, WO 00/18740, WO 00/18761, and WO 02/36570, compounds having an ether, thioether or sulfide linkage in addition to the possible aniline at the quinoline 4-position are described. However, none of these compounds have an attached quinone ring. International patent application WO 03/00266 discloses phosphorus-containing 4-anilino-3-cyanoquinolines. This patent application allows for additional substitution of a broad range on the quinoline at the 2, 6, and 7 positions as well as incorporating not just anilines at the 4-position, but also aliphatic amines and other heteroaliphatic or heteroaryl substituents. However, the compounds described do not have an attached quinone ring. International patent application WO 02/72578 describes a piperazine ring, with a urea functionality, directly linked to the quinazoline at the 4-position. Again, there is no disclosure of compounds with a quinine moiety attached at the 4-position disclosed in this application.

The core structures claimed in international patent applications DE 1990/8567, DE 1001/7539, and WO 00/55141 encompass quinolines, 3-cyanoquinolines and quinazolines with 4-anilino substituent and variations of the substituents at the 5, 6, 7, and 8 positions of the heterocyclic ring. However, none of the compounds described in these applications have an attached quinone ring.

Several patents teach compounds with quinolines and quinazolines in their generic core structures but do not included a quinone substitutent at the 4-position of the corresponding heterocycle like the compounds of the present invention. WO 00/78735, WO 02/18370, WO 02/18376, and WO 02/18372 disclose compounds containing 4-anilinoquinolines and 4-anilinoquinazolines, allowing additional substitution at the heterocycles 6 and 7 positions. Two additional patent applications (GB 2345486 and WO 99/35132) allow for extensive variation of the aniline moiety at the 4-position of the corresponding heterocycle, such as heterocyclic anilines, but the compounds described do not have an attached quinone ring. These two patent applications also allow for incorporation of an additional heteroatom at either the 6 or 7 positions of the heterocycle. Compounds with a cyclic aliphatic amine incorporated at the quinoline and quinazoline 4-position are disclosed in WO 98/14431 and U.S. Pat. No. 6,169,008. International patent application WO 97/17329 teaches compounds that exclude the typical aniline substitution at the 4-position of the corresponding heterocycle yet encompasses phenyl ethers, phenyl thioethers and carbon linkages with simple substitution at the 6 and 7 position of the corresponding heterocycle. This patent application also does not describe compounds that have an attached quinine ring.

In addition to quinolines, certain quinazoline derivatives that are similar in some respects to the compounds of this invention are known to be inhibitors of protein tyrosine kinases. The application WO 98/50370 contains a disclosure of 2,4,5-substituted quinazolines that inhibit serine threonine kinases. These compounds contain different functional groups and substitution pattern than the compounds of the present invention. The key component of the disclosed compounds of application WO 99/10349 is the pyrrolione ring substituted at the quinazoline 4-position, while the compounds of the present invention contain a novel quinone or quinone epoxide ring at the 4-position. International patent application WO 01/66099 teaches a compound containing a urea directly linked to the quinazoline at the 4-position, but again, no disclosure of a quinone moiety at this same position. Similarly other international patent applications (WO 02/16351, WO 02/16360, WO 02/16361, and WO 02/16362) contain a urea (or thiourea) moiety off the quinazoline 4-position. However, in these instances, an essential piperazine ring links the urea to the quinazoline.

While a large portion of the quinazoline patent literature concerns anilinoquinazolines, again the compounds of the present invention are unique because of the quinone or quinone epoxide substitutent at the 4-position of the quinazoline. The application, EP-520722, describes 4-anilinoquinazolines that contain simple substituents such as chloro, trifluoromethyl, or nitro groups at positions 5 to 8. The compounds in application EP-566226 are similar, but with a much larger variety of allowed substituents at positions 5 to 8. Application WO 96/09294 describes compounds with similar substituents at positions 5 to 8 and with the substituent at the 4-position consisting of some polycyclic ring systems. Some simple substituted quinazolines are also described in applications WO 95/24190, WO 95/21613, WO 95/15758, WO 97/32856, WO 98/13354 and WO 01/32651. The patent applications EP-602851 and WO 95/23141 cover similar quinazoline derivatives where the aryl group attached at position 4 can be a variety of heterocyclic ring structures. The application EP-635498 describes certain quinazoline derivatives that have alkenoylamino and alkynoylamino groups among the substituents at position 6 and a halogen atom at position 7. WO 96/33981 describes 4-anilinoquinazolines where the 6 and 7 position may contain polyether or amino substitution. None of these patent applications disclose or suggest quinazoline compounds with a quinone or quinone epoxide substituent at the 4-position like the quinazoline compounds of the present invention.

There are additional patents and patent applications that describe quinazolines that are inhibitors of various kinases such as WO 96/33978, WO 02/93577, WO 02/92579, WO 02/92578, WO 03/00188, WO 02/30924, WO 02/30926, WO 02/34744, WO 02/18351, WO 97/30044, EP-787722, WO 02/18373, WO 02/50043, WO 02/18375, EP-1230919, WO 02/50043, WO 97/30034, WO 99/01441, WO 02/02552, WO 97/30035, WO 01/77085, WO 00/21955, WO 00/47212, WO 01/21594, WO 01/21596, WO 01/21597, WO 02/85895, and U.S. Pat. No. 5,721,237. However, none of these patent documents describe compounds that have an attached quinone or quinone epoxide moiety, like the compound of the present invention.

The citation and/or discussion of a reference in this section and throughout the specification is provided merely to clarify the description of the present invention and is not an admission that any such reference is “prior art” to the invention described herein.

### 3. SUMMARY OF THE INVENTION

The present invention overcomes the problems in the art by providing compounds that irreversibly bind to tyrosine kinase enzymes, specifically KDR, or behave as if they are inhibiting in an irreversible manner and are therefore non-competitive with ATP or protein substrate. The compounds of this invention can function like irreversible binding inhibitors by virtue of the fact that they may form covalent bonds to amino acid residues located at the active site of the enzyme. In this respect, the compounds of the present invention differ from all other KDR inhibitors reported previously. In particular, it is shown that it is the unique nature and combination of substituents contained in the compounds of the present invention that may lead to the irreversible binding of the inhibitor to the enzyme. These unique properties of the compounds of this invention contribute to their ability to function as anti-angiogenic agents.

There are many advantages to an irreversible KDR inhibitor. For one, as discussed above, these inhibitors would not compete with ATP.

Secondly, since prolonged suppression of the kinase is most likely necessary for maximum tumor suppression, an irreversibly bound inhibitor provides an advantage by permanently eliminating the existing kinase activity, which should return only when a new receptor is synthesized.

Lower plasma levels of the inhibitor is also an advantage. The irreversible binding inhibitors require that plasma concentrations be attained only long enough to expose the inhibitor to the target. After the irreversible inhibitor binds, no more inhibitor is needed in the plasma in order to maintain inhibition. Thus, there is less likelihood of toxicity, which results from high or prolonged plasma levels.

Lastly, there may be possible cross-reactivity of the irreversible binding inhibitors with other kinases involved in angiogenesis that have homologous amino acids in their active site, e.g., platelet-derived growth factor receptor (PDGFR) and vascular endothelial growth factor receptor 1 (VEGFR-1).

This invention provides a compound of formula 1: 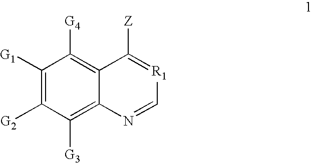
wherein:
R₁ is N, C—CN, C—H, C—F, C—Cl, C—Br, or C—I
G₁, G₂, G₃, and G₄ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, alkylamido of 2-7 carbon atoms, halomethyl, alkyl-N-alkylamido of 4-10 carbon atoms, alkanoyloxy of 2-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, trifluoromethoxy, phenylacetyl, cyano, nitro, carboxy, carboalkoxy of 2-7 carbon atoms, carboalkyl of 2-7 carbon atoms, phenoxy, phenyl, thiophenoxy, benzyl, amino, hydroxyamino, alkoxyamino of 1-4 carbon atoms, alkylamino of 1-6 carbon atoms, dialkylamino of 2 to 12 carbon atoms, N-alkylcarbamoyl, N,N-dialkylcarbamoyl, N-alkyl-N-alkenylamino of 4 to 12 carbon atoms, N,N-dialkenylamino of 6-12 carbon atoms, phenylamino, benzylamino, R₂NH, 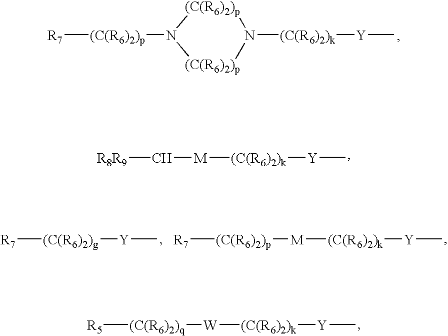
with the proviso that G₃ or G₄ are not R₂NH;
R₂, is selected from the group consisting of 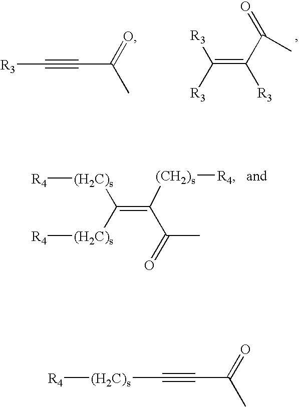
R₃ is, independently, hydrogen, alkyl of 1-6 carbon atoms, carboxy, carboalkoxy of 1-6 carbon atoms, phenyl, carboalkyl of 2-7 carbon atoms, 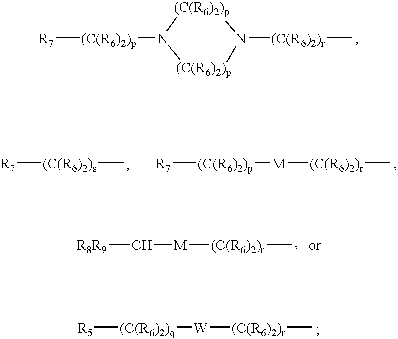
R₄ is Cl, Br, or I;
R₆ is hydrogen, alkyl of 1-6 carbon atoms, alkenyl of 2-6 carbon atoms, alkynyl of 2-6 carbon atoms, cycloalkyl of 1-6 carbon atoms, carboalkyl of 2-7 carbon atoms, carboxyalkyl 2-7 carbon atoms, phenyl, or phenyl optionally substituted with one or more halogen, alkoxy of 1-6 carbon atoms, trifluoromethyl, amino, alkylamino of 1-3 carbon atoms, dialkylamino of 2-6 carbon atoms, nitro, cyano, azido, halomethyl, alkoxymethyl of 2-7 carbon atoms, alkanoyloxymethyl of 2-7 carbon atoms, alkylthio of 1-6 carbon atoms, hydroxy, carboxyl, carboalkoxy of 2-7 carbon atoms, phenoxy, phenyl, thiophenoxy, benzoyl, benzyl, phenylamino, benzylamino, alkanoylamino of 1-6 carbon atoms, or alkyl of 1-6 carbon atoms; with the proviso that the alkenyl or alkynyl moiety is bound to a nitrogen or oxygen atom through a saturated carbon atom;
R₇ is —NR₆R₆, —OR₆, —R₄, —N(R₆)₃⁺ or —NR₆(OR₆);
M is >NR₆, —O—, >N—(C(R₆)₂)ₚNR₆R₆, or >N—(C(R₆)₂)ₚ—OR₆, or a divalent phenyl radical;
W is >NR₆, —O—, a divalent phenyl radical, or is a bond;
R₅ is a phenyl radical or a heterocyclic radical selected from the group consisting of morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane, tetrahydropyran, and 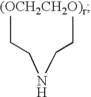
wherein the phenyl radical or the heterocylic radical may be optionally mono- or di-substituted on carbon with R₆, hydroxy, —N(R₆)₂, —OR₆—(C(R₆)₂)ₛOR₆, or —(C(R₆)₂)ₛN(R₆)₂ and
wherein the heterocylic radical may be optionally mono-substituted on nitrogen with R₆ and optionally mono or di-substituted on a saturated carbon with divalent radicals —O— or —O(C(R₆)₂)ₛO—;
R₈ and R₉ are each, independently, —(C(R₆)₂)ᵣNR₆R₆, or —(C(R₆)₂)ᵣOR₆;
Y is a divalent radical selected from the group consisting of 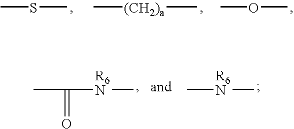
a=0-1;
g=1-6;
k=0-4;
p=2-4;
q=0-4;
r=1-4;
s=1-6;
provided that

when R₆ is alkenyl of 2-7 carbon atoms or alkynyl of 2-7 carbon atoms, such alkenyl or alkynyl moiety is bound to a nitrogen or oxygen atom through a saturated carbon atom;

and provided that

when Y is —NR₆— and R₇ is —NR₆R₆, —N(R₆)₃⁺, or —NR₆(OR₆), then g=2-6;

when M is —O— and R₇ is —OR₆ then p=1-4;

when Y is —NR₆— then k=2-4;

when Y is —O— and M or W is —O— then k=1-4;

when W is not a bond or a divalent phenyl radical with R₅ bonded through a nitrogen atom then q=2-4,

when M is a divalent phenyl radical then p=0-4 and r=0-4,

when W is a divalent phenyl radical then r=0-4,

and when W is a bond with R₅ bonded through a nitrogen atom and Y is —O— or —NR₆— then k=2-4;
Z is a radical selected from the group 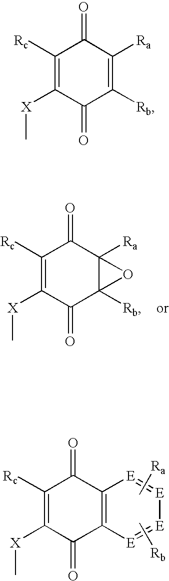
X is a divalent radical selected from the group —NH—, >NR₁₀, —O—, and —S—;
R₁₀ is an hydrogen, an alkyl group from 1-6 carbon atoms, phenyl or benzyl;
Rₐ, R_{b}, R_{c} are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxyalkyl of 1-6 carbon atoms, haloalkyl of 1-6 carbon atoms, alkanoyloxy of 2-6 carbon atoms, alkenoyloxy of 3-8 carbon atoms, alkynoyloxy of 3-8 carbon atoms, alkylamido of 2-7 carbon atoms, alkanoyloxymethyl of 2-7 carbon atoms, alkenoyloxymethyl of 4-9 carbon atoms, alkynoyloxymethyl of 4-9 carbon atoms, alkoxyalkyl of 2-14 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, phenylacetyl, alkenylsulfonamido of 2-6 carbon atoms, alkynylsulfonamido of 2-6 carbon atoms, hydroxy, trifluoromethyl, trifluoromethoxy, cyano, nitro, azido, carboxy, carboalkoxy of 2-7 carbon atoms, carboalkyl of 2-7 carbon atoms, phenoxy, phenyl, thiophenoxy, benzyl, benzyloxy, benzylthio, amino, hydroxyamino, alkoxyamino of 1-4 carbon atoms, alkylamino of 1-6 carbon atoms, dialkylamino of 2 to 12 carbon atoms, N-alkylcarbamoyl of 2 to 6 carbon atoms, N,N-dialkylcarbamoyl of 2 to 12 carbon atoms, N-alkyl-N-alkenylamino of 4 to 12 carbon atoms, N,N-dialkenylamino of 6-12 carbon atoms, phenylamino, benzylamino, 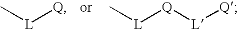
when attached to a double bond at contiguous carbon atoms, Rₐ and R_{b} can be taken together as the divalent radicals —(C(R₁₀)₂)₃—, —(C(R₁₀)₂)₄—, —X—(C(R₁₀)₂)₃—, —X—(C(R₁₀)₂)₂—X—, —C(R₁₀)₂—X—(C(R₁₀)₂)₂—, or —C(R₁₀)₂—X—C(R₁₀)₂—;
Q and Q′ are a phenyl mono or divalent radical which may be optionally substituted with 1-5 halogen atoms, or mono- di- or tri-substituted with a substituent selected from the group consisting of hydrogen, alkyl of 1-6 carbon atoms, alkenyl of 2-6 carbon atoms, alkynyl of 2-6 carbon atoms, azido, hydroxyalkyl of 1-6 carbon atoms, alkylamido of 2-7 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, benzoyl, amino, phenylacetyl, alkylamino of 1-6 carbon atoms, dialkylamino of 2 to 12 carbon atoms, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N-alkylcarbamoyl of 2 to 6 carbon atoms, N,N-dialkylcarbamoyl of 2 to 12 carbon atoms, N,N-dialkylaminoalkoxy of 3-10 carbon atoms, mercapto, and benzoylamino, or
Q and Q′ are a mono or divalent radical comprising a 3-8-membered heterocyclic ring where the heterocyclic ring contains 1 to 3 heteroatoms selected from N, O, and S; wherein the heterocyclic ring may be optionally substituted with 1-5 halogen atoms, or mono- or di-substituted with a substituent selected from the group consisting of oxo, thio, alkyl of 1-6 carbon atoms, alkenyl of 2-6 carbon atoms, alkynyl of 2-6 carbon atoms, azido, alkylamido of 2-7 carbon atoms, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, phenylacetyl, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylcarbamoyl of 2 to 6 carbon atoms, N,N-dialkylcarbamoyl of 2 to 12 carbon atoms, N-alkylaminoalkoxy of 2-9 carbon atoms, N,N-dialkylaminoalkoxy of 3-10 carbon atoms, mercapto, and benzoylamino, or
Q and Q′ are a mono or divalent radical comprising a fused or bridged bicyclic or tricyclic carbocyclic ring system or a fused or bridged bicyclic or tricyclic heterocyclic ring system of 6 to 18 atoms, where the bicyclic or tricyclic heterocyclic ring system contains 1 to 4 heteroatoms selected from N, O, and S; wherein the bicyclic or tricyclic carbocyclic ring system or the bicyclic or tricyclic heterocyclic ring system may be optionally substituted with 1-5 halogen atoms, or mono-, di-, tri-, or tetra-substituted with a substituent selected from the group consisting of oxo, thio, alkyl of 1-6 carbon atoms, alkenyl of 2-6 carbon atoms, alkynyl of 2-6 carbon atoms, azido, alkylamido of 2-7 carbon atoms, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenylacetyl, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylcarbamoyl of 2 to 6 carbon atoms, N,N-dialkylcarbamoyl of 2 to 12 carbon atoms, N-alkylaminoalkoxy of 2-9 carbon atoms, N,N-dialkylaminoalkoxy of 3-10 carbon atoms, mercapto, and benzoylamino, or
Q and Q′ are hydrogen or a mono or divalent radical comprising straight or cyclic alkyl groups of 1 to 10 carbon atoms, both of which can optionally be branched, substituted with 1-6 halogen groups, or contain sites of unsaturation, or be; L and L′ are divalent radicals selected from the group 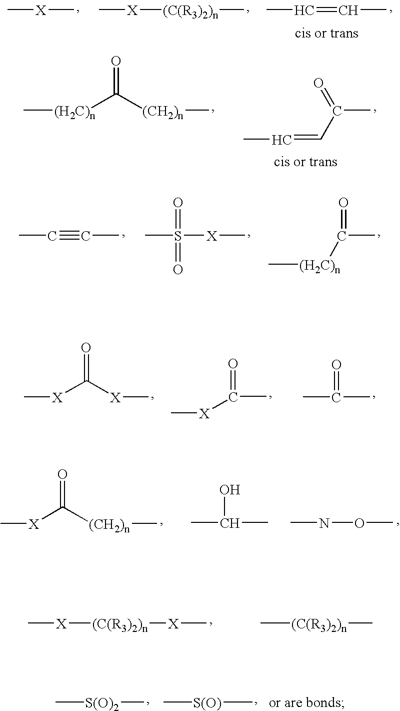
n is an integer from 1 to 4;
E is CH or N with the proviso that there be no more than 2 ring nitrogen atoms; it is provided that when Z is the moiety 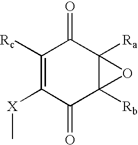
Rₐ and R_{b} are independently hydrogen or are attached to the ring only via carbon atoms;
or a pharmaceutically acceptable salt thereof.

The present invention also provides for compositions containing these compounds and methods of using these compounds and compositions to treat patients in need of treatment, prevention and/or suppression of excessive, abnormal or inappropriate angiogenesis related to such disease states as cancer, including, but not limited to, cancer of the breast, kidney, bladder, mouth, larynx, esophagus, stomach, prostate, colon, ovary and lung, diabetic retinopathy, macular degeneration and rheumatoid arthritis.

### 4. DETAILED DESCRIPTION OF THE INVENTION

The terms used in this specification generally have their ordinary meanings in the art, within the context of the invention, and in the specific context where each term is used. Certain terms are discussed below, or elsewhere in the specification, to provide additional guidance to the practitioner in describing the compounds, compositions, and methods of the invention and how to make and use them. For convenience, certain terms are highlighted, for example using italics and/or quotation marks. The use of highlighting has no influence on the scope and meaning of a term; the scope and meaning of a term is the same, in the same context, whether or not it is highlighted. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and in no way limits the scope and meaning of the invention or of any exemplified term. Likewise, the invention is not limited to the preferred embodiments.

As used herein, “about” or “approximately” shall generally mean within 20 percent, preferably within 10 percent, and more preferably within 5 percent of a given value or range.

The terms “prevent” or “prevention”, as used herein, refer to the partial or complete inhibition of the development of a condition that impairs the performance of a function of the human body. The terms “treat” or “treatment”, as used herein, refer to an attempt to ameliorate a disease problem. Further, the term “suppress” or “suppression” refers to a complete or partial inhibition of a condition, e.g., as evidenced by a lessening of the severity of the symptoms associated with that condition.

Still further, the terms “effective amount” and “therapeutically effective amount” refer to that amount of the compound or composition determined by the skilled artisan to effectively prevent, suppress or treat the targeted condition. The effective amount of a compound or composition will be determined empirically by administering a range of dosages to the patient and observing that dosage which is most effective for the treatment of the condition and best tolerated by the patient. The method of making such a determination will be readily understood by the skilled artisan and will necessarily take into account such factors as, inter alia, the route of administration, formulation, and the condition, age, sex, height, and weight of the patient.

The terms “irreversible” or “irreversibly” are used herein to mean an inhibitor of receptor tyrosine kinase activity that is permanently bound or associated with the receptor tyrosine kinase.

As discussed above, the present invention provides compounds having Formula 1 or pharmaceutically acceptable salts thereof. The preferred pharmaceutically acceptable salts are those derived from such organic and inorganic acids such as acetic, lactic, citric, tartaric, succinic, maleic, malonic, gluconic, hydrochloric, hydrobromic, phosphoric, nitric, sulfuric, methanesulfonic, and similarly known acceptable acids.

Either or all rings of the bicyclic or tricyclic carbocyclic ring systems or the bicyclic or tricyclic heterocyclic ring systems of Formula 1 may be fully unsaturated, partially saturated, or fully saturated. The bicyclic or tricyclic heterocyclic ring can be bound to a carbon atom via either a carbon or nitrogen atom. The bicyclic or tricyclic heterocyclic ring can be bound to a heteroatom via carbon atom. An oxo substituent on the bicyclic or tricyclic carbocyclic ring system or bicyclic or tricyclic heterocyclic ring system means that one of the carbon atoms has a carbonyl group. A thio substituent on the bicyclic or tricyclic carbocyclic ring system or the bicyclic or tricyclic heterocyclic ring system means that one of the carbon atoms has a thiocarbonyl group.

Moreover, when Q or Q′ is a 3-8-membered heterocyclic ring, it may be fully unsaturated, partially saturated, or fully saturated. The heterocyclic ring can be bound to a carbon atom via either a carbon or nitrogen atom. The heterocyclic ring can be bound to a heteroatom via carbon atom. An oxo substituent on the heterocyclic ring means that one of the carbon atoms has a carbonyl group. A thio substituent on the heterocyclic ring means that one of the carbon atoms has a thiocarbonyl group.

When a compound of this invention with Formula 1 has a moiety that contains a heterocyclic ring, either mono, bicyclic, or tricyclic, such heterocyclic ring does not contain O—O, S—S, or S—O bonds in the ring.

Preferred bicyclic or tricyclic carbocyclic ring systems and bicyclic or tricyclic heterocyclic ring systems include naphthalene, 1,2,3,4-tetrahydronaphthalene, indane, 1-oxo-indane, 1,2,3,4-tetrahydroquinoline, naphthyridine, benzofuran, 3-oxo-1,3-dihydro-isobenzofuran, benzothiophene, 1,1-dioxo-benzothiophene, indole, 2,3-dihydroindole, 1,3-dioxo-2,3-dihydro-1H-isoindole, benzotriazole, 1H-indazole, indoline, benzopyrazole, naphthyridine, 1,3-benzodioxole, benzooxazole, purine, phthalimide, coumarin, chromone, quinoline, terahydroquinoline, isoquinoline, benzimidazole, quinazoline, pyrido[2,3-b]pyridine, pyrido[3,4-b]pyrazine, pyrido[3,2-c]pyridazine, pyrido[3,4-b]pyridine, 1H-pyrazole[3,4-d]pyrimidine, 1,4-benzodioxane, pteridine, 2(1H)-quinolone, 1(2H)-isoquinolone, 2-oxo-2,3-dihydro-benzthiazole, 1,2-methylenedioxybenzene, 2-oxindole, 1,4-benzisoxazine, benzothiazole, quinoxaline, quinoline-N-oxide, isoquinoline-N-oxide, quinoxaline-N-oxide, quinazoline-N-oxide, benzoxazine, phthalazine, 1,4-dioxo-1,2,3,4-tetrahydro-phthalazine, 2-oxo-1,2-dihydro-quinoline, 2,4-dioxo-1,4-dihydro-2H-benzo[d][1,3]oxazine, carbazole, fluorene, dibenzofuran, 2,5-dioxo-2,3,4,5-tetrahydro-1H-benzo[e][1,4]diazepine and cinnoline.

When Q or Q′ is a 3-8-membered heterocyclic ring, preferred heterocyclic rings include pyridine, pyrimidine, imidazole, thiazole, aziridine, azetidine thiazolidine, pyrrole, furan, thiophene, oxazole, 1,2,4-triazole, morpholine, thiomorpholine, piperidine, pyrrolidine, oxiran, 1,2,3-triazole, tetrazole, piperazine, tetrahydrothiophene, tetrahydrofuran, triazine, dioxane, 1,3-dioxolane and tetrahydropyran.

The formula for when Z is 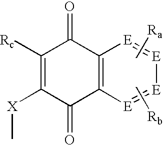
indicates that the right hand ring can be optionally substituted at any of the positions that are carbon atoms with Rₐ and R_{b} groups.

The alkyl portion of the alkyl, alkoxy, alkanoyloxy, alkylamido, alkoxymethyl, alkanoyloxymethyl, alkylsulphinyl, alkylsulphonyl, alkylsulfonamido, carboalkoxy, carboalkyl, carboxyalkyl, carboalkoxyalkyl, alkanoylamino, N-alkylcarbamoyl, N,N-dialkylcarbamoyl, N-alkylaminoalkoxy, N,N-dialkylaminoalkoxy, or where else it occurs in Formula 1, can include straight chain, cyclic, and branched carbon chains. The N,N-dialkylamino moiety includes cyclic amino radicals where the two alkyl groups form a saturated ring. The alkenyl portion of the alkenyl, alkenoyloxymethyl, alkenyloxy, and alkenylsulfonamido substituents include straight chain, cyclic, and branched carbon chains and one or more sites of unsaturation and all possible configurational isomers. The alkynyl portion of the alkynyl, alkynoyloxymethyl, alkynylsulfonamido and alkynyloxy substituents include both straight chain as well as branched carbon chains and one or more sites of unsaturation. Carboxy is defined as a —CO₂H radical. Carboalkoxy of 2-7 carbon atoms is defined as a —CO₂R″ radical, where R″ is an alkyl radical of 1-6 carbon atoms. Carboxyalkyl is defined as a HO₂C—R′″— radical where R′″ is a divalent alkyl radical of 1-6 carbon atoms. Carboalkoxyalkyl is defined as a R″O₂C—R′″— radical where R′″ is a divalent alkyl radical and where R″ and R′″ together have 2-7 carbon atoms. Carboalkyl is defined as a —COR″ radical, where R″ is an alkyl radical of 1-6 carbon atoms. Alkanoyloxy is defined as a —OCOR″ radical, where R″ is an alkyl radical of 1-6 carbon atoms. Alkanoyloxymethyl is defined as R″CO₂CH₂— radical, where R″ is an alkyl radical of 1-6 carbon atoms. Alkoxymethyl is defined as R″OCH₂— radical, where R″ is an alkyl radical of 1-6 carbon atoms. Alkylsulphinyl is defined as R″SO— radical, where R″ is an alkyl radical of 1-6 carbon atoms. Alkylsulphonyl is defined as R″SO₂— radical, where R″ is an alkyl radical of 1-6 carbon atoms. Alkylsulfonamido, alkenylsulfonamido and alkynylsulfonamido are defined as R″SO₂NH— radical, where R″ is an alkyl radical of 1-6 carbon atoms, an alkenyl radical of 2-6 carbon atoms or an alkynyl radical of 2-6 carbon atoms, respectively. N-alkylcarbamoyl is defined as R″NHCO— radical, where R″ is an alkyl radical of 1-6 carbon atoms. N,N-dialkylcarbamoyl is defined as R″R′NCO— radical, where R″ is an alkyl radical of 1-6 carbon atoms, R′ is an alkyl radical of 1-6 carbon atoms and R′ and R″ may be the same or different. It is preferred that of the substituents G₃ and G₄, at least one is hydrogen, and it is most preferred that both be hydrogen.

R₅ is a heterocycle, as defined above which may be optionally mono- or di-substituted on a carbon with R₆, optionally mono-substituted on nitrogen with R₆, optionally mono- or di-substituted on a carbon with hydroxy, —N(R₆)₂, or —OR₆, optionally mono or di-substituted on a carbon with —(C(R₆)₂)ₛOR₆ or —(C(R₆)₂)ₛN(R₆)₂ and optionally mono or di-substituted on a saturated carbon with divalent —O— or —O(C(R₆)₂)ₛO— (carbonyl and ketal groups, respectively). In some cases when R₅ is substituted with —O— (carbonyl), the carbonyl group can be hydrated. R₅ may be bonded to W when q=0 via a carbon atom on the heterocyclic ring, or when R₅ is a nitrogen containing heterocycle which also contains a saturated carbon-nitrogen bond. Such a heterocycle may be bonded to carbon, via the nitrogen when W is a bond. When q=0 and R₅ is a nitrogen containing heterocycle, which also contains an unsaturated carbon-nitrogen bond, that nitrogen atom of the heterocycle may be bonded to carbon when W is a bond and the resulting heterocycle will bear a positive charge. When R₅ is substituted with R₆, such substitution may be on a ring carbon, or in the case of a nitrogen containing heterocycle, which also contains a saturated carbon-nitrogen bond, such nitrogen may be substituted with R₆ or in the case of a nitrogen containing heterocycle, which also contains an unsaturated carbon-nitrogen bond, such nitrogen may be substituted with R₆. In such a case, the heterocycle will bear a positive charge.

The compounds of this invention may contain one or more asymmetric carbon atoms. In such cases, the compounds of this invention include the individual diasteromers, the racemates, and the individual R and S entantiomers thereof. Some of the compound of this invention may contain one or more double bonds. In such cases, the compounds of this invention include each of the possible configurational isomers as well as mixtures of these isomers. Some of the compounds of this invention may exist as separate tautomers. In such cases, the compounds of this invention include each tautomer and mixtures of these tautomers. When a compound of this invention contain a moiety containing the same substituent more than once (for example, when R₇ is —NR₆R₆), each substituent (R₆, in this example) may be the same or different. When the compounds of this invention contain a dialkylamino group (for example, when R₇ is —NR₆R₆), this dialkylamino group can also be a cyclic amino group (for example, for —NR₆R₆ the two R₆ groups are attached to each other to form a ring).

The compounds of this invention can be prepared from commercially available starting materials or starting materials that can be prepared using literature procedures. More specifically, the preparation of the compounds and intermediates of this invention encompassed by Formulas 3 and 5 is described below in Flowsheet 1 where R₁, G₁-G₄, X, Rₐ, R_{b}, and R_{c} are as described above. Oxidation of the dimethoxy derivatives having Formulas 2 or 6 with an oxidizing agent, such as ceric ammonium nitrate in aqueous acetonitrile, furnishes the quinone compounds 3 or 7, respectively. Alternatively, oxidation of the phenol derivative 4 with an oxidizing agent, such as Fremy's salt in the presence of base in a mixture of water and ethyl acetate (EtOAc), also furnishes compounds of this invention of formula 3. In those cases where both Rₐ and R_{b} are either hydrogen atoms or are bound to the quinone ring of 3 via carbon atoms, the molecule can be further oxidized to the quinone epoxide using hydrogen peroxide and a mixture of aqueous tetrahydrofurnan (THF) and acetonitrile in the presence of a weak base such as sodium bicarbonate. In those cases where the substituents such as G₁-G₄, X, Rₐ, R_{b}, and R_{c} are not stable to the oxidative reaction conditions, they can be protected using a suitable protecting group which can then be removed after the oxidation. The application of protecting groups is discussed in detail in Protective Groups in Organic Synthesis by T. W. Green and P. G. M. Wuts, John Wiley & Sons Inc., New York, 1991.

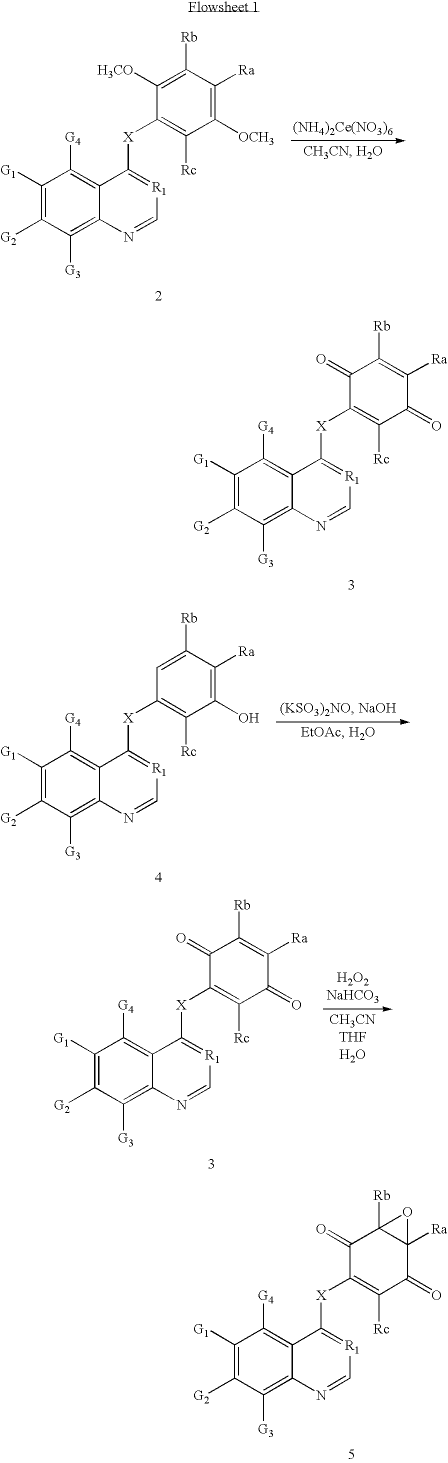

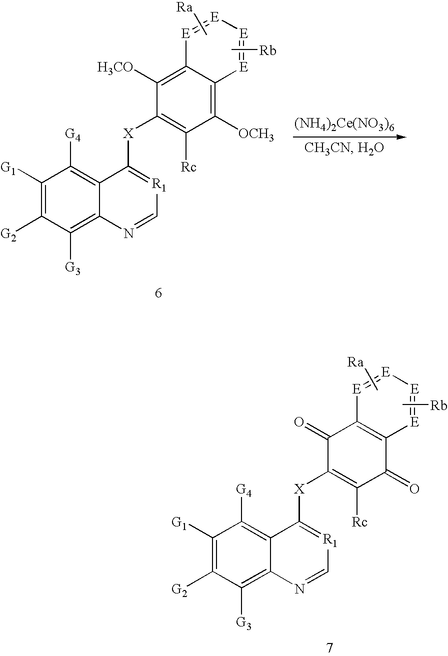

The starting materials represented by formulas 2, 4 and 6 and the intermediates needed to prepare these starting materials can be prepared using the methods outlined in the patent applications WO 00/18761, WO 00/18740, EP-93300270, WO 96/15118 and WO 96/09294, and U.S. Pat. No. 6,002,008 and the methods described below.

The intermediates represented by formulas 15-17, necessary for the preparation of some of the compounds of this invention, are prepared as shown below in Flowsheet 2, where E, G₁-G₄, Rₐ, R_{b}, and R_{c} are as described above. A substituted benzonitrile derivative 8 is nitrated using ammonium nitrate in a mixture of trifluoroacetic anhydride and chloroform. Nitration with nitric acid can also be used for this reaction. If the nitration of 8 results in isomers, the desired isomer can be separated by chromatography or fractional recrystallization. The nitro group of compound 9 is reduced by catalytic hydrogenation using a palladium catalyst and hydrogen gas or cyclohexene as the hydrogen source. The aniline 10 is heated with an excess of neat dimethylformamide-dimethylacetal to give the amidine 11. Refluxing 11 with the anilines 12-14 in acetic acid gives the intermediates 15-17, respectively.

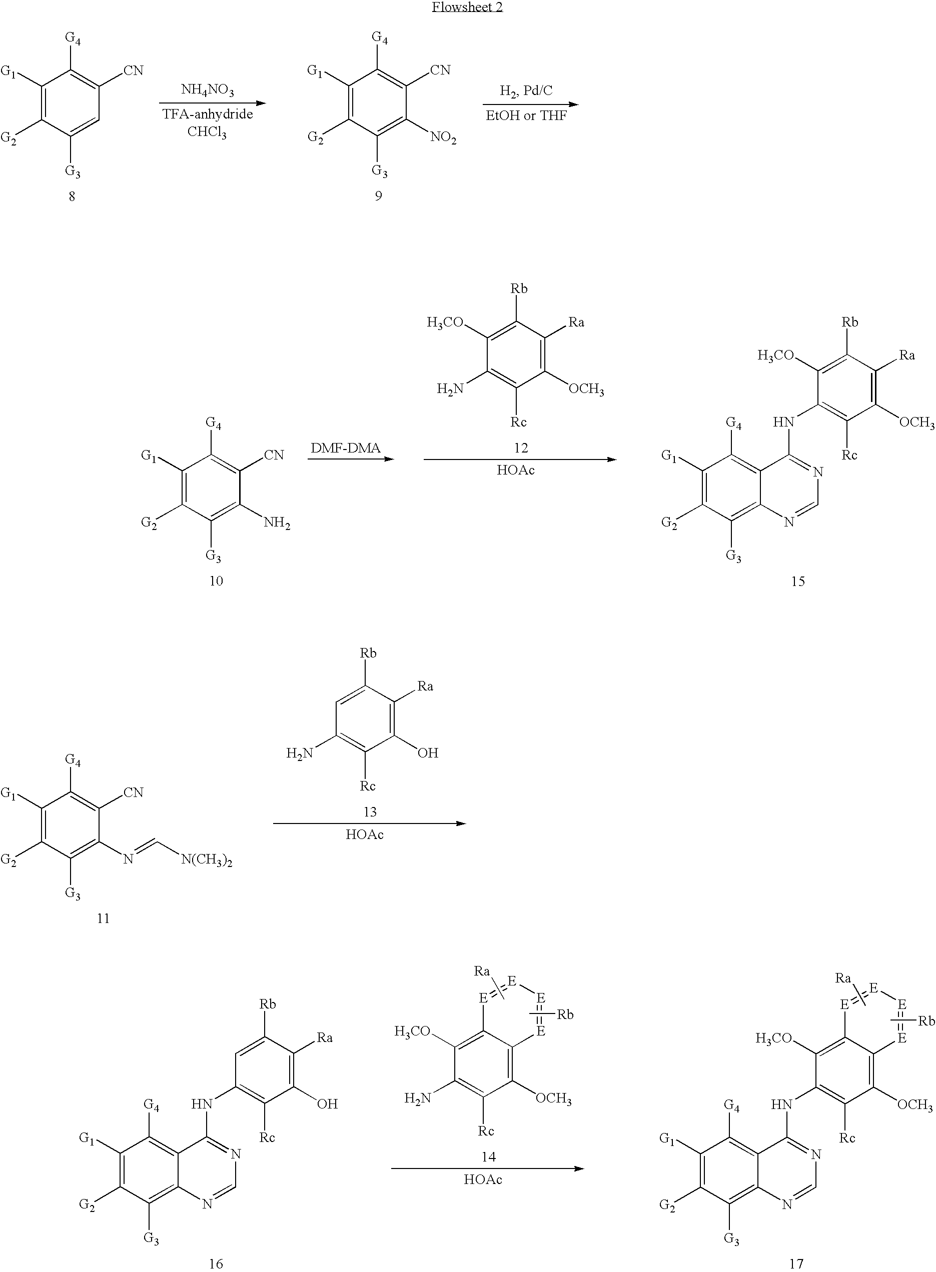

Alternatively, these intermediates can be prepared from 4-chloroquinazoline derivatives as shown below in Flowsheet 3 where E, R₁₀, G₁-G₄, X, Rₐ, R_{b}, and R_{c} are as described above. The ester 18 or the corresponding ethyl ester is nitrated using ammonium nitrate in a mixture of trifluoroacetic anhydride and chloroform. Nitration with nitric acid can also be used for this reaction. If the nitration of compound 18 results in isomers, the desired isomer can be separated by chromatography or fractional recrystallization. Catalytic hydrogenation of compound 19 gives compound 20. This reduction can also be accomplished using metals such as iron powder in refluxing ammonium chloride solution in methanol. Heating 20 with formamidine acetate, either neat or in a solvent such as isopropanol, gives the hydroxyquinazoline 21. Alternatively, reduction of 9 (from Flowsheet 2) with zinc in a mixture of refluxing acetic acid and methanol results in the reduction of the nitro group and hydrolysis of the nitrile group giving compound 22. This compound is then reacted with triethylorthoformate at reflux to give compound 21. In the next step, 21 is chlorinated by refluxing in either phosphorous oxychloride or thionyl chloride and catalytic dimethylformamide resulting in compound 23. In those cases where compounds 24 and 25 are anilines (X═NH or NR₁₀), heating these with 23 in an inert solvent such as isopropanol or ethoxyethanol results in compounds 26 and 27 (X═NH or NR₁₀), respectively. If needed, this reaction can be catalyzed using a small amount of pyridine hydrochloride. In those cases where 24 and 25 are phenols or thiophenols (X═O or S), they can be reacted with 23 using a base, such as sodium hydride, and an inert solvent, such as tetrahydrofuran, toluene, or dimethylformamide, to give 26 and 27 (X═O or S), respectively. If necessary, the reaction mixture can be heated up to the reflux temperature of the solvent.

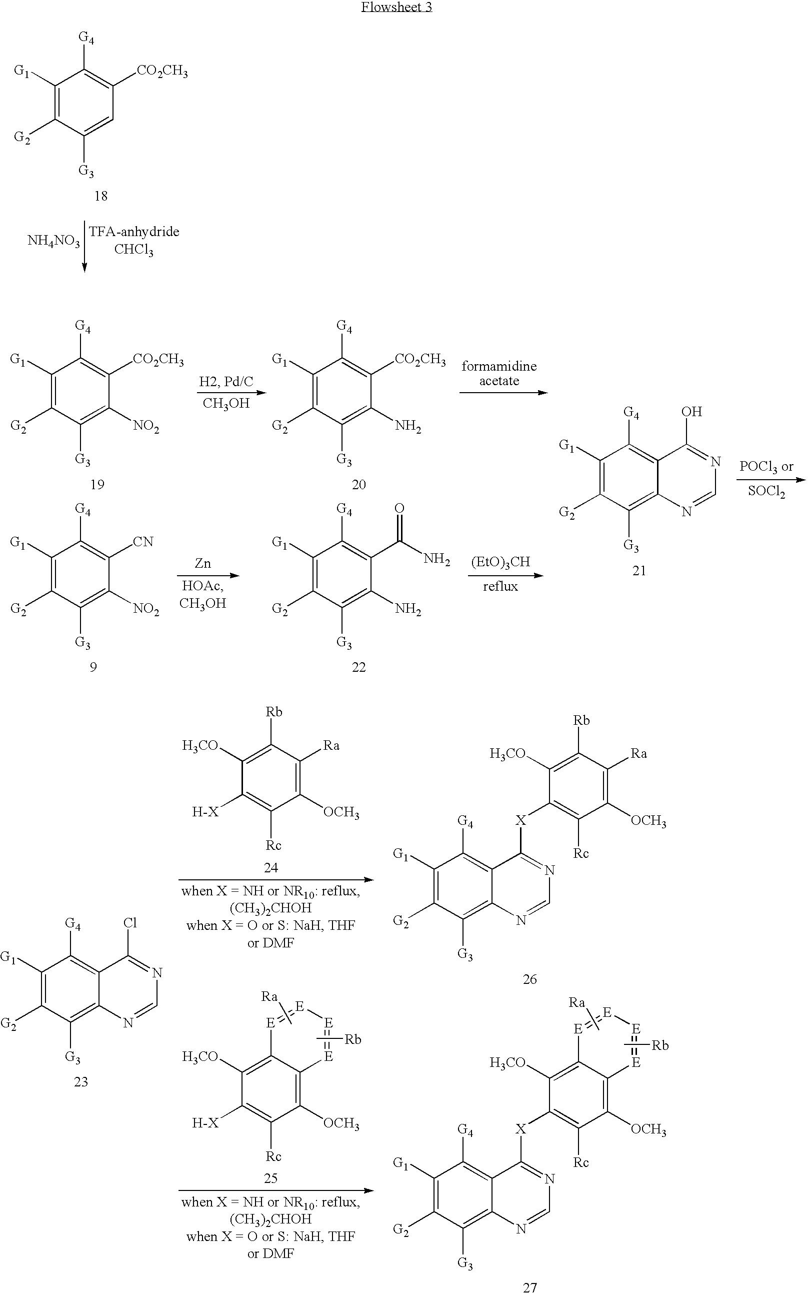

Intermediates needed to prepare the compounds of this invention that are 3-cyanoquinolines are prepared as shown in Flowsheet 4 where E, R₁₀, G₁-G₄, X, Rₐ, R_{b}, and R_{c} are as described above. The methods used to prepare the starting 4-chloro-3-cyanoquinolines represented by formula 28 are described in detail in international patent applications WO 98/43960, WO 00/18761 and WO 00/18740. In those cases where 24 and 25 are anilines (X═NH or NR₁₀), heating these with 28 in an inert solvent, such as isopropanol or ethoxyethanol, results in compounds 29 and 30 (X═NH or NR₁₀), respectively. If needed, this reaction can be catalyzed using a small amount of pyridine hydrochloride. In those cases where 24 and 25 are phenols or thiophenols (X═O or S), they can be reacted with 28 using a base, such as sodium hydride, and an inert solvent, such as tetrahydrofuran, toluene or dimethylformamide, to give 29 and 30 (X═O or S), respectively. If necessary, the reaction mixture can be heated up to the reflux temperature of the solvent.

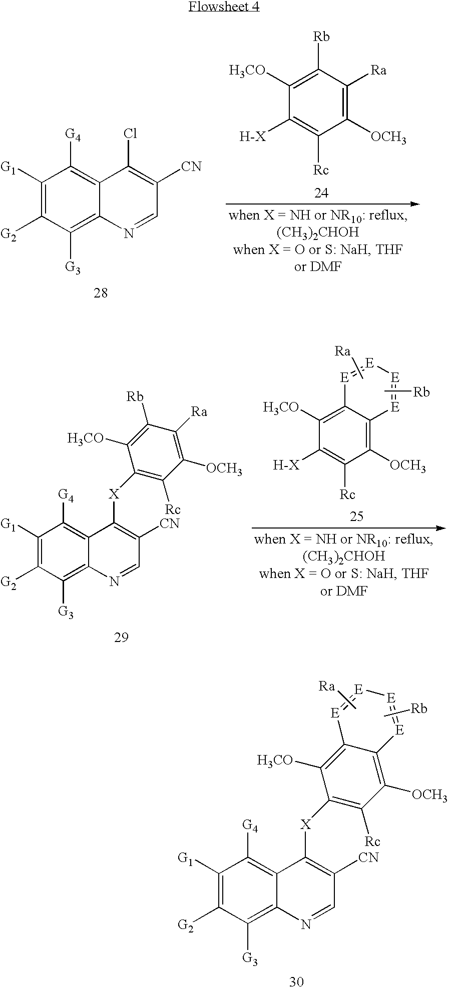

Certain compounds of this invention can be used as intermediates for the preparation of other compounds of this invention as shown below in Flowsheet 5 where R₁ and G₁-G₄ are as defined above. HO-Q, is H-L-Q or H-L-Q-L′-Q′ as defined above with L being restricted to —O—, —O—(CH₂)ₙ—, and —O—(CH₂)ₙ—X—. NH₂-Q₂ is H-L-Q or H-L-Q-L′-Q′ with L being restricted to —NH—, —NH—(CH₂)ₙ—, and —NH—(CH₂)ₙ—X—. NHR₁₀-Q₃ is H-L-Q or H-L-Q-L′-Q′ with L being restricted to —NR₁₀—, —NR₁₀—(CH₂)ₙ—, and —NR₁₀—(CH₂)ₙ—X—. HS-Q₄ is H-L-Q or H-L-Q-L′-Q′ with L being restricted to —S—, —S—(CH₂)ₙ—, and —S—(CH₂)ₙ—X—. Q5 is -Q or -Q-L′-Q′ as defined above where Q is a bicyclic, tricyclic heteroaryl, or heteroaryl moiety that has, as the reactive center, a —NH— as part of the heterocyclic ring.

The reaction of the chloroquinone 31 with a substituted phenol or heteroaryl moiety that contains an attached OH group in the presence of base and an inert solvent, such as methylene chloride, DMF or THF, results in displacement of the chlorine atom to give compound 33. Sometimes it is beneficial to do the displacement in the presence of a phase transfer catalyst, such as tricaprylylmethylammonium chloride. When the moiety HO-Q, is an alcohol, the reaction of the phenoxy substituted quinone 32 with an excess of this alcohol in an inert solvent such as methylene chloride in the presence of a base such as triethylamine also furnishes the compound of formula 33. This reaction proceeds at room temperature or at reflux.

The reaction of NH₂-Q₂ or NHR₁₀-Q₃ with 31 or 32 in an inert solvent such as glyme, DMF or THF results in the compounds 34 and 35, respectively. This reaction proceeds at room temperature or at reflux.

The reaction of HS-Q₄ with 31 or 32 in an inert solvent such as methylene chloride or THF results in the compound 36. This reaction proceeds at room temperature or at reflux. The reaction can sometimes be accelerated using base catalyst such as triethylamine. Due to quinone reduction, side products, in addition to 36, sometimes result in this reaction. These side products can be removed by chromatography.

The reaction Q₅ with 31 or 32 in an inert solvent such as glyme, DMF or THF results in the compound 37 where the nitrogen atom of Q₅ is bonded directly to the quinone ring. This reaction proceeds at room temperature or at reflux. Sometimes a base will accelerate this reaction.

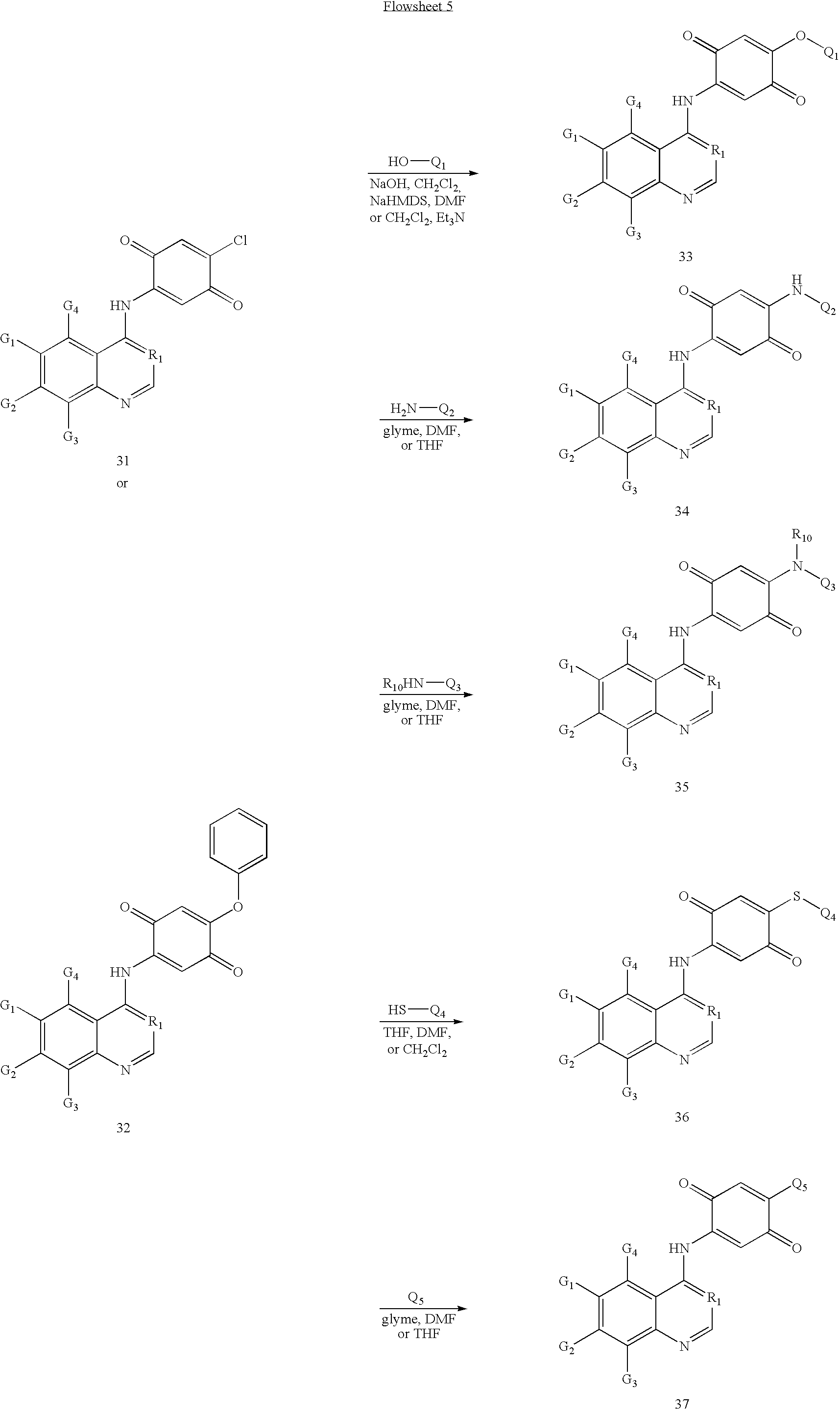

Other compounds of this invention can be used to make additional compounds of this invention as shown below in Flowsheet 6 where R₁, G₁-G₄, —S-Q₄, and —O-Q, are as described above. The reaction of a sulfhydryl species such as HS-Q₄ with quinone 38 in an inert solvent, such as methylene chloride or THF, results in reductive addition to give the hydroquinone 39. This compound can then be oxidized to the quinone 40 using an oxidizing agent such as 2,3-dichloro-5,6-dicyano-1,4-benzoquinone (DDQ). The reaction of a sulfhydryl species, such as HS-Q₄, with quinone 41 in an inert solvent, such as methylene chloride or THF, results in reductive addition to give the hydroquinone 42. This compound can then be oxidized to the quinone 43 using an oxidizing agent, such as 2,3-dichloro-5,6-dicyano-1,4-benzoquinone (DDQ).

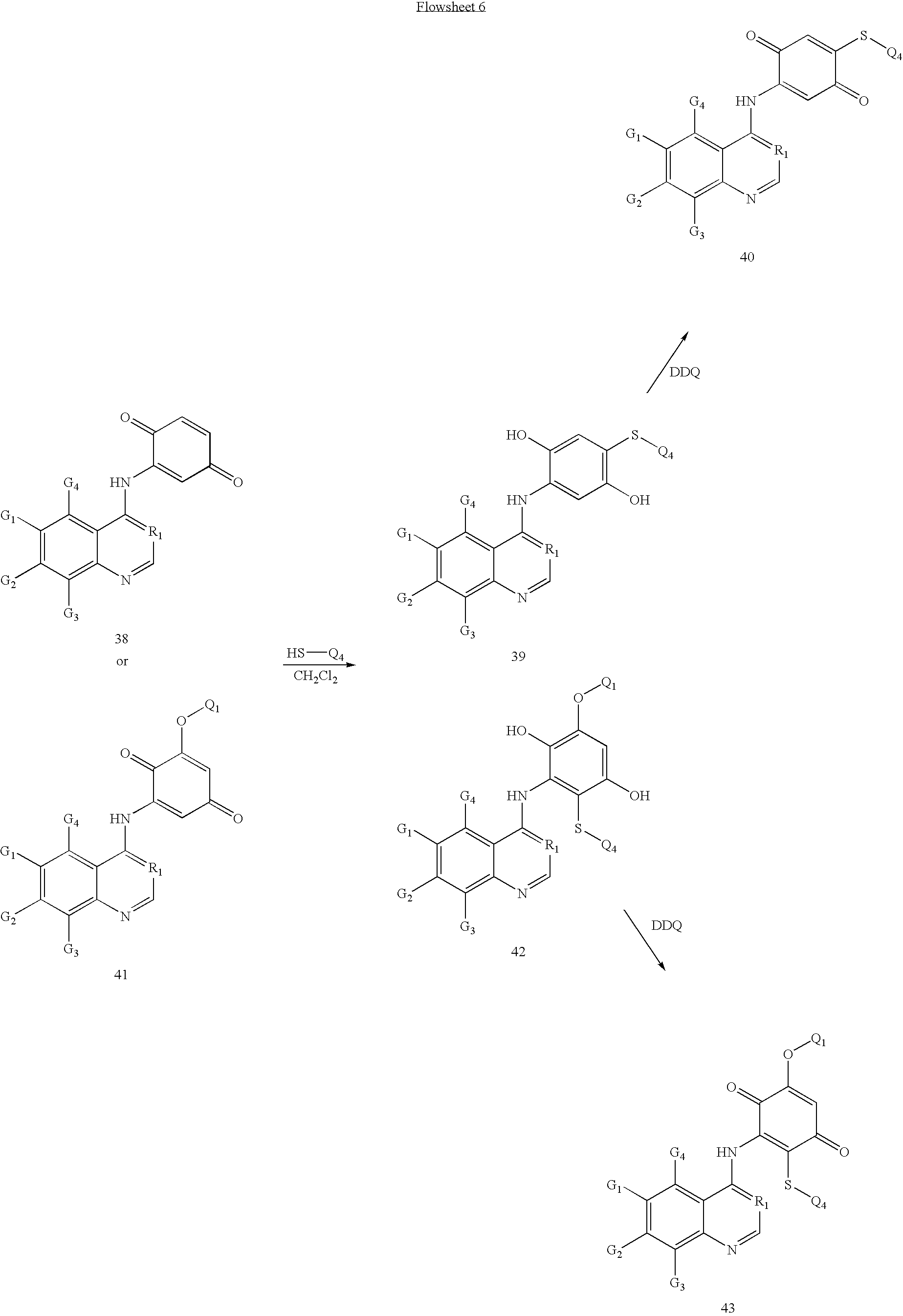

Additional compounds of this invention are prepared as shown below in Flowsheet 7 where R₁ and G₁-G₄ are as defined above. HO-Q, is H-L-Q or H-L-Q-L′-Q′ as defined above with L being restricted to —O—, —O—(CH₂)ₙ—, and —O—(CH₂)ₙ—X—. NH₂-Q₂ is H-L-Q or H-L-Q-L′-Q′ with L being restricted to —NH—, —NH—(CH₂)ₙ—, and —NH—(CH₂)ₙ—X—. NHR₁₀-Q₃ is H-L-Q or H-L-Q-L′-Q′ with L being restricted to —NR₁₀—, —NR₁₀—(CH₂)ₙ—, and —NR₁₀—(CH₂)ₙ—X—. HS-Q₄ is H-L-Q or H-L-Q-L′-Q′ with L being restricted to —S—, —S—(CH₂)ₙ—, and —S—(CH₂)ₙ—X—. Q₅ is -Q or -Q-L′-Q′ as defined above where Q is a bicyclic, tricyclic heteroaryl, or heteroaryl moiety that has as the reactive center, a —NH— as part of the heterocyclic ring.

Addition of hydrogen chloride to compound 44 in chloroform at room temperature affords the hydroquinone 45. Oxidation of 45 to the quinone 46 is accomplished using an oxidizing agent, such as DDQ, in an inert solvent, such as chloroform, acetonitrile or methylene chloride.

The reaction of the chloroquinone 46 with a substituted phenol or heteroaryl moiety that contains an attached OH group in the presence of base and an inert solvent, such as methylene chloride, DMF or THF, results in displacement of the chlorine atom to give compound 47. Sometimes it is beneficial to do the displacement in the presence of a phase transfer catalyst such as tricaprylylmethylammonium chloride. This reaction proceeds at room temperature or at reflux.

The reaction of NH₂-Q₂ or NHR₁₀-Q₃ with 46 in an inert solvent, such as glyme or THF, results in the compounds 48 and 49, respectively. This reaction proceeds at room temperature or at reflux. Sometimes it is beneficial to do this reaction using a base such as potassium carbonate or triethylamine.

The reaction of HS-Q₄ with 46 in an inert solvent, such as methylene chloride or THF, results in the compound 50. This reaction proceeds at room temperature or at reflux. The reaction can sometimes be accelerated using base catalyst such as triethylamine. Due to quinone reduction, side products, in addition to 50, sometimes result in this reaction. These side products can be removed by chromatography.

The reaction Q₅ with 46 in an inert solvent, such as glyme, methylene chloride, acetonitrile or THF, results in the compound 51, where the nitrogen atom of Q₅ is bonded directly to the quinone ring. This reaction proceeds at room temperature or at reflux. Sometimes a base will accelerate this reaction.

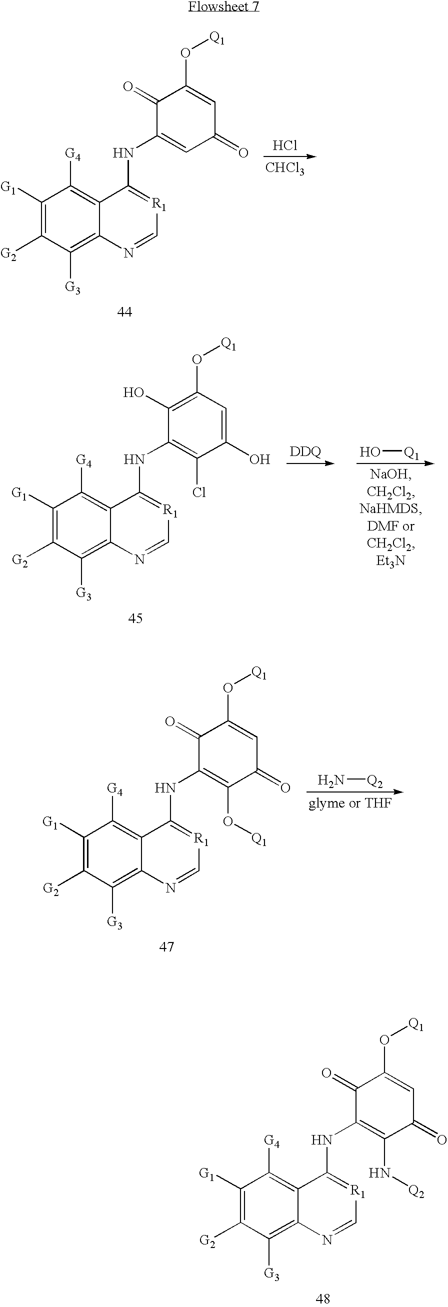

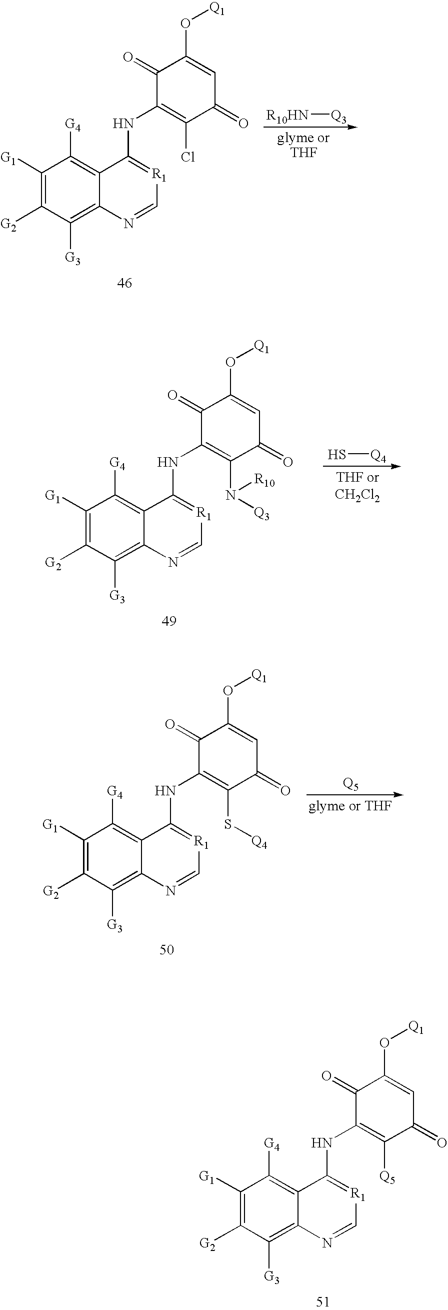

There are certain functional group manipulations that are useful to prepare the compounds of this invention that can be applied to various intermediate quinazoline or quinolines, such as compounds with Formulas 2, 4, and 6. These manipulations refer to the substituents G₁-G₄, which are located on the formulas shown in the above Flowsheets. Some of these functional group manipulations are described below.

Where one or more of G₁-G₄ is a nitro group, it can be converted to the corresponding amino group by reduction using a reducing agent such as iron in acetic acid, or by catalytic hydrogenation.

Where one or more of G₁-G₄ is an amino group, it can be converted to the corresponding dialkyamino group of 2-12 carbon atoms by alkylation with at least two equivalents of an alkyl halide of 1-6 carbon atoms by heating in an inert solvent or by reductive alkylation using an aldehyde of 1-6 carbon atoms and a reducing agent such as sodium cyanoborohydride.

Alternatively, where one or more of G₁-G₄ is an amino group, it can be converted to the corresponding alkylsulfonamido, alkenylsulfonamido or alkynylsulfonamido group of 2-6 carbon atoms by the reaction with an alkylsulfonyl chloride, alkenylsulfonyl chloride or alkynylsulfonyl chloride, respectively, in an inert solvent using a basic catalyst, such as triethylamine or pyridine.

Alternatively, where one or more of G₁-G₄ is an amino group, it can be converted to the corresponding alkyamino group of 1-6 carbon atoms by alkylation with one equivalent of an alkyl halide of 1-6 carbon atoms by heating in an inert solvent or by reductive alkylation using an aldehyde of 1-6 carbon atoms and a reducing agent such as sodium cyanoborohydride, in a protic solvent such as water or alcohol, or mixtures thereof.

Where one or more of G₁-G₄ is hydroxy, it can be converted to the corresponding alkanoyloxy group of 1-6 carbon atoms by reaction with an appropriate carboxylic acid chloride, anhydride, or mixed anhydride in a inert solvent using pyridine or a trialkylamine as a catalyst.

Alternatively, where one or more of G₁-G₄ is hydroxy, it can be converted to the corresponding alkenoyloxy group of 1-6 carbon atoms by reaction with an appropriate carboxylic acid chloride, anhydride or mixed anhydride in an inert solvent using pyridine or a trialkylamine as a catalyst.

Alternatively, where one or more of G₁-G₄ is hydroxy, it can be converted to the corresponding alkynoyloxy group of 1-6 carbon atoms by reaction with an appropriate carboxylic acid chloride, anhydride or mixed anhydride in a inert solvent using pyridine or a trialkylamine as a catalyst.

Where one or more of G₁-G₄ is carboxy or a carboalkoxy group of 2-7 carbon atoms, it can be converted to the corresponding hydroxymethyl group by reduction with an appropriate reducing agent, such as borane, lithium borohydride or lithium aluminum hydride in a inert solvent. The hydroxymethyl group, in turn, can be converted to the corresponding halomethyl group by reaction in an inert solvent with a halogenating reagent, such as phosphorous tribromide to give a bromomethyl group, or phosphorous pentachloride to give a chloromethyl group. The hydroxymethyl group can be acylated with an appropriate acid chloride, anhydride, or mixed anhydride in an inert solvent using pyridine or a trialkylamine as a catalyst to give the compounds of this invention with the corresponding alkanoyloxymethyl group of 2-7 carbon atoms, alkenoyloxymethyl group of 2-7 carbon atoms or alkynoyloxymethyl group of 2-7 carbon atoms.

Where one or more G₁-G₄ is a halomethyl group, it can be converted to an alkoxymethyl group of 2-7 carbon atoms by displacing the halogen atom with a sodium alkoxide in an inert solvent.

Alternatively, where one or more of G₁-G₄ is a halomethyl group, it can be converted to an aminomethyl group, N-alkylaminomethyl group of 2-7 carbon atoms or N,N-dialkylaminomethyl group of 3-14 carbon atoms by displacing the halogen atom with ammonia, a primary, or secondary amine, respectively, in an inert solvent.

In addition to the methods described herein above, there are a number of patent applications that describe methods that are useful for the preparation of the intermediates used to prepare compounds of this invention. The chemical procedures described in the application WO 96/33981 can be used to prepare the intermediates used in this invention wherein G₁-G₄ are alkoxyalkylamino groups. The chemical procedures described in the application WO 96/33980 can be used to prepare the intermediates used in this invention wherein G₁-G₄ are aminoalkylalkoxy groups. The chemical procedures described in the application WO 96/33979 can be used to prepare the intermediates used in this invention wherein G₁-G₄ are alkoxyalkylamino groups. The chemical procedures described in the application WO 96/33978 can be used to prepare the intermediates used in this invention wherein G₁-G₄ are aminoalkylamino groups. The chemical procedures described in the application WO 96/33977 can be used to prepare the 3-cyanoquinoline intermediates used in this invention wherein G₁-G₄ are aminoalkylalkoxy groups. Although these applications describe methods for the preparation of certain quinazolines, they are also applicable to the preparation of corresponding substituted quinolines and although they also describe compounds where the indicated functional group have been introduced onto the 6-position of a quinazoline, the same chemistry can be used to introduce the same groups onto positions occupied by any of the G₁-G₄ substituents of the compounds of this invention, represented by compounds with Formulas 2, 4, 6, 15-17, 26, 27, 29, and 30.

Methods described in the following publications can also be used to prepare intermediates that are used to prepare the compounds of this invention: Hennequin et al., J. Med. Chem. 42:5369-5389 (1999); Hennequin et al., J. Med. Chem. 45:1300-1312 (2002); Wissner et al., J. Med. Chem. 46:49-63 (2003); Wissner et al., J. Med. Chem., 43:3244-3256 (2002); and Wissner et al., Bioorg. Med. Chem. Lett. 12:2893-2897 (2002).

Representative compounds of this invention were evaluated in several standard pharmacological test procedures that showed that the compounds of this invention possess significant activity as inhibitors of certain tyrosine kinases and function as anti-angiogenic agents. Some of these test procedures are described in patent application, serial no. (TO BE ASSIGNED), entitled “ASSAYS TO IDENTIFY IRREVERSIBLY BINDING INHIBITORS OF RECEPTOR TYROSINE KINASES”, by inventors Frank Loganzo, Lee M. Greenberger, Xingzhi Tan and Allan Wissner, filed concurrently herewith.

Based on the activity shown in the standard pharmacological test procedures, the compounds of this invention are therefore useful as antineoplastic agents and as agents for the treatment of other disease states characterized by abnormal, excessive, or otherwise inappropriate blood vessel growth. The test procedures used and results obtained are shown below.

### Inhibition of KDR Kinase

Expression of Recombinant KDR Enzyme

The full cytoplasmic domain of human KDR (VEGF-receptor-2) was cloned by standard reverse transcription/polymerase chain reaction (RT-PCR) using total RNA isolated from human umbilical vein endothelial cells (HUVEC). Total RNA was isolated with the RNAgents Total Isolation System (Promega) and cDNA generated by RT-PCR (SuperScript II RnaseH⁻ Reverse Transcriptase and Platinum Pfx DNA Polymerase, Invitrogen) using primers specific for KDR (GenBank accession NM_{—}002253) beginning at Met-806 [underlined] (5′-ATG GAT CCA GAT GM CTC CCA TTG) and ending at Val-1356 [underlined] (5′-AAC AGG AGG AGA GCT CAG TGT GGT). Primers were designed with HindIII/XhoI terminal sites, respectively, to allow for subcloning. The cDNA product was cloned in-frame into the pCMV-Tag4 vector (Stratagene) at the HindIII/XhoI sites such that a FLAG sequence (DYKDDDDK) is expressed at the C-terminus to allow for protein purification.

Human embryonic kidney (HEK) 293 cells (American Type Culture Collection) were transiently transfected with the KDR-Flag vector and cells were harvested 48 hour post-transfection to confirm protein expression. Stable clones were then selected in geneticin G₄₁₈ (500 ug/ml) for approximately 3 weeks and used for moderate-scale protein preparations.

Cells (36×150 mm dishes of sub-confluent monolayers) were lysed in 72 ml of lysis buffer containing protease inhibitors (50 mM HEPES, 150 mM NaCl, 2 mM EDTA, 1% Igepal CA-630, pH 7.5, 1 mM Na₃VO₄, 1 mM PMSF, 20 KIU/ml aprotinin, 10 ug/ml pepstatin, 10 ug/ml leupeptin) and then centrifuged at 12,000 rpm for 20 minutes at 4° C. to remove insoluble debris.

KDR protein was isolated from cell lysate by batch purification on anti-FLAG M2 affinity resin (Sigma) for 2 hour at 4° C. followed by sequential washing and centrifugation. Resin was applied to a column and protein eluted with 200 ug/ml FLAG peptide in 50 mM HEPES, 100 mM NaCl, 10% glycerol, 1 mM Na₃VO₄, 1 mM EDTA. Fractions were collected and evaluated for KDR content by SDS-PAGE/immunoblot analyses using anti-KDR antibody (Dougher, M. and Terman, B. I., Oncogene 18: 1619-1627 (1999)) or anti-FLAG M2 antibody (Sigma). KDR purity is typically 20-40%. Bovine serum albumin was added to a final concentration of 1 mg/ml and glycerol is added to 50% (v/v). Small-volume aliquots are stored at −70° C.

The recombinant cytoplasmic (intracellular) protein product is designated KDR-IC-Flag.

KDR Kinase Enzyme Assay

The kinase activity of KDR-IC-Flag was evaluated using a DELFIA® (dissociation-enhanced lanthanide fluorescent immunoassay) (PerkinElmer Life Sciences, Boston) as described by PerkinElmer and Loganzo, F. and Hardy, C. American Biotechnology Laboratory 16:26-28 (1998).

Nunc Maxisorb 96-well plates were coated at room temperature for 1 to 2 hours with 100 μl per well of 25 μg/ml poly(Glu₄-Tyr) peptide (Sigma) in tris-buffered saline (TBS) (25 mM Tris pH 7.2, 150 mM NaCl). Unbound peptide was washed three times with TBS.

KDR-IC-Flag enzyme was diluted (depending on the batch, from 10- to 20-fold) in 0.1% BSA/4 mM HEPES. A master mix of enzyme plus kinase buffer was prepared: (per well) 10 μl of diluted enzyme, 10 μl of 5× kinase buffer (5×=20 mM HEPES, pH 7.4, 5 mM MnCl₂, 100 uM Na₃VO₄), and 911 of water. Master mix (29 μl) was added to each well and compounds (1 μl) prepared in 100% dimethyl sulfoxide (DMSO) were added to appropriate wells. Test compounds were added as 50× stocks as necessary for single point or dose-response analyses. Controls were done by adding DMSO alone, i.e., no test compound, to wells containing the master mix of enzyme plus kinase buffer.

After 15 minutes at room temperature, ATP/MgCl₂ (20 μl of 25 μM ATP, 25 mM MgCl₂, 10 mM HEPES, pH 7.4) was added to each well to initiate the reaction. Final concentrations of the assay components were: 10 μM ATP, 10 mM MgCl₂, 1 mM MnCl₂, 4 mM HEPES, pH 7.4, 20 μM Na₃VO₄, 20 ug/ml BSA, 2% DMSO.

After 40 minutes at room temperature, the liquid was removed and plates were washed three times with TBST (TBS with 0.05% Tween-20). The wells were then incubated for 1 hour at room temperature with 75 μl of approximately 0.1 ug/ml europium-conjugated anti-phosphotyrosine antibody (PT66; PerkinElmer) prepared in Assay Buffer (PerkinElmer). Plates were washed three times with TBST, then incubated for 15 minutes in the dark with 100 μl of Enhancement Solution (PerkinElmer). Plates were read in a Victor-V multi-label counter (PerkinElmer) using the default europium detection protocol. Percent inhibition or IC₅₀ of compounds were calculated by comparison with DMSO-treated control wells. The results are shown in Table 1.

When multiple entries for an inhibitor appear in Table 1, it indicates that the inhibitor was evaluated multiple times using the conditions stated in the table. The data in Table 1 shows that the compounds of this invention are effective inhibitors of KDR kinase and are therefore useful for the treatment of the aforementioned disease states.

TABLE 1
Inhibition of KDR Kinase

| | IC50 | Concentration | | ATP conc. |
|---|---|---|---|---|
| Example | (nM) | (nM) | % Inhibition | (μM) |
| 2 | | 100 | 83 | 10 |
| 2 | | 100 | 77 | 10 |
| 2 | | 1000 | 96 | 10 |
| 2 | | 1000 | 98 | 1000 |
| 2 | 5.1 | | | 10 |
| 4 | | 100 | 3 | 10 |
| 4 | | 100 | 13 | 10 |
| 4 | 80.5 | | | 10 |
| 4 | 285.2 | | | 10 |
| 4 | | 1000 | 15 | 10 |
| 4 | | 1000 | 12 | 1000 |
| 4 | 706.5 | | | 10 |
| 5 | | 100 | 18 | 10 |
| 5 | | 100 | 16 | 10 |
| 5 | | 1000 | 49 | 10 |
| 5 | | 1000 | 60 | 1000 |
| 7 | | 100 | 85 | 10 |
| 7 | | 100 | 83 | 10 |
| 7 | 2.3 | | | 10 |
| 7 | | 1000 | 96 | 10 |
| 7 | | 100 | 94 | 10 |
| 7 | | 1000 | 94 | 10 |
| 7 | | 10000 | 96 | 10 |
| 7 | | 100 | 96 | 10 |
| 7 | | 1000 | 96 | 10 |
| 7 | | 10000 | 97 | 10 |
| 7 | | 100 | 97 | 10 |
| 7 | | 100 | 96 | 10 |
| 7 | | 1000 | 96 | 10 |
| 7 | | 1000 | 95 | 10 |
| 7 | | 1000 | 97 | 1000 |
| 7 | | 1000 | 97 | 1000 |
| 7 | | 1000 | 97 | 1000 |
| 7 | 1.3 | | | 10 |
| 8 | | 1000 | 82 | 10 |
| 8 | | 100 | 51 | 10 |
| 8 | | 100 | 45 | 10 |
| 8 | | 1000 | 85 | 10 |
| 8 | 175.1 | | | 10 |
| 8 | 199.6 | | | 100 |
| 8 | 238.8 | | | 1000 |
| 8 | | 1000 | 80 | 1000 |
| 8 | | 1000 | 76 | 1000 |
| 8 | 176.7 | | | 10 |
| 9 | | 1000 | 58 | 10 |
| 9 | | 100 | 24 | 10 |
| 9 | | 1000 | 33 | 1000 |
| 15 | | 100 | 32 | 10 |
| 15 | | 100 | 40 | 10 |
| 15 | 197 | | | 10 |
| 15 | 157.2 | | | 10 |
| 15 | 154.2 | | | 10 |
| 15 | | 1000 | 63 | 10 |
| 15 | | 100 | 54 | 10 |
| 15 | | 1000 | 78 | 10 |
| 15 | 176.6 | | | 10 |
| 15 | 321.4 | | | 100 |
| 15 | 681.3 | | | 1000 |
| 15 | 251.4 | | | 1 |
| 15 | 372.8 | | | 10 |
| 15 | 789.2 | | | 100 |
| 15 | >1000 | | | |
| 15 | | 1000 | 42 | 1000 |
| 15 | | 1000 | 54 | 1000 |
| 15 | | 1000 | 53 | 1000 |
| 15 | | 100 | 43 | 10 |
| 15 | | 1000 | 70 | 10 |
| 17 | | 100 | 96 | 10 |
| 17 | | 1000 | 95 | 10 |
| 17 | | 1000 | 96 | 1000 |
| 18 | | 100 | 13 | 10 |
| 18 | | 1000 | 2 | 10 |
| 18 | | 1000 | −30 | 1000 |
| 19 | | 100 | 41 | 10 |
| 19 | | 1000 | 88 | 10 |
| 19 | | 1000 | 88 | 1000 |
| 20 | 17.9 | | | 10 |
| 20 | | 1000 | 90 | 10 |
| 20 | | 100 | 92 | 10 |
| 20 | | 100 | 80 | 10 |
| 20 | | 1000 | 94 | 10 |
| 20 | | 1000 | 97 | 1000 |
| 20 | | 1000 | 95 | 1000 |
| 22 | | 100 | 97 | 10 |
| 22 | | 1000 | 96 | 10 |
| 22 | | 1000 | 98 | 1000 |
| 24 | | 100 | 96 | 10 |
| 24 | | 1000 | 92 | 10 |
| 24 | | 1000 | 96 | 1000 |
| 25 | | 1000 | 95 | 10 |
| 25 | | 100 | 94 | 10 |
| 25 | | 1000 | 99 | 1000 |
| 25 | 4.8 | | | 10 |
| 26 | | 100 | 83 | 10 |
| 26 | | 100 | 81 | 10 |
| 26 | | 100 | 76 | 10 |
| 26 | | 100 | 70 | 10 |
| 26 | | 100 | 81 | 10 |
| 26 | | 100 | 80 | 10 |
| 26 | | 100 | 82 | 10 |
| 26 | | 100 | 83 | 10 |
| 26 | 30 | | | 10 |
| 26 | 10 | | | 10 |
| 26 | 10 | | | 10 |
| 26 | 11.1 | | | 10 |
| 26 | 12 | | | 10 |
| 26 | 8.1 | | | 10 |
| 26 | | 1000 | 96 | 10 |
| 26 | | 1000 | 95 | 10 |
| 26 | | 1000 | 96 | 10 |
| 26 | | 1000 | 95 | 10 |
| 26 | | 1000 | 93 | 10 |
| 26 | | 1000 | 94 | 10 |
| 26 | | 1000 | 93 | 10 |
| 26 | | 100 | 92 | 10 |
| 26 | | 100 | 94 | 10 |
| 26 | | 100 | 91 | 10 |
| 26 | | 100 | 93 | 10 |
| 26 | | 1000 | 95 | 10 |
| 26 | | 10000 | 96 | 10 |
| 26 | | 100 | 96 | 10 |
| 26 | | 1000 | 96 | 10 |
| 26 | | 10000 | 96 | 10 |
| 26 | 2.3 | | | 10 |
| 26 | | 100 | 96 | 10 |
| 26 | | 100 | 95 | 10 |
| 26 | | 100 | 97 | 10 |
| 26 | | 1000 | 95 | 10 |
| 26 | | 1000 | 95 | 10 |
| 26 | | 1000 | 95 | 10 |
| 26 | 5.1 | | | 10 |
| 26 | 5.7 | | | 100 |
| 26 | 5.2 | | | 1000 |
| 26 | 12.7 | | | 1 |
| 26 | 7.4 | | | 10 |
| 26 | 6 | | | 100 |
| 26 | 6.3 | | | 1000 |
| 26 | 12.7 | | | 1 |
| 26 | 7.4 | | | 10 |
| 26 | 6 | | | 100 |
| 26 | 6.3 | | | 1000 |
| 26 | | 1000 | 97 | 1000 |
| 26 | | 1000 | 97 | 1000 |
| 26 | | 1000 | 96 | 1000 |
| 26 | | 1000 | 96 | 1000 |
| 26 | | 1000 | 97 | 1000 |
| 26 | | 1000 | 97 | 1000 |
| 26 | | 1000 | 98 | 1000 |
| 26 | | 1000 | 97 | 1000 |
| 26 | | 1000 | 97 | 1000 |
| 26 | | 1000 | 97 | 1000 |
| 26 | | 100 | 93 | 10 |
| 26 | | 1000 | 96 | 10 |
| 26 | | 100 | 92 | 10 |
| 26 | | 100 | 92 | 10 |
| 26 | | 1000 | 93 | 10 |
| 26 | | 1000 | 94 | 10 |
| 26 | 6 | | | 10 |
| 27 | | 100 | 63 | 10 |
| 27 | | 100 | 65 | 10 |
| 27 | 34.4 | | | 10 |
| 27 | | 1000 | 96 | 10 |
| 27 | | 1000 | 98 | 1000 |
| 28 | | 100 | 83 | 10 |
| 28 | | 100 | 84 | 10 |
| 28 | 12.1 | | | 10 |
| 28 | | 1000 | 96 | 10 |
| 28 | | 1000 | 98 | 1000 |
| 29 | | 100 | 62 | 10 |
| 29 | | 100 | 54 | 10 |
| 29 | 37.7 | | | 10 |
| 29 | | 1000 | 92 | 10 |
| 29 | | 1000 | 95 | 1000 |
| 30 | | 100 | 83 | 10 |
| 30 | | 100 | 82 | 10 |
| 30 | | 1000 | 96 | 10 |
| 30 | | 1000 | 98 | 1000 |
| 31 | | 100 | 82 | 10 |
| 31 | | 100 | 77 | 10 |
| 31 | | 1000 | 96 | 10 |
| 31 | | 1000 | 98 | 1000 |
| 32 | | 100 | 88 | 10 |
| 32 | | 100 | 83 | 10 |
| 32 | | 1000 | 96 | 10 |
| 32 | | 100 | 97 | 10 |
| 32 | | 1000 | 95 | 10 |
| 32 | | 1000 | 98 | 1000 |
| 32 | | 1000 | 97 | 1000 |
| 32 | 4.8 | | | 10 |
| 33 | | 100 | 84 | 10 |
| 33 | | 100 | 75 | 10 |
| 33 | | 1000 | 96 | 10 |
| 33 | | 1000 | 98 | 1000 |
| 34 | | 100 | 55 | 10 |
| 34 | | 100 | 37 | 10 |
| 34 | | 1000 | 95 | 10 |
| 34 | | 1000 | 97 | 1000 |
| 35 | | 100 | 87 | 10 |
| 35 | | 100 | 77 | 10 |
| 35 | | 1000 | 96 | 10 |
| 35 | | 1000 | 98 | 1000 |
| 36 | | 100 | 79 | 10 |
| 36 | | 100 | 72 | 10 |
| 36 | | 1000 | 96 | 10 |
| 36 | | 1000 | 98 | 1000 |
| 37 | | 100 | 89 | 10 |
| 37 | | 1000 | 93 | 10 |
| 38 | | 100 | 93 | 10 |
| 38 | | 1000 | 95 | 10 |
| 39 | | 100 | 91 | 10 |
| 39 | | 1000 | 94 | 10 |
| 39 | 3.5 | | | 10 |
| 40 | | 100 | 92 | 10 |
| 40 | | 1000 | 94 | 10 |
| 41 | | 100 | 92 | 10 |
| 41 | | 1000 | 92 | 10 |
| 41 | 2 | | | 10 |
| 42 | | 100 | 92 | 10 |
| 42 | | 1000 | 94 | 10 |
| 43 | | 100 | 45 | 10 |
| 43 | | 1000 | 89 | 10 |
| 43 | 121 | | | 10 |
| 44 | | 100 | 92 | 10 |
| 44 | | 1000 | 94 | 10 |
| 45 | | 100 | 92 | 10 |
| 45 | | 1000 | 93 | 10 |
| 45 | 2.6 | | | 10 |
| 46 | | 100 | 93 | 10 |
| 46 | | 1000 | 94 | 10 |
| 47 | | 100 | 86 | 10 |
| 47 | | 1000 | 94 | 10 |
| 48 | | 100 | 86 | 10 |
| 48 | | 1000 | 93 | 10 |
| 49 | | 100 | 88 | 10 |
| 49 | | 1000 | 93 | 10 |
| 49 | 148.9 | | | 10 |
| 50 | | 100 | 93 | 10 |
| 50 | | 1000 | 94 | 10 |
| 51 | | 100 | 91 | 10 |
| 51 | | 1000 | 93 | 10 |
| 52 | | 100 | 91 | 10 |
| 52 | | 1000 | 95 | 10 |
| 53 | | 100 | 90 | 10 |
| 53 | | 1000 | 94 | 10 |
| 54 | | 100 | −2 | 10 |
| 54 | | 1000 | 10 | 10 |
| 55 | | 100 | 90 | 10 |
| 55 | | 1000 | 93 | 10 |
| 55 | 5.4 | | | 10 |
| 56 | | 100 | 69 | 10 |
| 56 | | 1000 | 81 | 10 |
| 56 | 62.2 | | | 10 |
| 71 | | 1000 | 85 | 10 |
| 71 | | 100 | 41 | 10 |
| 71 | | 1000 | 58 | 1000 |
| 71 | 273.3 | | | 10 |
| 72 | 53.7 | | | 10 |
| 72 | | 1000 | 93 | 10 |
| 72 | | 100 | 73 | 10 |
| 72 | | 1000 | 94 | 1000 |
| 72 | 72.4 | | | 10 |
| 73 | | 100 | −20 | 10 |
| 73 | | 100 | 7 | 10 |
| 73 | 1844 | | | 10 |
| 73 | | 1000 | 30 | 10 |
| 73 | | 1000 | −2 | 1000 |
| 73 | >1000 | | 13 | 10 |
| 74 | | 100 | 27 | 10 |
| 74 | | 100 | 37 | 10 |
| 74 | | 1000 | 89 | 10 |
| 74 | | 1000 | 84 | 1000 |
| 75 | | 100 | 47 | 10 |
| 75 | | 100 | 38 | 10 |
| 75 | | 1000 | 92 | 10 |
| 75 | 93 | | | 10 |
| 75 | | 1000 | 93 | 1000 |
| 76 | | 100 | 45 | 10 |
| 76 | | 100 | 43 | 10 |
| 76 | | 1000 | 91 | 10 |
| 76 | | 1000 | 92 | 10 |
| 76 | | 100 | 60 | 10 |
| 76 | 75.7 | | | 10 |
| 76 | | 1000 | 77 | 1000 |
| 76 | | 1000 | 97 | 1000 |
| 78 | 146.5 | | | 10 |
| 78 | | 1000 | 90 | 10 |
| 78 | | 1000 | 62 | 10 |
| 78 | | 100 | 53 | 10 |
| 78 | | 100 | 27 | 10 |
| 78 | 95.9 | | | 10 |
| 78 | | 1000 | 74 | 1000 |
| 78 | | 1000 | 95 | 1000 |
| 78 | 406.4 | | | 10 |
| 80 | | 1000 | 28 | 10 |
| 80 | | 1000 | 69 | 10 |
| 80 | | 100 | 11 | 10 |
| 80 | | 100 | 32 | 10 |
| 80 | | 100 | 17 | 10 |
| 80 | | 1000 | 74 | 10 |
| 80 | | 1000 | 9 | 1000 |
| 80 | | 1000 | 74 | 1000 |
| 80 | | 1000 | 67 | 1000 |
| 80 | 654.2 | | | 10 |
| 82 | | 1000 | 30 | 10 |
| 82 | | 100 | 15 | 10 |
| 82 | | 1000 | 45 | 1000 |
| 84 | | 100 | 5 | 10 |
| 84 | | 100 | 1 | 10 |
| 84 | | 1000 | 88 | 10 |
| 84 | | 1000 | 82 | 1000 |
| 85 | | 1000 | 62 | 10 |
| 85 | | 100 | 20 | 10 |
| 85 | | 1000 | 64 | 1000 |
| 86 | | 1000 | 93 | 10 |
| 86 | | 100 | 58 | 10 |
| 86 | 40.7 | | | 10 |
| 86 | | 1000 | 96 | 1000 |
| 87 | | 1000 | 72 | 10 |
| 87 | | 100 | 19 | 10 |
| 87 | | 1000 | 85 | 1000 |
| 88 | | 1000 | 63 | 10 |
| 88 | | 100 | 28 | 10 |
| 88 | | 1000 | 48 | 1000 |
| 89 | | 1000 | 18 | 10 |
| 89 | | 100 | 11 | 10 |
| 89 | | 1000 | −12 | 1000 |
| 90 | | 100 | 74 | 10 |
| 90 | | 100 | 82 | 10 |
| 90 | 30 | | | 10 |
| 90 | 7.5 | | | 10 |
| 90 | 8.8 | | | 10 |
| 90 | | 1000 | 94 | 10 |
| 90 | | 100 | 92 | 10 |
| 90 | | 100 | 95 | 10 |
| 90 | | 1000 | 96 | 10 |
| 90 | | 1000 | 95 | 10 |
| 90 | | 1000 | 96 | 1000 |
| 90 | | 1000 | 97 | 1000 |
| 90 | | 1000 | 97 | 1000 |
| 91 | | 100 | 76 | 10 |
| 91 | | 100 | 85 | 10 |
| 91 | 18.9 | | | 10 |
| 91 | | 1000 | 96 | 10 |
| 91 | | 1000 | 97 | 1000 |
| 92 | | 1000 | 93 | 10 |
| 92 | | 100 | 92 | 10 |
| 92 | | 1000 | 97 | 1000 |
| 93 | | 100 | 84 | 10 |
| 93 | | 100 | 71 | 10 |
| 93 | | 100 | 89 | 10 |
| 93 | | 100 | 81 | 10 |
| 93 | 30 | | | 10 |
| 93 | 2.8 | | | 10 |
| 93 | 2.8 | | | 10 |
| 93 | 4.2 | | | 10 |
| 93 | | 1000 | 96 | 10 |
| 93 | | 1000 | 95 | 10 |
| 93 | | 1000 | 98 | 1000 |
| 93 | | 1000 | 95 | 1000 |
| 94 | | 1000 | 94 | 10 |
| 94 | | 100 | 93 | 10 |
| 94 | | 1000 | 96 | 1000 |
| 94 | 4 | | | 10 |
| 95 | | 100 | 85 | 10 |
| 95 | | 100 | 83 | 10 |
| 95 | 3.7 | | | 10 |
| 95 | | 1000 | 96 | 10 |
| 95 | | 1000 | 98 | 1000 |
| 96 | | 1000 | 91 | 10 |
| 96 | | 100 | 67 | 10 |
| 96 | | 1000 | 96 | 1000 |
| 96 | 52.8 | | | 10 |
| 97 | | 1000 | 91 | 10 |
| 97 | | 100 | 38 | 10 |
| 97 | | 1000 | 95 | 1000 |
| 99 | | 1000 | 92 | 10 |
| 99 | | 100 | 93 | 10 |
| 99 | | 1000 | 96 | 1000 |
| 101 | | 100 | 30 | 10 |
| 101 | | 100 | 21 | 10 |
| 101 | 492 | | | 10 |
| 101 | | 1000 | 66 | 10 |
| 101 | 375.1 | | | 10 |
| 101 | | 1000 | 69 | 1000 |
| 106 | | 100 | 23 | 10 |
| 106 | | 100 | −12 | 10 |
| 106 | | 100 | 16 | 10 |
| 106 | | 100 | 20 | 10 |
| 106 | 339 | | | 10 |
| 106 | | 1000 | 69 | 10 |
| 106 | | 1000 | 77 | 10 |
| 106 | | 1000 | 70 | 1000 |
| 106 | | 1000 | 69 | 1000 |

### Irreversible Inhibition of KDR Kinase

Enzyme Assay Wash-Out Experiments

To determine if compounds bound irreversibly to enzyme, plates were washed after the enzyme plus compound pre-incubation step and prior to the addition of ATP. Parallel plates were tested wherein one plate was processed exactly as above while a second plate was washed three times in 100 μl of 4 mM HEPES, pH 7.4, to remove unbound compound. 1× Kinase buffer (30 μl of 1 mM MnCl₂, 4 mM HEPES, pH 7.4, 20 μM Na₃VO₄) and 20 μl of ATP/MgCl₂ were added to the wash-out plate. Detection of phosphotyrosinylated peptide for both plates was performed as described above. “Irreversible” compounds are considered to be those where the IC₅₀ differs by approximately three-fold or less between the unwashed and the wash-out plates. The results are shown in Table 2.

For each inhibitor shown in Table 2, two IC₅₀ determinations are shown, one under normal conditions and the other where an intermediate wash-out step is carried out. If there was little change in the IC₅₀ value in the wash-out experiment (3-fold or less) compared to the experiment where there was no wash-out, then it was determined that the compound is as an irreversible binding inhibitor or is behaving like an irreversible binding inhibitor. If there was a large increase in the IC₅₀ value in the wash-out experiment compared to the experiment where there was no wash-out, then it was determined that the compound was behaving as a conventional reversible binding inhibitor. In order to determine the behavior of conventional reversible binding KDR inhibitors in this test, the reference inhibitors Compound A and Compound B were also tested. Compound A is a quinazoline-based inhibitor reported to be a conventional ATP competitive inhibitor described in Hennequin et al., J. Med. Chem. 42:5369-5389 (1999) and Hennequin et al., J. Med. Chem. 45:1300-1312 (2002). Compound B is a phthalazine-based inhibitor reported to be a conventional ATP competitive inhibitor (Bold et al., J. Med. Chem. 43:2310-2323 (2000).

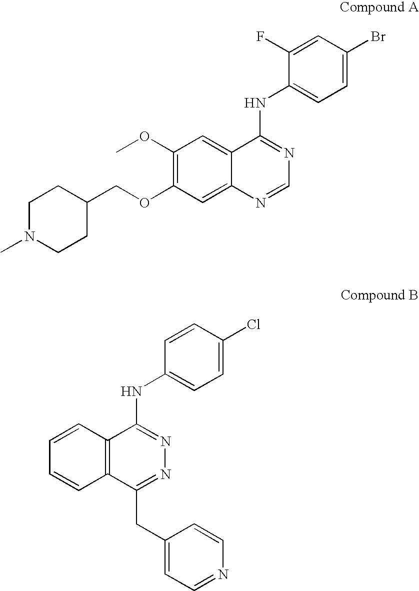

For the reference inhibitors Compound A and Compound B, it is evident from the data in Table 2 that there is a large increase in the IC₅₀ values in the experiment where there is a wash-out step compared to the experiment with no wash-out step indicating that these compounds are functioning as conventional reversible binding inhibitors. In contrast, for many of the compounds of this invention, there is a minimal change in the IC₅₀ values between the wash-out and no wash-out experiments suggesting that these inhibitors function as irreversible binding inhibitors of the enzyme or like irreversible binding inhibitors. Some of the compounds of this invention, such as the compounds of Examples 4 and 15, appear to act like reversible binding inhibitors but are nevertheless potent.

The data in Table 2 again shows that the compounds of this invention are effective inhibitors of KDR kinase that can differ fundamentally from other KDR kinase inhibitors known previously in the art in that they may function as irreversible binding inhibitors of the enzyme or like irreversible binding inhibitors. They therefore are useful for the treatment of the aforementioned disease states.

TABLE 2
Inhibition of KDR Kinase with and without the addition of a washout step

| Example | IC₅₀ (nM) | Experiment |
|---|---|---|
| 4 | 285.2 | no wash |
| 4 | >1000 | wash out |
| 7 | 2.3 | no wash |
| 7 | 1.2 | wash out |
| 15 | 154.2 | no wash |
| 15 | >1000 | wash out |
| 93 | 3.7 | no wash |
| 93 | 5.2 | wash out |
| 86 | 40.7 | no wash |
| 86 | 57.1 | wash out |
| 78 | 146.5 | no wash |
| 78 | 513.5 | wash out |
| 78 | 95.9 | no wash |
| 78 | 150 | wash out |
| 90 | 8.8 | no wash |
| 90 | 18.5 | wash out |
| 101 | 375.1 | no wash |
| 101 | 693.7 | wash out |
| 91 | 18.9 | no wash |
| 91 | 18.9 | wash out |
| 76 | 75.7 | no wash |
| 76 | 155 | wash out |
| 75 | 93 | no wash |
| 75 | 160.9 | wash out |
| 95 | 4.2 | no wash |
| 95 | 6.5 | wash out |
| 26 | 12 | no wash |
| 26 | 27.5 | wash out |
| 26 | 8.1 | no wash |
| 26 | 14.1 | wash out |
| 26 | 2.3 | no wash |
| 26 | 5.3 | wash out |
| 20 | 17.9 | no wash |
| 20 | 33.1 | wash out |
| 72 | 53.7 | no wash |
| 72 | 73.7 | wash out |
| 105 | 483 | no wash |
| 105 | 692.5 | wash out |
| 108 | 59.8 | no wash |
| 108 | 82.6 | wash out |
| 153 | 69.8 | no wash |
| 153 | 119.1 | wash out |
| 184 | 5.9 | no wash |
| 184 | 14.2 | wash out |
| 213 | 4.7 | no wash |
| 213 | 16.9 | wash out |
| 221 | 603.6 | no wash |
| 221 | 832.1 | wash out |
| 222 | 78.6 | no wash |
| 222 | 127.5 | wash out |
| Compound A | 122.8 | no wash |
| Compound A | >1000 | wash out |
| Compound B | 438.5 | no wash |
| Compound B | >1000 | wash out |

### Inhibition of KDR Kinase Autophosphorylation in KDR15 Cells

Cellular Autophosphorylation Assay

Human embryonic kidney 293 cells were transfected with full length KDR and designated KDR15 cells. Cells were maintained in 10% fetal calf serum (FCS) in DMEM (LifeTechnologies), penicillin/streptomycin, plus 0.4 μg/ml puromycin. Cells were plated in 24-well dishes (approximately 4000 cells per well) and allowed to adhere for 1 day. Compounds prepared in DMSO were diluted into cold serum-free DMEM media at appropriate final concentrations. Growth media was aspirated from each well and the cells were washed one time with serum-free DMEM. The serum-free media was replaced with 0.5 ml of compound-containing serum-free media. Cells were incubated for 1 hour on ice, then 55 μl of 500 ng/ml VEGF (final 50 ng/ml; VEGF₁₆₅, R&D Systems) was added to each well and incubated for 30 minutes on ice. Cells were resuspended during VEGF incubation and transferred to 1.5 ml tubes, then centrifuged at 12,000 rpm for 10 minutes and the supernatants discarded. Pellets were lysed in 50 μl of NP40 lysis buffer (150 mM NaCl, 50 mM Tris, pH 7.5, 2 mM EDTA, 1% NP-40 [Ipegal CA-630], 1 mM Na₃VO₄, 1 mM PMSF, 20 KIU/ml aprotinin, 1 μg/ml pepstatin, 0.5 ug/ml leupeptin). Lysates were centrifuged for 10 minutes at 12,000 rpm at 4° C. and the supernatants transferred to fresh tubes and frozen until use.

Equal volumes of lysates were fractionated by SDS-PAGE (7.5% acrylamide or 4-15% gradient) and transferred to PVDF membranes (BioRad). Blots were blocked in 8% BSA/TBST for 1 hour at room temperature, then incubated overnight at 4° C. with 1:1000 anti-phospho-KDR-Y996 antibody (specifically detects phosphorylated tyrosine-996 on KDR; Cell Signaling) in 4% BSA/TBST. Blots were washed three times with TBST, followed by incubation with secondary antibody (1:1000 HRP-conjugated goat anti-rabbit IgG) in 5% milk/TBST. Blots were washed six times, 10 minutes each in TBST, then detected with enhanced chemiluminescent reagents (Amersham) and exposed to film. Autoradiographs were quantified by scanning on a Fluor S imager (BioRad) and data normalized to untreated controls. To confirm equal loading of protein, blots were occasionally stripped in SDS/Tris at 50° C., followed by immunoblot analysis in 1:1000 anti-KDR antibody in 5% milk/TBST. The results are shown in Table 3.

When multiple entries for an inhibitor appear in Table 3, it indicates that the inhibitor was evaluated multiple times using the conditions stated in the table. The data in Table 3 shows that the compounds of this invention are effective inhibitors of KDR kinase in intact cells and are therefore useful for the treatment of the aforementioned disease states.

TABLE 3
Inhibition of KDR Kinase Autophosphorylation in KDR15 Cells

| | | Compound | |
|---|---|---|---|
| Example | IC50 (nM) | conc. (nM) | % Inhibition |
| 2 | | 1000 | 18 |
| 2 | | 1000 | 12 |
| 4 | | 1000 | 100 |
| 4 | | 1000 | 106 |
| 4 | <62.5 | | |
| 4 | <15.6 | | |
| 5 | | 1000 | 56 |
| 7 | 250 | | |
| 7 | | 1000 | 22 |
| 7 | | 1000 | 41 |
| 7 | | 1000 | 35 |
| 7 | | 1000 | 41 |
| 7 | | 1000 | 38 |
| 7 | | 1000 | 72 |
| 7 | | 1000 | 60 |
| 7 | | 1000 | 114 |
| 7 | | 1000 | 95 |
| 7 | | 1000 | 93 |
| 8 | 172 | | |
| 8 | | 1000 | 65 |
| 8 | | 1000 | 54 |
| 8 | | 1000 | 82 |
| 8 | | 1000 | 93 |
| 8 | | 1000 | 45 |
| 8 | | 1000 | 103 |
| 8 | | 1000 | 98 |
| 8 | | 1000 | 97 |
| 8 | | 500 | 25 |
| 8 | | 1000 | 102 |
| 8 | | 1000 | 86 |
| 9 | | 1000 | 55 |
| 9 | | 1000 | 94 |
| 15 | <15.6 | | |
| 15 | <62.5 | | |
| 15 | | 1000 | 108 |
| 15 | | 200 | 61 |
| 15 | | 1000 | 73 |
| 15 | | 1000 | 99 |
| 15 | | 1000 | 100 |
| 15 | | 1000 | 89 |
| 15 | | 1000 | 104 |
| 15 | | 1000 | 103 |
| 15 | | 500 | 89 |
| 15 | | 1000 | 95 |
| 15 | | 1000 | 110 |
| 15 | | 1000 | 69 |
| 15 | | 1000 | 111 |
| 15 | <62.5 | | |
| 17 | | 1000 | 16 |
| 18 | | 1000 | 27 |
| 19 | | 1000 | −10 |
| 20 | | 1000 | 5 |
| 20 | | 1000 | −1 |
| 20 | | 1000 | 23 |
| 22 | | 1000 | 31 |
| 24 | | 1000 | 0 |
| 25 | 1000 | | |
| 25 | | 1000 | 64 |
| 25 | | 1000 | 49 |
| 25 | | 1000 | 60 |
| 26 | 207 | | |
| 26 | 250 | | |
| 26 | 221 | | |
| 26 | | 1000 | 50 |
| 26 | | 1000 | 138 |
| 26 | | 200 | 84 |
| 26 | | 600 | 94 |
| 26 | | 200 | 36 |
| 26 | | 1000 | 83 |
| 26 | | 1000 | 69 |
| 26 | | 1000 | 90 |
| 26 | | 1000 | 80 |
| 26 | | 1000 | 85 |
| 26 | | 1000 | 87 |
| 26 | | 1000 | 62 |
| 26 | | 1000 | 67 |
| 26 | | 1000 | 31 |
| 26 | | 500 | 57 |
| 26 | | 500 | 63 |
| 26 | | 1000 | 91 |
| 26 | | 1000 | 91 |
| 26 | | 1000 | 68 |
| 26 | | 1000 | 92 |
| 26 | | 1000 | 75 |
| 26 | | 1000 | 29 |
| 26 | | 1000 | 113 |
| 26 | <62.5 | | |
| 27 | | 1000 | 25 |
| 28 | | 1000 | 35 |
| 29 | | 1000 | 30 |
| 30 | | 1000 | 54 |
| 30 | | 1000 | 23 |
| 31 | | 1000 | 37 |
| 31 | | 1000 | 18 |
| 32 | | 1000 | 62 |
| 32 | | 1000 | 32 |
| 33 | | 1000 | 19 |
| 34 | | 1000 | 12 |
| 35 | | 1000 | 39 |
| 36 | | 1000 | 27 |
| 36 | | 1000 | 70 |
| 36 | | 1000 | 25 |
| 37 | | 1000 | 11 |
| 38 | | 1000 | 97 |
| 39 | | 1000 | 140 |
| 40 | | 1000 | 110 |
| 41 | | 1000 | 120 |
| 42 | | 1000 | 64 |
| 105 | | 1000 | 124 |
| 44 | | 1000 | 81 |
| 45 | | 1000 | 99 |
| 46 | | 1000 | 79 |
| 47 | | 1000 | 66 |
| 48 | | 1000 | 74 |
| 49 | | 1000 | 90 |
| 50 | | 1000 | 100 |
| 51 | | 1000 | 105 |
| 52 | | 1000 | 94 |
| 53 | | 1000 | 79 |
| 54 | | 1000 | −5 |
| 71 | | 1000 | 26 |
| 71 | | 1000 | 2 |
| 72 | | 1000 | 10 |
| 72 | | 1000 | −6 |
| 72 | | 1000 | −4 |
| 73 | <15.6 | | |
| 73 | | 1000 | 90 |
| 73 | | 1000 | 143 |
| 73 | | 1000 | 113 |
| 74 | | 1000 | 4 |
| 75 | | 1000 | 45 |
| 75 | | 1000 | 61 |
| 76 | 1000 | | |
| 76 | | 1000 | 75 |
| 76 | | 1000 | 42 |
| 76 | | 1000 | 48 |
| 76 | | 1000 | 47 |
| 76 | | 1000 | 31 |
| 76 | | 1000 | 60 |
| 78 | | 1000 | 3 |
| 78 | | 1000 | 61 |
| 78 | | 1000 | 117 |
| 78 | | 1000 | 61 |
| 80 | <62.5 | | |
| 80 | | 1000 | 92 |
| 80 | | 1000 | 52 |
| 80 | | 1000 | 58 |
| 80 | | 1000 | 15 |
| 80 | | 1000 | 52 |
| 80 | | 1000 | 21 |
| 80 | | 1000 | 71 |
| 82 | | 1000 | 12 |
| 84 | | 1000 | 4 |
| 86 | | 1000 | 5 |
| 86 | | 1000 | −17 |
| 88 | | 1000 | 32 |
| 90 | 236 | | |
| 90 | | 1000 | 53 |
| 90 | | 1000 | 84 |
| 90 | | 200 | 29 |
| 90 | | 1000 | 29 |
| 90 | | 1000 | 35 |
| 90 | | 1000 | 52 |
| 90 | | 1000 | 34 |
| 90 | | 1000 | 54 |
| 90 | | 1000 | 75 |
| 90 | | 1000 | 55 |
| 90 | | 1000 | 42 |
| 90 | | 1000 | 73 |
| 90 | 147 | | |
| 91 | | 1000 | 55 |
| 92 | | 1000 | −22 |
| 92 | | 1000 | 29 |
| 93 | 184 | | |
| 93 | | 1000 | 49 |
| 93 | | 1000 | 131 |
| 93 | | 1000 | 82 |
| 93 | | 1000 | 40 |
| 93 | | 1000 | 35 |
| 94 | 386 | | |
| 94 | | 1000 | 40 |
| 96 | | 1000 | 26 |
| 94 | | 1000 | 54 |
| 96 | | 1000 | 22 |
| 94 | | 1000 | 64 |
| 97 | | 1000 | 44 |
| 97 | | 1000 | 15 |
| 99 | | 1000 | 20 |
| 99 | | 1000 | 29 |
| 101 | | 1000 | 112 |
| 101 | | 1000 | 71 |
| 101 | | 1000 | 36 |
| 101 | 219 | | |
| 106 | | 1000 | 78 |
| 106 | | 1000 | 100 |
| 106 | | 1000 | 59 |
| 106 | 1000 | | |
| 105 | 4-15 | 1000 | |
| 108 | | 1000 | 68 |
| 153 | | 1000 | 17 |
| 184 | | 1000 | 0 |
| 213 | 236-400 | 1000 | |
| 221 | 15 | 1000 | |
| 222 | 175-210 | 1000 | |

### Inhibition of Cellular VEGF-Dependent HUVEC Proliferation

HUVEC Proliferation Assay

Human umbilical vein endothelial cells (HUVEC), obtained from Clonetics, were maintained at 37° C. in EGM2 media (Endothelial Cell Basal Media (EBM) supplemented with components suggested by the distributor: 2% serum, VEGF, hFGFb, EGF, heparin, R3-IGF-1, hydrocortisone, gentamicin sulfate and penicillin/streptomycin). Cells were plated into 96-well dishes (4000 cells per well) and allowed to attach overnight. HUVEC were rinsed one time with 100 μl of EBMc-V (EBM supplemented with all above components except serum or VEGF), then 50 ul of EBMc-V was added to cells. Compounds were prepared at 200× stocks in DMSO, diluted into EBMc-V media as 4× stocks, then 50 ul added to appropriate wells. Finally, 100 μl of 2×VEGF (100 ng/ml prepared in EBMc-V; final 50 ng/ml VEGF) was added to all VEGF-treated wells. EBMc-V (no VEGF) was added to negative control wells. Parallel compound-treated plates were prepared except that 100 μl of EGM2 media containing 2% serum but lacking VEGF (EGMc-V) was added. Cells were incubated for 4-5 days at 37° C.

DNA synthesis was assessed by thymidine incorporation. Cells were incubated for 5 hours in 1 μCi of [³H]-thymidine (PerkinElmer) by addition of 10 ul of 0.1 μCi/ul thymidine prepared in PBS to each well. After incubation, media was aspirated and the cells trypsinized and collected onto a mat using a vacuum-based Micro Cell Harvester (Skatron). [³H]-thymidine was counted in a liquid scintillation counter. Compounds evaluated for ability to inhibit VEGF-dependent growth of human umbilical vein endothelial cells (HUVEC). The results are shown in Table 4.

When multiple entries for an inhibitor appear in Table 4, it indicates that the inhibitor was evaluated multiple times using the conditions stated in the table. The data in Table 4 shows that the compounds of this invention are effective inhibitors of VEGF-dependent growth of human umbilical vein endothelial cells (HUVEC) and therefore can function as anti-angiogenic agents and are useful for the treatment of the aforementioned disease states.

TABLE 4
Inhibition VEGF-dependent Growth of Human Umbilical
Vein Endothelial Cells (HUVEC)

| Example | Conc. (nM) | % Inhibition |
|---|---|---|
| 7 | 100 | 23 |
| 7 | 1000 | 79 |
| 7 | 10000 | 97 |
| 7 | 100 | 23 |
| 7 | 1000 | 79 |
| 7 | 10000 | 97 |
| 8 | 100 | −18 |
| 8 | 1000 | 30 |
| 8 | 10000 | 87 |
| 8 | 100 | 35 |
| 8 | 1000 | 51 |
| 8 | 10000 | 98 |
| 8 | 100 | 21 |
| 8 | 1000 | 37 |
| 8 | 10000 | 89 |
| 8 | 100 | −18 |
| 8 | 1000 | 30 |
| 8 | 10000 | 87 |
| 8 | 100 | 35 |
| 8 | 1000 | 51 |
| 8 | 10000 | 98 |
| 8 | 100 | 21 |
| 8 | 1000 | 37 |
| 8 | 10000 | 89 |
| 15 | 100 | 24 |
| 15 | 1000 | 86 |
| 15 | 10000 | 96 |
| 15 | 100 | 40 |
| 15 | 1000 | 95 |
| 15 | 10000 | 98 |
| 15 | 100 | 42 |
| 15 | 1000 | 67 |
| 15 | 10000 | 97 |
| 15 | 100 | 24 |
| 15 | 1000 | 86 |
| 15 | 10000 | 96 |
| 15 | 100 | 40 |
| 15 | 1000 | 95 |
| 15 | 10000 | 98 |
| 15 | 100 | 42 |
| 15 | 1000 | 67 |
| 15 | 10000 | 97 |
| 25 | 100 | 29 |
| 25 | 1000 | 51 |
| 25 | 10000 | 81 |
| 25 | 100 | 29 |
| 25 | 1000 | 51 |
| 25 | 10000 | 81 |
| 26 | 100 | −5 |
| 26 | 1000 | 12 |
| 26 | 10000 | 89 |
| 26 | 100 | 10 |
| 26 | 1000 | 15 |
| 26 | 10000 | 96 |
| 26 | 100 | 43 |
| 26 | 1000 | 61 |
| 26 | 10000 | 99 |
| 26 | 100 | 9 |
| 26 | 1000 | 34 |
| 26 | 10000 | 95 |
| 26 | 100 | 10 |
| 26 | 1000 | 13 |
| 26 | 10000 | 87 |
| 26 | 100 | 7 |
| 26 | 1000 | 20 |
| 26 | 10000 | 91 |
| 26 | 100 | −5 |
| 26 | 1000 | 12 |
| 26 | 10000 | 89 |
| 26 | 100 | 10 |
| 26 | 1000 | 15 |
| 26 | 10000 | 96 |
| 26 | 100 | 43 |
| 26 | 1000 | 61 |
| 26 | 10000 | 99 |
| 26 | 100 | 9 |
| 26 | 1000 | 34 |
| 26 | 10000 | 95 |
| 26 | 100 | 10 |
| 26 | 1000 | 13 |
| 26 | 10000 | 87 |
| 26 | 100 | 7 |
| 26 | 1000 | 20 |
| 26 | 10000 | 91 |
| 32 | 100 | 10 |
| 32 | 1000 | 41 |
| 32 | 10000 | 98 |
| 32 | 100 | 10 |
| 32 | 1000 | 41 |
| 32 | 10000 | 98 |
| 39 | 100 | 18 |
| 39 | 1000 | 58 |
| 39 | 10000 | 99 |
| 39 | 100 | 18 |
| 39 | 1000 | 58 |
| 39 | 10000 | 99 |
| 41 | 100 | −5 |
| 41 | 1000 | 42 |
| 41 | 10000 | 98 |
| 41 | 100 | −5 |
| 41 | 1000 | 42 |
| 41 | 10000 | 98 |
| 105 | 100 | −1 |
| 105 | 1000 | 36 |
| 105 | 10000 | 88 |
| 105 | 100 | −1 |
| 43 | 1000 | 36 |
| 43 | 10000 | 88 |
| 55 | 100 | 7 |
| 55 | 1000 | 61 |
| 55 | 10000 | 99 |
| 55 | 100 | 7 |
| 55 | 1000 | 61 |
| 55 | 10000 | 99 |
| 75 | 100 | −22 |
| 75 | 1000 | 41 |
| 75 | 10000 | 91 |
| 75 | 100 | −22 |
| 75 | 1000 | 41 |
| 75 | 10000 | 91 |
| 76 | 100 | −30 |
| 76 | 1000 | 63 |
| 76 | 10000 | 96 |
| 76 | 100 | −30 |
| 76 | 1000 | 63 |
| 76 | 10000 | 96 |
| 78 | 100 | 6 |
| 78 | 1000 | 71 |
| 78 | 10000 | 94 |
| 78 | 100 | 9 |
| 78 | 1000 | 81 |
| 78 | 10000 | 99 |
| 78 | 100 | 9 |
| 78 | 1000 | 25 |
| 78 | 10000 | 74 |
| 78 | 100 | 6 |
| 78 | 1000 | 71 |
| 78 | 10000 | 94 |
| 78 | 100 | 9 |
| 78 | 1000 | 81 |
| 78 | 10000 | 99 |
| 78 | 100 | 9 |
| 78 | 1000 | 25 |
| 78 | 10000 | 74 |
| 80 | 100 | 39 |
| 80 | 1000 | 85 |
| 80 | 10000 | 97 |
| 80 | 100 | 39 |
| 80 | 1000 | 85 |
| 80 | 10000 | 97 |
| 90 | 100 | 22 |
| 90 | 1000 | 15 |
| 90 | 10000 | 95 |
| 90 | 100 | 22 |
| 90 | 1000 | 15 |
| 90 | 10000 | 95 |
| 91 | 100 | 0 |
| 91 | 1000 | 15 |
| 91 | 10000 | 93 |
| 91 | 100 | 0 |
| 91 | 1000 | 15 |
| 91 | 10000 | 93 |
| 94 | 100 | 28 |
| 94 | 1000 | 60 |
| 94 | 10000 | 94 |
| 94 | 100 | 20 |
| 94 | 1000 | 52 |
| 94 | 10000 | 78 |
| 94 | 100 | 28 |
| 94 | 1000 | 60 |
| 94 | 10000 | 94 |
| 94 | 100 | 20 |
| 94 | 1000 | 52 |
| 94 | 10000 | 78 |
| 101 | 100 | 12 |
| 101 | 1000 | 36 |
| 101 | 10000 | 94 |
| 101 | 100 | 12 |
| 101 | 1000 | 36 |
| 101 | 10000 | 94 |

In Vivo Evaluation of the Compounds of Examples 26, 105 and 190

The compound described in Example 26 was evaluated in vivo using standard pharmacological test procedures which measures the ability to inhibit the growth of human tumor xenografts. Human colon carcinoma DLD-1 cells (American Type Culture Collection, Rockville, Md.) were grown in tissue culture in DMEM (Gibco/BRL, Gaithersburg, Md.) supplemented with 10% FBS (Gemini Bio-Products Inc., Calabasas, Calif.). Athymic nu/nu female mice (Charles River, Wilmington, Mass.) were injected subcutaneously (SC) in the flank area with 6×10⁶ DLD-1 cells. When tumors attained a mass of between 75 and 100 mg, the mice were randomly assigned into treatment groups with 5 animals per group. Animals were treated orally (PO) once daily on days 1 through 20 post staging (day zero) with a dose range (50 to 100 mg/kg/dose) of compound prepared in 0.5% Methocel/0.5% Tween 80 or 0.5% Methocel/0.4% Tween 80 as the vehicle control. Tumor mass was determined every 7 days [(length×width²)/2] for 21 days post-staging. Relative tumor growth (mean tumor mass on days 7, 14 and 21 divided by the mean tumor mass on day zero) was determined for each treatment group. The results are shown in Table 5.

The results in Table 5 show that after the 21-day course of the experiment the tumors in the drug treated animals are much smaller than those in the animals that did not receive the drug, indicating that the compounds of this invention are useful anti-tumor agents for the treatment of cancer.

TABLE 5
The Effect of the Compound of Example 26 on the Growth of Human Colon
Carcinoma DLD1 in the Nude Mouse Model

| a | | | | | | | | | | |
|---|---|---|---|---|---|---|---|---|---|---|
| Drug | | | | | | | | | | |
| Treat- | | | | | | | | | | |
| ment | b | c | | b | c | | b | c | | |
| mg/kg/ | Day | % | d | Day | % | d | Day | % | d | e |
| dose | 7 | T/C | (p) | 14 | T/C | (p) | 21 | T/C | (p) | S/T |
| Saline | 4.99 | | | 11.14 | | | 17.21 | | | 10/10 |
| (control) | | | | | | | | | | |
| Ex. 26 | 3.36 | 67 | 0.09 | 6.10 | 55 | 0.04 | 8.80 | 51 | 0.06 | 5/5 |
| (100 PO) | | | | | | | | | | |
| Ex. 26 | 4.21 | 84 | 0.25 | 6.93 | 62 | 0.08 | 10.50 | 61 | 0.12 | 5/5 |
| (50 PO) | | | | | | | | | | |

a) The compound is administered on days 1 through 20.
$b ) Relative Tumor Growth = \frac{Mean Tumor Mass on Day 7 , 14 , 21}{Mean Tumor Mass on Day 0}$
$c ) \% T / C = \frac{Relative Tumor Growth of Treated Group}{Relative Tumor Growth of Placebo Group} ⨯ 100$
d) Statistical Analysis (Student's T-test) of Log Relative Tumor Growth. A p-value (p ≦
0.05 indicates a statistically significant reduction in Relative Tumor Growth of Treated
Group compared to the Placebo control.
e) S/T =# of Survivors/# of Treated on Day +21 post tumor staging.

The compounds described in Examples 105 and 190 were also tested using the above protocol. The results are shown in Table 6.

The results in Table 6 also show that after the 21-day course of the experiment, the tumors in the drug treated animals are much smaller than those in the animals that did not receive the drug, indicating that the compounds of this invention are useful anti-tumor agents for the treatment of cancer.

TABLE 6
The Effect of the Compounds of Examples 105 and 190 on the Growth of
Human Colon Carcinoma DLD1 in the Nude Mouse Model

| a | b | c | | b | c | | b | c | |
|---|---|---|---|---|---|---|---|---|---|
| Drug Treatment | Day | % | d | Day | % | d | Day | % | d |
| mg/kg/dose | 7 | T/C | (p) | 14 | T/C | (p) | 21 | T/C | (p) |
| 0/5% Methocel | 2.58 | | | 7.86 | | | 14.22 | | |
| 0/4% Tween 80 | | | | | | | | | |
| Ex. 190 | 1.58 | 61 | 0.35 | 7.41 | 94 | 0.48 | 10.74 | 76 | 0.35 |
| Ex.150 | 1.62 | 63 | 0.33 | 6.45 | 82 | 0.39 | 12.54 | 88 | 0.39 |

a) Compounds were adminstered on days 1 through 20.
$b ) Relative Tumor Growth = \frac{Mean Tumor Mass on Day 7 , 14 , 21 , 28}{Mean Tumor Mass on Day 0}$
$c ) \% T / C = \frac{Relative Tumor Growth of Treated Group}{Relative Tumor Growth of Placebo Group} ⨯ 100$
d) Statistical Analysis (Student's T-test) of Log Relative Tumor Growth. A p-value
(p ≦ 0.05) indicates a statistically significant reduction in Relative Tumor Growth of
Treated Group compared to the Placebo Control.

The compositions and dosage forms of the invention are administered to a patient in need of treatment or prevention of a condition characterized, at least in part by, excessive, abnormal or inappropriate angiogenesis. The patient may be an animal, and is preferably a mammal, and more preferably, human.

The compounds of this invention may be formulated neat or may be combined with one or more pharmaceutically acceptable carriers for administration, as example, solvents, diluents and the like, and may be administered orally in such forms as tablets, capsules (including time release and sustained release formulations), dispersible powders, granules, or suspensions containing, for example, from about 0.05 to 5% of suspending agent, syrups containing, for example, from about 10 to 50% of sugar, and elixirs containing, for example, from about 20 to 50% ethanol, and the like, or parentally in the form of sterile injectable solution or suspension containing from about 0.05 to 5% suspending agent in an isotonic medium. Such pharmaceutical preparations may contain, for example, from about 0.05 up to about 90% of the active ingredient in combination with the carrier, more usually between about 5% and 60% by weight.

The effective dosage of active ingredient employed may vary depending on the particular compound employed, the mode of administration and the severity of the condition being treated. However, in general, satisfactory results are obtained when the compounds of the invention are administered at a daily dosage of from about 0.5 to about 1000 mg/kg of body weight, optionally given in divided doses two to four times a day, or in sustained release form. The total daily dosage is projected to be from about 1 to 1000 mg, preferably from about 2 to 500 mg. Dosage forms suitable for internal use comprise from about 0.5 to 1000 mg of the active compound in intimate admixture with a solid or liquid pharmaceutically acceptable carrier. This dosage regimen may be adjusted to provide the optimal therapeutic response. For example, several divided doses may be administered daily or the dose may be proportionally reduced as indicated by the exigencies of the therapeutic situation.

The compounds of this invention may be administered orally as well as by intravenous, intramuscular, or subcutaneous routes. Solid carriers include starch, lactose, dicalcium phosphate, microcrystalline cellulose, sucrose and kaolin, while liquid carriers include sterile water, polyethylene glycols, non-ionic surfactants and edible oils such as corn, peanut and sesame oils, as are appropriate to the nature of the active ingredient and the particular form of administration desired. Adjuvants customarily employed in the preparation of pharmaceutical compositions may be advantageously included, such as flavoring agents, coloring agents, preserving agents and antioxidants, for example, vitamin E, ascorbic acid, BHT and BHA.

The preferred pharmaceutical compositions from the standpoint of ease of preparation and administration are solid compositions, particularly tablets and hard-filled or liquid-filled capsules. Oral administration of the compounds is preferred.

In some cases it may be desirable to administer the compounds directly to the airways in the form of an aerosol.

The compounds of this invention may also be administered parenterally or intraperitoneally. Solutions or suspensions of these active compounds as a free base or pharmacologically acceptable salt can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy injectability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

For the treatment of cancer, the compounds of this invention can be administered in combination with other antitumor substances or with radiation therapy. These other substances or radiation treatments can be given at the same or at different times as the compounds of this invention. These combined therapies may effect synergy and result in improved efficacy. For example, the compounds of this invention can be used in combination with mitotic inhibitors such as taxol or vinblastine, alkylating agents such as cisplatin or cyclophosamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, and antiestrogens such as tamoxifen.

The compounds of this invention are tyrosine kinase inhibitors and can be used in combination with other tyrosine kinase inhibitors. The compounds of this invention can be used in combination with antibodies that target deregulated receptors involved in malignancy.

The preparation of representative examples of the compounds of this invention is described below.

### EXAMPLE 1

### N-(4-chloro-2,5-dimethoxyphenyl)-6,7-dimethoxy-4-quinazolinamine

A mixture of 15.0 g (84.2 mmol) of 2-amino-4,5-dimethoxy-benzonitrile and 13.3 g (112 mmol) of dimethylformamide dimethylacetal was stirred at 100° C. for 2 hours. The excess reagents were removed at reduced pressure at 100° C. The residue was dissolved in methylene chloride and the solution was passed through a short column of Magnesol™ eluting with methylene chloride. The solvent was removed and ether was added. After storage in the cold, the ether was decanted from an orange solid that was dried under vacuum giving 17.7 g of amidine intermediate, N′-(2-cyano-4,5-dimethoxy-phenyl)-N,N-dimethyl-formamidine. A 3 g (12.9 mmol) portion of this intermediate and 2.5 (13.5 mmol) of 4-chloro-2,5-dimethoxy-aniline in 12 ml of acetic acid was refluxed for 1.5 hours. The mixture was cooled to room temperature and ether was added. Solid was collected and washed with ether giving 3.9 g of the title compound: mass spectrum (electrospray, m/e): M+H 376.16.

### EXAMPLE 2

### 2-chloro-5-[(6,7-dimethoxy-4-quinazolinyl)amino]benzo-1,4-quinone

A solution of 2 g (5.32 mmol) of N-(4-chloro-2,5-dimethoxyphenyl)-6,7-dimethoxy-4-quinazolinamine in 70 ml acetonitrile and 10 ml of water was prepared by warming on a steam bath. While still slightly warm, 5.84 g (10.6 mmol) of ceric ammonium nitrate was added over 5 minutes. After stirring 1 hour, the solid was collected and washed several times with water and ether. The solid was refluxed in acetonitrile and the mixture was cooled. Solid was collected giving 1.38 g of the title compound as a red crystalline solid: mass spectrum (electrospray, m/e): M+H 346.07.

### EXAMPLE 3

### 5-(6,7-dimethoxy-quinazolin-4-ylamino)-2-methyl-phenol

A mixture of 3.0 g of 4-chloro-6,7-dimethoxy-quinazoline (12.9 mmol) and 1.66 g (13.5 mmol) of 3-hydroxy-4-methyl aniline was refluxed in 12 ml of acetic acid for 1.5 hours. The mixture was cooled and diluted with an equal volume of ether. The solid was collected and washed with ether yielding 4.0 g of the title compound.

### EXAMPLE 4

### 2-[(6,7-dimethoxy-4-quinazolinyl)amino]-5-methylbenzo-1,4-quinone

To a solution of 2.38 g (22.5 mmol) of sodium carbonate and 22.5 ml of 1 N sodium hydroxide in 176 ml of water, 3.5 g (11.2 mmol) of 5-(6,7-dimethoxy-quinazolin-4-ylamino)-2-methyl-phenol and 70 ml of ethyl acetate was added. The mixture was stirred as 9 g of Fremy's salt was added over 10 minutes. The mixture was then stirred overnight. The mixture was neutralized with solid ammonium chloride and extracted with a THF-ethyl acetate mixture. The organic solution was dried over magnesium sulfate and filtered through a short column of Magnesol™. The solvent was removed and the residue was refluxed in 70 ml of acetonitile. The mixture was cooled to room temperature and the solid was collected and washed with ether, yielding 1.8 g of the title compound as a red powder: mass spectrum (electrospray, m/e): M+H 326.10

### EXAMPLE 5

### 4-[(6,7-dimethoxy-4-quinazolinyl)amino]-1-methyl-7-oxabicyclo[4.1.0]hept-3-ene-2,5-dione

A suspension of 1.2 g (3.7 mmol) of 2-[(6,7-dimethoxy-4-quinazolinyl)amino]-5-methylbenzo-1,4-quinone in 70 ml of acetonitrile and 10 ml of water containing 1.55 g (18.4 mmol) of sodium bicarbonate was stirred as 2.09 g of 30% hydrogen peroxide was added. After 4 hours, the solid was collected via filtration and washed with water and then with ether. The solid was dried under vacuum at 90° C. yielding the title compound as a yellow powder: mass spectrum (electrospray, m/e): M+H 342.11.

### EXAMPLE 6

### 3-[(6,7-dimethoxy-4-quinazolinyl)amino]-5-methylphenol

A mixture of 3.0 g of 5-(6,7-dimethoxy-quinazolin-4-ylamino)-2-methyl-phenol (12.9 mmol) and 1.66 g (13.5 mmol) of 3-hydroxy-5-methyl aniline was refluxed in 12 ml of acetic acid for 1.5 hour. The mixture was cooled and diluted with an equal volume of ether. The solid was collected and washed with dilute sodium bicarbonate and then with water. The solid was boiled in methanol and then cooled and collected giving 1.1 g of the title compound: mass spectrum (electrospray, m/e): M+H 312.16.

### EXAMPLE 7

### 2-[(6,7-dimethoxy-4-quinazolinyl)amino]-6-methylbenzo-1,4-quinone

To a solution of 1.1 g (10.4 mmol) of sodium carbonate and 8 ml of 1 N sodium hydroxide in 125 ml of water, 2.5 g (8 mmol) of 3-[(6,7-dimethoxy-4-quinazolinyl)amino]-5-methylphenol and 50 ml of ethyl acetate were added. The mixture was stirred as 6.5 g of Fremy's salt was added over 10 minutes. The mixture was then stirred 2 hours. The mixture was neutralized with solid ammonium chloride and extracted with ethyl acetate. The organic solution was dried over magnesium sulfate and filtered through a short column of Magnesol™. The solvent was removed and the residue was refluxed in 70 ml of acetonitile. The mixture was concentrated, cooled to room temperature and diluted with ether. The solid was collected and washed with ether yielding 0.31 g of the title compound as a red powder: mass spectrum (electrospray, m/e): M+H 326.10

### EXAMPLE 8

### 2-[(6,7-dimethoxy-4-quinazolinyl)amino]-5-ethylbenzo-1,4-quinone

The title compound was prepared from N-(4-ethyl-2,5-dimethoxyphenyl)-6,7-dimethoxy-4-quinazolinamine using the method described above in Example 2. The N-(4-ethyl-2,5-dimethoxyphenyl)-6,7-dimethoxy-4-quinazolinamine is prepared as described above in Example 1: mass spectrum (electrospray, m/e): M+H 340.14

### EXAMPLE 9

### 2-[(6,7-dimethoxy-4-quinazolinyl)amino]-5-isopropylbenzo-1,4-quinone

The title compound was prepared from N-(4-isopropyl-2,5-dimethoxyphenyl)-6,7-dimethoxy-4-quinazolinamine using the method described above in Example 2. The N-(4-isopropyl-2,5-dimethoxyphenyl)-6,7-dimethoxy-4-quinazolinamine was prepared as described above in Example 1: mass spectrum (electrospray, m/e): M+H 354.0.

### EXAMPLE 10

### 3-methoxy-4-(2-methoxyethoxy)benzonitrile

To a suspension of 7.5 g (187.7 mmol) of 60% sodium hydride in 100 ml of dimethylformamide (DMF), 24.2 g (174.3 mmol) of 1-bromo-2-methoxy-ethane was added. A solution of 20 g (134.1 mmol) of 4-hydroxy-3-methoxy-benzonitrile in 100 ml DMF was added dropwise over 20 minutes. The mixture was stirred at 70° C. for 5.5 hours and at room temperature overnight. The mixture was poured into water. The solid was collected and washed with water and hexanes yielding 19.5 g of the title compound as a white solid: mass spectrum (electrospray, m/e): M+H 207.00.

### EXAMPLE 11

### 5-methoxy-4-(2-methoxyethoxy)-2-nitrobenzonitrile

To a stirred solution of 16.7 g (80.6 mmol) of 3-methoxy-4-(2-methoxyethoxy)benzonitrile in 100 ml of trifluoroacetic anhydride and 70 ml of chloroform, 9.7 g (120.9 mmol) of solid ammonium nitrate was added portionwise over 10 minutes. The solid was separated and the mixture was warmed to a gentle boil. After 2 hours, the mixture was diluted with hexanes and the solid was collected. The solid was washed with hexanes, water, dilute sodium bicarbonate solution and then with water. This solid was air dried to yield 18.4 g of the title compound as a light yellow solid: mass spectrum (electrospray, m/e): M+H 251.97

### EXAMPLE 12

### 2-amino-5-methoxy-4-(2-methoxyethoxy)benzonitrile

To a mixture of 17 g (67.4 mmol) of 5-methoxy-4-(2-methoxyethoxy)-2-nitrobenzonitrile, 83 g (1 mol) of cyclohexene in 180 ml of ethyl acetate, and 180 ml of methanol, 1.7 g of 10% palladium on carbon catalyst was added. The mixture was stirred at reflux for 4 hours. The mixture was filtered and the solvent was evaporated. The residue was boiled in ethanol, cooled to 35° C., and filtered from a solid that was discarded. The solvent was evaporated from the filtrate and the residue was recrystallized from a mixture of carbon tetrachloride and hexanes yielding 7.5 g of the title compound: mass spectrum (electrospray, m/e): M+H 223.15

### EXAMPLE 13

### N′-[2-cyano-4-methoxy-5-(2-methoxyethoxy)phenyl]-N,N-dimethylimidoformamide

A mixture of 7.45 g (33.5 mmol) of 2-amino-5-methoxy-4-(2-methoxyethoxy)benzonitrile and 5.3 g (44.6 mmol) of dimethylformamide dimethylacetal was heated at 100° C. for 2 hours. Excess reagent was removed at reduced pressure leaving a solid which was washed with ether-hexanes 1:1 yielding 8.8 g of the title compound: mass spectrum (electrospray, m/e): M+H 278.16

### EXAMPLE 14

### 5-{[6-methoxy-7-(2-methoxyethoxy)-4-quinazolinyl]amino}-2-methylphenol

A mixture of 3 g (10.82 mmol) of N′-[2-cyano-4-methoxy-5-(2-methoxyethoxy)phenyl]-N,N-dimethylimidoformamide and 1.4 g (11.36 mmol) of 3-hydroxy-4-methyl aniline was heated in 12 ml of acetic acid for 1 hour 15 minutes. The mixture was cooled and diluted with 35 ml of ether. After stirring, solid was collected yielding 3.8 g of the title compound as a light yellow solid: mass spectrum (electrospray, m/e): M+H 356.15.

### EXAMPLE 15

### 2-{[6-methoxy-7-(2-methoxyethoxy)-4-quinazolinyl]amino}-5-methylbenzo-1,4-quinone

A mixture of 3.7 g (10.4 mmol) of 5-{[6-methoxy-7-(2-methoxyethoxy)-4-quinazolinyl]amino}-2-methylphenol, 1.1 g of sodium carbonate, 13 ml of 1 N sodium hydroxide, and 8.38 g (31.2 mmol) of Fremy's salt was stirred at room temperature for 17 hours. To this mixture, 70 ml of THF was added and the mixture was stirred at 50° C. for 2 hours. The organic layer with suspended solid was separated and filtered. The solid was washed with water and ethyl acetate. This was recrystallized from a mixture of ethyl acetate, THF and methanol yielding the title compound as red needles: mass spectrum (electrospray, m/e): M+H 370.14.

### EXAMPLE 16

### 6-methoxy-7-(2-methoxyethoxy)-N-(2,3,5-trimethoxyphenyl)quinazolin-4-amine

A mixture of 8 g (28.85 mmol) of N′-[2-cyano-4-methoxy-5-(2-methoxyethoxy)phenyl]-N,N-dimethylimidoformamide and 5.8 g (31.7 mmol) of 2,3,5-trimethyloxyaniline (Monatsh Chem. 20:398 (1899) and Chem. Ber. 408 (1948)) was heated in 35 ml of acetic acid for 1 hour. The mixture was cooled and diluted with ether. After stirring, solid was collected and washed with ether yielding 9.05 g of the title compound as a solid: mass spectrum (electrospray, m/e): M+H 416.10.

### EXAMPLE 17

### 2-methoxy-6-{[6-methoxy-7-(2-methoxyethoxy)quinazolin-4-yl]amino}benzo-1,4-quinone

A solution of 8.65 g (20.8 mmol) of 6-methoxy-7-(2-methoxyethoxy)-N-(2,3,5-trimethoxyphenyl)quinazolin-4-amine in 261 ml acetonitrile and 39 ml of water was prepared by warming on a steam bath. While still slightly warm, 34.24 g (62.5 mmol) of ceric ammonium nitrate was added over 15 minutes. After stirring for 1 hour 10 minutes, the mixture was poured into water and extracted with methylene chloride. The solution was dried over magnesium sulfate and filtered through a short column of Magnesol™. The solvent was evaporated. The solid residue was refluxed in ethyl acetate and the mixture was cooled. Solid was collected giving 2.27 g of the title compound as a red crystalline solid: mass spectrum (electrospray, m/e): M+H 386.10.

### EXAMPLE 18

### 5-methoxy-3-{[6-methoxy-7-(2-methoxyethoxy)quinazolin-4-yl]amino}-2-(phenylthio)benzo-1,4-quinone

A solution of 0.45 g (1.17 mmol) of 2-methoxy-6-{[6-methoxy-7-(2-methoxyethoxy)quinazolin-4-yl]amino}benzo-1,4-quinone in 62 ml of acetronitrile was prepared by boiling. While still warm, a solution of 0.12 g (1.28 mmol) of thiophenol in 12 ml of acetonitrile was added. After stirring for 1 hour, 0.277 g (1.34 mmol) of DDQ was added. After 30 minutes, the mixture was diluted with 500 ml of methylene chloride. The solution was washed with dilute sodium carbonate and then with water. The solution was dried over magnesium sulfate and passed through a short column of Magnesol™. The product was eluted from the column using ethyl acetate-methanol 9:1. The solvent was removed from the combined product fractions and recrystallized from acetonitrile-ether to yield 0.51 g of the title compound as an orange solid: mass spectrum (electrospray, m/e): M+H 494.10.

### EXAMPLE 19

### 2-(benzylthio)-5-methoxy-3-{[6-methoxy-7-(2-methoxyethoxy)quinazolin-4-yl]amino}benzo-1,4-quinone

This compound was prepared from 2-methoxy-6-{[6-methoxy-7-(2-methoxyethoxy)quinazolin-4-yl]amino}benzo-1,4-quinone and benzyl mercaptan using the method described in Example 18 above using a 2.5 hour initial reaction time. The title compound was obtained as a red powder: mass spectrum (electrospray, m/e): M+H 508.10.

### EXAMPLE 20

### 3-methoxy-5-{[6-methoxy-7-(2-methoxyethoxy)quinazolin-4-yl]amino}-2-(1,3-thiazol-5-ylthio)benzo-1,4-quinone

This compound was prepared from 2-methoxy-6-{[6-methoxy-7-(2-methoxyethoxy)quinazolin-4-yl]amino}benzo-1,4-quinone and thiazole-2-thiol using the method described in Example 18 above using a 10 hr initial reaction time at 100° C. The title compound was obtained as a red powder: mass spectrum (electrospray, m/e): M+H 501.1.

### EXAMPLE 21

### N-(3,4-dichloro-2,5-dimethoxyphenyl)-6-methoxy-7-(2-methoxyethoxy)quinazolin-4-amine

A mixture of 6.4 g (23 mmol) of N′-[2-cyano-4-methoxy-5-(2-methoxyethoxy)phenyl]-N,N-dimethylimidoformamide, 2.1 g (25.4 mmol) of sodium acetate and 6.56 g (25.4 mmol) of 3,4-dichloro-2,5-dimethoxy aniline hydrochloride was heated in 27 ml of acetic acid for 1 hour. The mixture was cooled and diluted with ether. After stirring, the solid was collected and washed with ether. The solid was boiled in isopropanol, cooled, and collected, yielding 3.9 g of the title compound as a solid: mass spectrum (electrospray, m/e): M+H 454.1, 456.1.

### EXAMPLE 22

### 2,3-dichloro-5-{[6-methoxy-7-(2-methoxyethoxy)quinazolin-4-yl]amino}benzo-1,4-quinone

This compound was prepared from N-(3,4-dichloro-2,5-dimethoxyphenyl)-6-methoxy-7-(2-methoxyethoxy)quinazolin-4-amine using the method described above in Example 17. The product was purified by chromatography on silica gel eluting with chloroform: mass spectrum (electrospray, m/e): M+H 424.0, 426.1.

### EXAMPLE 23

### 2-{[6-methoxy-7-(2-methoxyethoxy)quinazolin-4-yl]amino}benzo-1,4-quinone

This was prepared from of N′-[2-cyano-4-methoxy-5-(2-methoxyethoxy)phenyl]-N,N-dimethylimidoformamide and 2,5-dimethyoxy aniline using the combined methods described above in Examples 16 and 17: mass spectrum (electrospray, m/e): M+H 356.1, 426.1.

### EXAMPLE 24

### 2-{[6-methoxy-7-(2-methoxyethoxy)quinazolin-4-yl]amino}-5-(phenylthio)benzo-1,4-quinone

To a warm solution of 0.33 g (0.93 mmol) 2-{[6-methoxy-7-(2-methoxyethoxy)quinazolin-4-yl]amino}benzo-1,4-quinone in 49 ml of acetonitrile, 0.112 g (1 mmol) of thiophenol in 10 ml acetonitrile was added. After stirring 30 minutes, 0.22 g (1.07 mmol) of DDQ was added. The mixture was poured into methylene chloride and the solution washed with dilute sodium carbonate. The solution was filtered through a Magnesol™ T plug and solvent was removed from the filtrate. The residue was chromatographed on silica gel eluting product with ethyl acetate. The title compound was obtained (0.097 g) as a brown solid after recrystallization from acetonitrile-ether: mass spectrum (electrospray, m/e): M+H 464.

### EXAMPLE 25

### 2-chloro-5-{[6-methoxy-7-(2-methoxyethoxy)quinazolin-4-yl]amino}benzo-1,4-quinone

This compound was prepared from N′-[2-cyano-4-methoxy-5-(2-methoxyethoxy)phenyl]-N,N-dimethylimidoformamide and 4-chloro-2,5-dimethyoxy aniline using the combined methods described above in Examples 16 and 17.

### EXAMPLE 26

### 2-[4-(1H-imidazol-1-yl)phenoxy]-5-{[6-methoxy-7-(2-methoxyethoxy)quinazolin-4-yl]amino}benzo-1,4-quinone

To a solution of 0.0534 g (0.33 mmol) of 4-(imidazol-1-yl)phenol and 0.01 g of the phase transfer catalyst tricaprylylmethylammonium chloride in 4 ml of methylene chloride, 0.3 ml of 1 N sodium hydroxide solution and 0.1 g (0.26 mmol) of 2-chloro-5-{[6-methoxy-7-(2-methoxyethoxy)quinazolin-4-yl]amino}benzo-1,4-quinone were added. The mixture was stirred vigorously for 30 minutes, poured into water and extracted with methylene chloride. The organic layer was dried over magnesium sulfate and poured onto a short column of Magnesol™. The product was eluted with methylene chloride-methanol 4:1 yielding 0.11 g of the title compound as a red solid: mass spectrum (electrospray, m/e): M+H 514.1, (M+2H)⁺² 257.7; mp=124-132° C.

### EXAMPLES 27-36

100 mg of 2-chloro-5-{[6-methoxy-7-(2-methoxyethoxy)quinazolin-4-yl]amino}benzo-1,4-quinone, 100 mg of a phenol, 100 mg of potassium carbonate and 2.5 ml of acetone were added to a reaction vial. The vials were agitated with a vortex shaker for 16 hours. The contents of the vials were filtered and the solids washed with water. The solids were assayed by LC-MS and those containing the desired products were purified by using a Gilson semi-prep HPLC and a gradient of acetonitrile-water. The fractions from this chromatography were assayed using LC-MS, and those containing the desired individual products in pure form were combined and concentrated to solids. By using this method, the compounds of this invention listed in Table 7 were prepared starting with the indicated phenol.

TABLE 7

| | | | MS m/e |
|---|---|---|---|
| Example | Phenol | Compound Name | M + H |
| 27 | 3-hydroxy- | 3-[(4-{[6-methoxy-7-(2- | 473.15 |
| | benzonitrile | methoxyethoxy)quinazolin-4-yl]amino}-3,6- | |
| | | dioxocyclohexa-1,4-dien-1-yl)oxy]benzonitrile | |
| 28 | 3-methoxyphenol | 2-{[6-methoxy-7-(2-methoxyethoxy)quinazolin- | 478.16 |
| | | 4-yl]amino}-5-(3-methoxyphenoxy)benzo-1,4- | |
| | | quinone | |
| 29 | 4-phenoxyphenol | 2-{[6-methoxy-7-(2-methoxyethoxy)quinazolin- | 540.17 |
| | | 4-yl]amino}-5-(4- | |
| | | phenoxyphenoxy)benzo-1,4-quinone | |
| 30 | 4-fluorophenol | 2-(4-fluorophenoxy)-5-{[6-methoxy-7-(2- | 466.14 |
| | | methoxyethoxy)quinazolin-4-yl]amino}benzo- | |
| | | 1,4-quinone | |
| 31 | 4-hydroxy- | 4-[(4-{[6-methoxy-7-(2- | 473.14 |
| | benzonitrile | methoxyethoxy)quinazolin-4-yl]amino}-3,6- | |
| | | dioxocyclohexa-1,4-dien-1-yl)oxy]benzonitrile | |
| 32 | 4-methoxyphenol | 2-{[6-methoxy-7-(2-methoxyethoxy)quinazolin- | 478.16 |
| | | 4-yl]amino}-5-(4- | |
| | | methoxyphenoxy)benzo-1,4-quinone | |
| 33 | 3-chlorophenol | 2-(3-chlorophenoxy)-5-{[6-methoxy-7-(2- | 482.11 |
| | | methoxyethoxy)quinazolin-4-yl]amino}benzo- | |
| | | 1,4-quinone | |
| 34 | 3-hydroxy- | 2-(3-acetylphenoxy)-5-{[6-methoxy-7-(2- | 490.16 |
| | acetophenone | methoxyethoxy)quinazolin-4-yl]amino}benzo- | |
| | | 1,4-quinone | |
| 35 | 4-methylsulfanyl- | 2-{[6-methoxy-7-(2-methoxyethoxy)quinazolin- | 494.14 |
| | phenol | 4-yl]amino}-5-[4- | |
| | | (methylthio)phenoxy]benzo-1,4-quinone | |
| 36 | 4-trifluoromethyl- | 2-{[6-methoxy-7-(2-methoxyethoxy)quinazolin- | 516.14. |
| | phenol | 4-yl]amino}-5-[4- | |
| | | (trifluoromethyl)phenoxy]benzo-1,4-quinone | |

### EXAMPLES 37-56

A phenol (0.152 mmol) and the phase transfer catalyst tricaprylylmethylammonium chloride (0.01 mmol) were treated with an equivalent amount of 1 N NaOH. Methylene chloride (2 ml) and water (1 ml) were added and this mixture was stirred for 15 minutes. The biphasic mixture was then treated with the 2-chloro-5-{[6-methoxy-7-(2-methoxyethoxy)quinazolin-4-yl]amino}benzo-1,4-quinone (0.101 mmol) in a methylene chloride solution to give a total volume of 8 ml in the reaction. The reactions were agitated with a vortex shaker for a time ranging from 2 hours to 48 hours. Completion of the reaction was determined by LC-MS. The organic layers were then separated and the aqueous solution was extracted further with methylene chloride (2×2 ml). The organic layers were combined and dried over magnesium sulfate and concentrated. The reactions, which showed only desired quinone as the major component, were purified by either recrystallization from acetonitrile or silica gel chromatography. Some reactions showed a substantial amount of the desired product in reduced form. These reactions were treated with an excess of DDQ in methylene chloride (2 ml) and agitated with a vortex shaker overnight. The reactions were washed with a saturated potassium carbonate solution (3×2 ml), and the organic layers dried over magnesium sulfate and concentrated. Again, the reactions which showed only desired quinone as the major component were purified by either recrystallization from acetonitrile or silica gel chromatography. By using this method, the compounds of this invention listed in Table 8 were prepared starting with the indicated phenol.

TABLE 8

| | | | MS m/e |
|---|---|---|---|
| Example | Phenol | Compound Name | M + H |
| 37 | (4-hydroxy- | ethyl {4-[(4-{[6-methoxy-7-(2- | 534.18 |
| | phenyl)-acetic | methoxyethoxy)quinazolin-4-yl]amino}-3,6- | |
| | acid ethyl ester | dioxocyclohexa-1,4-dien-1- | |
| | | yl)oxy]phenyl}acetate | |
| 38 | 4-hydroxy- | 4-[(4-{[6-methoxy-7-(2- | 527.12 |
| | benzenesulfonamide | methoxyethoxy)quinazolin-4-yl]amino}-3,6- | |
| | | dioxocyclohexa-1,4-dien-1-yl)oxy] | |
| | | benzenesulfonamide | |
| 39 | (4-hydroxy- | 2-(4-benzoylphenoxy)-5-{[6-methoxy-7-(2- | 552.18 |
| | phenyl)-phenyl- | methoxyethoxy)quinazolin-4- | |
| | methanone | yl]amino}benzo-1,4-quinone | |
| 40 | 3-(4-hydroxy- | methyl 3-{4-[(4-{[6-methoxy-7-(2- | 534.19 |
| | phenyl)-propionic | methoxyethoxy)quinazolin-4-yl]amino}-3,6- | |
| | acid methyl ester | dioxocyclohexa-1,4-dien-1- | |
| | | yl)oxy]phenyl}propanoate | |
| 41 | 9H-carbazol-2-ol | 2-(9H-carbazol-2-yloxy)-5-{[6-methoxy-7-(2- | 537.18 |
| | | methoxyethoxy)quinazolin-4- | |
| | | yl]amino}benzo-1,4-quinone | |
| 42 | 4-hydroxy- | methyl 4-[(4-{[6-methoxy-7-(2- | 506.16 |
| | benzoic acid | methoxyethoxy)quinazolin-4-yl]amino}-3,6- | |
| | methyl ester | dioxocyclohexa-1,4-dien-1-yl)oxy]benzoate | |
| 43 | 3-trifluromethyl | 2-{[6-methoxy-7-(2- | 516.14 |
| | phenol | methoxyethoxy)quinazolin-4-yl]amino}-5-[3- | |
| | | (trifluoromethyl)phenoxy]benzo-1,4-quinone | |
| 44 | 3-flurophenol | 2-(3-fluorophenoxy)-5-{[6-methoxy-7-(2- | 466.14 |
| | | methoxyethoxy)quinazolin-4- | |
| | | yl]amino}benzo-1,4-quinone | |
| 45 | 5-hydroxy-2- | ethyl 5-[(4-{[6-methoxy-7-(2- | 573.20 |
| | methyl-1H-indole- | methoxyethoxy)quinazolin-4-yl]amino}-3,6- | |
| | 3-carboxylic acid | dioxocyclohexa-1,4-dien-1-yl)oxy]-2-methyl- | |
| | ethyl ester | 1H-indole-3-carboxylate | |
| 46 | 4-bromophenol | 2-(4-bromophenoxy)-5-{[6-methoxy-7-(2- | 526.06 |
| | | methoxyethoxy)quinazolin-4- | |
| | | yl]amino}benzo-1,4-quinone | |
| 47 | 2-isoxazol-5-yl-4- | 2-(2-isoxazol-5-yl-4-methylphenoxy)-5-{[6- | 529.17 |
| | methyl-phenol | methoxy-7-(2-methoxyethoxy)quinazolin- | |
| | | 4-yl]amino}benzo-1,4-quinone | |
| 48 | 4-hydroxy- | benzyl 4-[(4-{[6-methoxy-7-(2- | 582.19 |
| | benzoic acid | methoxyethoxy)quinazolin-4-yl]amino}-3,6- | |
| | benzyl ester | dioxocyclohexa-1,4-dien-1-yl)oxy]benzoate | |
| 49 | 1-(4-hydroxy- | 2-{[6-methoxy-7-(2- | 566.19 |
| | phenyl)-2-phenyl- | methoxyethoxy)quinazolin-4-yl]amino}-5-[4- | |
| | ethanone | (phenylacetyl)phenoxy]benzo-1,4-quinone | |
| 50 | 3-ethylamino- | 2-[3-(ethylamino)phenoxy]-5-{[6-methoxy-7- | 491.19 |
| | phenol | (2-methoxyethoxy)quinazolin-4- | |
| | | yl]amino}benzo-1,4-quinone | |
| 51 | 6-bromo- | 2-[(6-bromo-2-naphthyl)oxy]-5-{[6-methoxy- | 576.08 |
| | naphthalen-2-ol | 7-(2-methoxyethoxy)quinazolin-4- | |
| | | yl]amino}benzo-1,4-quinone | |
| 52 | 2-benzyloxy- | 2-[2-(benzyloxy)phenoxy]-5-{[6-methoxy-7- | 554.19 |
| | phenol | (2-methoxyethoxy)quinazolin-4- | |
| | | yl]amino}benzo-1,4-quinone | |
| 53 | 9H-fluoren-2-ol | 2-(9H-fluoren-2-yloxy)-5-{[6-methoxy-7-(2- | 536.18 |
| | | methoxyethoxy)quinazolin-4- | |
| | | yl]amino}benzo-1,4-quinone | |
| 54 | 4-(2-amino-ethyl)- | 2-[4-(2-aminoethyl)phenoxy]-5-{[6-methoxy- | 491.19 |
| | phenol | 7-(2-methoxyethoxy)quinazolin-4- | |
| | | yl]amino}benzo-1,4-quinone | |
| 55 | 1-(4-hydroxy- | 2-{[6-methoxy-7-(2- | 578.19 |
| | phenyl)-3-phenyl- | methoxyethoxy)quinazolin-4-yl]amino}-5-{4- | |
| | propenone | [(2E)-3-phenylprop-2-enoyl]phenoxy}benzo- | |
| | | 1,4-quinone | |
| 56 | 4-(1-methyl-1- | 2-{[6-methoxy-7-(2- | 566.23 |
| | phenyl-ethyl)- | methoxyethoxy)quinazolin-4-yl]amino}-5-[4- | |
| | phenol | (1-methyl-1-phenylethyl)phenoxy]benzo- | |
| | | 1,4-quinone | |

### EXAMPLE 57

### 3-methoxy-4-[(1-methylpiperidin-4-yl)methoxy]benzonitrile

53.3 ml of 1 N sodium bis(trimethylsilyl)amide was added to a stirred solution of 6.63 g (51.3 mmol) of (1-methyl-piperidin-4-yl)-methanol in 14 ml of THF. After 20 minutes, solid 4-fluoro-3-methoxy benzonitrile was added. The mixture was refluxed for 20 minutes, cooled to room temperature and poured into water. The mixture was extracted with ethyl acetate. The organic extracts were dried over magnesium sulfate. The solvent was removed and the residue was recrystallized from ethyl acetate-hexanes yielding 8.9 g of the title compound as a white solid: mass spectrum (electrospray, m/e): M+H 261.2.

### EXAMPLE 58

### 5-methoxy-4-[(1-methylpiperidin-4-yl)methoxy]-2-nitrobenzonitrile

To a stirred solution of 8.8 g (33.8 mmol) 3-methoxy-4-[(1-methylpiperidin-4-yl)methoxy]benzonitrile in 34 ml of trifluoroacetic anhydride and 34 ml of chloroform cooled in a ice bath, 4.06 g (50.7 mmol) of solid ammonium nitrate was added portionwise over 15 minutes. The mixture was stirred at room temperature for 30 minutes. The solvent was removed and the residue was diluted with chloroform. The solution was washed with sodium bicarbonate solution until neutral. The mixture was dried over magnesium sulfate, filtered, concentrated and chromatographed on a silica gel column. The product fraction was eluted with a mixture of ethyl acetate, methanol and triethylamine to yield 5.2 g of the title compound as a colored solid mass spectrum (electrospray, m/e): M+H 306.2.

### EXAMPLE 59

### 2-amino-5-methoxy-4-[(1-methylpiperidin-4-yl)methoxy]benzonitrile

A solution of 4 g (13.1 mmol) 5-methoxy-4[(1-methylpiperidin-4-yl)methoxy]-2-nitrobenzonitrile in 200 ml of tetrahydrofuran containing 1.2 g of 10% palladium on carbon catalyst was hydrogenated in a Parr apparatus overnight. The mixture was filtered and the solvent evaporated. The product was purified by chromatography on silica gel eluting with ethyl acetate-methanol-triethylamine 80:20:0.5 to give 2.83 g of the title compound as a tan solid: mass spectrum (electrospray, m/e): M+H 276.2.

### EXAMPLE 60

### (4-chloro-2,5-dimethoxy-phenyl)-[6-methoxy-7-(1-methyl-piperidin-4-ylmethoxy)-quinazolin-4-yl]-amine

To a stirred solution of 32.6 g (118.4 mmol) of 2-amino-5-methoxy-4-[(1-methylpiperidin-4-yl)methoxy]benzonitrile in 100 ml of isopropanol, 25.8 g (148 mmol) of t-butoxy-bis(dimethylamino)methane was added and the mixture heated until the reaction was complete. The solvent and excess reagent were evaporated at reduced pressure give the amidine intermediate. A portion of the intermediate (18.2 g (55.17 mmol)) and 10.9 g (57.9 mmol) of 4-chloro-2,5-dimethoxy-phenylamine in 75 ml of acetic acid was refluxed for 2.5 hour. The solvent was evaporated at reduced pressure at 100° C. The residue was dissolved in chloroform and the solution was washed with saturated sodium bicarbonate. The colored solution was dried over magnesium sulfate and filtered through a pad of Magnesol™. The solvent was evaporated and the residue purified by chromatography on silica gel eluting with ethyl acetate-methanol-triethylamine mixtures to yield 4.6 g of the title compound as a grey powder: mass spectrum (electrospray, m/e): M+H 473.2.

### EXAMPLE 61

### 2-chloro-5-([6-methoxy-7-[(1-methylpiperidin-4-yl)methoxy]quinazolin-4-yl]amino)benzo-1,4-quinone

To a warm stirred solution of 7.5 g (15.86 mmol) of (4-chloro-2,5-dimethoxy-phenyl)-[6-methoxy-7-(1-methyl-piperidin-4-ylmethoxy)-quinazolin-4-yl]-amine in 200 ml of acetonitrile and 30 ml of water, 26.1 g (47.57 mmol) of ceric ammonium nitrate was added over 40 minutes. After 15 minutes, 100 ml of chloroform and 60 ml of saturated sodium bicarbonate were added. The organic layer was separated, washed with water and dried over magnesium sulfate. This solution was poured onto a short Magnesol™ column. The product was eluted with chloroform-isopropanol mixtures. The solvent was removed from product fractions giving a brown solid that was extracted many times with ethyl acetate. The solvent was concentrated from the extract and ether was added. The title compound, 0.74 g, was collected as a red powder: mass spectrum (electrospray, m/e): M+H 443.1.

### EXAMPLE 62

### methyl 3-methoxy-4-(2-methoxyethoxy)benzoate

A mixture of 101.2 g (0.56 mol) of 4-hydroxy-3-methoxy-benzoic acid methyl ester (methyl vanillate), 77.2 g (0.56 mol) of 2-bromoethyl methyl ether and 102.1 g (0.74 mol) of potassium carbonate was refluxed and stirred in 1 L of acetone for 23 hours. The hot mixture was filtered. The solvent was evaporated and the residue was dissolved in ethyl acetate. The solution was washed with 1 N sodium hydroxide and then with water. The solution was dried over magnesium sulfate, filtered and the solvent evaporated yielding 95.6 g of the title compound as a solid: mass spectrum (electrospray, m/e): M+H 241.

### EXAMPLE 63

### methyl 5-methoxy-4-(2-methoxyethoxy)-2-nitrobenzoate

To a stirred solution of 24.0 g (0.1 mmol) of methyl 3-methoxy-4-(2-methoxyethoxy)benzoate in 70 ml of acetic acid, 26 ml of 70% nitric acid was added dropwise. After stirring 2 hours, the mixture was heated to 50° C. for 15 minutes. The mixture was poured onto ice water and filtered. The solid was washed with water and dried, yielding 26.3 g of the title compound.

### EXAMPLE 64

### methyl 2-amino-5-methoxy-4-(2-methoxyethoxy)benzoate

A mixture of 26.3 g (92 mmol) of methyl 5-methoxy-4-(2-methoxyethoxy)-2-nitrobenzoate, 15.4 g (280 mmol) of iron powder, 44.3 g (829 mmol) of ammonium chloride, 75 ml of water, and 300 ml of ethanol was stirred at reflux for 30 minutes. The mixture was filtered while hot. The solids were washed with additional hot ethanol. The solvent was evaporated from the combined filtrate. The residue was dissolved in methylene chloride and filtered through a short column of Magnesol™. The solvent was evaporated giving 21.7 g of the title compound as a solid: mass spectrum (electrospray, m/e): M+H 256.4

### EXAMPLE 65

### 4-hydroxy-6-methoxy-7-(2-methoxy-ethoxy)-quinoline-3-carbonitrile

A mixture of 21.7 g (85.1 mmol) of methyl 2-amino-5-methoxy-4-(2-methoxyethoxy)benzoate and 45 ml of dimethylformamide dimethylacetal was heated at 100° C. for several hours. The excess reagent was removed at reduced pressure. The residue was dissolved in methylene chloride and filtered through a pad of Magnesol™. The solvent was evaporated and the residue was dried in vacuum yielding 26.2 g of the intermediate formamide derivative.

A solution of 10.86 ml of 2.5 M n-butyl lithium in hexanes in 300 ml of dry THF was stirred under nitrogen at −78° C. as 9.25 ml (177 mmol) of acetonitrile in 300 ml of THF was added dropwise. After 30 minutes, a solution of the amidine prepared above in 300 ml of THF was added dropwise. After 1 hour, 24 ml of acetic acid was added and the mixture was allowed to warm to room temperature. The solvent was evaporated and the resulting solid was washed with water and air dried giving the title compound.

### EXAMPLE 66

### 4-chloro-6-methoxy-7-(2-methoxyethoxy)-quinoline-3-carbonitrile

To a suspension of 11.4 g (41.6 mmol) of 4-hydroxy-6-methoxy-7-(2-methoxy-ethoxy)-quinoline-3-carbonitrile in 200 ml of methylene chloride, 18 ml (208 mmol) of oxalyl chloride and 0.5 ml of dimethylformamide were added with stirring. The mixture was stirred for 4 hours. The solvent was evaporated at reduced pressure and the residue was redissolved in methylene chloride. The solution was passed through a short column of Magnesol™. The solvent was evaporated and the residue washed with ether yielding 10.3 g of the title compound as a solid.

### EXAMPLE 67

### 4-[(3-hydroxy-4-methylphenyl)amino]-6-methoxy-7-(2-methoxyethoxy)quinoline-3-carbonitrile

A solution of 2.93 g (10 mmol) of 4-chloro-6-methoxy-7-(2-methoxyethoxy)quinoline-3-carbonitrile, 1.35 g (11 mmol) of 5-amino-2-methyl-phenol and 1.27 of pyridine hydrochloride in 25 ml of isopropanol was stirred at reflux for 1 hour. The mixture was cooled and the solid was collected as the hydrochloride salt and washed with cold isopropanol and ether. The solid was stirred in a mixture of saturated sodium bicarbonate and methylene chloride overnight. The solid was collected and washed with water and ether giving after drying yielding 3.1 g of the title compound: mp 230-233° C.; mass spectrum (electrospray, m/e, negative mode): M−H 378.2.

### EXAMPLE 68

### 6-methoxy-7-(2-methoxyethoxy)-4-[(4-methyl-3,6-dioxocyclohexa-1,4-dien-1-yl)amino]quinoline-3-carbonitrile

A mixture of 2.96 g (7.8 mmol) of 4-[(3-hydroxy-4-methylphenyl)amino]-6-methoxy-7-(2-methoxyethoxy)quinoline-3-carbonitrile, 0.83 g of sodium carbonate, 9.75 ml of 1 N sodium hydroxide, 100 ml of water, and 60 ml of ethyl acetate was stirred as 6.3 g (23.4 mmol) of Fremy's salt was added. After stirring overnight at room temperature, 65 ml of THF was added and the mixture was heated to 50° C. for 2 h. A solid was collected by filtration. The filtrate was extracted with methylene chloride and this extract was combined with the solid. Solvent was evaporated. The residue was redissolved in methylene chloride and filtered. The filtrate was chromatographed on silica gel. Product was eluted with methylene chloride-methanol 39:1. The solvent was evaporated from the product fractions and the residue was washed with ether, yielding 0.93 g of the title compound as an orange solid: mp 174-177° C.; mass spectrum (electrospray, m/e): M−H 394.1.

### EXAMPLE 69

### 4-[(4-chloro-2,5-dimethoxyphenyl)amino]-6-methoxy-7-(2-methoxyethoxy)quinoline-3-carbonitrile

A solution of 7.3 g (24.9 mmol) of 4-chloro-6-methoxy-7-(2-methoxyethoxy)-3-quinolinecarbonitrile and 4.3 g (24.9 mmol) of 4-chloro-2,5-dimethoxy-phenylamine in 200 ml of methyoxyethanol was stirred at reflux for 3.5 hours and then allowed to stand at room temperature overnight. The solid was collected and washed with ether giving the hydrochloride salt. This was heated in 700 ml of ethyl acetate and sodium hydroxide solution until the solid dissolved. The organic layer was dried over magnesium sulfate. The solvent was evaporated and the product recrystallized from ethyl acetate-hexanes yielding 9.7 g of the title compound: mass spectrum (electrospray, m/e): M−H 444.2.

### EXAMPLE 70

### 4-[(4-chloro-3,6-dioxocyclohexa-1,4-dien-1-yl)amino]-6-methoxy-7-(2-methoxyethoxy)quinoline-3-carbonitrile

A solution of 7.7 g (19 mmol) of 4-[(4-chloro-2,5-dimethoxyphenyl)amino]-6-methoxy-7-(2-methoxyethoxy)quinoline-3-carbonitrile in 322 ml of acetonitrile was prepared by boiling. To this solution, 65 ml of water was added. The mixture was stirred and when the temperature reached 30° C., 19 g (34.7 mmol) of ceric ammonium nitrate was added over 5 minutes. After 45 minutes, the mixture was diluted with dilute sodium bicarbonate. The solid was collected by filtration and washed with water. This solid was suspended in 300 ml of water and 35 ml of concentrated hydrochloride acid was added. After stirring for 15 minutes, the precipitated solid was collected. The solid was stirred with 700 ml of methylene chloride and saturated sodium bicarbonate solution. The organic layer was dried over magnesium sulfate and the solution was passed onto a column of Magnesol™. The product was eluted from the column using ethyl acetate. The solvent was evaporated from the product fractions to give a solid that was washed with ether, yielding 1.8 g of the title compound as a red powder: mass spectrum (electrospray, m/e): M−H 414.2.

### EXAMPLE 71

### 4-[(3,6-dioxo-4-phenoxycyclohexa-1,4-dien-1-yl)amino]-6-methoxy-7-(2-methoxyethoxy)quinoline-3-carbonitrile

To a stirred solution of 0.5 g (1.21 mmol) of 4-[(4-chloro-3,6-dioxocyclohexa-1,4-dien-1-yl)amino]-6-methoxy-7-(2-methoxyethoxy)quinoline-3-carbonitrile in 10 ml of dimethylformamide in an ice bath, 0.43 g (2.54 mmol) of sodium phenoxide was added. The mixture was stirred for 30 minutes at room temperature and then diluted with 200 ml of ether and a blue solid collected. This solid was stirred in 200 ml of methylene chloride containing 0.15 ml of acetic acid until the solids dissolved. The solution was poured onto a silica gel column and the product was eluted with chloroform-methanol mixtures. The solvents were evaporated from product fractions yielding 0.4 g of the title compound as an orange solid: mass spectrum (electrospray, m/e): M−H 472.2.

### EXAMPLE 72

### 4-({4-[4-(1H-imidazol-1-yl)phenoxy]-3,6-dioxocyclohexa-1,4-dien-1-yl}amino)-6-methoxy-7-(2-methoxyethoxy)quinoline-3-carbonitrile

This compound was prepared from 4-[(4-chloro-3,6-dioxocyclohexa-1,4-dien-1-yl)amino]-6-methoxy-7-(2-methoxyethoxy)quinoline-3-carbonitrile and the sodium salt of 4-imidazol-1-yl-phenol using the method described above in Example 71. The title compound was obtained as an orange-brown solid: mass spectrum (electrospray, m/e): M−H 538.2, (M+2H)⁺² 269.8.

### EXAMPLE 73

### 2-[(3,4-dimethoxyphenyl)(methyl)amino]-5-{[6-methoxy-7-(2-methoxyethoxy)quinazolin-4-yl]amino}benzo-1,4-quinone

A solution of 0.49 g (1.25 mmol) of 2-chloro-5-{[6-methoxy-7-(2-methoxyethoxy)quinazolin-4-yl]amino}benzo-1,4-quinone and 0.5 ml of N-methyl-3,4-dimethoxy aniline in 10 ml of glyme was stirred at 85° C. for 2 hours. The solvent was evaporated and the residue suspended in ether. The solid was collected via filtration and chromatographed on silica gel eluting with ethyl acetate-methanol 49:1 to give 0.29 g of the title compound as a colored solid: mass spectrum (electrospray, m/e): M−H 521.3

### EXAMPLE 74

### 2-[(3-fluorophenyl)(methyl)amino]-5-{[6-methoxy-7-(2-methoxyethoxy)quinazolin-4-yl]amino}benzo-1,4-quinone

This compound was prepared using the method described above in Example 73 from 2-chloro-5-{[6-methoxy-7-(2-methoxyethoxy)quinazolin-4-yl]amino}benzo-1,4-quinone and N-methyl-3-fluoroaniline. The title compound was obtained as a red solid: mass spectrum (electrospray, m/e): M−H 479.29.

### EXAMPLE 75

### 2-[[4-(dimethylamino)phenyl](methyl)amino]-5-{[6-methoxy-7-(2-methoxyethoxy)quinazolin-4-yl]amino}benzo-1,4-quinone

This compound was prepared using the method described above in Example 73 from 2-chloro-5-{[6-methoxy-7-(2-methoxyethoxy)quinazolin-4-yl]amino}benzo-1,4-quinone and N,N,N′-trimethyl-benzene-1,4-diamine. The title compound was obtained as a dark solid: mass spectrum (electrospray, m/e): M−H 504.1, (M+2H)⁺² 252.6.

### EXAMPLE 76

### 2-{[6-methoxy-7-(2-methoxyethoxy)quinazolin-4-yl]amino}-5-[methyl(phenyl)amino]benzo-1,4-quinone

This compound was prepared using the method described above in Example 73 from 2-chloro-5-{[6-methoxy-7-(2-methoxyethoxy)quinazolin-4-yl]amino}benzo-1,4-quinone and N-methylaniline. The title compound was obtained as the colored acetate salt after recrystallization from acetic acid: mass spectrum (electrospray, m/e): M−H 479.0; mp=239-243° C.

### EXAMPLE 77

### 2-[(4-fluorophenyl)(methyl)amino]-5-{[6-methoxy-7-(2-methoxyethoxy)quinazolin-4-yl]amino}benzo-1,4-quinone

This compound was prepared using the method described above in Example 73 from 2-chloro-5-{[6-methoxy-7-(2-methoxyethoxy)quinazolin-4-yl]amino}benzo-1,4-quinone and N-methyl-4-fluoroaniline. The title compound was obtained as a dark solid: mass spectrum (electrospray, m/e): M−H 479.0.

### EXAMPLE 78

### 2-{[6-methoxy-7-(2-methoxyethoxy)quinazolin-4-yl]amino}-5-[(4-methoxyphenyl)(methyl)amino]benzo-1,4-quinone

This compound was prepared using the method described above in Example 73 from 2-chloro-5-{[6-methoxy-7-(2-methoxyethoxy)quinazolin-4-yl]amino}benzo-1,4-quinone and N-methyl-4-methoxyaniline. The title compound was obtained as a brown solid: mass spectrum (electrospray, m/e): M−H 491.3; mp=197-198° C.

### EXAMPLE 79

### 2-{[6-methoxy-7-(2-methoxyethoxy)quinazolin-4-yl]amino}-5-morpholin-4-ylbenzo-1,4-quinone

A solution of 1.13 g (2.5 mmol) of 2-chloro-5-{[6-methoxy-7-(2-methoxyethoxy)quinazolin-4-yl]amino}benzo-1,4-quinone and 1 ml of morpholine in 30 ml of THF was stirred for 3 hours. The solid was collected via filtration and washed with THF and water to yield, after drying, 1.1 g of the title compound as a red solid: mass spectrum (electrospray, m/e): M−H 441.1; mp=239-243° C.

### EXAMPLE 80

### 2-[cyclohexyl(methyl)amino]-5-{[6-methoxy-7-(2-methoxyethoxy)quinazolin-4-yl]amino}benzo-1,4-quinone

A solution of 1.13 g (2.5 mmol) of 2-chloro-5-{[6-methoxy-7-(2-methoxyethoxy)quinazolin-4-yl]amino}benzo-1,4-quinone and 1 ml cyclohexyl-methyl-amine in 5 ml of glyme was stirred at 85° C. for 4 hours. The solid was collected via filtration and washed with THF to give 0.87 g of the title compound as a red solid: mass spectrum (electrospray, m/e): M−H 467.1; mp=178-180° C.

### EXAMPLE 81

### 2-(dimethylamino)-5-{[6-methoxy-7-(2-methoxyethoxy)quinazolin-4-yl]amino}benzo-1,4-quinone

A solution of 0.97 g (2.5 mmol) of 2-chloro-5-{[6-methoxy-7-(2-methoxyethoxy)quinazolin-4-yl]amino}benzo-1,4-quinone, 0.29 g of pyridine hydrochloride, and 5 ml of 2 M dimethylamine in THF, in 15 ml of THF, was stirred for 3 hours. The solid was collected via filtration and washed with water to give, after drying, 0.94 g of the title compound as a light brown solid: mass spectrum (electrospray, m/e): M−H 399.2.

### EXAMPLE 82

### 2-[benzyl(methyl)amino]-5-{[6-methoxy-7-(2-methoxyethoxy)quinazolin-4-yl]amino}benzo-1,4-quinone

This compound was prepared using the method described above in Example 81 from 2-chloro-5-{[6-methoxy-7-(2-methoxyethoxy)quinazolin-4-yl]amino}benzo-1,4-quinone and N-methyl-benzylamine. The title compound was obtained as a red solid: mass spectrum (electrospray, m/e): M−H 475.2.

### EXAMPLE 83

### 2-{[6-methoxy-7-(2-methoxyethoxy)quinazolin-4-yl]amino}-5-[(3-methylbenzyl)amino]benzo-1,4-quinone

This compound was prepared using the method described above in Example 81 from 2-chloro-5-{[6-methoxy-7-(2-methoxyethoxy)quinazolin-4-yl]amino}benzo-1,4-quinone and 3-methyl benzylamine. The title compound was obtained as an orange solid: mass spectrum (electrospray, m/e): M−H 475.2; mp=241-242° C.

### EXAMPLE 84

### 2-[(6,7-dimethoxyquinazolin-4-yl)amino]-5-morpholin-4-ylbenzo-1,4-quinone

A solution of 1.35 g (4 mmol) of 2-chloro-5-[(6,7-dimethoxy-4-quinazolinyl)amino]benzo-1,4-quinone and 0.696 g (8 mmol) of morpholine in 80 ml of toluene was stirred overnight. The solution is filtered through Magnesol™ using methylene chloride. The resulting solid was collected and washed with ether yielding 0.71 g of the title compound as a red solid: mass spectrum (electrospray, m/e): M−H 397.2; mp=249-250° C.

### EXAMPLE 85

### 2-[(6,7-dimethoxyquinazolin-4-yl)amino]-5-[methyl(phenyl)amino]benzo-1,4-quinone

This compound was prepared using the method described above in Example 73 from 2-chloro-5-[(6,7-dimethoxy-4-quinazolinyl)amino]benzo-1,4-quinone and N-methylaniline and THF as the solvent. The title compound was obtained as a red solid: mass spectrum (electrospray, m/e): M−H 417.3; mp=204-206° C.

### EXAMPLE 86

### 2-anilino-5-[(6,7-dimethoxyquinazolin-4-yl)amino]benzo-1,4-quinone

This compound was prepared using the method described above in Example 73 from 2-chloro-5-[(6,7-dimethoxy-4-quinazolinyl)amino]benzo-1,4-quinone and aniline and THF as the solvent. The title compound was obtained as a red solid: mass spectrum (electrospray, m/e): M−H 403.1; mp=258-261° C.

### EXAMPLE 87

### 2-[(6,7-dimethoxyquinazolin-4-yl)amino]-5-piperidin-1-ylbenzo-1,4-quinone

This compound was prepared using the method described above in Example 84 from 2-chloro-5-[(6,7-dimethoxy-4-quinazolinyl)amino]benzo-1,4-quinone and piperidine and THF as the solvent. The title compound was obtained as a red solid: mass spectrum (electrospray, m/e): M−H 395.2; mp=226-227° C.

### EXAMPLE 88

### 2-[(6,7-dimethoxyquinazolin-4-yl)amino]-5-(dimethylamino)benzo-1,4-quinone

A solution of 1.73 g (5 mmol) of 2-chloro-5-[(6,7-dimethoxy-4-quinazolinyl)amino]benzo-1,4-quinone and 5 ml of 2 M dimethylamine in THF in 20 ml of THF was stirred for 30 hours. The solid was collected via filtration and washed with water to yield, after drying, 1.3 g of the title compound as a light brown solid: mass spectrum (electrospray, m/e): M−H 355.16; mp=245-250° C.

### EXAMPLE 89

### 2-[(6,7-dimethoxyquinazolin-4-yl)amino]-5-(methylamino)benzo-1,4-quinone

This compound was prepared using the method described above in Example 88 from 2-chloro-5-[(6,7-dimethoxy-4-quinazolinyl)amino]benzo-1,4-quinone and 2 M methylamine in THF. The title compound was obtained as a brown solid: mass spectrum (electrospray, m/e): M−H 341.2; mp=283-285° C.

### EXAMPLE 90

### 2-{[6-methoxy-7-(2-methoxyethoxy)quinazolin-4-yl]amino}-5-(2-methylphenoxy)benzo-1,4-quinone

A mixture of 1.03 g (2.5 mmol) of 2-chloro-5-{[6-methoxy-7-(2-methoxyethoxy)quinazolin-4-yl]amino}benzo-1,4-quinone, 1.05 g (9.7 mmol) of 2-methylphenol and 1 g (7.17 mmol) of potassium carbonate in 20 ml of acetone was stirred for 40 hours. The mixture was filtered and solvent evaporated from the filtrate. The original solid and the residue from the filtrate were extracted with methylene chloride. The solvent was evaporated and the resulting solid washed with ether yielding 0.91 g of the title compound as a brown solid: mass spectrum (electrospray, m/e): M−H 462.2; mp 134-137° C.

### EXAMPLE 91

### 2-{[6-methoxy-7-(2-methoxyethoxy)quinazolin-4-yl]amino}-5-(pyridin-3-yloxy)benzo-1,4-quinone

This compound was prepared using the method described above in Example 90 from 2-chloro-5-{[6-methoxy-7-(2-methoxyethoxy)quinazolin-4-yl]amino}benzo-1,4-quinone and 3-hydroxy pyridine. The title compound was obtained as a solid: mass spectrum (electrospray, m/e): M−H 449.1; mp=189-190° C.

### EXAMPLE 92

### 2-{[6-methoxy-7-(2-methoxyethoxy)quinazolin-4-yl]amino}-5-(4-methylphenoxy)benzo-1,4-quinone

This compound was prepared using the method described above in Example 90 from 2-chloro-5-{[6-methoxy-7-(2-methoxyethoxy)quinazolin-4-yl]amino}benzo-1,4-quinone and 4-methylphenol. The title compound was obtained as a red solid: mass spectrum (electrospray, m/e): M−H 462.2; mp=171-172° C.

### EXAMPLE 93

### 2-{[6-methoxy-7-(2-methoxyethoxy)quinazolin-4-yl]amino}-5-phenoxybenzo-1,4-quinone

This compound was prepared using the method described above in Example 90 from 2-chloro-5-{[6-methoxy-7-(2-methoxyethoxy)quinazolin-4-yl]amino}benzo-1,4-quinone and phenol. The title compound is obtained as a red solid: mass spectrum (electrospray, m/e): M−H 448.2; mp=177-180° C.

### EXAMPLE 94

### 2-(4-chlorophenoxy)-5-[(6,7-dimethoxyquinazolin-4-yl)amino]benzo-1,4-quinone

This compound was prepared using the method described above in Example 90 from 2-chloro-5-{[6-methoxy-7-(2-methoxyethoxy)quinazolin-4-yl]amino}benzo-1,4-quinone and 4-chlorophenol. The product was purified by chromatography using a methylene chloride methanol mixture (99:1). The title compound was obtained as a red solid: mass spectrum (electrospray, m/e): M−H 438.25, 440.26.

### EXAMPLE 95

### 2-[(6,7-dimethoxyquinazolin-4-yl)amino]-5-phenoxybenzo-1,4-quinone

This compound was prepared using the method described above in Example 90 from 2-chloro-5-[(6,7-dimethoxy-4-quinazolinyl)amino]benzo-1,4-quinone and phenol. The title compound was obtained as a red solid: mass spectrum (electrospray, m/e): M−H 404.13; mp=228-234° C.

### EXAMPLE 96

### 2-(benzyloxy)-5-{[6-methoxy-7-(2-methoxyethoxy)quinazolin-4-yl]amino}benzo-1,4-quinone

A solution of 0.67 g (1.5 mmol) of 2-{[6-methoxy-7-(2-methoxyethoxy)quinazolin-4-yl]amino}-5-phenoxybenzo-1,4-quinone, 20 ml of benzyl alcohol and 0.5 ml of triethylamine in 20 ml methylene chloride was stirred for 16 hours. The solvent was evaporated and the residue diluted with ether. The solid was collected and washed with ether giving 0.66 g of the title compound as an orange solid: mass spectrum (electrospray, m/e): M−H 462.4; mp=218-220° C.

### EXAMPLE 97

### 2-(2-methoxyethoxy)-5-{[6-methoxy-7-(2-methoxyethoxy)quinazolin-4-yl]amino}benzo-1,4-quinone

This compound was prepared using the method described above in Example 94 from 2-{[6-methoxy-7-(2-methoxyethoxy)quinazolin-4-yl]amino}-5-phenoxybenzo-1,4-quinone, methoxyethanol and triethylamine. The title compound was obtained as a brown solid: mass spectrum (electrospray, m/e): M−H 430.3; mp=211-212° C.

### EXAMPLE 98

### N-(2,5-dimethoxy-1,1′-biphenyl-4-yl)-6,7-dimethoxyquinazolin-4-amine

This compound was prepared by the method of Example 1 given above using 2-amino-4,5-dimethoxy-benzonitrile and 2,5-dimethoxy-biphenyl-4-ylamine. The title compound was obtained as an off-white solid: mass spectrum (electrospray, m/e): M−H 418.1; mp=226-229° C.

### EXAMPLE 99

### 2-[(6,7-dimethoxyquinazolin-4-yl)amino]-5-phenylbenzo-1,4-quinone

This compound was prepared by the method of Example 2 given above from N-(2,5-dimethoxy-1,1′-biphenyl-4-yl)-6,7-dimethoxyquinazolin-4-amine and ceric ammonium nitrate. The title compound was obtained as a brown solid: mass spectrum (electrospray, m/e): M−H 388.1; mp=200-205° C.

### EXAMPLE 100

### 4-[(6,7-dimethoxyquinazolin-4-yl)amino]-1-phenyl-7-oxabicyclo[4.1.0]hept-3-ene-2,5-dione

This compound was prepared from 2-[(6,7-dimethoxyquinazolin-4-yl)amino]-5-phenylbenzo-1,4-quinone and hydrogen peroxide using the method described above in Example 5. The title compound was obtained as a yellow solid: mass spectrum (electrospray, m/e): M+H 404.1; mp=252-253° C.

### EXAMPLE 101

### 2-{[6-methoxy-7-(2-methoxyethoxy)quinazolin-4-yl]amino}-5-piperidin-1-yl-benzo-1,4-quinone

This compound was prepared using the method described above in Example 81 from 2-chloro-5-{[6-methoxy-7-(2-methoxyethoxy)quinazolin-4-yl]amino}benzo-1,4-quinone and piperidine. The title compound was obtained as a solid: mass spectrum (electrospray, m/e): M−H 439.3; mp=197-200° C.

### EXAMPLE 102

### (1,4-dimethoxy-naphthalen-2-yl)-[6-methoxy-7-(2-methoxy-ethoxy)-quinazolin-4-yl]-amine

This compound was prepared by the method described above in Example 16 using N′-[2-cyano-4-methoxy-5-(2-methoxyethoxy)phenyl]-N,N-dimethylimidoformamide and 1,4-dimethoxy-naphthalen-2-ylamine (Syn. Comm., 16:81-687 (1986)). The product was recrystallized from isopropanol yielding the title compound as a light grey solid: mass spectrum (electrospray, m/e): M+H 436.2.

### EXAMPLE 103

### 2-[6-methoxy-7-(2-methoxyethoxy)-quinazolin-4-ylamino]-[1,4]naphthoquinone

This compound was prepared by the method of Example 17 described above using (1,4-dimethoxy-naphthalen-2-yl)-[6-methoxy-7-(2-methoxy-ethoxy)-quinazolin-4-yl]-amine and ceric ammonium nitrate. After passing the solution through Magnesol™, the filtrate was concentrated and the solid collected and washed with ether. The title compound was obtained as an orange solid: mass spectrum (electrospray, m/e): M+H 406.2.

### EXAMPLE 104

### 2-(2-hydroxyethyl)thio)-3-[6-methoxy-7-(2-methoxyethoxy)-quinazolin-4-ylamino]-[1,4]naphthoquinone

A solution of 0.7 g (1.73 mmol) of 2-[6-methoxy-7-(2-methoxyethoxy)-quinazolin-4-ylamino]-[1,4]naphthoquinone and 0.27 g (3.45 mmol) of mercaptoethanol was stirred at room temperature for 5 days. To the solution, 0.21 g of 2,3-dichloro-5,6-dicyano-1,4-benzoquinone (DDQ) was added. After 10 minutes, the mixture was poured into dilute sodium carbonate and extracted with chloroform. The organic layer was dried over magnesium sulfate and passed through a column of Magnesol™ eluting with a mixture of chloroform and isopropanol. The solvent was evaporated and the residue chromatographed on silica gel eluting with chloroform and then with chloroform-isopropanol mixtures. The solvent was evaporated from product fractions and the residue recrystallized from isopropanol to give 0.47 g of the title compound as an orange solid: mass spectrum (electrospray, m/e): M+H 482.1.

### EXAMPLE 105

### 2-(methoxy)-5-{[6-methoxy-7-(2-methoxyethoxy)quinazolin-4-yl]amino}benzo-1,4-quinone

This compound was prepared by the method of Example 94 using 2-{[6-methoxy-7-(2-methoxyethoxy)quinazolin-4-yl]amino}-5-phenoxybenzo-1,4-quinone, methanol, and triethylamine in methylene chloride. The product was purified on silica gel eluting with methylene chloride-methanol 39:1, to yield the title compound as a red solid.

### EXAMPLE 106

### 4-{[6-methoxy-7-(2-methoxyethoxy)-4-quinazolinyl]amino}-1-methyl-7-oxabicyclo[4.1.0]hept-3-ene-2,5-dione

This compound was prepared by the method of Example 5 using 2-{[6-methoxy-7-(2-methoxyethoxy)-4-quinazolinyl]amino}-5-methylbenzo-1,4-quinone (Example 15) and hydrogen peroxide. The title compound was obtained as a yellow solid: mass spectrum (electrospray, m/e): M+H 386.13.

### EXAMPLE 107

### 4-[(6,7-dimethoxy-4-quinazolinyl)amino]-1-isopropyl-7-oxabicyclo[4.1.0]hept-3-ene-2,5-dione

This compound was prepared from N-(4-chloro-2,5-dimethoxyphenyl)-6,7-dimethoxy-4-quinazolinamine and 4-isopropyl-2,5-dimethoxy-phenylamine using the methods of Examples 2, 3 and 5, sequentially. The title compound was obtained as a solid: mass spectrum (electrospray, m/e): M+H 370.21; mp=188-190° C.

### EXAMPLE 108

### 1-benzyl-4-{[6-methoxy-7-(2-methoxyethoxy)quinazolin-4-yl]amino}-7-oxabicyclo[4.1.0]hept-3-ene-2,5-dione

This compound was prepared from N-(4-chloro-2,5-dimethoxyphenyl)-6,7-dimethoxy-4-quinazolinamine and 4-benzyl-2,5-dimethoxy-phenylamine using the methods Examples 2, 3 and 5, sequentially. The title compound was obtained as a solid: mass spectrum (electrospray, m/e): M+H 462.2; mp=104-108° C.

### EXAMPLE 109

### 4-[(6,7-dimethoxy-4-quinazolinyl)amino]-1-ethyl-7-oxabicyclo[4.1.0]hept-3-ene-2,5-dione

This compound was prepared from N-(4-chloro-2,5-dimethoxyphenyl)-6,7-dimethoxy-4-quinazolinamine and 4-ethyl-2,5-dimethoxy-phenylamine using the methods of Examples 2, 3 and 5, sequentially. The title compound was obtained as a solid: mp=202-204° C.

### EXAMPLE 110

### 2-chloro-5-methoxy-3-[6-methoxy-7-(2-methoxyethoxy)-quinazolin-4-ylamino]-benzo-1,4-quinone

A solution of 2 g of 2-methoxy-6-{[6-methoxy-7-(2-methoxyethoxy)quinazolin-4-yl]amino}benzo-1,4-quinone was prepared by boiling in 50 ml of chloroform. The solution was cooled to room temperature and 2 ml of chloroform saturated with hydrogen chloride was added. This mixture was stirred overnight. The solvent was evaporated giving the hydroquinone as a yellow-brown solid. This solid was dissolved in 50 ml of acetonitrile and 10 ml of water to which 1.2 g of DDQ was added. After 1 hour, the mixture was poured into saturated sodium bicarbonate and extracted several times with methylene chloride. The extract was dried over magnesium sulfate and solvent evaporated. The product was purified by chromatography eluting with ethyl acetate-isopropanol mixtures. Product fractions were combined and solvent evaporated yielding 1.2 g of the title compound as a yellow solid: mass spectrum (electrospray, m/e): M+H 420.0.

### EXAMPLE 111

### 5-methoxy-3-{[6-methoxy-7-(2-methoxyethoxy)quinazolin-4-yl]amino}-2-(pyridin-2-ylthio)benzo-1,4-quinone

This compound was prepared from 2-methoxy-6-{[6-methoxy-7-(2-methoxyethoxy)quinazolin-4-yl]amino}benzo-1,4-quinone and 2-mercaptopyridine using the method described in Example 18 above using a 30 minute initial reaction time. The title compound was obtained as a red powder: mass spectrum (electrospray, m/e): M+H 495.0.

### EXAMPLE 112

### 2-[ethyl(methyl)amino]-5-{[6-methoxy-7-(2-methoxyethoxy)quinazolin-4-yl]amino}benzo-1,4-quinone

A solution of 0.20 g (0.51 mmol) of 2-chloro-5-{[6-methoxy-7-(2-methoxyethoxy)quinazolin-4-yl]amino}benzo-1,4-quinone, 0.06 g of pyridine hydrochloride and 0.12 g methylethylamine in 2 ml of tetrahydrofuran was sonicated for 0.5 hour at 40° C., then shaken at 40° C. for 3 hours. The solid was collected via filtration and washed with water to give, after drying, 0.165 g of the title compound as a light brown solid: mass spectrum (electrospray, m/e): M+H 413.2.

### EXAMPLES 113-143

The following examples in Table 9 were prepared from 2-chloro-5-{[6-methoxy-7-(2-methoxyethoxy)quinazolin-4-yl]amino}benzo-1,4-quinone and the appropriate amine using the procedure outlined above in Example 112.

TABLE 9

| | | MS m/e | MS m/e |
|---|---|---|---|
| Example | Compound Name | (M + H)⁺ | (M + 2H)²⁺ |
| 113 | 2-(diisobutylamino)-5-{[6-methoxy-7-(2- | 483.3 | |
| | methoxyethoxy)quinazolin-4-yl]amino}benzo-1,4- | | |
| | quinone | | |
| 114 | 2-(2,5-dimethylpyrrolidin-1-yl)-5-{[6-methoxy-7-(2- | 453.2 | 227.1 |
| | methoxyethoxy)quinazolin-4-yl]amino}benzo-1,4- | | |
| | quinone | | |
| 115 | 2-(3,5-dimethylpiperidin-1-yl)-5-{[6-methoxy-7-(2- | 467.2 | |
| | methoxyethoxy)quinazolin-4-yl]amino}benzo-1,4- | | |
| | quinone | | |
| 116 | 2-{[6-methoxy-7-(2-methoxyethoxy)quinazolin-4- | 453.2 | |
| | yl]amino}-5-(3-methylpiperidin-1-yl)benzo-1,4- | | |
| | quinone | | |
| 117 | 2-[(2,3-dihydroxypropyl)(methyl)amino]-5-{[6- | 459.2 | |
| | methoxy-7-(2-methoxyethoxy)quinazolin-4- | | |
| | yl]amino}benzo-1,4-quinone | | |
| 118 | 2-{[6-methoxy-7-(2-methoxyethoxy)quinazolin-4- | 411.2 | |
| | yl]amino}-5-(2-methylaziridin-1-yl)benzo-1,4- | | |
| | quinone | | |
| 119 | 2-[(2R,6S)-2,6-dimethylmorpholin-4-yl]-5-{[6- | 469.2 | |
| | methoxy-7-(2-methoxyethoxy)quinazolin-4- | | |
| | yl]amino}benzo-1,4-quinone | | |
| 120 | 2-(dipropylamino)-5-{[6-methoxy-7-(2- | 455.2 | |
| | methoxyethoxy)quinazolin-4-yl]amino}benzo-1,4- | | |
| | quinone | | |
| 121 | 2-{[6-methoxy-7-(2-methoxyethoxy)quinazolin-4- | 516.2 | 258.6 |
| | yl]amino}-5-(2-pyridin-3-ylpiperidin-1-yl)benzo-1,4- | | |
| | quinone | | |
| 122 | tert-butyl 1-(4-{[6-methoxy-7-(2- | 525.2 | |
| | methoxyethoxy)quinazolin-4-yl]amino}-3,6- | | |
| | dioxocyclohexa-1,4-dien-1-yl)-L-prolinate | | |
| 123 | 2-azocan-1-yl-5-{[6-methoxy-7-(2- | 467.2 | |
| | methoxyethoxy)quinazolin-4-yl]amino}benzo-1,4- | | |
| | quinone | | |
| 124 | 2-{[6-methoxy-7-(2-methoxyethoxy)quinazolin-4- | 455.2 | |
| | yl]amino}-5-[methyl(pentyl)amino]benzo-1,4- | | |
| | quinone | | |
| 125 | 2-{4-[4-chloro-3-(trifluoromethyl)phenyl]piperazin-1- | 618.2 | |
| | yl}-5-{[6-methoxy-7-(2-methoxyethoxy)quinazolin-4- | | |
| | yl]amino}benzo-1,4-quinone | | |
| 126 | 2-{[6-methoxy-7-(2-methoxyethoxy)quinazolin-4- | 508.2 | 254.6 |
| | yl]amino}-5-[(2S)-2-(pyrrolidin-1-ylmethyl)pyrrolidin- | | |
| | 1-yl]benzo-1,4-quinone | | |
| 127 | 2-[4-(2-fluoro-4-nitrophenyl)piperazin-1-yl]-5-{[6- | 579.2 | |
| | methoxy-7-(2-methoxyethoxy)quinazolin-4- | | |
| | yl]amino}benzo-1,4-quinone | | |
| 128 | 2-[[(3S)-1-benzylpyrrolidin-3-yl](methyl)amino]-5- | 544.2 | 272.6 |
| | {[6-methoxy-7-(2-methoxyethoxy)quinazolin-4- | | |
| | yl]amino}benzo-1,4-quinone | | |
| 129 | 2-(4-benzylpiperidin-1-yl)-5-{[6-methoxy-7-(2- | 529.2 | |
| | methoxyethoxy)quinazolin-4-yl]amino}benzo-1,4- | | |
| | quinone | | |
| 130 | 2-[4-(2-hydroxyethyl)piperazin-1-yl]-5-{[6-methoxy- | 484.2 | 242.6 |
| | 7-(2-methoxyethoxy)quinazolin-4-yl]amino}benzo- | | |
| | 1,4-quinone | | |
| 131 | 2-{[6-methoxy-7-(2-methoxyethoxy)quinazolin-4- | 518.2 | 259.6 |
| | yl]amino}-5-(4-pyrazin-2-ylpiperazin-1-yl)benzo-1,4- | | |
| | quinone | | |
| 132 | 2-[[2-(1H-indol-3-yl)ethyl](methyl)amino]-5-{[6- | 528.2 | |
| | methoxy-7-(2-methoxyethoxy)quinazolin-4- | | |
| | yl]amino}benzo-1,4-quinone | | |
| 133 | ethyl 1-(4-{[6-methoxy-7-(2- | 511.2 | |
| | methoxyethoxy)quinazolin-4-yl]amino}-3,6- | | |
| | dioxocyclohexa-1,4-dien-1-yl)piperidine-4- | | |
| | carboxylate | | |
| 134 | 2-{[6-methoxy-7-(2-methoxyethoxy)quinazolin-4- | 545.2 | |
| | yl]amino}-5-[4-(2-methoxyphenyl)piperidin-1- | | |
| | yl]benzo-1,4-quinone | | |
| 135 | 2-(4-benzyl-1,4-diazepan-1-yl)-5-{[6-methoxy-7-(2- | 544.2 | 272.6 |
| | methoxyethoxy)quinazolin-4-yl]amino}benzo-1,4- | | |
| | quinone | | |
| 136 | 2-(1,4′-bipiperidin-1′-yl)-5-{[6-methoxy-7-(2- | 522.3 | 261.6 |
| | methoxyethoxy)quinazolin-4-yl]amino}benzo-1,4- | | |
| | quinone | | |
| 137 | 2-[[2-(3,4-dimethoxyphenyl)ethyl](methyl)amino]-5- | 549.2 | |
| | {[6-methoxy-7-(2-methoxyethoxy)quinazolin-4- | | |
| | yl]amino}benzo-1,4-quinone | | |
| 138 | tert-butyl N-(4-{[6-methoxy-7-(2- | 499.2 | |
| | methoxyethoxy)quinazolin-4-yl]amino}-3,6- | | |
| | dioxocyclohexa-1,4-dien-1-yl)-N-methylglycinate | | |
| 139 | 2-{[6-methoxy-7-(2-methoxyethoxy)quinazolin-4- | 537.2 | 269.1 |
| | yl]amino}-5-[4-(2-pyrrolidin-1-ylethyl)piperazin-1- | | |
| | yl]benzo-1,4-quinone | | |
| 140 | 2-{[6-methoxy-7-(2-methoxyethoxy)quinazolin-4- | 537.3 | 269.1 |
| | yl]amino}-5-[4-(1-methylpiperidin-4-yl)piperazin-1- | | |
| | yl]benzo-1,4-quinone | | |
| 141 | 2-{[6-methoxy-7-(2-methoxyethoxy)quinazolin-4- | 489.2 | |
| | yl]amino}-5-[methyl(2-phenylethyl)amino]benzo- | | |
| | 1,4-quinone | | |
| 142 | 2-[4-(ethylsulfonyl)piperazin-1-yl]-5-{[6-methoxy-7- | 532.1 | |
| | (2-methoxyethoxy)quinazolin-4-yl]amino}benzo-1,4- | | |
| | quinone | | |
| 143 | 2-{[6-methoxy-7-(2-methoxyethoxy)quinazolin-4- | 425.2 | |
| | yl]amino}-5-pyrrolidin-1-ylbenzo-1,4-quinone | | |

### EXAMPLE 144

### 2-(2,3-dihydro-1,4-benzooxazepin-4(5H)-yl)-5-[6-methoxy-7-(2-methoxyethoxy)quinazolin-4-yl]amino}-benzo-1,4-quinone

A slurry of 0.075 g (0.19 mmol) of 2-chloro-5-{[6-methoxy-7-(2-methoxyethoxy)quinazolin-4-yl]amino}benzo-1,4-quinone, 0.04 g of pyridine hydrochloride, 0.1 ml of N,N-diisopropylethylamine and 0.14 g of 2,3,4,5-tetrahydro-benzo[f][1,4]oxazepine hydrochloride in 2 ml of tetrahydrofuran was sonicated for 0.5 hour at 40° C., then shaken at 40° C. for 3 hours. The solid was collected via filtration, washed with tetrahydrofuran, then water and dried in vacuo to give 0.05 g of the title compound as a tan solid: mass spectrum (electrospray, m/e): M+H 503.2.

### EXAMPLES 145-146

The following examples in Table 10 were prepared from 2-chloro-5-{[6-methoxy-7-(2-methoxyethoxy)quinazolin-4-yl]amino}benzo-1,4-quinone and the appropriate amine hydrochloride salt using the procedure outlined above for Example 144.

TABLE 10

| | | MS m/e |
|---|---|---|
| Example | Compound Name | (M + H)⁺ |
| 145 | 2-{4-hydroxy-4-[3-(trifluormethyl)phenyl- | 599.2 |
| | ]piperidin-1-yl]-5-{[6-methoxy-7- | |
| | (2-methoxyethoxy)quinazolin-4- | |
| | yl]amino}benzo-1,4-quinone | |
| 146 | 2-[(1R,4R)-5-(4-chlorophenyl)-2,5- | 562.2 |
| | diazabicyclo[2.2.1]hept-2-yl]-5-{[6-methoxy- | |
| | 7-(2-methoxyethoxy)quinazolin-4- | |
| | yl]amino}benzo-1,4-quinone | |

### EXAMPLE 147

### 1-{4-[6-methoxy-7-(2-methoxyethoxy)-quinazolin-4-ylamino]-3,6-dioxo-cyclohexa-1,4-dien-1-yl}-piperidine-4-carboxylic acid

A slurry of 0.10 g (0.26 mmol) of 2-chloro-5-{[6-methoxy-7-(2-methoxyethoxy)quinazolin-4-yl]amino}benzo-1,4-quinone, 0.001-0.002 g of 4-(dimethylamino)pyridine and 0.105 g of piperidine-4-carboxylic acid in 2 ml of N,N-dimethylformamide was stirred for 24 hours. The reaction mixture was then diluted with water and the precipitated solid was collected by filtration, washed with water and dried in vacuo to give 0.11 g of the title compound as a red-brown solid: mass spectrum (electrospray, m/e): M+H 483.2.

### EXAMPLE 148

### 1-(4-{[6-methoxy-7-(2-methoxyethoxy)quinazolin-4-yl]amino}-3,6-dioxocyclohexa-1,4-dien-1-yl)azetidine-3-carboxylic acid

The title compound was prepared from 2-chloro-5-{[6-methoxy-7-(2-methoxyethoxy)quinazolin-4-yl]amino}benzo-1,4-quinone and azetidine-3-carboxylic acid using the procedure described above in Example 147: mass spectrum (electrospray, m/e): M+H 455.1.

### EXAMPLE 149

### 2-[[2-(diethylamino)ethyl](methyl)amino]-5-{[6-methoxy-7-(2-methoxyethoxy)quinazolin-4-yl]amino}benzo-1,4-quinone

A solution of 0.12 g (0.31 mmol) of 2-chloro-5-{[6-methoxy-7-(2-methoxyethoxy)quinazolin-4-yl]amino}benzo-1,4-quinone and 0.52 ml (3.2 mmol) of N,N-diethyl-N-methylethylenediamine in 1.5 ml of dioxane was treated with either 0.11 g (0.93 mmol) of pyridine hydrochloride or 0.86 ml (4.9 mmol) N,N-diisopropylethylamine. The mixture was then heated via microwave irradiation at 75 to 125° C. for 5 minutes. The crude product was then directly purified by reverse phase chromatography using gradient elution with acetonitrile and water containing 0.05% trifluoroacetic acid to give 0.11 g of the title compound: mass spectrum (electrospray, m/e): M+H 484.3.

### EXAMPLES 150-173

The following examples in Table 11 were prepared from 2-chloro-5-{[6-methoxy-7-(2-methoxyethoxy)quinazolin-4-yl]amino}benzo-1,4-quinone and the appropriate amine using the procedure outlined above for Example 149.

TABLE 11

| | | MS m/e | MS m/e |
|---|---|---|---|
| Example | Compound Name | (M + H)⁺ | (M + 2H)²⁺ |
| 150 | 2-{[6-methoxy-7-(2-methoxyethoxy)quinazolin-4- | 493.2 | |
| | yl]amino}-5-[2-(trifluoromethyl)pyrrolidin-1-yl]benzo- | | |
| | 1,4-quinone | | |
| 151 | N,N-diethyl-1-(4-{[6-methoxy-7-(2- | 538.2 | 269.6 |
| | methoxyethoxy)quinazolin-4-yl]amino}-3,6- | | |
| | dioxocyclohexa-1,4-dien-1-yl)piperidine-3- | | |
| | carboxamide | | |
| 152 | ethyl 1-(4-{[6-methoxy-7-(2- | 511.2 | |
| | methoxyethoxy)quinazolin-4-yl]amino}-3,6- | | |
| | dioxocyclohexa-1,4-dien-1-yl)piperidine-3- | | |
| | carboxylate | | |
| 153 | 2-(4-benzylpiperazin-1-yl)-5-{[6-methoxy-7-(2- | 530.2 | 265.6 |
| | methoxyethoxy)quinazolin-4-yl]amino}benzo-1,4- | | |
| | quinone | | |
| 154 | 2-[(1,3-dioxolan-2-ylmethyl)(methyl)amino]-5-{[6- | 471.2 | |
| | methoxy-7-(2-methoxyethoxy)quinazolin-4- | | |
| | yl]amino}benzo-1,4-quinone | | |
| 155 | 2-[[2-(dimethylamino)ethyl(methyl)amino]-5-{[6- | 456.2 | 228.6 |
| | methoxy-7-(2-methoxyethoxy)quinazolin-4- | | |
| | yl]amino}benzo-1,4-quinone | | |
| 156 | 2-[(cyclopropylmethyl)(propyl)amino]-5-{[6- | 467.2 | |
| | methoxy-7-(2-methoxyethoxy)quinazolin-4- | | |
| | yl]amino}benzo-1,4-quinone | | |
| 157 | 2-[(2-methoxyethyl)(methyl)amino]-5-{[6-methoxy- | 443.2 | |
| | 7-(2-methoxyethoxy)quinazolin-4-yl]amino}benzo- | | |
| | 1,4-quinone | | |
| 158 | 2-[6-methoxy-7-(3-methoxy-propyl)-quinazolin-4- | 454.2 | 227.6 |
| | ylamino]-5-(3-methylamino-pyrrolidin-1-yl)- | | |
| | [1,4]benzoquinone | | |
| 159 | 2-[isobutyl(methyl)amino]-5-{[6-methoxy-7-(2- | 441.2 | |
| | methoxyethoxy)quinazolin-4-yl]amino}benzo-1,4- | | |
| | quinone | | |
| 160 | 2-(4-ethylpiperazin-1-yl)-5-{[6-methoxy-7-(2- | 468.2 | 234.6 |
| | methoxyethoxy)quinazolin-4-yl]amino}benzo-1,4- | | |
| | quinone | | |
| 161 | 2-[butyl(methyl)amino]-5-{[6-methoxy-7-(2- | 441.2 | |
| | methoxyethoxy)quinazolin-4-yl]amino}benzo-1,4- | | |
| | quinone | | |
| 162 | 2-{[6-methoxy-7-(2-methoxyethoxy)quinazolin-4- | 482.5 | |
| | yl]amino}-5-[methyl(1-methylpiperidin-4- | | |
| | yl)amino]benzo-1,4-quinone | | |
| 163 | 2-[3-(hydroxymethyl)piperidin-1-yl]-5-{[6-methoxy- | 469.2 | |
| | 7-(2-methoxyethoxy)quinazolin-4-yl]amino}benzo- | | |
| | 1,4-quinone | | |
| 164 | 2-(4-acetylpiperazin-1-yl)-5-{[6-methoxy-7-(2- | 482.2 | |
| | methoxyethoxy)quinazolin-4-yl]amino}benzo-1,4- | | |
| | quinone | | |
| 165 | 2-{[6-methoxy-7-(2-methoxyethoxy)quinazolin-4- | 468.5 | |
| | yl]amino}-5-[methyl(1-methylpyrrolidin-3- | | |
| | yl)amino]benzo-1,4-quinone | | |
| 166 | 2-[[3-(dimethylamino)propyl](methyl)amino]-5-{[6- | 470.5 | 235.7 |
| | methoxy-7-(2-methoxyethoxy)quinazolin-4- | | |
| | yl]amino}benzo-1,4-quinone | | |
| 167 | 2-(diallylamino)-5-{[6-methoxy-7-(2- | 451.2 | |
| | methoxyethoxy)quinazolin-4-yl]amino}benzo-1,4- | | |
| | quinone | | |
| 168 | 2-[(2-furylmethyl)(methyl)amino]-5-{[6-methoxy-7- | 465.1 | |
| | (2-methoxyethoxy)quinazolin-4-yl]amino}benzo-1,4- | | |
| | quinone | | |
| 169 | 2-{[6-methoxy-7-(2-methoxyethoxy)quinazolin-4- | 532.2 | |
| | yl]amino}-5-[(4-morpholin-4-ylphenyl)amino]benzo- | | |
| | 1,4-quinone | | |
| 170 | 2-[allyl(methyl)amino]-5-{[6-methoxy-7-(2- | 425.2 | |
| | methoxyethoxy)quinazolin-4-yl]amino}benzo-1,4- | | |
| | quinone | | |
| 171 | 2-(2,3-dihydro-1,4-benzodioxin-6-ylamino)-5-{[6- | 505.2 | |
| | methoxy-7-(2-methoxyethoxy)quinazolin-4- | | |
| | yl]amino}benzo-1,4-quinone | | |
| 172 | 2-[(4-isopropylphenyl)amino]-5-{[6-methoxy-7-(2- | 489.5 | |
| | methoxyethoxy)quinazolin-4-yl]amino}benzo-1,4- | | |
| | quinone | | |
| 173 | 2-[(2-ethylphenyl)amino]-5-{[6-methoxy-7-(2- | 475.2 | |
| | methoxyethoxy)quinazolin-4-yl]amino}benzo-1,4- | | |
| | quinone | | |

### EXAMPLE 174

### 2-[(9-ethyl-9H-carbazol-3-yl)amino]-5-{[6-methoxy-7-(2-methoxyethoxy)quinazolin-4-yl]amino}benzo-1,4-quinone

A solution of 0.10 g (0.26 mmol) of 2-chloro-5-{[6-methoxy-7-(2-methoxyethoxy)quinazolin-4-yl]amino}benzo-1,4-quinone, 0.095 g (0.77 mmol) of pentafluorophenol, 0.16 g (0.77 mmol) of 3-amino-9-ethylcarbazole and 0.11 g (0.77 mmol) of potassium carbonate in 4.0 ml of acetone was heated to 45° C. for 3 hours. The crude product was then diluted with water and extracted three times with methylene chloride. The combined extracts were dried over anhydrous sodium sulfate, filtered, concentrated in vacuo, and purified by reverse phase chromatography to yield 0.04 g of the title compound: mass spectrum (electrospray, m/e): M+H 564.2.

### EXAMPLES 175-180

The following examples in Table 12 were prepared from 2-chloro-5-{[6-methoxy-7-(2-methoxyethoxy)quinazolin-4-yl]amino}benzo-1,4-quinone and the appropriate amine using the procedure outlined above for Example 174.

TABLE 12

| | | MS |
|---|---|---|
| Example | Compound Name | m/e (M + H)⁺ |
| 175 | 2-[ethyl(3-methylphenyl)amino]-5-{[6-methoxy-7-(2- | 489.2 |
| | methoxyethoxy)quinazolin-4-yl]amino}benzo-1,4-quinone | |
| 176 | 2-[(3,5-di-tert-butylphenyl)amino]-5-{[6-methoxy-7- | 559.3 |
| | (2-methoxyethoxy)quinazolin-4-yl]amino}benzo-1,4- | |
| | quinone | |
| 177 | 2-{[4-(4-chlorophenoxy)phenyl]amino}-5-{[6- | 574.2 |
| | methoxy-7-(2-methoxyethoxy)quinazolin-4- | |
| | yl]amino}benzo-1,4-quinone | |
| 178 | ethyl 5-{4-[(4-{[6-methoxy-7-(2- | 599.2 |
| | methoxyethoxy)quinazolin-4-yl]amino}-3,6- | |
| | dioxocyclohexa-1,4-dien-1-yl)amino]phenyl}-2- | |
| | methyl-3-furoate | |
| 179 | 2-(4-imidazol-1-yl-phenylamino)-5-[6-methoxy-7-(3- | 513.5 |
| | methoxypropyl)-quinazolin-4-ylamino]benzo-1,4- | |
| | quinone | |
| 180 | N-(4-{[6-methoxy-7-(2-methoxyethoxy)quinazolin-4- | 471.2 |
| | yl]amino}-3,6-dioxocyclohexa-1,4-dien-1-yl)-L- | |
| | valine | |

### EXAMPLE 181

### 2-{[6-methoxy-7-(2-methoxyethoxy)quinazolin-4-yl]amino}-5-(pentafluorophenoxy)benzo-1,4-quinone

A solution of 0.11 g (0.28 mmol) of 2-chloro-5-{[6-methoxy-7-(2-methoxyethoxy)quinazolin-4-yl]amino}benzo-1,4-quinone, 0.10 g (0.56 mmol) of pentafluorophenol and 0.11 g (0.83 mmol) of potassium carbonate in 3.0 ml of acetone was heated to 45° C. for 1.5 hours. The crude product was then diluted with water and extracted three times with methylene chloride. The combined extracts were dried over anhydrous sodium sulfate, filtered, concentrated in vacuo, and purified by chromatography over silica gel to give 0.03 g of the title compound: mass spectrum (electrospray, m/e): M+H 538.1.

### EXAMPLE 182

### 2-{[6-methoxy-7-(2-methoxyethoxy)quinazolin-4-yl]amino}-5-[(2-methoxypropyl)amino]benzo-1,4-quinone

To 0.05 g (0.12 mmol) of 2-{[6-methoxy-7-(2-methoxyethoxy)quinazolin-4-yl]amino}-5-(2-methylaziridin-1-yl)benzo-1,4-quinone, 56 ml each of tetrahydrofuran, methanol, and water were added. After stirring for 18 hours, the solution was concentrated and the aqueous layer extracted three times with methylene chloride. The mixture was dried over anhydrous sodium sulfate, filtered, concentrated in vacuo, and the desired product was isolated by chromatography over silica gel using a mixture of methylene chloride and isopropyl alcohol as eluant, to give 0.017 g of the title compound as a red-brown solid: mass spectrum (electrospray, m/e): M+H⁺ 443.2.

### EXAMPLE 183

### 2-[(2-hydroxypropyl)amino]-5-{[6-methoxy-7-(2-methoxyethoxy)quinazolin-4-yl]amino}benzo-1,4-quinone

To a stirred solution of 0.25 g (0.64 mmol) 2-{[6-methoxy-7-(2-methoxyethoxy)quinazolin-4-yl]amino}-5-(2-methylaziridin-1-yl)benzo-1,4-quinone in 250 ml tetrahydrofuran and 250 ml water, concentrated hydrochloric acid was added until the reaction mixture reached a pH of 4. After 18 hours, the solution was concentrated and the aqueous layer extracted three times with methylene chloride. The mixture was dried over anhydrous sodium sulfate, filtered, concentrated in vacuo, and the product was purified by chromatography over silica gel, using a mixture of methylene chloride and isopropyl alcohol as eluant, to give 0.19 g of the title compound as a red-brown solid: mass spectrum (electrospray, m/e): M+H⁺ 429.2.

### EXAMPLE 184

### 2-{[6-methoxy-7-(2-methoxyethoxy)quinazolin-4-yl]amino}-5-(5-methyl-2-oxo-1,3-oxazolidin-3-yl)benzo-1,4-quinone

To a solution of 0.13 g (0.30 mmol) 2-[(2-hydroxypropyl)amino]-5-{[6-methoxy-7-(2-methoxyethoxy)quinazolin-4-yl]amino}benzo-1,4-quinone, 0.49 g 1,1″-carbonyl diimidazole in 10 ml of 1-methyl-2-pyrrolidinone was added. After stirring for 27 hours at 80° C. under an atmosphere of nitrogen, the solution was poured into 100 ml of water and extracted three times with ethyl acetate. The mixture was dried over anhydrous sodium sulfate, filtered, concentrated in vacuo, and the product was purified by chromatography over silica gel using a mixture of methylene chloride and isopropyl alcohol as eluant, to give 0.06 g of the title compound as a red solid: mass spectrum (electrospray, m/e): M+H⁺ 455.1.

### EXAMPLE 185

### 3-iodo-2-{[6-methoxy-7-(2-methoxyethoxy)quinazolin-4-yl]amino}-5-methylbenzo-1,4-quinone

A solution of 1 g (2.71 mmol) of 2-{[6-methoxy-7-(2-methoxyethoxy)-4-quinazolinyl]amino}-5-methylbenzo-1,4-quinone (Example 15) and 0.755 g (2.98 mmol) of iodine in 10 ml of pyridine was stirred for 2 hours. The mixture was poured onto a column of Magnasol™ and product was eluted with chloroform-isopropanol mixtures to give 1.12 g of the title compound as a black powder: mass spectrum (electrospray, m/e): M+H⁺ 495.9.

### EXAMPLE 186

### 3-[(2-hydroxyethyl)thiol]-2-{[6-methoxy-7-(2-methoxyethoxy)quinazolin-4-yl]amino}-5-methylbenzo-1,4-quinone

A solution of 0.625 g (1.26 mmol) of 3-iodo-2-{[6-methoxy-7-(2-methoxyethoxy)quinazolin-4-yl]amino}-5-methylbenzo-1,4-quinone (Example 185) and 0.14 g (1.77 mmol) of mercaptoethanol in 20 ml of methylene chloride was stirred for 3 hours. To the solution was added 0.34 g of 2,3-dichloro-5,6-dicyano-1,4-benzoquinone (DDQ). After 15 minutes, the mixture was diluted with methylene chloride and washed with dilute potassium carbonate. The organic solution was dried and passed through a column of silica gel. The product was eluted with ethyl acetate-isopropanol mixtures: mass spectrum (electrospray, m/e): M+H⁺ 446.1.

### EXAMPLE 187

### 2-iodo-5-methoxy-3-{[6-methoxy-7-(2-methoxyethoxy)quinazolin-4-yl]amino}benzo-1,4-quinone

A solution of 1 g of 2-methoxy-6-{[6-methoxy-7-(2-methoxyethoxy)quinazolin-4-yl]amino}benzo-1,4-quinone (Example 17) and 0.79 g of iodine in 10 ml of methylene chloride was stirred for 7 days. The mixture was poured unto a column of Magnasol™ and product was eluted with ethyl acetate-isopropanol 10:1 giving 0.58 g of an orange powder: mass spectrum (electrospray, m/e): M+H⁺ 511.9.

### EXAMPLE 188

### 2-amino-5-{[6-methoxy-7-(2-methoxyethoxy)quinazolin-4-yl]amino}benzo-1,4-quinone

A solution of 0.7 g of 2-chloro-5-{[6-methoxy-7-(2-methoxyethoxy)quinazolin-4-yl]amino}benzo-1,4-quinone (Example 25) was prepared by warming. After cooling, ammonia was bubbled in for 3 minutes. The mixture was stirred for 20 minutes and diluted with ether. The solid was collected, dissolved in chloroform and poured onto a column of Magnasol™. The product was eluted with chloroform-isopropanol mixtures to yield 0.19 g of product as a orange-brown powder: mass spectrum (electrospray, m/e): M+H⁺ 371.0.

### EXAMPLE 189

### 2-chloro-3-methoxy-5-{[6-methoxy-7-(2-methoxyethoxy)quinazolin-4-yl]amino}benzo-1,4-quinone

This compound was prepared from of N′-[2-cyano-4-methoxy-5-(2-methoxyethoxy)phenyl]-N,N-dimethylimidoformamide and 5-amino-2-chloro-3,4-dimethoxy-phenol using the combined methods described above in Examples 16 and 17: mass spectrum (electrospray, m/e): M+H⁺ 436.1.

### EXAMPLE 190

### 2-chloro-3-methoxy-5-{[6-methoxy-7-(2-methoxyethoxy)quinazolin-4-yl]amino}-6-(methylthio)benzo-1,4-quinone

Methyl mercaptan was bubbled into a solution of 2-chloro-3-methoxy-5-{[6-methoxy-7-(2-methoxyethoxy)quinazolin-4-yl]amino}benzo-1,4-quinone (Example 189) in 50 ml of methylene chloride containing 1 drop of triethylamine. After 1.5 hours, the solvent was removed, the residue stirred with ether and the solid collected. The solid was dissolved in hot acetonitrile (50 ml) and 0.3 g of DDC was added. After 10 minutes, the mixture was diluted with chloroform and the solution was passed through a column of Magnasol™. The solvent was removed and the product was purified by chromatography yielding 0.36 g of a blue-black powder: mass spectrum (electrospray, m/e): M+H⁺ 468.0.

### EXAMPLE 191

### 5-methoxy-3-{[6-methoxy-7-(2-methoxyethoxy)quinazolin-4-yl]amino}-2-(methylthio)benzo-1,4-quinone

This compound was prepared from 2-methoxy-6-{[6-methoxy-7-(2-methoxyethoxy)quinazolin-4-yl]amino}benzo-1,4-quinone (Example 17) and methyl mercaptan using the method described above for Example 190: mass spectrum (electrospray, m/e): M+H⁺ 432.1.

### EXAMPLE 192

### 2-bromo-6-{[6-methoxy-7-(2-methoxyethoxy)quinazolin-4-yl]amino}benzo-1,4-quinone

This compound was prepared from of N′-[2-cyano-4-methoxy-5-(2-methoxyethoxy)phenyl]-N,N-dimethylimidoformamide and 3-bromo-2,5-dimethoxy-aniline using the combined methods described above in Examples 16 and 17: mass spectrum (electrospray, m/e): M+H⁺ 434.0

### EXAMPLES 193-211

A phenol (0.152 mmol) and the phase transfer catalyst tricaprylylmethylammonium chloride (0.01 mmol) were treated with an equivalent amount of 1 N NaOH, to which methylene chloride (2 ml) and water (1 ml) were added. This solution was stirred for 15 minutes. The biphasic mixture was then treated with the 2-chloro-5-{[6-methoxy-7-(2-methoxyethoxy)quinazolin-4-yl]amino}benzo-1,4-quinone (0.101 mmol) in a methylene chloride solution to give a total volume of 8 ml in the reaction. The reactions were agitated with a vortex shaker for a time ranging from 2 to 48 hours. Completion of the reaction was determined by LC-MS. The organic layers were then separated and the aqueous solution was extracted further with methylene chloride (2×2 ml). The organic layers were combined and dried over magnesium sulfate and concentrated. The reactions, which showed only desired quinone as the major component, were purified by either recrystallization from acetonitrile or silica gel chromatography. Some reactions showed a substantial amount of the desired product in reduced form. These reactions were treated with an excess of DDQ in methylene chloride (2 ml) and were agitated with a vortex shaker overnight. The reactions were washed with a saturated potassium carbonate solution (3×2 ml) and the organic layers dried over magnesium sulfate and concentrated. Again, the reactions which showed only desired quinone as the major component were purified by either recrystallization from acetonitrile or silica gel chromatography. By using this method, the compounds of this invention listed in Table 13 were prepared starting with the indicated phenol.

TABLE 13

| | | | Mass |
|---|---|---|---|
| | | | spectrum |
| Example | Phenol | Compound Name | (M + H) |
| 193 | 4-hydroxybenzamide | 4-[(4-{[6-methoxy-7-(2- | 491.5 |
| | | methoxyethoxy)quinazolin-4-yl]amino}-3,6- | |
| | | dioxocyclohexa-1,4-dien-1- | |
| | | yl)oxy]benzamide | |
| 194 | m-cresol | 2-{[6-methoxy-7-(2- | 462.5 |
| | | methoxyethoxy)quinazolin-4-yl]amino}-5-(3- | |
| | | methylphenoxy)benzo-1,4-quinone | |
| 195 | 4-benzyloxyphenol | 2-[4-(benzyloxy)phenoxy]-5-{[6-methoxy-7- | 554.6 |
| | | (2-methoxyethoxy)quinazolin-4- | |
| | | yl]amino}benzo-1,4-quinone | |
| 196 | 3-acetamidophenol | N-{3-[(4-{[6-methoxy-7-(2- | 505.5 |
| | | methoxyethoxy)quinazolin-4-yl]amino}-3,6- | |
| | | dioxocyclohexa-1,4-dien-1- | |
| | | yl)oxy]phenyl}acetamide | |
| 197 | 5-hydroxyisoquinoline | 2-(isoquinolin-5-yloxy)-5-{[6-methoxy-7-(2- | 499.5 |
| | | methoxyethoxy)quinazolin-4- | |
| | | yl]amino}benzo-1,4-quinone | |
| 198 | 2-allylphenol | 2-(2-allylphenoxy)-5-{[6-methoxy-7-(2- | 488.5 |
| | | methoxyethoxy)quinazolin-4- | |
| | | yl]amino}benzo-1,4-quinone | |
| 199 | trifluoromethyl-m-cresol | 2-{[6-methoxy-7-(2- | 516.5 |
| | | methoxyethoxy)quinazolin-4-yl]amino}-5-[3- | |
| | | (trifluoromethyl)phenoxy]benzo-1,4-quinone | |
| 200 | o-hydroxybenzophenone | 2-(2-benzoylphenoxy)-5-{[6-methoxy-7-(2- | 552.5 |
| | | methoxyethoxy)quinazolin-4- | |
| | | yl]amino}benzo-1,4-quinone | |
| 201 | 2-bromophenol | 2-(2-bromophenoxy)-5-{[6-methoxy-7-(2- | 526.4 |
| | | methoxyethoxy)quinazolin-4- | |
| | | yl]amino}benzo-1,4-quinone | |
| 202 | 2-chlorophenol | 2-(2-chlorophenoxy)-5-{[6-methoxy-7-(2- | 482.9 |
| | | methoxyethoxy)quinazolin-4- | |
| | | yl]amino}benzo-1,4-quinone | |
| 203 | 2-cyanophenol | 2-[(4-{[6-methoxy-7-(2- | 473.5 |
| | | methoxyethoxy)quinazolin-4-yl]amino}-3,6- | |
| | | dioxocyclohexa-1,4-dien-1- | |
| | | yl)oxy]benzonitrile | |
| 204 | 6-hydroxylquinoline | 2-{[6-methoxy-7-(2- | 499.5 |
| | | methoxyethoxy)quinazolin-4-yl]amino}-5- | |
| | | (quinolin-6-yloxy)benzo-1,4-quinone | |
| 205 | 2′-hydroxy-1′- | 2-[(1-acetyl-2-naphthyl)oxy]-5-{[6-methoxy- | 540.6 |
| | acetonaphthone | 7-(2-methoxyethoxy)quinazolin-4- | |
| | | yl]amino}benzo-1,4-quinone | |
| 206 | 1′-hydroxy-2′- | 2-[(2-acetyl-1-naphthyl)oxy]-5-{[6-methoxy- | 540.6 |
| | acetonaphthone | 7-(2-methoxyethoxy)quinazolin-4- | |
| | | yl]amino}benzo-1,4-quinone | |
| 207 | 4-(4-hydroxy phenyl)-2- | 2-{[6-methoxy-7-(2- | 518.5 |
| | butanone | methoxyethoxy)quinazolin-4-yl]amino}-5-[4- | |
| | | (3-oxobutyl)phenoxy]benzo-1,4-quinone | |
| 208 | 2-hydroxydibenzofuran | 2-(dibenzo[b,d]furan-2-yloxy)-5-{[6- | 538.5 |
| | | methoxy-7-(2-methoxyethoxy)quinazolin-4- | |
| | | yl]amino}benzo-1,4-quinone | |
| 209 | 6-hydro-1,3- | 2-{[6-methoxy-7-(2- | 538.5 |
| | benzoxathiol-2-one | methoxyethoxy)quinazolin-4-yl]amino}-5- | |
| | | [(2-oxo-1,3-benzoxathiol-6-yl)oxy]benzo- | |
| | | 1,4-quinone | |
| 210 | 4-chloro-1-naphthol | 2-[(4-chloro-1-naphthyl)oxy]-5-{[6-methoxy- | 532.9 |
| | | 7-(2-methoxyethoxy)quinazolin-4- | |
| | | yl]amino}benzo-1,4-quinone | |
| 211 | methyl 3-hydroxy-2- | methyl 3-[(4-{[6-methoxy-7-(2- | 556.6 |
| | naphthoate | methoxyethoxy)quinazolin-4-yl]amino}-3,6- | |
| | | dioxocyclohexa-1,4-dien-1-yl)oxy]-2- | |
| | | naphthoate | |

### EXAMPLES 212-222

2-chloro-5-{[6-methoxy-7-(2-methoxyethoxy)quinazolin-4-yl]amino}benzo-1,4-quinone was dissolved in methylene chloride and treated with sodium phenoxide (trihydrate, 2.0 equivalents) and the listed alcohol in a 10-fold excess. The reaction was then agitated with a vortex shaker overnight. The reactions that were determined to be complete by LC-MS were washed with water, saturated sodium carbonate and dried over sodium sulfate. The solutions were concentrated. The resulting residues were purified by either HPLC or crystallization from acetonitrile. By using this method, the compounds of this invention listed in Table 14 were prepared starting with the indicated alcohol.

TABLE 14

| | | | Mass |
|---|---|---|---|
| | | | Spectrum |
| Example | Alcohol | Compound Name | (M + H) |
| 212 | 1,3-difluoro-2-propanol | 2-[2-fluoro-1-(fluoromethyl)ethoxy]-5-{[6- | 450.5 |
| | | methoxy-7-(2-methoxyethoxy)quinazolin-4- | |
| | | yl]amino}benzo-1,4-quinone | |
| 213 | cyclopropane methanol | 2-(cyclopropylmethoxy)-5-{[6-methoxy-7-(2- | 426.5 |
| | | methoxyethoxy)quinazolin-4-yl]amino}benzo- | |
| | | 1,4-quinone | |
| 214 | cyclopentanol | 2-(cyclopentyloxy)-5-{[6-methoxy-7-(2- | 440.5 |
| | | methoxyethoxy)quinazolin-4-yl]amino}benzo- | |
| | | 1,4-quinone | |
| 215 | cyclohexylmethanol | 2-(cyclohexylmethoxy)-5-{[6-methoxy-7-(2- | 468.5 |
| | | methoxyethoxy)quinazolin-4-yl]amino}benzo- | |
| | | 1,4-quinone | |
| 216 | 2-cyanoethanol | 3-[(4-{[6-methoxy-7-(2- | 425.2 |
| | | methoxyethoxy)quinazolin-4-yl]amino}-3,6- | |
| | | dioxocyclohexa-1,4-dien-1-yl)oxy]propanenitrile | |
| 217 | 2-phenoxyethanol | 2-{[6-methoxy-7-(2-methoxyethoxy)quinazolin- | 492.6 |
| | | 4-yl]amino}-5-(2-phenoxyethoxy)benzo-1,4- | |
| | | quinone | |
| 218 | 3-methoxybenzyl alcohol | 2-[(3-methoxybenzyl)oxy]-5-{[6-methoxy-7-(2- | 492.5 |
| | | methoxyethoxy)quinazolin-4-yl]amino}benzo- | |
| | | 1,4-quinone | |
| 219 | 2,2,2-trifluoroethanol | 2-{[6-methoxy-7-(2-methoxyethoxy)quinazolin- | 454.5 |
| | | 4-yl]amino}-5-(2,2,2-trifluoroethoxy)benzo-1,4- | |
| | | quinone | |
| 220 | 3-hydroxy | 2-{[6-methoxy-7-(2-methoxyethoxy)quinazolin- | 442.5 |
| | tetrahydrofuran | 4-yl]amino}-5-(tetrahydrofuran-3-yloxy)benzo- | |
| | | 1,4-quinone | |
| 221 | 3-(hydroxymethyl) | 2-{[6-methoxy-7-(2-methoxyethoxy)quinazolin- | 463.1 |
| | pyridine | 4-yl]amino}-5-(pyridin-3-ylmethoxy)benzo-1,4- | |
| | | quinone | |
| 222 | 2-(methylphenyl | 2-{[6-methoxy-7-(2-methoxyethoxy)quinazolin- | 505.5 |
| | amino)ethanol | 4-yl]amino}-5-{2- | |
| | | [methyl(phenyl)amino]ethoxy}benzo-1,4- | |
| | | quinone | |

### EXAMPLE 223

### 5-({[4-methoxy-3-(2-methoxyethoxy)phenyl]amino}methylene)-2,2-dimethyl-1,3-dioxane-4,6-dione

To a stirred solution of 4-methoxy-3-(2-methoxyethoxy)aniline (16.04 g, 81.41 mmol), Meldrum's acid (12.89 g, 89.55 mmol) and trimethyl ortho formate (11 mL, 97.69 mmol) were added neat and sequentially. The solution was refluxed for 5 hours. The reaction was cooled to room temperature and the resulting solid was collected by vacuum filtration, 19.47 g (68%) of the title compound as a white solid, mass spectrum (electrospray, m/e): M+H 352.2.

### EXAMPLE 224

### 6-methoxy-7-(2-methoxyethoxy)quinolin-4(1H)-one

To a refluxing solution of dowtherm (10 ml), 5-({[4-methoxy-3-(2-methoxyethoxy)phenyl]amino}methylene)-2,2-dimethyl-1,3-dioxane-4,6-dione (2.5 g, 7.12 mmol) was added neat. The reaction was refluxed for 1 hour. The reaction was then cooled to room temperature. The resulting solid was collected by vacuum filtration and washed with hexanes, yielding 1.68 g of the title compound as a tan solid (94%), mass spectrum (electrospray, m/e): M+H 250.1.

### EXAMPLE 225

### 4-chloro-6-methoxy-7-(2-methoxyethoxy)quinoline

6-methoxy-7-(2-methoxyethoxy)quinolin-4(1H)-one (1.11 g, 4.47 mmol) was refluxed in POCl₃ (30 ml) neat for 5 hours. The reaction was cooled to room temperature and concentrated. The brown residue was cooled 0° C. and was partitioned with saturated sodium bicarbonate and ethyl acetate. The layers were separated and the organic layer was washed with saturated sodium bicarbonate. The organic solution was passed through a magnesol plug and was concentrated to yield 583.0 mg of the title compound as a white solid (49%), mass spectrum (electrospray, m/e): M+H 268.07.

### EXAMPLE 226

### N-(4-chloro-2,5-dimethoxyphenyl)-6-methoxy-7-(2-methoxyethoxy)quinolin-4-amine

4-chloro-6-methoxy-7-(2-methoxyethoxy)quinoline (222.0 mg, 0.83 mmol) and 4-chloro-2,5-dimethoxy-aniline (468.9 mg, 2.49 mmol) were refluxed in methoxyethanol (20 mL) for several hours. The solvent was removed and the residue was partitioned with saturated sodium bicarbonate and ethyl acetate. The layers were separated and the organic layer was washed with saturated sodium bicarbonate, dried over sodium sulfate and concentrated to give 228.1 mg (66%) of the title compound, mass spectrum (electrospray, m/e): M+H 419.1.

### EXAMPLE 227

### 2-chloro-5-{[6-methoxy-7-(2-methoxyethoxy)quinolin-4-yl]amino}benzo-1,4-quinone

N-(4-chloro-2,5-dimethoxyphenyl)-6-methoxy-7-(2-methoxyethoxy)quinolin-4-amine (228.3 mg, 0.55 mmol) was refluxed in the presence of ceric ammonium nitrate (658.5 mg, 1.2 mmol) in acetonitrile (10 ml)/water (2 ml) for 1 hour. The aqueous solution was extracted with methylene chloride (3×). The organic layers were combined washed with water, dried over sodium sulfate and concentrated to give 129.5 mg of a red solid (61%), mass spectrum (electrospray, m/e): M+H 389.08.

### EXAMPLE 228

### 2-{[6-methoxy-7-(2-methoxyethoxy)quinolin-4-yl]amino}-5-[4-(1-methyl-1-Phenylethyl)phenoxy]benzo-1,4-quinone

2-chloro-5-{[6-methoxy-7-(2-methoxyethoxy)quinolin-4-yl]amino}benzo-1,4-quinone (205.4 mg, 0.529 mmol) was dissolved in methylene chloride (4 ml), treated with water (2 ml), 1N NaOH (530 μl), a catalytic amount of aliquot and 4-(1-methyl-1-phenyl-ethyl)-phenol (145.9 mg, 0.69 mmol). The biphasic mixture was stirred at room temperature for 2 hours. The phases were separated and the aqueous layer was extracted with methylene chloride (3×). The organic layers were combined and passed through a magnesol plug and concentrated to give 230.7 mg of the title compound as a red solid (77%), mass spectrum (electrospray, m/e): M+H 565.2.

### EXAMPLE 229

### 2-(dimethylamino)-5-{[6-methoxy-7-(2-methoxyethoxy)quinolin-4-yl]amino}benzo-1,4-quinone

2-chloro-5-{[6-methoxy-7-(2-methoxyethoxy)quinolin-4-yl]amino}benzo-1,4-quinone (160.6 mg, 0.414 mmol) was dissolved in tetrahydrofuran (5 ml) and was treated with pyridinium hydrochloride (47.83 mg, 0.414 mmol) and a solution of dimethylamine (2.1 ml, 2.0 M, 4.14 mmol) in tetrahydrofuran. The mixture was stirred for 3 hours. The resulting solid was collected by vacuum filtration and washed with water, yielding 128.9 mg (78%) of the title compound as red solid, mass spectrum (electrospray, m/e): M−H 396.15.

### EXAMPLE 230

### N-(4-bromo-2,5-dimethoxyphenyl)-6-methoxy-7-(2-methoxyethoxy)quinazolin-4-amine

This compound was prepared from N′-[2-cyano-4-methoxy-5-(2-methoxyethoxy)phenyl]-N,N-dimethylimidoformamide (8.32 g, 30 mmol) and 4-bromo-2,5-dimethoxy-phenylamine (7.66 g, 33 mmol) in HOAc (30 mL) using the procedure described above for Example 14 to give 12.17 g (87%) of the title compound as a grey solid: mp 217-221° C.; MS (ESI) m/z 464; ¹H NMR (400 MHz, DMSO-D₆) δ ppm 3.34 (s, 3H) 3.71-3.77 (m, 5H) 3.78-3.80 (m, 3H) 3.94 (s, 3H) 4.22-4.28 (m, 2H) 7.18 (s, 1H) 7.34 (s, 1H) 7.37 (s, 1H) 7.79 (s, 1H) 8.32 (s, 1H) 9.18 (s, 1H).

### EXAMPLE 231

### 2-bromo-5-{[6-methoxy-7-(2-methoxyethoxy)quinazolin-4-yl]amino}benzo-1,4-quinone

This compound was prepared from N-(4-bromo-2,5-dimethoxyphenyl)-6-methoxy-7-(2-methoxyethoxy)quinazolin-4-amine (300 mg, 0.65 mmol) and CAN (0.78 g, 1.43 mmol) in CH₃CN (8.6 mL) and H₂O (1.1 mL) to give 256 mg (90.6%) of the product as a purple red solid: mp 200-210° C.; HRMS: calcd for C₁₈H₁₆BrN₃O₅+H+, 434.03461; found (ESI-FTMS, [M+H]¹⁺), 434.03449; ¹H NMR (400 MHz, CHLOROFORM-D) δ ppm 3.49 (s, 3H) 3.88-3.90 (m, 2H) 4.07 (s, 3H) 4.33-4.35 (m, 2H) 7.03 (s, 1H) 7.40 (s, 1H) 8.34 (s, 1H) 8.46 (s, 1H) 8.83 (s, 1H). HPLC purity 84.4% at 215 nm, 10.9 min.; Prodigy ODS3, 0.46×15 cm column, 1.0 mL/min, 20 min Gradient ACN in H₂O/TFA.

### EXAMPLE 232

### 2-[benzyl(4-methoxyphenyl)amino]-5-{[6-methoxy-7-(2-methoxyethoxy)quinazolin-4-yl]amino}benzo-1,4-quinone

To a suspension of 2-chloro-5-{[6-methoxy-7-(2-methoxyethoxy)quinazolin-4-yl]amino}benzo-1,4-quinone (150 mg, 0.4 mmol) and Et₃N (263 mL, 1.92 mmol) in 3 mL CH₂Cl₂ at 60° C., 4-(2-methoxy-benzyl)piperidine (791.4 mg, 3.86 mmol) was added. The reaction mixture was stirred at 60° C. for 2 hours and then filtered through a pad of magnesol with CH₂Cl₂. The solvent was removed in vacuo. The residue was triturated with Et₂O. The resulting solid was purified by silica gel column (3% MeOH/CH₂Cl₂) and Gilson HPLC to give 161.4 mg (55%) of the title compound: MS (ESI) m/z 567.2; HRMS: calcd for C₃₂H₃₀N₄O₆+H+, 567.22381; found (ESI-FTMS, [M+H]¹⁺), 567.2231.

### EXAMPLES 233-235

To a suspension of 2-chloro-5-{[6-methoxy-7-(2-methoxyethoxy)quinazolin-4-yl]amino}benzo-1,4-quinone (150 mg, 0.4 mmol) and Et₃N (263 mL, 1.92 mmol) in 3 mL CH₂Cl₂ at 60° C., the appropriate aniline (˜3.9 mmol) was added. The reaction mixture was stirred at 60° C. for 2 hours and filtered through a pad of magnesol with CH₂Cl₂. The solvent was removed in vacuo. The residue was triturated with Et₂O. The resulting solid was purified by silica gel column (3% MeOH/CH₂Cl₂) and Gilson HPLC to give the title compound. The compounds of the invention made by this method are listed in Table 15.

TABLE 15

| Example | Compound Name | MS | HRMS |
|---|---|---|---|
| 233 | 2-[ethyl(4-methylphenyl)amino]-5- | MS (ESI) m/z | HRMS: calcd for |
| | {[6-methoxy-7-(2- | 489.2 | C₂₇H₂₈N₄O₅ + H+, |
| | methoxyethoxy)quinazolin-4- | | 489.21325; found (ESI- |
| | yl]amino}benzo-1,4-quinone | | FTMS, [M + H]¹⁺), |
| | | | 489.21332 |
| 234 | 2-[butyl(phenyl)amino]-5-{[6- | MS (ESI-FTMS) | HRMS: calcd for |
| | methoxy-7-(2- | m/z 503.22849; | C₂₈H₃₀N₄O₅ + H+, |
| | methoxyethoxy)quinazolin-4- | MS (ESI-FTMS) | 503.22890; found (ESI- |
| | yl]amino}benzo-1,4-quinone | m/z 503.2289 | FTMS, [M + H]¹⁺), |
| | | | 503.22849 |
| 235 | 2-[ethyl(phenyl)amino]-5-{[6- | MS (ESI) m/z | HRMS: calcd for |
| | methoxy-7-(2- | 475.2 | C₂₆H₂₆N₄O₅ + H+, |
| | methoxyethoxy)quinazolin-4- | | 475.19760; found (ESI- |
| | yl]amino}benzo-1,4-quinone | | FTMS, [M + H]¹⁺), |
| | | | 475.19711 |

### EXAMPLE 236

### 2-(5-bromo-2,3-dihydro-1H-indol-1-yl)-5-{[6-methoxy-7-(2-methoxyethoxy)quinazolin-4-yl]amino}benzo-1,4-quinone

A solution of 2-chloro-5-{[6-methoxy-7-(2-methoxyethoxy)quinazolin-4-yl]amino}benzo-1,4-quinone (150 mg, 0.38 mmol) and 18-crown-6 (10 mg, 0.4 mmol) in 4 mL DMF was stirred at 60° C. for 1 hour. 5-bromoindoline (191 mg, 0.96 mmol) in 2 mL DMF was added. The reaction mixture was stirred at 60° C. for 3 hours. It was filtered through a pad of magnesol with CH₂Cl₂/THF. The solvent was removed in vacuo. The residue was purified by column eluting with CH₂Cl₂ and 30% CH₂Cl₂/THF. The solvent of the product fraction was evaporated to yield 116.9 mg (55%) of the title compound: HRMS: calcd for C₂₆H₂₃BrN₄O₅+H+, 551.09246; found (ESI-FTMS, [M+H]¹⁺), 551.09118.

### EXAMPLES 237-242

A solution of 2-chloro-5-{[6-methoxy-7-(2-methoxyethoxy)quinazolin-4-yl]amino}benzo-1,4-quinone (150 mg, 0.38 mmol) and 18-crown-6 (10 mg, 0.4 mmol) in 4 mL DMF was stirred at 60° C. for 1 hour. The appropriate aniline (˜1.0 mmol) in 2 mL DMF was added and the reaction mixture was stirred at 60° C. for 3 hours and filtered through a pad of magnesol with CH₂Cl₂/THF. The solvent was removed in vacuo. The residue was purified by column eluting with CH₂Cl₂ and 30% CH₂Cl₂/THF. The solvent of the product fraction was evaporated to yield the title compound. The compounds of the invention are listed in Table 16.

TABLE 16

| Example | Compound Name | MS | HRMS |
|---|---|---|---|
| 237 | 2-{[6-methoxy-7-(2- | MS (ESI) m/z | HRMS: calcd for |
| | methoxyethoxy)quinazolin-4- | 475.1 | C₂₆H₂₆N₄O₅ + H+, |
| | yl]amino}-5-[methyl(3- | | 475.19760; found (ESI- |
| | methylphenyl)amino]benzo-1,4- | | FTMS, [M + H]¹⁺), |
| | quinone | | 475.19806 |
| 238 | 2-{[6-methoxy-7-(2- | MS (ESI) m/z | HRMS: calcd for |
| | methoxyethoxy)quinazolin-4- | 517.2 | C₂₉H₃₂N₄O₅ + H+, |
| | yl]amino}-5- | | 517.24455; found (ESI- |
| | [pentyl(phenyl)amino]benzo-1,4- | | FTMS, [M + H]¹⁺), |
| | quinone | | 517.24497 |
| 239 | 2-(2,3-dihydro-1H-indol-1-yl)-5-{[6- | MS (ESI+) m/z | HRMS: calcd for |
| | methoxy-7-(2- | 473.1 | C₂₆H₂₄N₄O₅ + H+, |
| | methoxyethoxy)quinazolin-4- | | 473.18195; found (ESI- |
| | yl]amino}benzo-1,4-quinone | | FTMS, [M + H]¹⁺), |
| | | | 473.18255 |
| 240 | 2-[(4-chlorophenyl)(methyl)amino]-5- | MS (ESI+) m/z | HRMS: calcd for |
| | {[6-methoxy-7-(2- | 495.1 | C₂₅H₂₃ClN₄O₅ + H+, |
| | methoxyethoxy)quinazolin-4- | | 495.14297; found (ESI- |
| | yl]amino}benzo-1,4-quinone | | FTMS, [M + H]¹⁺), |
| | | | 495.14368 |
| 241 | 2-[1,3-benzodioxol-5- | MS (ESI+) m/z | HRMS: calcd for |
| | yl(ethyl)amino]-5-{[6-methoxy-7-(2- | 519.1 | C₂₇H₂₆N₄O₇ + H+, |
| | methoxyethoxy)quinazolin-4- | | 519.18743; found (ESI- |
| | yl]amino}benzo-1,4-quinone | | FTMS, [M + H]¹⁺), |
| | | | 519.18768 |
| 242 | 2-[ethyl(1-naphthyl)amino]-5-{[6- | MS (ESI) m/z | HRMS: calcd for |
| | methoxy-7-(2- | 525.2; MS (ESI) | C₃₀H₂₈N₄O₅ + H+, |
| | methoxyethoxy)quinazolin-4- | m/z 283.6; | 525.21325; found (ESI- |
| | yl]amino}benzo-1,4-quinone | MS (ESI) m/z 263.1 | FTMS, [M + H]¹⁺), |
| | | | 525.2124 |

### EXAMPLES 243-257

A solution of 0.97 g (2.5 mmol) of 2-chloro-5-{[6-methoxy-7-(2-methoxyethoxy)quinazolin-4-yl]amino}benzo-1,4-quinone, 0.29 g of pyridine hydrochloride, and the appropriate amine or piperazine in THF, in 15 ml of THF, was stirred for 3 hours. The solid was collected via filtration and washed with water and dried to give the title compound. The compounds of the invention made by this method are listed in Table 17.

TABLE 17

| Example | Compound Name | MS | HRMS |
|---|---|---|---|
| 243 | 3-chloro-2-[4-(3- | MS (ESI) m/z | HRMS: calcd for |
| | chlorobenzyl)piperazin-1-yl]-5-{[6- | 598; MS (ESI) | C₂₉H₂₉Cl₂N₅O₅ + H+, |
| | methoxy-7-(2- | m/z 299.5; | 598.16185; found (ESI- |
| | methoxyethoxy)quinazolin-4- | MS (ESI) m/z 320 | FTMS, [M + H]¹⁺), |
| | yl]amino}benzo-1,4-quinone | | 598.16231 |
| 244 | 2-[(3-hydroxy-3- | MS (ESI) m/z | HRMS: calcd for |
| | phenylpropyl)(methyl)amino]-5-{[6- | 519.2 | C₂₈H₃₀N₄O₆ + H+, |
| | methoxy-7-(2- | | 519.22381; found |
| | methoxyethoxy)quinazolin-4- | | (ESI_FTMS, [M + H]¹⁺), |
| | yl]amino}benzo-1,4-quinone | | 519.22376 |
| 245 | 2-[4-(2,4- | MS (ESI) m/z | HRMS: calcd for |
| | dimethoxybenzyl)piperazin-1-yl]-5- | 590.1; | C₃₁H₃₅N₅O₇ + H+, |
| | {[6-methoxy-7-(2- | MS (ESI) m/z 295.6 | 590.26093; found (ESI- |
| | methoxyethoxy)quinazolin-4- | | FTMS, [M + H]¹⁺), |
| | yl]amino}benzo-1,4-quinone | | 590.26109 |
| 246 | 2-{[6-methoxy-7-(2- | MS (ESI) m/z | HRMS: calcd for |
| | methoxyethoxy)quinazolin-4- | 490.2; | C₂₆H₂₇N₅O₅ + H+, |
| | yl]amino}-5-[methyl(2-pyridin-2- | MS (ESI) m/z 245.6 | 490.20850; found |
| | ylethyl)amino]benzo-1,4-quinone | | (ESI_FTMS, [M + H]¹⁺), |
| | | | 490.20811 |
| 247 | 4-{[4-(4-{[6-methoxy-7-(2- | MS (ESI) m/z | HRMS: calcd for |
| | methoxyethoxy)quinazolin-4- | 555.3; | C₃₀H₃₀N₆O₅ + H+, |
| | yl]amino}-3,6-dioxocyclohexa-1,4- | MS (ESI) m/z | 555.23505; found (ESI- |
| | dien-1-yl)piperazin-1- | 278.2; | FTMS, [M + H]¹⁺), |
| | yl]methyl}benzonitrile | MS (ESI) m/z 298.7 | 555.23495 |
| 248 | 2-{4-[4- | MS (ESI) m/z | HRMS: calcd for |
| | (dimethylamino)benzyl]piperazin-1- | 573.3 | C₃₁H₃₆N₆O₅ + H+, |
| | yl}-5-{[6-methoxy-7-(2- | | 573.28200; found (ESI- |
| | methoxyethoxy)quinazolin-4- | | FTMS, [M + H]¹⁺), |
| | yl]amino}benzo-1,4-quinone | | 573.28393 |
| 249 | 2-{[6-methoxy-7-(2- | MS (ESI) m/z | HRMS: calcd for |
| | methoxyethoxy)quinazolin-4- | 510.2; | C₂₇H₃₅N₅O₅ + H+, |
| | yl]amino}-5-[4-(2- | MS (ESI) m/z 255.6 | 510.27110; found (ESI- |
| | methylbutyl)piperazin-1-yl]benzo- | | FTMS, [M + H]¹⁺), |
| | 1,4-quinone | | 510.2696 |
| 250 | 2-[4-(1,3-benzodioxol-5- | MS (ESI) m/z | HRMS: calcd for |
| | ylmethyl)piperazin-1-yl]-5-{[6- | 574.2; | C₃₀H₃₁N₅O₇ + H+, |
| | methoxy-7-(2- | MS (ESI) m/z | 574.22963; found (ESI- |
| | methoxyethoxy)quinazolin-4- | 287.6; | FTMS, [M + H]¹⁺), |
| | yl]amino}benzo-1,4-quinone | MS (ESI) m/z 308.1 | 574.22908 |
| 251 | 2-[4-(3-fluorobenzyl)piperazin-1-yl]- | MS (ESI) m/z | HRMS: calcd for |
| | 5-{[6-methoxy-7-(2- | 548.2; | C₂₉H₃₀FN₅O₅ + H+, |
| | methoxyethoxy)quinazolin-4- | MS (ESI) m/z | 548.23037; found (ESI- |
| | yl]amino}benzo-1,4-quinone | 274.6; | FTMS, [M + H]¹⁺), |
| | | MS (ESI) m/z 295.1 | 548.22888 |
| 252 | 2-{[6-methoxy-7-(2- | MS (ESI) m/z | HRMS: calcd for |
| | methoxyethoxy)quinazolin-4- | 536.2; | C₂₇H₂₉N₅O₅S + H+, |
| | yl]amino}-5-[4-(2- | MS (ESI) m/z | 536.19622; found (ESI- |
| | thienylmethyl)piperazin-1-yl]benzo- | 268.6; | FTMS, [M + H]¹⁺), |
| | 1,4-quinone | MS (ESI) m/z 289.1 | 536.19525 |
| 253 | 2-[4-(3,7-dimethyloct-6-en-1- | MS (ESI) m/z | HRMS: calcd for |
| | yl)piperazin-1-yl]-5-{[6-methoxy-7- | 578.3; | C₃₂H₄₃N₅O₅ + H+, |
| | (2-methoxyethoxy)quinazolin-4- | MS (ESI) m/z 289.6 | 578.33370; found (ESI- |
| | yl]amino}benzo-1,4-quinone | | FTMS, [M + H]¹⁺), |
| | | | 578.33375 |
| 254 | 2-[4-(2-furylmethyl)piperazin-1-yl]-5- | MS (ESI) m/z | HRMS: calcd for |
| | {[6-methoxy-7-(2- | 520.2; | C₂₇H₂₉N₅O₆ + H+, |
| | methoxyethoxy)quinazolin-4- | MS (ESI) m/z 260.6 | 520.21906; found (ESI- |
| | yl]amino}benzo-1,4-quinone | | FTMS, [M + H]¹⁺), |
| | | | 520.21863 |
| 255 | 2-{[6-methoxy-7-(2- | MS (ESI) m/z | HRMS: calcd for |
| | methoxyethoxy)quinazolin-4- | 531.2; | C₂₈H₃₀N₆O₅ + H+, |
| | yl]amino}-5-[4-(pyridin-3- | MS (ESI) m/z | 531.23505; found (ESI- |
| | ylmethyl)piperazin-1-yl]benzo-1,4- | 266.1; | FTMS, [M + H]¹⁺), |
| | quinone | MS (ESI) m/z 286.6 | 531.23518 |
| 256 | 2-[4-(2,4-dimethoxybenzyl)-1,4- | MS (ESI) m/z | HRMS: calcd for |
| | diazepan-1-yl]-5-{[6-methoxy-7-(2- | 604.2; | C₃₂H₃₇N₅O₇ + H+, |
| | methoxyethoxy)quinazolin-4- | MS (ESI) m/z 302.6 | 604.27658; found (ESI- |
| | yl]amino}benzo-1,4-quinone | | FTMS, [M + H]¹⁺), |
| | | | 604.2777 |
| 257 | 2-{[6-methoxy-7-(2- | MS (ESI) m/z | HRMS: calcd for |
| | methoxyethoxy)quinazolin-4- | 524.2; | C₂₈H₃₇N₅O₅ + H+, |
| | yl]amino}-5-[4-(2-methylbutyl)-1,4- | MS (ESI) m/z 262.6 | 524.28675; found (ESI- |
| | diazepan-1-yl]benzo-1,4-quinone | | FTMS, [M + H]¹⁺), |
| | | | 524.2864 |

### EXAMPLES 258-260

A solution of 0.20 g (0.51 mmol) of 2-chloro-5-{[6-methoxy-7-(2-methoxyethoxy)quinazolin-4-yl]amino}benzo-1,4-quinone, 0.06 g of pyridine hydrochloride and the appropriate amine in 2 ml of THF was sonicated for 0.5 hour at 40° C., then shaken at 40° C. for 3 hours. The solid was collected via filtration and washed with water and dried to give the title compound. The compounds of the invention made by this method are listed in Table 18.

TABLE 18

| Example | Compound Name | MS | HRMS |
|---|---|---|---|
| 258 | 2-{[6-methoxy-7-(2- | MS (ESI) m/z | HRMS: calcd for |
| | methoxyethoxy)quinazolin-4- | 580.1; MS (ESI) | C₃₃H₃₃N₅O₅ + H+, |
| | yl]amino}-5-[4-(2- | m/z 290.6 | 580.25545; found (ESI- |
| | naphthylmethyl)piperazin-1- | | FTMS, [M + H]¹⁺), |
| | yl]benzo-1,4-quinone | | 580.25435 |
| 259 | 2-{[6-methoxy-7-(2- | MS (ESI) m/z | HRMS: calcd for |
| | methoxyethoxy)quinazolin-4- | 580.2; MS (ESI) | C₃₃H₃₃N₅O₅ + H+, |
| | yl]amino}-5-[4-(1- | m/z 290.6 | 580.25545; found (ESI- |
| | naphthylmethyl)piperazin-1- | | FTMS, [M + H]¹⁺), |
| | yl]benzo-1,4-quinone | | 580.25619 |
| 260 | 2-[4-(3-chlorobenzyl)piperazin-1-yl]- | MS (ESI) m/z | HRMS: calcd for |
| | 5-{[6-methoxy-7-(2- | 564.1; MS (ESI) | C₂₉H₃₀ClN₅O₅ + H+, |
| | methoxyethoxy)quinazolin-4- | m/z 282.5; | 564.20082; found (ESI- |
| | yl]amino}benzo-1,4-quinone | MS (ESI) m/z 303.1 | FTMS, [M + H]¹⁺), |
| | | | 564.2007 |

### EXAMPLE 261

### 2-[4-(2-methoxybenzyl)piperidin-1-yl]-5-{[6-methoxy-7-(2-methoxyethoxy)quinazolin-4-yl]amino}benzo-1,4-quinone

To a suspension of 2-chloro-5-{[6-methoxy-7-(2-methoxyethoxy)quinazolin-4-yl]amino}benzo-1,4-quinone (150 mg, 0.4 mmol) and Et₃N (263 mL, 1.92 mmol) in 3 mL CH₂Cl₂ at 60° C. was added the appropriate amine (˜3.9 mmol). The reaction mixture was stirred at 60° C. for 2 hours. It was filtered through a pad of magnesol with CH₂Cl₂. The solvent was removed in vacuo. The residue was triturated with Et₂O. The resulting solid was purified by silica gel column (3% MeOH/CH₂Cl₂) and Gilson HPLC to give the title compound:MS (ESI) m/z 559.2; HRMS: calcd for C₃₁H₃₄N₄O₆+H+, 559.25511; found (ESI-FTMS, [M+H]¹⁺), 559.25342.

### EXAMPLE 262

### 5-[4-(1,3-benzodioxol-5-ylmethyl)piperazin-1-yl]-3-(ethylthio)-2-{[6-methoxy-7-(2-methoxyethoxy)quinazolin-4-yl]amino}benzo-1,4-quinone

To a degassed stirred solution of acetonitrile:deionized (MilliQ) water (1:1, 1000 mL) of the quinone (˜0.1 mmol, 40 mg) under N₂, ethanethiol (10 equiv., ˜0.1 mL) was added. The solution was stirred until starting material was consumed as shown by TLC or LCMS (1 hour-5 days). At the end of the reaction, 2.9 g of 0.7 mmol/g loading maleimide resin (Silicycle, Si-maleimide) was added to scavenge the ethanethiol. The suspension was stirred overnight, then filtered (medium frit) and extracted with 3×150 mL EtOAc, dried with Na₂SO₃, and concentrated in vacuo (30-40° C.). The crude residue was purified by RP-HPLC (C18 Phenomenex Luna 150×30 mm, 20-80% MeCN:water 0.02% TFA). NaCl was added to the isolated fractions and extracted into DCM, dried with Na₂SO₃ and concentrated in vacuo (30-40° C.) giving 3 mg of title compound: MS (ESI) m/z 634.3; MS (ESI) m/z 317.6; MS (ESI) m/z 338.1.

### EXAMPLE 263

### N-[2,5-dimethoxy-4-(methylthio)phenyl]-6-methoxy-7-(2-methoxyethoxy)quinazolin-4-amine

Compound N′-[2-cyano-4-methoxy-5-(2-methoxyethoxy)phenyl]-N,N-dimethylimidoformamide (885 mg, 3.19 mmol) and 2,5-dimethoxy-4-methylsulfanyl-phenylamine (700 mg, 3.51 mmol) (Chem. Ber. 1964, 285-294) were heated to 110° C. in AcOH (4 mL) for 3 hours. The reaction was partitioned in water/EtOAc, the brown solid precipitates filtered and washed with water and EtOAc. The solids were dissolved in MeOH and purified in silica gel column, eluted with 2.5% MeOH/CH₂Cl₂ to yield 485 mg (35%) of the title compound as pink solids: MS (ESI) m/z 432.1; HRMS: calcd for C₂₁H₂₅N₃O₅S+H+, 432.15877; found (ESI-FTMS, [M+H]¹⁺), 432.15853; ¹H NMR (400 MHz, CHLOROFORM-D) δ ppm 2.44-2.47 (m, 3H) 3.47-3.50 (m, 3H) 3.84-3.92 (m, 2H) 3.97 (s, 6H) 4.01-4.10 (m, 3H) 4.28-4.38 (m, 2H) 6.93 (s, 1H) 7.03 (s, 1H) 7.28-7.32 (m, 1H) 8.55 (s, 1H) 8.71 (s, 1H).

### EXAMPLE 264

### 2-{[6-methoxy-7-(2-methoxyethoxy)quinazolin-4-yl]amino}-5-(methylthio)benzo-1,4-quinone

This compound was prepared from N-[2,5-dimethoxy-4-(methylthio)phenyl]-6-methoxy-7-(2-methoxyethoxy)quinazolin-4-amine (130 mg, 0.3 mmol) and CAN (345 mg, 21.0 mmol) in CHCl₃ (1.5 mL), CH₃CN (3.0 mL) and H₂O (0.6 mL) using the procedure described above for Example 17 to give 102 mg (84%) of the title compound as a red solid: MS (ESI) m/z 402; HRMS: calcd for C₁₉H₁₉N₃O₅S+H+, 402.11182; found (ESI-FTMS, [M+H]¹⁺), 402.11222; ¹H NMR (400 MHz, CHLOROFORM-D) δ ppm 2.39 (s, 3H) 3.49 (s, 3H) 3.88-3.90 (m, 2H) 4.08 (s, 3H) 4.33-4.35 (m, 2H) 6.39 (s, 1H) 7.07 (s, 1H) 7.33 (s, 1H) 8.14 (s, 1H) 8.72 (s, 1H) 8.83 (s, 1H).

### EXAMPLES 265-292

2-chloro-5-{[6-methoxy-7-(2-methoxyethoxy)quinazolin-4-yl]amino}benzo-1,4-quinone was dissolved in methylene chloride and treated with sodium phenoxide (trihydrate, 2.0 equivalents) and the appropriate alcohol in a 10-fold excess. The reaction was then agitated with a vortex shaker overnight. The reactions that were determined to be complete by LC-MS were washed with water and saturated sodium carbonate, dried over sodium sulfate and concentrated. The resulting residues were purified by either HPLC or crystallization from acetonitrile. The compounds of the invention made by this method are listed in Table 19.

TABLE 19

| Example | Compound Name | MS | HRMS |
|---|---|---|---|
| 265 | 2-[(2-chlorobenzyl)oxy]-5-{[6- | MS (ESI) m/z | HRMS: calcd for |
| | methoxy-7-(2- | 496.1 | C₂₅H₂₂ClN₃O₆ + H+, |
| | methoxyethoxy)quinazolin-4- | | 496.12699; found (ESI- |
| | yl]amino}benzo-1,4-quinone | | FTMS, [M + H]¹⁺), |
| | | | 496.12636 |
| 266 | 2-isopropoxy-5-{[6-methoxy-7-(2- | MS (ESI) m/z | HRMS: calcd for |
| | methoxyethoxy)quinazolin-4- | 414.1 | C₂₁H₂₃N₃O₆ + H+, |
| | yl]amino}benzo-1,4-quinone | | 414.16596; found (ESI- |
| | | | FTMS, [M + H]¹⁺), |
| | | | 414.16758 |
| 267 | 2-{[6-methoxy-7-(2- | MS (ESI) m/z | HRMS: calcd for |
| | methoxyethoxy)quinazolin-4- | 442.1 | C₂₃H₂₇N₃O₆ + H+, |
| | yl]amino}-5-(1-methylbutoxy)benzo- | | 442.19726; found (ESI- |
| | 1,4-quinone | | FTMS, [M + H]¹⁺), |
| | | | 442.1989 |
| 268 | 2-(cycloheptyloxy)-5-{[6-methoxy-7- | MS (ESI) m/z | HRMS: calcd for |
| | (2-methoxyethoxy)quinazolin-4- | 468.1 | C₂₅H₂₉N₃O₆ + H+, |
| | yl]amino}benzo-1,4-quinone | | 468.21291; found (ESI- |
| | | | FTMS, [M + H]¹⁺), |
| | | | 468.21393 |
| 269 | 2-sec-butoxy-5-{[6-methoxy-7-(2- | MS (ESI) m/z | HRMS: calcd for |
| | methoxyethoxy)quinazolin-4- | 428.1 | C₂₂H₂₅N₃O₆ + H+, |
| | yl]amino}benzo-1,4-quinone | | 428.18161; found (ESI- |
| | | | FTMS, [M + H]¹⁺), |
| | | | 428.18304 |
| 270 | 2-(1-ethylpropoxy)-5-{[6-methoxy-7- | MS (ESI) m/z | HRMS: calcd for |
| | (2-methoxyethoxy)quinazolin-4- | 442.1 | C₂₃H₂₇N₃O₆ + H+, |
| | yl]amino}benzo-1,4-quinone | | 442.19726; found (ESI- |
| | | | FTMS, [M + H]¹⁺), |
| | | | 442.19858 |
| 271 | 2-[(1,4-dimethylpentyl)oxy]-5-{[6- | MS (ESI) m/z | HRMS: calcd for |
| | methoxy-7-(2- | 470.2 | C₂₅H₃₁N₃O₆ + H+, |
| | methoxyethoxy)quinazolin-4- | | 470.22856; found (ESI- |
| | yl]amino}benzo-1,4-quinone | | FTMS, [M + H]¹⁺), |
| | | | 470.22845 |
| 272 | 2-{[6-methoxy-7-(2- | MS (ESI) m/z | HRMS: calcd for |
| | methoxyethoxy)quinazolin-4- | 469.1; | C₂₄H₂₈N₄O₆ + H+, |
| | yl]amino}-5-[(1-methylpiperidin-3- | MS (ESI) m/z 235 | 469.20816; found (ESI- |
| | yl)oxy]benzo-1,4-quinone | | FTMS, [M + H]¹⁺), |
| | | | 469.20801 |
| 273 | 2-[(2-fluorobenzyl)oxy]-5-{[6-methoxy- | MS (ESI) m/z | HRMS: calcd for |
| | 7-(2-methoxyethoxy)quinazolin-4- | 480.1 | C₂₅H₂₂FN₃O₆ + H+, |
| | yl]amino}benzo-1,4-quinone | | 480.15654; found (ESI- |
| | | | FTMS, [M + H]¹⁺), |
| | | | 480.1564 |
| 274 | 2-[(3-fluorobenzyl)oxy]-5-{[6-methoxy- | MS (ESI) m/z | HRMS: calcd for |
| | 7-(2-methoxyethoxy)quinazolin-4- | 480.2 | C₂₅H₂₂FN₃O₆ + H+, |
| | yl]amino}benzo-1,4-quinone | | 480.15654; found (ESI- |
| | | | FTMS, [M + H]¹⁺), |
| | | | 480.15514 |
| 275 | 2-{[6-methoxy-7-(2- | MS (ESI+) m/z | HRMS: calcd for |
| | methoxyethoxy)quinazolin-4- | 470.2 | C₂₄H₂₇N₃O₇ + H+, |
| | yl]amino}-5-(tetrahydro-2H-pyran-2- | | 470.19218; found (ESI- |
| | ylmethoxy)benzo-1,4-quinone | | FTMS, [M + H]¹⁺), |
| | | | 470.19192 |
| 276 | 2-[(4-fluorobenzyl)oxy]-5-{[6-methoxy- | MS (ESI) m/z | HRMS: calcd for |
| | 7-(2-methoxyethoxy)quinazolin-4- | 480.2 | C₂₅H₂₂FN₃O₆ + H+, |
| | yl]amino}benzo-1,4-quinone | | 480.15654; found (ESI- |
| | | | FTMS, [M + H]¹⁺), |
| | | | 480.15548 |
| 277 | 2-[(4-methoxybenzyl)oxy]-5-{[6- | MS (ESI) m/z | |
| | methoxy-7-(2- | 492.2 | |
| | methoxyethoxy)quinazolin-4- | | |
| | yl]amino}benzo-1,4-quinone | | |
| 278 | 2-(2,3-dihydro-1H-inden-2-yloxy)-5- | MS (ESI) m/z | |
| | {[6-methoxy-7-(2- | 488.2 | |
| | methoxyethoxy)quinazolin-4- | | |
| | yl]amino}benzo-1,4-quinone | | |
| 279 | 2-{[6-methoxy-7-(2- | MS (ESI) m/z | |
| | methoxyethoxy)quinazolin-4- | 506.2 | |
| | yl]amino}-5-(3- | | |
| | phenoxypropoxy)benzo-1,4-quinone | | |
| 280 | 2-ethoxy-5-{[6-methoxy-7-(2- | MS (ESI) m/z | HRMS: calcd for |
| | methoxyethoxy)quinazolin-4- | 400.1 | C₂₀H₂₁N₃O₆ + H+, |
| | yl]amino}benzo-1,4-quinone | | 400.15031; found (ESI- |
| | | | FTMS, [M + H]¹⁺), |
| | | | 400.15058 |
| 281 | 2-{[6-methoxy-7-(2- | MS (ESI) m/z | HRMS: calcd for |
| | methoxyethoxy)quinazolin-4- | 530.1 | C₂₆H₂₂F₃N₃O₆ + H+, |
| | yl]amino}-5-(2,2,2-trifluoro-1- | | 530.15335; found (ESI- |
| | phenylethoxy)benzo-1,4-quinone | | FTMS, [M + H]¹⁺), |
| | | | 530.15321 |
| 282 | 2-{[6-methoxy-7-(2- | MS (ESI) m/z | HRMS: calcd for |
| | methoxyethoxy)quinazolin-4- | 442.1 | C₂₂H₂₃N₃O₇ + H+, |
| | yl]amino}-5-[(3R)-THF-3-yloxy]benzo- | | 442.16088; found (ESI- |
| | 1,4-quinone | | FTMS, [M + H]¹⁺), |
| | | | 442.16066 |
| 283 | 2-{[6-methoxy-7-(2- | MS (ESI) m/z | HRMS: calcd for |
| | methoxyethoxy)quinazolin-4- | 442.1 | C₂₂H₂₃N₃O₇ + H+, |
| | yl]amino}-5-[(3S)-THF-3-yloxy]benzo- | | 442.16088; found (ESI- |
| | 1,4-quinone | | FTMS, [M + H]¹⁺), |
| | | | 442.16092 |
| 284 | 2-{[1-(4- | MS (ESI) m/z | |
| | chlorophenyl)cyclopropyl]methoxy}-5- | 536.1 | |
| | {[6-methoxy-7-(2- | | |
| | methoxyethoxy)quinazolin-4- | | |
| | yl]amino}benzo-1,4-quinone | | |
| 285 | 2-{[6-methoxy-7-(2- | MS (ESI) m/z | HRMS: calcd for |
| | methoxyethoxy)quinazolin-4- | 552.1 | C₂₅H₁₈F₅N₃O₆ + H+, |
| | yl]amino}-5- | | 552.11885; found (ESI- |
| | [(pentafluorobenzyl)oxy]benzo-1,4- | | FTMS, [M + H]¹⁺), |
| | quinone | | 552.1169 |
| 286 | 2-(2,2-difluoroethoxy)-5-{[6-methoxy- | MS (ESI) m/z | HRMS: calcd for |
| | 7-(2-methoxyethoxy)quinazolin-4- | 436.1; | C₂₀H₁₉F₂N₃O₆ + H+, |
| | yl]amino}benzo-1,4-quinone | MS (ESI) m/z | 436.13147; found (ESI- |
| | | 871.2 | FTMS, [M + H]¹⁺), |
| | | | 436.13104 |
| 287 | 2-[(2,3,3,4,4,5- | MS (ESI) m/z | HRMS: calcd for |
| | hexafluorocyclopentyl)oxy]-5-{[6- | 548.1 | C₂₃H₁₉F₆N₃O₆ + H+, |
| | methoxy-7-(2- | | 548.12508; found (ESI- |
| | methoxyethoxy)quinazolin-4- | | FTMS, [M + H]¹⁺), |
| | yl]amino}benzo-1,4-quinone | | 548.12599 |
| 288 | 2-(1,3-benzodioxol-5-ylmethoxy)-5- | MS (ESI) m/z | HRMS: calcd for |
| | {[6-methoxy-7-(2- | 506.1 | C₂₆H₂₃N₃O₈ + H+, |
| | methoxyethoxy)quinazolin-4- | | 506.15579; found (ESI- |
| | yl]amino}benzo-1,4-quinone | | FTMS, [M + H]¹⁺), |
| | | | 506.15597 |
| 289 | 2-{[4-(benzyloxy)-3- | MS (ESI) m/z | HRMS: calcd for |
| | methoxybenzyl]oxy}-5-{[6-methoxy-7- | 598.2 | C₃₃H₃₁N₃O₈ + H+, |
| | (2-methoxyethoxy)quinazolin-4- | | 598.21839; found (ESI- |
| | yl]amino}benzo-1,4-quinone | | FTMS, [M + H]¹⁺), |
| | | | 598.21829 |
| 290 | 2-{[4-(benzyloxy)benzyl]oxy}-5-{[6- | MS (ESI) m/z | HRMS: calcd for |
| | methoxy-7-(2- | 568.2 | C₃₂H₂₉N₃O₇ + H+, |
| | methoxyethoxy)quinazolin-4- | | 568.20783; found (ESI- |
| | yl]amino}benzo-1,4-quinone | | FTMS, [M + H]¹⁺), |
| | | | 568.20692 |
| 291 | 2-{[6-methoxy-7-(2- | MS (ESI) m/z | HRMS: calcd for |
| | methoxyethoxy)quinazolin-4- | 486.1 | C₂₇H₂₃N₃O₆ + H+, |
| | yl]amino}-5-[(3-phenylprop-2-yn-1- | | 486.16596; found (ESI- |
| | yl)oxy]benzo-1,4-quinone | | FTMS, [M + H]¹⁺), |
| | | | 486.16532 |
| 292 | 2-{[6-methoxy-7-(2- | MS (ESI) m/z | HRMS: calcd for |
| | methoxyethoxy)quinazolin-4- | 554.1 | C₃₁H₂₇N₃O₇ + H+, |
| | yl]amino}-5-[(3- | | 554.19218; found (ESI- |
| | phenoxybenzyl)oxy]benzo-1,4- | | FTMS, [M + H]¹⁺), |
| | quinone | | 554.19197 |

### EXAMPLES 293-296

A solution of 0.67 g (1.5 mmol) of 2-{[6-methoxy-7-(2-methoxyethoxy)quinazolin-4-yl]amino}-5-phenoxybenzo-1,4-quinone, 0.5 ml of triethylamine, and the appropriate alcohol (˜20 mL) in 20 ml methylene chloride was stirred for 16 hours. The solvent was evaporated and the residue diluted with ether. The solid was collected and washed with ether giving the appropriate compound. The compounds of the invention made by this method are listed in Table 20.

TABLE 20

| Example | Compound Name | MS | HRMS |
|---|---|---|---|
| 293 | 2-{[6-methoxy-7-(2- | MS (ESI+) m/z | HRMS: calcd for |
| | methoxyethoxy)quinazolin-4- | 456.2 | C₂₃H₂₅N₃O₇ + H+, |
| | yl]amino}-5-(tetrahydro-2H-pyran-4- | | 456.17653; found (ESI- |
| | yloxy)benzo-1,4-quinone | | FTMS, [M + H]¹⁺), |
| | | | 456.17691 |
| 294 | 2-[2-(dimethylamino)-1- | MS (ESI) m/z | HRMS: calcd for |
| | methylethoxy]-5-{[6-methoxy-7-(2- | 457.1; MS (ESI) | C₂₃H₂₈N₄O₆ + H+, |
| | methoxyethoxy)quinazolin-4- | m/z 229.1 | 457.20816; found (ESI- |
| | yl]amino}benzo-1,4-quinone | | FTMS, [M + H]¹⁺), |
| | | | 457.20793 |
| 295 | 2-{[6-methoxy-7-(2- | MS (ESI) m/z | HRMS: calcd for |
| | methoxyethoxy)quinazolin-4- | 456.1 | C₂₃H₂₅N₃O₇ + H+, |
| | yl]amino}-5-(THF-3- | | 456.17653; found (ESI- |
| | ylmethoxy)benzo-1,4-quinone | | FTMS, [M + H]¹⁺), |
| | | | 456.17542 |
| 296 | 2-{[6-methoxy-7-(2- | MS (ESI+) m/z | HRMS: calcd for |
| | methoxyethoxy)quinazolin-4- | 456.2 | C₂₃H₂₅N₃O₇ + H+, |
| | yl]amino}-5-[(3-methyloxetan-3- | | 456.17653; found (ESI- |
| | yl)methoxy]benzo-1,4-quinone | | FTMS, [M + H]¹⁺), |
| | | | 456.17556 |

### EXAMPLE 297

### 2-{[6-methoxy-7-(2-methoxyethoxy)quinazolin-4-yl]amino}-5-[(1-methylpyrrolidin-3-yl)oxy]benzo-1,4-quinone

A solution of 0.97 g (2.5 mmol) of 2-chloro-5-{[6-methoxy-7-(2-methoxyethoxy)quinazolin-4-yl]amino}benzo-1,4-quinone, 0.29 g of pyridine hydrochloride, and 5 ml of the 3-hydroxy-1-methylpyrrolidine in THF, in 15 ml of THF, was stirred for 3 hours. The solid was collected via filtration and washed with water and dried to yield 0.94 g of the title compound as a light brown solid: mass spectrum (electrospray, m/e): M−H 399.2; MS (ESI+) m/z 455.2; HRMS: calcd. for C₂₃H₂₆N₄O₆+H+, 455.19251; found (ESI-FTMS, [M+H]¹⁺), 455.19148.

### EXAMPLE 298

### 2-[(3-fluorobenzyl)oxy]-5-{[6-methoxy-7-(3-pyrrolidin-1-ylpropoxy)quinazolin-4-yl]amino}benzo-1,4-quinone

To a suspension of N-(4-chloro-2,5-dimethoxyphenyl)-6-methoxy-7-(3-pyrrolidin-1-ylpropoxy)quinazolin-4-amine (890 mg, 1.88 mmol) in 45 mL CH₃CN and 22 mL H₂O, ammonium cerium (IV) nitrate (1.84 g, 5.65 mmol) was added. The reaction mixture was diluted with CH₂Cl₂. A saturated solution of Na₂CO₃ was added. The aqueous layer was extracted (3×) with CH₂Cl₂. The solution containing the quinone intermediate (a final volume of 500 mL CH₂Cl₂) was dried over MgSO₄. NaOPh(3H₂O) (595.1 mg, 3.76 mmol) was dissolved in warm 3-fluorobenzyl alcohol (2.85 g, 22.58 mmol) and then added to the solution of quinone. About 150 mL of solvent was removed at 45° C. over 15 minutes. The reaction mixture was filtered through a plug of magnesol, eluting with CHCl₃, EtOAc, EtOAc/isopropanol and EtOAc/isopropanol/Et₃N=80:20:1. The solvent was removed from product fractions to yield 0.163 g (16.2%) of title compound as an orange solid: MS (ESI) m/z 533.1; MS (ESI) m/z 267; MS (ESI) m/z 287.6; ¹H NMR (400 MHz, CDCL₃) δ ppm 1.80 (s, 4H) 2.09-2.22 (m, 2H) 2.55 (s, 4H) 2.68 (t, J=7.30 Hz, 2H) 4.07 (s, 3H) 4.27 (t, J=6.55 Hz, 2H) 5.12 (s, 2H) 6.02 (s, 1H) 7.05 (s, 1H) 7.07-7.11 (m, 1H) 7.16 (d, J=9.06 Hz, 1H) 7.21 (d, J=8.06 Hz, 1H) 7.33 (s, 1H) 7.36-7.43 (m, 1H) 8.09 (s, 1H) 8.71 (s, 1H) 8.82 (s, 1H); Anal. (C₂₉H₂₉FN₄O₅ 0.5H₂O)C, H, N.

### EXAMPLES 299-323

To a suspension of N-(4-chloro-2,5-dimethoxyphenyl)-6-methoxy-7-(3-pyrrolidin-1-ylpropoxy)quinazolin-4-amine (890 mg, 1.88 mmol) in 45 mL CH₃CN and 22 mL H₂O, ammonium cerium (IV) nitrate (1.84 g, 5.65 mmol) was added. The reaction mixture was diluted with CH₂Cl₂. A saturated solution of Na₂CO₃ was added. The aqueous layer was extracted (3×) with CH₂Cl₂. The solution containing the quinone intermediate (a final volume of 500 mL CH₂Cl₂) was dried over MgSO₄. NaOPh(3H₂O) (595.1 mg, 3.76 mmol) was dissolved in the appropriate warm alcohol (˜23 mmol) and then added to the solution of quinone. About 150 mL of solvent was removed at 45° C. over 15 minutes. The reaction mixture was filtered through a plug of magnesol, eluting with CHCl₃, EtOAc, EtOAc/isopropanol and EtOAc/isopropanol/Et₃N=80:20:1. The solvent was removed from product fractions to yield the title compound. The compounds of the invention made by this method are listed in Table 21.

TABLE 21

| Example | Compound Name | MS | HRMS |
|---|---|---|---|
| 299 | 2-[(2-hydroxyethyl)amino]-5-{[6- | MS (ESI) m/z | |
| | methoxy-7-(3-pyrrolidin-1- | 468.1; | |
| | ylpropoxy)quinazolin-4- | MS (ESI) m/z 234.5 | |
| | yl]amino}benzo-1,4-quinone | | |
| 300 | 2-{[6-methoxy-7-(3-pyrrolidin-1- | MS (ESI) m/z | |
| | ylpropoxy)quinazolin-4-yl]amino}-5- | 477.2; | |
| | [(1-methylprop-2-yn-1-yl)oxy]benzo- | MS (ESI) m/z | |
| | 1,4-quinone | 239.1; | |
| | | MS (ESI) m/z 259.6 | |
| 301 | 2-(allyloxy)-5-{[6-methoxy-7-(3- | MS (ESI) m/z | |
| | pyrrolidin-1-ylpropoxy)quinazolin-4- | 465.1; | |
| | yl]amino}benzo-1,4-quinone | MS (ESI) m/z | |
| | | 233.1; | |
| | | MS (ESI) m/z 253.6 | |
| 302 | 2-{[6-methoxy-7-(3-pyrrolidin-1- | MS (ESI) m/z | |
| | ylpropoxy)quinazolin-4-yl]amino}-5- | 463.1; | |
| | (prop-2-yn-1-yloxy)benzo-1,4- | MS (ESI) m/z 232; | |
| | quinone | MS (ESI) m/z | |
| | | 252.6 | |
| 303 | 2-{[6-methoxy-7-(3-pyrrolidin-1- | MS (ESI) m/z | |
| | ylpropoxy)quinazolin-4-yl]amino}-5- | 539.1; | |
| | [(1-phenylprop-2-yn-1-yl)oxy]benzo- | MS (ESI) m/z 270.1 | |
| | 1,4-quinone | | |
| 304 | 2-{[6-methoxy-7-(3-pyrrolidin-1- | MS (ESI) m/z | HRMS: calcd for |
| | ylpropoxy)quinazolin-4-yl]amino}-5- | 495.2; | C₂₆H₃₀N₄O₆ + H+, |
| | (THF-3-yloxy)benzo-1,4-quinone | MS (ESI) m/z | 495.22381; found (ESI- |
| | | 248.1; | FTMS, [M + H]¹⁺), |
| | | MS (ESI) m/z 268.6 | 495.22402 |
| 305 | 2-{[6-methoxy-7-(3-pyrrolidin-1- | MS (ESI) m/z | HRMS: calcd for |
| | ylpropoxy)quinazolin-4-yl]amino}-5- | 529.2; | C₃₀H₃₂N₄O₅ + H+, |
| | [(2-methylbenzyl)oxy]benzo-1,4- | MS (ESI) m/z | 529.24455; found (ESI- |
| | quinone | 265.1; | FTMS, [M + H]¹⁺), |
| | | MS (ESI) m/z 285.6 | 529.24463 |
| 306 | 2-{[6-methoxy-7-(3-pyrrolidin-1- | MS (ESI) m/z | HRMS: calcd for |
| | ylpropoxy)quinazolin-4-yl]amino}-5- | 593.1; | C₃₀H₃₂N₄O₇S + H+, |
| | {[4- | MS (ESI) m/z 297.1 | 593.20645; found (ESI- |
| | (methylsulfonyl)benzyl]oxy}benzo- | | FTMS, [M + H]¹⁺), |
| | 1,4-quinone | | 593.20469 |
| 307 | 2-{[6-methoxy-7-(3-pyrrolidin-1- | MS (ESI) m/z | HRMS: calcd for |
| | ylpropoxy)quinazolin-4-yl]amino}-5- | 605.1; | C₂₉H₂₅F₅N₄O₅ + H+, |
| | [(pentafluorobenzyl)oxy]benzo-1,4- | MS (ESI) m/z 323.5 | 605.18179; found (ESI- |
| | quinone | | FTMS, [M + H]¹⁺), |
| | | | 605.1804 |
| 308 | 2-({4-[(4- | MS (ESI) m/z | HRMS: calcd for |
| | fluorobenzyl)oxy]benzyl}oxy)-5-{[6- | 639.2 | C₃₆H₃₅FN₄O₆ + H+, |
| | methoxy-7-(3-pyrrolidin-1- | | 639.26134; found (ESI- |
| | ylpropoxy)quinazolin-4- | | FTMS, [M + H]¹⁺), |
| | yl]amino}benzo-1,4-quinone | | 639.26041 |
| 309 | 2-{[6-methoxy-7-(3-pyrrolidin-1- | MS (ESI) m/z | HRMS: calcd for |
| | ylpropoxy)quinazolin-4-yl]amino}-5- | 558.2; | C₃₁H₃₅N₅O₅ + H+, |
| | {2- | MS (ESI) m/z 279.6 | 558.27110; found (ESI- |
| | [methyl(phenyl)amino]ethoxy}benzo- | | FTMS, [M + H]¹⁺), |
| | 1,4-quinone | | 558.27113 |
| 310 | 2-(benzyloxy)-5-{[6-methoxy-7-(3- | MS (ESI) m/z | |
| | pyrrolidin-1-ylpropoxy)quinazolin-4- | 515.1; | |
| | yl]amino}benzo-1,4-quinone | MS (ESI) m/z | |
| | | 278.5; | |
| | | MS (ESI) m/z 278.6 | |
| 311 | 2-[(4-chlorobenzyl)oxy]-5-{[6- | MS (ESI) m/z | HRMS: calcd for |
| | methoxy-7-(3-pyrrolidin-1- | 549.1; | C₂₉H₂₉ClN₄O₅ + H+, |
| | ylpropoxy)quinazolin-4- | MS (ESI) m/z 275; | 549.18992; found (ESI- |
| | yl]amino}benzo-1,4-quinone | MS (ESI) m/z | FTMS, [M + H]¹⁺), |
| | | 295.5 | 549.18971 |
| 312 | 2-{[6-methoxy-7-(3-pyrrolidin-1- | MS (ESI) m/z | HRMS: calcd for |
| | ylpropoxy)quinazolin-4-yl]amino}-5- | 516.1; | C₂₈H₂₉N₅O₅ + H+, |
| | (pyridin-3-ylmethoxy)benzo-1,4- | MS (ESI) m/z | 516.22415; found (ESI- |
| | quinone | 258.5; | FTMS, [M + H]¹⁺), |
| | | MS (ESI) m/z 279.1 | 516.22542 |
| 313 | 2-{[6-methoxy-7-(3-pyrrolidin-1- | MS (ESI) m/z | |
| | ylpropoxy)quinazolin-4-yl]amino}-5- | 516.1; | |
| | (pyridin-2-ylmethoxy)benzo-1,4- | MS (ESI) m/z 258.5 | |
| | quinone | | |
| 314 | 3-{[(4-{[6-methoxy-7-(3-pyrrolidin-1- | MS (ESI) m/z | |
| | ylpropoxy)quinazolin-4-yl]amino}- | 540.1; | |
| | 3,6-dioxocyclohexa-1,4-dien-1- | MS (ESI) m/z | |
| | yl)oxy]methyl}benzonitrile | 270.5; | |
| | | MS (ESI) m/z 291.1 | |
| 315 | 2-[2-chloro-1-(fluoromethyl)ethoxy]- | MS (ESI) m/z | HRMS: calcd for |
| | 5-{[6-methoxy-7-(3-pyrrolidin-1- | 519.1; | C₂₅H₂₈ClFN₄O₅ + H+, |
| | ylpropoxy)quinazolin-4- | MS (ESI) m/z 260; | 519.18050; found (ESI- |
| | yl]amino}benzo-1,4-quinone | MS (ESI) m/z | FTMS, [M + H]¹⁺), |
| | | 280.5 | 519.1819 |
| 316 | 2-{[6-methoxy-7-(3-pyrrolidin-1- | MS (ESI) m/z | |
| | ylpropoxy)quinazolin-4-yl]amino}-5- | 539.1; | |
| | [(3-phenylprop-2-yn-1-yl)oxy]benzo- | MS (ESI) m/z | |
| | 1,4-quinone | 270.1; | |
| | | MS (ESI) m/z 290.6 | |
| 317 | 2-(2-furylmethoxy)-5-{[6-methoxy-7- | MS (ESI) m/z | HRMS: calcd for |
| | (3-pyrrolidin-1-ylpropoxy)quinazolin- | 505.2 | C₂₇H₂₈N₄O₆ + H+, |
| | 4-yl]amino}benzo-1,4-quinone | | 505.20816; found (ESI- |
| | | | FTMS, [M + H]¹⁺), |
| | | | 505.2077 |
| 318 | 2-(2,2-difluoroethoxy)-5-{[6-methoxy- | MS (ESI+) m/z | HRMS: calcd for |
| | 7-(3-pyrrolidin-1- | 489.1 | C₂₄H₂₆F₂N₄O₅ + H+, |
| | ylpropoxy)quinazolin-4- | | 489.19440; found (ESI- |
| | yl]amino}benzo-1,4-quinone | | FTMS, [M + H]¹⁺), |
| | | | 489.1956 |
| 319 | 2-[2-fluoro-1-(fluoromethyl)ethoxy]-5- | MS (ESI) m/z | HRMS: calcd for |
| | {[6-methoxy-7-(3-pyrrolidin-1- | 503.1; | C₂₅H₂₈F₂N₄O₅ + H+, |
| | ylpropoxy)quinazolin-4- | MS (ESI) m/z 252; | 503.21005; found (ESI- |
| | yl]amino}benzo-1,4-quinone | MS (ESI) m/z | FTMS, [M + H]¹⁺), |
| | | 272.6 | 503.21081 |
| 320 | 2-{[6-methoxy-7-(3-pyrrolidin-1- | MS (ESI) m/z | HRMS: calcd for |
| | ylpropoxy)quinazolin-4-yl]amino}-5- | 559.2; | C₃₁H₃₄N₄O₆ + H+, |
| | (3-phenoxypropoxy)benzo-1,4- | MS (ESI) m/z | 559.25511; found (ESI- |
| | quinone | 280.1; | FTMS, [M + H]¹⁺), |
| | | MS (ESI) m/z 300.6 | 559.2544 |
| 321 | 2-{[6-methoxy-7-(3-pyrrolidin-1- | MS (ESI) m/z | HRMS: calcd for |
| | ylpropoxy)quinazolin-4-yl]amino}-5- | 541.2; | C₃₁H₃₂N₄O₅ + H+, |
| | {[(2E)-3-phenylprop-2-en-1- | MS (ESI) m/z | 541.24455; found (ESI- |
| | yl]oxy}benzo-1,4-quinone | 271.1; | FTMS, [M + H]¹⁺), |
| | | MS (ESI) m/z 291.6 | 541.24582 |
| 322 | 2-{[6-methoxy-7-(3-pyrrolidin-1- | MS (ESI) m/z | HRMS: calcd for |
| | ylpropoxy)quinazolin-4-yl]amino}-5- | 545.2 | C30H32N4O6 + H+, |
| | (2-phenoxyethoxy)benzo-1,4- | | 545.23946; found (ESI- |
| | quinone | | FTMS, [M + H]1+), |
| | | | 545.24125 |
| 323 | 2-methoxy-5-{[6-methoxy-7-(3- | MS (ESI) m/z | HRMS: calcd for |
| | pyrrolidin-1-ylpropoxy)quinazolin-4- | 439.1; | C23H26N4O5 + H+, |
| | yl]amino}benzo-1,4-quinone | MS (ESI) m/z 220; | 439.19760; found (ESI- |
| | | MS (ESI) m/z | FTMS, [M + H]1+), |
| | | 240.5 | 439.1995 |

### EXAMPLE 324

### N-(4-chloro-2,5-dimethoxyphenyl)-6-methoxy-7-(3-pyridin-4-ylpropoxy)quinazolin-4-amine

This compound was prepared from N-(4-chloro-2,5-dimethoxyphenyl)-7-fluoro-6-methoxy-N-(4-methoxybenzyl)quinazolin-4-amine (726 mg, 1.5 mmol), 4-pyridine propanol (0.62 g, 4.5 mmol) and sodium bis(trimethylsilyl)amide (1.0 M in THF) (3.75 mL, 3.75 mmol) in THF (1.5 mL). The residue was purified on a flash column of silica gel (2×20 cm), eluting with 10:10:1 CH₂Cl₂/EtOAc/MeOH and 10:1 CH₂Cl₂/MeOH to yield 625 mg (86.8%) of the title compound as a white solid: mp 205-208° C.; MS (ESI) m/z 481.1; MS (ESI) m/z 241; MS (ESI) m/z 261.5; ¹H NMR (400 MHz, DMSO-D6) δ ppm 2.11-2.18 (m, 2H) 2.79-2.83 (m, 2H) 3.75 (s, 3H) 3.80 (s, 3H) 3.94 (s, 3H) 4.15 (t, J=6.42 Hz, 2H) 7.15 (s, 1H) 7.22 (s, 1H) 7.28-7.31 (m, 2H) 7.38 (s, 1H) 7.80 (s, 1H) 8.32 (s, 1H) 8.47 (dd, J=4.53, 1.51 Hz, 2H) 9.19 (s, 1H).

### EXAMPLE 325

### 2-chloro-5-({6-methoxy-7-[(1-methylpiperidin-4-yl)methoxy]quinazolin-4-yl}amino)benzo-1,4-quinone

This compound was prepared from N-(4-chloro-2,5-dimethoxyphenyl)-6-methoxy-7-(3-pyridin-4-ylpropoxy)quinazolin-4-amine (4.37 g, 9.23 mmol) and CAN (11.1 g, 20.3 mmol) in CH₃CN (92 mL) and H₂O (37 mL) using the procedure described above for Example 17. The reaction mixture was stirred in CHCl₃ and Na₂CO₃ (0.67 M, 100 mL,) and filtered through a pad of Celite. The CHCl₃ layer was washed with brine, dried over MgSO₄, and concentrated in the presence of hexane at 25° C. to yield 4.2 g (100%) of the title compound as a red solid: MS (ESI) m/z 443.1; MS (ESI) m/z 222.1; MS (ESI) m/z 242.6; HRMS: calcd for C₂₂H₂₃ClN₄O₄+H+, 443.14806; found (ESI-FTMS, [M+H]¹⁺), 443.14908; ¹H NMR (400 MHz, CHLOROFORM-D) δ ppm 1.90-2.10 (m, 5H) 2.12-2.28 (m, 2H) 2.38-2.49 (s, 3H) 3.09 (s, 2H) 4.01-4.12 (m, 5H) 7.03 (s, 1H) 7.10 (s, 1H) 7.29 (s, 1H) 8.29 (s, 1H) 8.49 (s, 1H) 8.82-8.84 (m, 1H); Anal. (C₂₂H₂₃ClN₄O₄.0.1H₂O) C, H, N.

### EXAMPLES 326-327

To a suspension of 2-chloro-5-{[6-methoxy-7-(1-methylpiperidin-4-yl)methoxy)quinazolin-4-yl]amino}benzo-1,4-quinone (150 mg, 0.4 mmol) and Et₃N (263 mL, 1.92 mmol) in 3 mL CH₂Cl₂ at 60° C., the appropriate piperidine or piperazine (˜3.9 mmol) was added. The reaction mixture was stirred at 60° C. for 2 hours and then filtered through a pad of magnesol with CH₂Cl₂. The solvent was removed in vacuo. The residue was triturated with Et₂O. The resulting solid was purified by silica gel column (3% MeOH/CH₂Cl₂) and Gilson HPLC to give the title compound. The compounds of the invention made by this method are listed in Table 22.

TABLE 22

| Example | Compound Name | MS | HRMS |
|---|---|---|---|
| 326 | 2-(4-benzylpiperazin-1-yl)-5-({6- | MS (ESI) m/z | HRMS: calcd for |
| | methoxy-7-[(1-methylpiperidin-4- | 583.3; MS (ESI) | C₃₃H₃₈N₆O₄ + H+, |
| | yl)methoxy]quinazolin-4- | m/z 292.1; | 583.30273; found (ESI- |
| | yl}amino)benzo-1,4-quinone | MS (ESI) m/z 222.4 | FTMS, [M + H]¹⁺), |
| | | | 583.30388 |
| 327 | 2-[4-(2-methoxybenzyl)piperidin-1- | MS (ESI) m/z | HRMS: calcd for |
| | yl]-5-({6-methoxy-7-[(1- | 612.3; MS (ESI) | C₃₅H₄₁N₅O₅ + H+, |
| | methylpiperidin-4- | m/z 306.6; | 612.31805; found (ESI- |
| | yl)methoxy]quinazolin-4- | MS (ESI) m/z 327.1 | FTMS, [M + H]¹⁺), |
| | yl}amino)benzo-1,4-quinone | | 612.31905 |

### EXAMPLES 328-331

A solution of 0.67 g (1.5 mmol) of 2-{[6-methoxy-7-(1-methylpiperidin-4-yl)quinazolin-4-yl]amino}-5-phenoxybenzo-1,4-quinone, 20 ml of the appropriate alcohol and 0.5 ml of triethylamine in 20 ml methylene chloride was stirred for 16 hours. The solvent was evaporated and the residue diluted with ether. The solid was collected and washed with ether giving the title compound. The compounds of the invention made by this method are listed in Table 23.

TABLE 23

| Example | Compound Name | MS | HRMS |
|---|---|---|---|
| 328 | 2-[2-fluoro-1-(fluoromethyl)ethoxy]- | MS (ESI) m/z | |
| | 5-({6-methoxy-7-[3-(4- | 532.2; MS (ESI) | |
| | methylpiperazin-1- | m/z 287.1; | |
| | yl)propoxy]quinazolin-4- | MS (ESI) m/z 266.6 | |
| | yl}amino)benzo-1,4-quinone | | |
| 329 | 2-ethoxy-5-({6-methoxy-7-[(1- | MS (ESI) m/z | HRMS: calcd for |
| | methylpiperidin-4- | 453.1; MS (ESI) | C₂₄H₂₈N₄O₅ + H+, |
| | yl)methoxy]quinazolin-4- | m/z 247.6; | 453.21325; found (ESI- |
| | yl}amino)benzo-1,4-quinone | MS (ESI) m/z 227 | FTMS, [M + H]¹⁺), |
| | | | 453.21421 |
| 330 | 2-[2-fluoro-1-(fluoromethyl)ethoxy]- | MS (ESI) m/z | |
| | 5-({6-methoxy-7-[1-methylpiperadin- | 503.2; MS (ESI) | |
| | 4-yl)methoxy]quinazolin-4- | m/z 272.6; | |
| | yl}amino)benzo-1,4-quinone | MS (ESI) m/z 252.1 | |
| 331 | 2-methoxy-5-({6-methoxy-7-[(1- | MS (ESI) m/z | |
| | methylpiperidin-4- | 439.2; MS (ESI) | |
| | yl)methoxy]quinazolin-4- | m/z 240.6; | |
| | yl}amino)benzo-1,4-quinone | MS (ESI) m/z 220.1 | |

### EXAMPLES 332-334

To a solution of 2-chloro-5-{[6-methoxy-7-(1-methylpiperidin-4-yl)quinazolin-4-yl)quinazolin-4-yl]amino}benzo-1,4-quinone (800 mg, 1.91 mmol) in dichloromethane (115 mL), CsCO₃ (800 mg, 1.91 mmol) and the appropriate alcohol (˜1.5 mmol) were added. The reaction mixture was stirred at room temperature for 2.5 hours and filtered through a short column of silica gel. The solvent was removed in rotary evaporator. The residue was chromatographed on silica gel, eluting with CHCl₃/EtOAc from 7:3 to 5:5. The product fraction was collected and concentrated in rotary evaporator. The residue was stirred in small amount of CH₃CN. The resulting solid was filtered to yield title compound. The compounds of the invention made by this method are listed in Table 24.

TABLE 24

| Example | Compound Name | MS | HRMS |
|---|---|---|---|
| 332 | 2-({6-methoxy-7-[(1-methylpiperidin- | MS (ESI) m/z | HRMS: calcd for |
| | 4-yl)methoxy]quinazolin-4-yl}amino)- | 545.2; MS (ESI) | C₃₀H₃₂N₄O₆ + H+, |
| | 5-(2-phenoxyethoxy)benzo-1,4- | m/z 293.6; | 545.23946; found (ESI- |
| | quinone | MS (ESI) m/z 273.1 | FTMS, [M + H]¹⁺), |
| | | | 545.23931 |
| 333 | 2-(benzyloxy)-5-({6-methoxy-7-[(1- | MS (ESI) m/z | HRMS: calcd for |
| | methylpiperidin-4- | 515.1; MS (ESI) | C₂₉H₃₀N₄O₅ + H+, |
| | yl)methoxy]quinazolin-4- | m/z 278.5 | 515.22890; found (ESI- |
| | yl}amino)benzo-1,4-quinone | | FTMS, [M + H]¹⁺), |
| | | | 515.22821 |
| 334 | 2-({6-methoxy-7-[(1-methylpiperidin- | MS (ESI) m/z | HRMS: calcd for |
| | 4-yl)methoxy]quinazolin-4-yl}amino)- | 558.2; MS (ESI) | C₃₁H₃₅N₅O₅ + H+, |
| | 5-{2- | m/z 279.6; | 558.27110; found (ESI- |
| | [methyl(phenyl)amino]ethoxy}benzo- | MS (ESI) m/z 300.1 | FTMS, [M + H]¹⁺), 558.272 |
| | 1,4-quinone | | |

### EXAMPLE 335

### 2-chloro-5-{[6-methoxy-7-(3-pyridin-4-ylpropoxy)quinazolin-4-yl]amino}benzo-1,4-quinone

A solution of 7.7 g (19 mmol) of 4-[(4-chloro-2,5-dimethoxyphenyl)amino]-6-methoxy-7-(3-pyridin-4-ylpropoxy)quinoline-3-carbonitrile in 322 ml of acetonitrile was heated to reflux and to this solution, 65 ml of water was added. The mixture was stirred and when the temperature reached 30° C., 19 g (34.7 mmol) of ceric ammonium nitrate was added over 5 minutes. After 45 minutes, the mixture was diluted with dilute sodium bicarbonate. The solid was collected by filtration and washed with water. This solid was suspended in 300 ml of water and 35 ml of concentrated hydrochloride acid was added. After stirring for 15 minutes, the precipitated solid was collected. The solid was stirred with 700 ml of methylene chloride and saturated sodium bicarbonate solution. The organic layer was dried over magnesium sulfate and the solution was passed onto a column of Magnesol™. The product was eluted from the column using ethyl acetate. The solvent was evaporated from the product fractions to give a solid that was washed with ether, yielding the title compound: MS (ESI+) m/z 451.2; HRMS: calcd for C₂₃H₁₉ClN₄O₄+H+, 451.11676; found (ESI-FTMS, [M+H]¹⁺), 451.11643.

### EXAMPLE 336

### 2-methoxy-5-{[6-methoxy-7-(3-pyridin-4-ylpropoxy)quinazolin-4-yl]amino}benzo-1,4-quinone

A solution of 0.67 g (1.5 mmol) of 2-{[6-methoxy-7-(3-pyridin-4-ylpropoxy)quinazolin-4-yl]amino}-5-phenoxybenzo-1,4-quinone, 20 ml of methanol and 0.5 ml of triethylamine in 20 ml methylene chloride was stirred for 16 hours. The solvent was evaporated and the residue diluted with ether. The solid was collected and washed with ether giving title compound: MS (ESI+) m/z 447.1.

### EXAMPLES 337-338

A solution of 7.7 g (19 mmol) of the appropriate carbonitrile in 322 ml of acetonitrile was heated to reflux and to this solution, 65 ml of water was added. The mixture was stirred and when the temperature reached 30° C., 19 g (34.7 mmol) of ceric ammonium nitrate was added over 5 minutes. After 45 minutes, the mixture was diluted with dilute sodium bicarbonate. The solid was collected by filtration and washed with water. This solid was suspended in 300 ml of water and 35 ml of concentrated hydrochloride acid was added. After stirring for 15 minutes, the precipitated solid was collected. The solid was stirred with 700 ml of methylene chloride and saturated sodium bicarbonate solution. The organic layer was dried over magnesium sulfate and the solution was passed onto a column of Magnesol™. The product was eluted from the column using ethyl acetate. The solvent was evaporated from the product fractions to give a solid that was washed with ether, yielding the title compound. The compounds of the invention made by this method are listed in Table 25.

TABLE 25

| Example | Compound Name | MS | HRMS |
|---|---|---|---|
| 337 | 2-{[6,7-bis(2- | MS | |
| | methoxyethoxy)quinazolin-4- | (ESI+) | |
| | yl]amino}benzo-1,4-quinone | m/z | |
| | | 400.1 | |
| 338 | 2-{[6,7-bis(2- | MS | HRMS: calcd for |
| | methoxyethoxy)quinazolin-4- | (ESI+) | C₂₀H₂₀ClN₃O₆ + |
| | yl]amino}-5-chlorobenzo-1,4- | m/z | H+, 434.11134; |
| | quinone | 434.1 | found (ESI-FTMS, |
| | | | [M + H]¹⁺), |
| | | | 434.11147 |

### EXAMPLES 338-340

2-chloro-5-{[6,7-bis(2-methoxyethoxyquinazolin-4-yl]amino}benzo-1,4-quinone was dissolved in methylene chloride and treated with sodium phenoxide (trihydrate, 2.0 equivalents) and the appropriate alcohol in a 10-fold excess. The reaction was then agitated with a vortex shaker overnight. The reactions that were determined to be complete by LC-MS were washed with water and saturated sodium carbonate, and dried over sodium sulfate, then concentrated. The resulting residues were purified by either HPLC or crystallization from acetonitrile. The compounds of the invention made by this method are listed in Table 26.

TABLE 26

| Example | Compound Name | MS | HRMS |
|---|---|---|---|
| 338 | 2-{[6,7-bis(2- | MS (ESI+) m/z | |
| | methoxyethoxy)quinazolin-4- | 507.1 | |
| | yl]amino}-5-(pyridin-3- | | |
| | ylmethoxy)benzo-1,4-quinone | | |
| 339 | 2-{[6,7-bis(2- | MS (ESI) m/z | HRMS: calcd for |
| | methoxyethoxy)quinazolin-4- | 494.1 | C₂₃H₂₅F₂N₃O₇ + H+, |
| | yl]amino}-5-[2-fluoro-1- | | 494.17333; found (ESI- |
| | (fluoromethyl)ethoxy]benzo-1,4- | | FTMS, [M + H]¹⁺), |
| | quinone | | 494.17237 |
| 340 | 2-{[6,7-bis(2- | MS (ESI) m/z | HRMS: calcd for |
| | methoxyethoxy)quinazolin-4- | 430.1 | C₂₁H₂₃N₃O₇ + H+, |
| | yl]amino}-5-methoxybenzo-1,4- | | 430.16088; found (ESI- |
| | quinone | | FTMS, [M + H]¹⁺), |
| | | | 430.16079 |

### EXAMPLE 341

### 2-{[6,7-bis(2-methoxyethoxy)quinazolin-4-yl]amino}-5-[4-(1H-imidazol-1-yl)phenoxy]benzo-1,4-quinone

To a stirred mixture of 2,4-(imidazol-1-yl)phenol (83 mg, 0.52 mmol), aliquot 336 (16 mg, 0.04 mmol), 1N NaOH (46 mL, 0.46 mmol) in CH₂Cl₂ at 25° C., 2-{[6,7-bis(2-methoxyethoxy)quinazolin-4-yl]amino}-5-chlorobenzo-1,4-quinone (174 mg, 0.40 mmol) was added. The reaction mixture was stirred for 30 minutes and diluted with CH₂Cl₂, washed with H₂O, and dried over MgSO₄. The CH₂Cl₂ solution was passed through a pad of magnesol, eluting with CH₂Cl₂, 5:1=CH₂Cl₂/isopropanol. The product fraction was evaporated. The residue was stirred in 8 mL MeOH and filtered to give 107 mg (48%) of title compound as red solid: mp 109-115° C.; MS (ESI+) m/z 558.1.

### EXAMPLE 342

### (4-chloro-2,5-dimethoxyphenyl)(4-methoxybenzyl)amine

To a stirred solution of the p-anisaldehyde (35.4 g, 260 mmol) in dichloroethane (750 mL), 4-chloro-2,5-dimethoxyaniline (46.9 g, 250 mmol), sodium triacetoxyborohydride (79.5 g, 375 mmol) and acetic acid (21.5 mL, 375 mmol) under nitrogen was added at room temperature. The reaction mixture was stirred at room temperature for 2.5 hours, stirred in CH₂Cl₂ and water, and basified with K₂CO₃ to pH 9-10. The CH₂Cl₂ layer was washed with water, dried, and concentrated. The residue was dissolved in 3:1 hexane-ethyl acetate (500 mL) and passed through a 8.0×4.0 cm pad of silica gel. The solvent was evaporated to yield 77.1 g (96%) of the title compound as a white solid: mp 53-63° C.; MS (ESI) m/z 308; ¹H NMR (400 MHz, CHLOROFORM-D) δ ppm 3.77 (d, J=5.54 Hz, 6H) 3.80 (s, 3H) 4.26 (s, 2H) 4.57 (bs, 1H) 6.25 (s, 1H) 6.75 (s, 1H) 6.84-6.94 (m, 2H) 7.24-7.34 (m, 2H).

### EXAMPLE 343

### N-(4-chloro-2,5-dimethoxyphenyl)-7-fluoro-6-methoxy-N-(4-methoxybenzyl)quinazolin-4-amine

A mixture of (4-chloro-2,5-dimethoxyphenyl)(4-methoxybenzyl)amine (30.8 g, 100 mmol), 4-chloro-7-fluoro-6-methoxyquinazoline (17.0 g, 80 mmol), pyridine (0.65 mL, 8 mmol) and t-BuOH (240 mL) under nitrogen was stirred at reflux temperature for 24 hours. The t-BuOH was evaporated, and the residue stirred with CH₂Cl₂ and dilute NH₄OH. The insoluble material was filtered and washed with CH₂Cl₂ and water. The CH₂Cl₂ layer of filtrate was washed with brine, dried over MgSO₄, and evaporated to give 148.4 g of dark red gum. The gum was dissolved into 40:1 CH₂Cl₂/EtOAc (30 mL) and chromatographed in a silica gel column (3.6×42 cm), eluting with 40:1 CH₂Cl₂/EtOAc, followed by 3:1 CH₂Cl₂/EtOAc to yield 31 g (80%) of the title compound as a white amorphous solid: ¹H NMR (400 MHz, DMSO-D₆) δ ppm 3.34 (d, J=2.01 Hz, 6H) 3.70 (d, J=5.29 Hz, 6H) 5.27 (s, 2H) 6.63 (d, J=9.57 Hz, 1H) 6.78-6.86 (m, 2H) 7.12 (s, 1H) 7.21 (s, 1H) 7.26-7.36 (m, 2H) 7.56 (d, J=12.34 Hz, 1H) 8.65 (s, 1H).

### EXAMPLE 344

### N-(4-chloro-2,5-dimethoxyphenyl)-6-methoxy-7-(tetrahydro-2H-pyran-2-ylmethoxy)quinazolin-4-amine

To a stirred mixture of N-(4-chloro-2,5-dimethoxyphenyl)-7-fluoro-6-methoxy-N-(4-methoxybenzyl)quinazolin-4-amine (0.725 g, 1.5 mmol), tetrahydropyran-2-methanol (0.35 g, 3.0 mmol) in THF (2.0 mL) under nitrogen at 25° C., sodium bis (trimethylsilyl)amide (1.0 M in THF, 2.5 mL, 2.5 mmol) was added over 30 seconds. The reaction mixture was refluxed for 2 hours, cooled, and partitioned with CH₂Cl₂ and water. The CH₂Cl₂ layer was washed with brine, dried over MgSO₄, and evaporated. A solution of the resulting gum in TFA (15 mL) was stirred at 55-60° C. for 60 minutes and concentrated to dryness. The residue was partitioned with CH₂Cl₂ and aqueous NaHCO₃. The CH₂Cl₂ layer was washed with brine, dried over MgSO₄, and evaporated. The residue was purified on a flash column of silica gel (2×20 cm), eluting with 3:1 CH₂Cl₂/EtOAc and 25:25:1 CH₂Cl₂/EtOAc/MeOH to yield 313 mg (45%) of the title compound as a white solid: mp 203-209° C.; ¹H NMR (400 MHz, DMSO-D₆) δ ppm 1.36-1.43 (m, 2H) 1.50-1.53 (m, 2H) 1.67-1.72 (m, 1H) 1.83-1.84 (m, 1H) 3.38-3.45 (m, 1H) 3.72-3.75 (m, 4H) 3.80 (s, 3H) 3.90 (d, J=1.26 Hz, 1H) 3.94 (s, 3H) 4.06-4.10 (m, 2H) 7.16 (s, 1H) 7.22 (s, 1H) 7.38 (s, 1H) 7.79 (s, 1H) 8.32 (s, 1H) 9.18 (s, 1H).

### EXAMPLE 345

### 2-chloro-5-{[6-methoxy-7-(tetrahydro-2H-pyran-2-ylmethoxy)quinazolin-4-yl]amino}benzo-1,4-quinone

This compound was prepared from N-(4-chloro-2,5-dimethoxyphenyl)-6-methoxy-7-(tetrahydro-2H-pyran-2-ylmethoxy)quinazolin-4-amine (391 mg, 0.85 mmol) and CAN (345 mg, 21.0 mmol) in CHCl₃ (5.6 mL), CH₃CN (11.2 mL) and H₂O (1.4 mL) using the procedure described above for Example 17. The reaction was filtered through a pad of magnesol (eluted with 9:1 CH₂Cl₂/isopropanol). The solvent was removed by rotary evaporator to give 336 mg (92%) of the title compound as a red solid: mp 215-220° C.; HRMS: calcd for C₂₁H₂₀ClN₃O₅+H+, 430.11643; found (ESI-FTMS, [M+H]¹⁺), 430.11652; The purity of the title compound was evaluated on two HPLC systems and found to be 97% (system A, retention time=8.21 min) and 97% (system B, retention time=15.12 min); ¹H NMR (400 MHz, CHLOROFORM-D) δ ppm 1.41-1.70 (m, 4H) 1.75 (d, J=11.58 Hz, 1H) 1.86-2.01 (m, 1H) 3.43-3.67 (m, 1H) 3.76-3.93 (m, 1H) 4.02-4.14 (m, 5H) 4.14-4.28 (m, 1H) 7.02 (s, 1H) 7.10 (s, 1H) 7.32 (s, 1H) 8.29 (s, 1H) 8.49 (s, 1H) 8.79-8.86 (s, 1H).

### EXAMPLE 346-348

A solution of 0.67 g (1.5 mmol) of 2-{[6-methoxy-7-(tetrahydropyran-2-ylmethoxy)quinazolin-4-yl]amino}-5-phenoxybenzo-1,4-quinone, 20 ml of the appropriate alcohol and 0.5 ml of triethylamine in 20 ml methylene chloride was stirred for 16 hours. The solvent was evaporated and the residue diluted with ether. The solid was collected and washed with ether giving title compound. The compounds of the invention made by this method are listed in Table 27.

TABLE 27

| Example | Compound Name | MS | HRMS |
|---|---|---|---|
| 346 | 2-methoxy-5-{[6-methoxy-7- | MS (ESI+) m/z | HRMS: calcd for |
| | (tetrahydro-2H-pyran-2- | 426.1 | C₂₂H₂₃N₃O₆ + H+, |
| | ylmethoxy)quinazolin-4- | | 426.16596; found (ESI- |
| | yl]amino}benzo-1,4-quinone | | FTMS, [M + H]¹⁺), |
| | | | 426.16578 |
| 347 | 2-[2-fluoro-1-(fluoromethyl)ethoxy]- | MS (ESI+) m/z | HRMS: calcd for |
| | 5-{[6-methoxy-7-(tetrahydro-2H- | 490.1 | C₂₄H₂₅F₂N₃O₆ + H+, |
| | pyran-2-ylmethoxy)quinazolin-4- | | 490.17842; found (ESI- |
| | yl]amino}benzo-1,4-quinone | | FTMS, [M + H]¹⁺), |
| | | | 490.17771 |
| 348 | 2-methoxy-5-{[6-methoxy-7- | MS (ESI+) m/z | HRMS: calcd for |
| | (tetrahydro-2H-pyran-2- | 426.1 | C₂₂H₂₃N₃O₆ + H+, |
| | ylmethoxy)quinazolin-4- | | 426.16596; found (ESI- |
| | yl]amino}benzo-1,4-quinone | | FTMS, [M + H]¹⁺), |
| | | | 426.16578 |

### EXAMPLE 349

### 2-chloro-4-hydroxy-3-methoxy-5-nitrobenzaldehyde

To a stirred solution of 2-chloro-3-formyl-6-methoxy-5-nitrophenyl acetate (Helv. Chem. Acta 952 (1989)) (21.33 g, 77.95 mmol) and dimethylsulfate (90 mL, 0.952 mol) in EtOH (192 mL) at 40° C., a 40% KOH (140 mL, 98.2 mol) solution was added drop wise over 45 minutes. The reaction was then stirred at 55° C. for 1 hour. The solvent was removed by rotary evaporator and the resulting residue was extracted with ether (2×). The ether solution was dried (MgSO₄) and was passed through a column of magnesol. The solvent was removed to give 22.3 g of 2-chloro-3,4-dimethoxy-5-nitro-benzaldehyde as a nearly colorless oil. 2-chloro-3,4-dimethoxy-5-nitro-benzaldehyde (22.33 g, 90.91 mmol), H₂O (1.12 mL) and LiCl (23.12 g, 0.545 mol) in DMF was heated at 110° C. for 3 hours. The dark red mixture was cooled and treated with a solution of saturated NaHCO₃ (59 mL) and H₂O (800 mL). The aqueous solution was washed with ether (2×), was then made acidic with H₂SO₄ and cooled to 4° C. The resulting solid was collected by vacuum filtration, washed with H₂O and dried in air to give 18.3 g (87%) of the title compound as an off white solid: MS (ESI) m/z 230; ¹H NMR (400 MHz, CHLOROFORM-D) δ ppm 4.01 (s, 3H) 8.54 (s, 1H) 10.35 (s, 1H) 11.14 (s, 1H).

### EXAMPLE 350

### 2-chloro-3-methoxy-5-nitrobenzene-1,4-diol

To compound 2-chloro-4-hydroxy-3-methoxy-5-nitrobenzaldehyde (17.8 g, 72.47 mmol), 1 N NaOH (72.5 mL, 72.5 mmol), H₂O (158 mL), 30% H₂O₂ (45 mL), and MeOH (158 mL) was added and the mixture was stirred at 50° C. for 3.5 hours. The MeOH was removed by rotary evaporator and the solution was then cooled. The resulting solid was collected by vacuum filtration, washed with H₂O, and air dried to yield 7.9 g (50%) of the title compound as an orange solid: MS (ESI) m/z 218; ¹H NMR (400 MHz, CHLOROFORM-D) δ ppm 4.01 (s, 3H) 5.43 (s, 1H) 7.56 (s, 1H) 10.37 (d, J=11.33 Hz, 1H).

### EXAMPLE 351

### 2-chloro-1,3,4-trimethoxy-5-nitrobenzene

Compound 2-chloro-3-methoxy-5-nitrobenzene-1,4-diol (7.8 g, 35.53 mmol) in DMF (77 mL) was treated with dimethylsulfate (11.2 g, 88.81 mmol) and K₂CO₃ (14.73 g, 106.57 mmol) and was heated to 80° C. for 1 hour. The reaction was then poured into H₂O. The resulting solid was collected by vacuum filtration, washed with H₂O, and air dried to give 8.0 g (91%) of the title compound as a gray solid: MS (APCI) m/z 247.1; MS (APCI) m/z 247.1; ¹H NMR (400 MHz, CHLOROFORM-D) δ ppm 3.96 (t, J=8.0 Hz, 9H) 7.17 (s, 1H).

### EXAMPLE 352

### 4-chloro-2,3,5-trimethoxy-phenylamine

Compound 2-chloro-1,3,4-trimethoxy-5-nitrobenzene (8.0 g, 32.31 mmol) was dissolved in MeOH (429 mL), was treated with Fe (10.83 g, 193.33 mmol), and AcOH (11.1 mL, 193.83 mmol) and was refluxed with mechanical stirring for 2 hours. The reaction was then treated with NaOH (10 M, 19.38 mL, 193.83 mmol) and filtered. The solid was washed with EtOAc. The filtrate was concentrated and then redissolved in EtOAc, washed with saturated NaHCO₃, and dried (MgSO4) and concentrated to give 6.17 g of the title compound as a light tan oil. This material was used without additional purification.

### EXAMPLE 353

### N-(4-chloro-2,3,5-trimethoxyphenyl)-6-methoxy-7-(2-methoxyethoxy)quinazolin-4-amine

A solution of N′-[2-cyano-4-methoxy-5-(2-methoxyethoxy)phenyl]-N,N-dimethylimidoformamide (7.8 g, 28.13 mmol) and 4-chloro-2,3,5-trimethoxy-phenylamine (6.12 g, 28.13 mmol) in AcOH (246 mL) was heated for 3.5 hours. The reaction was cooled to room temperature and diluted with ether. The resulting solid was collected by vacuum filtration to yield 12.03 g of the title compound as a beige powder (95%): MS (ESI) m/z 450.1; ¹H NMR (400 MHz, DMSO-D₆) δ ppm 3.34 (s, 3H) 3.68 (s, 3H) 3.72-3.77 (m, 2H) 3.81 (s, 3H) 3.85 (s, 3H) 3.95 (s, 3H) 4.23-4.30 (m, 2H) 7.17 (s, 1H) 7.20 (s, 1H) 7.83 (s, 1H) 8.37 (s, 1H) 9.30 (s, 1H).

### EXAMPLE 354

### 2-chloro-3-methoxy-5-{[6-methoxy-7-(2-methoxyethoxy)quinazolin-4-yl]amino}benzo-1,4-quinone

Compound N-(4-chloro-2,3,5-trimethoxyphenyl)-6-methoxy-7-(2-methoxyethoxy)quinazolin-4-amine (1.0 g, 2.22 mmol) was boiled to dissolve in CH₃CN (20 mL) and then diluted with H₂O (2 mL). While still hot, the solution was treated with Ce(NH₄)₂(NO₃)₄ (2.86 g, 5.22 mmol) in portions over 2 minutes. The reaction was then stirred at room temperature for 1 hour, diluted with H₂O (300 mL) and extracted with CHCl₃ (5×800 mL). The organic solution was dried (Na₂SO₄) and filtered through a pad of magnesol (eluted with CH₃Cl/EtOAc). The solvent was removed by rotary evaporator. The resulting solid was dissolved in boiling MeCN (200 mL) and diluted with ether (200 mL). A red solid formed upon cooling and was collected by vacuum filtration (0.59 g, 63%): MS (ESI) m/z 420; ¹H NMR (400 MHz, CHLOROFORM-D) δ ppm 3.49 (s, 3H) 3.82-3.95 (m, 2H) 4.08 (s, 3H) 4.20 (s, 3H) 4.26-4.40 (m, 2H) 7.03 (s, 1H) 7.32 (s, 1H) 8.20 (s, 1H) 8.47 (s, 1H) 8.81 (s, 1H).

### EXAMPLE 355

### 2-chloro-3-isopropoxy-5-{[6-methoxy-7-(2-methoxyethoxy)quinazolin-4-yl]amino}benzo-1,4-quinone

To a solution of 2-chloro-3-methoxy-5-{[6-methoxy-7-(2-methoxyethoxy)quinazolin-4-yl]amino}benzo-1,4-quinone (600 mg, 1.43 mmol) in dichloromethane (86 mL) was added CsCO₃ (931.31 mg, 2.86 mmol) and isopropanol (42 mL, 548.5 mmol). The reaction mixture was stirred at room temperature for 2.5 hours and filtered through a short column of silica gel. The solvent was removed in rotary evaporator. The residue was chromatographed on silica gel, eluting with CHCl₃/EtOAc from 1:1. The product fractions were combined and concentrated in rotary evaporator. The residue was stirred in ether. The resulting solid was filtered to yield 0.07 g (10.9%) of the title compound as a red powder: MS (ESI) m/z 448; ¹H NMR (400 MHz, CHLOROFORM-D) δ ppm 1.43 (d, J=6.30 Hz, 6H) 3.46-3.51 (s, 3H) 3.85-3.91 (m, 2H) 4.06-4.10 (s, 3H) 4.31-4.35 (m, 2H) 4.88-5.03 (m, 1H) 7.04 (s, 1H) 7.31-7.34 (s, 1H) 8.21 (s, 1H) 8.50 (s, 1H) 8.81-8.83 (s, 1H); Anal. (C₂₁H₂₂ClN₃O₆) C, H, N.

### EXAMPLE 356

### 2-chloro-3-(cyclopropylmethoxy)-5-{[6-methoxy-7-(2-methoxyethoxy)quinazolin-4-yl]amino}benzo-1,4-quinone

To a solution of 2-chloro-3-methoxy-5-{[6-methoxy-7-(2-methoxyethoxy)quinazolin-4-yl]amino}benzo-1,4-quinone (650 mg, 1.55 mmol) in dichloromethane (100 mL), CsCO₃ (1.01 g, 3.1 mmol) and cyclopropylmethanol (3.35 g, 46.45 mmol) was added. The reaction mixture was stirred at room temperature overnight, and then filtered through a short column of silica gel, eluting with CHCl₃/EtOAc=1:1. The solvent was removed in rotary evaporator. The residue was purified by thin layer chromatography, eluting with EtOAc. The major red band was collected, the silica was extracted with EtOAc/isopropanol. The solvent was removed to yield 0.143 g (20.1%) of the title compound as a red solid: MS (ESI) m/z 460; ¹H NMR (400 MHz, CHLOROFORM-D) 8 ppm 0.37-0.39 (m, 2H) 0.63-0.69 (m, 2H) 1.27-1.34 (m, 1H) 3.49 (s, 3H) 3.87-3.91 (m, 2H) 4.07 (s, 3H) 4.24 (d, J=7.30 Hz, 2H) 4.31-4.36 (m, 2H) 7.03 (s, 1H) 7.33 (s, 1H) 8.21 (s, 1H) 8.48 (s, 1H) 8.82 (s, 1H); Anal. (C₂₂H₂₂ClN₃O₆) C, H, N.

### EXAMPLE 357

### 3-chloro-2-methoxy-5-{[6-methoxy-7-(2-methoxyethoxy)quinazolin-4-yl]amino}benzo-1,4-quinone

A solution of 7.7 g (19 mmol) of 4-[(3-chloro-4-methoxy-2,5-dimethoxyphenyl)amino]-6-methoxy-7-(2-methoxyethoxy)quinoline-3-carbonitrile in 322 ml of acetonitrile was heated to reflux and to this solution, 65 ml of water was added. The mixture was stirred and when the temperature reached 30° C., 19 g (34.7 mmol) of ceric ammonium nitrate was added over 5 minutes. After 45 minutes, the mixture was diluted with dilute sodium bicarbonate. The solid was collected by filtration and washed with water. This solid was suspended in 300 ml of water and 35 ml of concentrated hydrochloride acid was added. After stirring for 15 minutes, the precipitated solid was collected. The solid was stirred with 700 ml of methylene chloride and saturated sodium bicarbonate solution. The organic layer was dried over magnesium sulfate and the solution was passed onto a column of Magnesol™. The product was eluted from the column using ethyl acetate. The solvent was evaporated from the product fractions to give a solid that was washed with ether, yielding the title compound: MS (ESI) m/z 420.

### EXAMPLE 358

### 3-chloro-2-[2-fluoro-1-(fluoromethyl)ethoxy]-5-{[6-methoxy-7-(2-methoxyethoxy)quinazolin-4-yl]amino}benzo-1,4-quinone

To a solution of 2-chloro-3-methoxy-5-{[6-methoxy-7-(2-methoxyethoxy)quinazolin-4-yl]amino}benzo-1,4-quinone (650 mg, 1.55 mmol) in dichloromethane (93 mL), CsCO₃ (670.93 mg, 2.06 mmol) and 1,3-difluoro-2-propanol (4.46 g, 46.45 mmol) was added. The reaction mixture was stirred at room temperature overnight and then filtered through a short column of silica gel, eluting with CHCl₃/EtOAc=1:1. The solvent was removed in a rotary evaporator. The residue was stirred with ether. The resulting solid was filtered to yield 0.25 g (33.6%) of the title compound as a red solid: MS (ESI) m/z 480.1; HRMS: calcd for C₂₂H₂₃F₂N₃O₇+H+, 480.15768; found (ESI-FTMS, [M+H]¹⁺), 480.15833. The purity of the title compound was evaluated on two HPLC systems and found to be 100% (system C, retention time=3.89 min) and 89% (system D, retention time=12.2 min). MS (ESI) m/z 484; HRMS: calcd for C₂₁H₂₀ClF₂N₃O₆+H+, 484.10815; found (ESI-FTMS, [M+H]¹⁺), 484.10815; ¹H NMR (400 MHz, CHLOROFORM-D) δ ppm 3.45-3.50 (s, 3H) 3.87-3.91 (m, 2H) 4.06 (s, 6H) 4.28-4.39 (m, 2H) 4.65-4.73 (m, 2H) 4.78-4.87 (m, 2H) 5.07-5.22 (m, 1H) 7.04 (s, 1H) 7.33 (s, 1H) 7.91-7.94 (s, 1H) 8.59 (s, 1H) 8.82 (s, 1H).

### EXAMPLES 359-361

2-chloro-3-methoxy-5-{[6-methoxy-7-(2-methoxyethoxy)quinazolin-4-yl]amino}benzo-1,4-quinone was dissolved in methylene chloride and treated with sodium phenoxide (trihydrate, 2.0 equivalents) and the appropriate alcohol in a 10-fold excess. The reaction was then agitated with a vortex shaker overnight. The reactions that were determined to be complete by LC-MS were washed with water and saturated sodium carbonate, dried over sodium sulfate and concentrated. The resulting residues were purified by either HPLC or crystallization from acetonitrile. The compounds of the invention made by this method are listed in Table 28.

TABLE 28

| Example | Compound Name | MS | HRMS |
|---|---|---|---|
| 359 | 3-chloro-2-[(3-fluorobenzyl)oxy]-5- | MS (ESI) m/z | |
| | {[6-methoxy-7-(2- | 514 | |
| | methoxyethoxy)quinazolin-4- | | |
| | yl]amino}benzo-1,4-quinone | | |
| 360 | 3-chloro-2-ethoxy-5-{[6-methoxy-7- | MS (ESI) m/z | HRMS: calcd for |
| | (2-methoxyethoxy)quinazolin-4- | 434.1 | C₂₀H₂₀ClN₃O₆ + H+, |
| | yl]amino}benzo-1,4-quinone | | 434.11134; found (ESI- |
| | | | FTMS, [M + H]¹⁺), |
| | | | 434.11093 |
| 361 | 3-chloro-5-{[6-methoxy-7-(2- | MS (ESI) m/z | |
| | methoxyethoxy)quinazolin-4- | 476 | |
| | yl]amino}-2-(THF-3-yloxy)benzo-1,4- | | |
| | quinone | | |

### EXAMPLES 362-364

A solution of 1.13 g (2.5 mmol) of 2-chloro-3-methoxy-5-{[6-methoxy-7-(2-methoxyethoxy)quinazolin-4-yl]amino}benzo-1,4-quinone and 1 ml of the appropriate amine in 30 ml of THF was stirred for 3 hours. The solid was collected via filtration and washed with THF and water and dried to yield the title compound. The compounds of the invention made using this method are listed in Table 29.

TABLE 29

| Example | Compound Name | MS | HRMS |
|---|---|---|---|
| 362 | 2-(4-benzylpiperazin-1-yl)-3-chloro- | MS (ESI) m/z | HRMS: calcd for |
| | 5-{[6-methoxy-7-(2- | 564.2; MS (ESI) | C₂₉H₃₀ClN₅O₅ + H+, |
| | methoxyethoxy)quinazolin-4- | m/z 282.6; | 564.20082; found (ESI- |
| | yl]amino}benzo-1,4-quinone | MS (ESI) m/z 303.1 | FTMS, [M + H]¹⁺), |
| | | | 564.19966 |
| 363 | 3-chloro-2-(3,5-dimethylpiperidin-1- | MS (ESI) m/z | HRMS: calcd for |
| | yl)-5-{[6-methoxy-7-(2- | 501.2 | C₂₅H₂₉ClN₄O₅ + H+, |
| | methoxyethoxy)quinazolin-4- | | 501.18992; found (ESI- |
| | yl]amino}benzo-1,4-quinone | | FTMS, [M + H]¹⁺), |
| | | | 501.1892 |
| 364 | 3-chloro-2-(dimethylamino)-5-{[6- | MS (ESI) m/z | HRMS: calcd for |
| | methoxy-7-(2- | 433.1 | C₂₀H₂₁ClN₄O₅ + H+, |
| | methoxyethoxy)quinazolin-4- | | 433.12732; found (ESI- |
| | yl]amino}benzo-1,4-quinone | | FTMS, [M + H]¹⁺), |
| | | | 433.1278 |

### EXAMPLES 365-366

To a solution of 2-chloro-3-methoxy-5-{[6-methoxy-7-(2-methoxyethoxy)quinazolin-4-yl]amino}benzo-1,4-quinone (800 mg, 1.91 mmol) in dichloromethane (115 mL), CsCO₃ (800 mg, 1.91 mmol) and the appropriate alcohol (1.45 mol) were added. The reaction mixture was stirred at room temperature for 2.5 hours and filtered through a short column of silica gel. The solvent was removed in rotary evaporator. The residue was chromatographed on silica gel, eluting with CHCl₃/EtOAc from 7:3 to 5:5. Product fraction was collected and concentrated in rotary evaporator. The residue was stirred in small amount of CH₃CN. The resulting solid was filtered to yield the title compound. The compounds of the invention made by this method are listed in Table 30.

TABLE 30

| Ex- | | | |
|---|---|---|---|
| am- | | | |
| ple | Compound Name | MS | HRMS |
| 365 | 2,3-dimethoxy-5-{[6- | MS (ESI) m/z | |
| | methoxy-7-(2- | 416.1 | |
| | methoxyethoxy)quinazolin- | | |
| | 4-yl]amino}benzo-1,4- | | |
| | quinone | | |
| 366 | 2-[2-fluoro-1- | MS (ESI) m/z | HRMS: calc'd for |
| | (fluoromethyl)ethoxy]-3- | 480.1 | C₂₂H₂₃F₂N₃O₇ + |
| | methoxy-5-{[6-methoxy-7- | | H+, 480.15768; |
| | (2- | | found (ESI-FTMS, |
| | methoxyethoxy)quinazolin- | | [M + H]¹⁺), |
| | 4-yl]amino}benzo-1,4- | | 480.15833 |
| | quinone | | |

### EXAMPLE 367

### (2E)-N-{4-[(4-chloro-2,5-dimethoxyphenyl)amino]-7-ethoxyquinazolin-6-yl}-4-(dimethylamino)but-2-enamide

Compound N-(4-chloro-2,5-dimethoxyphenyl)-7-ethoxyquinazoline-4,6-diamine was prepared by the methods described in U.S. Pat. Nos. 6,251,912 and 6,288,082. The (E)-4-(dimethylamino)-2-butenoic acid hydrochloride salt (4.42 g, 26.68 mmol) and oxalyl chloride (4.42 g, 26.68 mmol) in CH₃CN (57 mL) was stirred at 55° C. for 20 minutes. A trace of DMF was used after all solid dissolved. About half of the solvent was removed at reduced pressure at 50° C. and this solution was cooled. A solution of compound N-(4-chloro-2,5-dimethoxyphenyl)-7-ethoxyquinazoline-4,6-diamine (5 g, 13.34 mmol) in warm N-methylpyrolidone (57 mL) was added over 10 minutes. The reaction mixture was stirred at 0° C. for 2 hours and diluted with dilute NaHCO₃. The resulting solid was collected and dissolved in hot THF, diluted with EtOAc, dried over MgSO₄ and filtered. The solid was washed with hot THF-EtOAc. The filtrate was passed through a column of silica gel, eluting with EtOAc, EtOAc/MeOH and 700:300:10 EtOAc/MeOH/Et₃N. The solvent was removed from product fractions. The resulting solid was stirred in ether and collected to yield 5 g (77%) of (2E)-N-{4-[(4-chloro-2,5-dimethoxyphenyl)amino]-7-ethoxyquinazolin-6-yl}-4-(dimethylamino)but-2-enamide as a white solid: MS (ESI) m/z 486.1; MS (ESI) m/z 264; MS (ESI) m/z 243.5; ¹H NMR (400 MHz, DMSO-D₆) δ ppm 1.46 (t, J=6.92 Hz, 3H) 2.19 (s, 6H) 3.08 (d, J=4 Hz, 2H) 3.78 (d, J=4 Hz, 6H) 4.25-4.31 (m, 2H) 6.59 (d, J=16 Hz, 1H) 6.76-6.83 (m, 1H) 7.21 (d, J=8 Hz, 2H) 7.57 (s, 1H) 8.39 (s, 1H) 8.90 (s, 1H) 9.18 (s, 1H) 9.48 (s, 1H).

### EXAMPLE 368

### (2E)-4-(dimethylamino)-N-[7-ethoxy-4-[(4-methoxy-3,6-dioxocyclohexa-1,4-dien-1-yl)amino]quinazolin-6-yl]but-2-enamide

Compound (2E)-N-{4-[(4-chloro-2,5-dimethoxyphenyl)amino]-7-ethoxyquinazolin-6-yl}-4-(dimethylamino) (1.57 g, 3.23 mmol) was dissolved in CH₃CN (80 mL) and water (36 mL) and treated with ceric ammonium nitrate (4.25 g, 7.75 mmol). The reaction mixture was stirred at room temperature for 2.5 hours and then diluted with CHCl₃ (700 mL) and saturated Na₂CO₃ (50 mL). The solution was filtered through celite. The solid was washed many times with CHCl₃ to give a volume of 1400 mL organic layer. The solvent was evaporated, washed with water and diluted with MeOH (300 mL). The solution was dried over MgSO₄, filtered and treated with Et₃N (50 mL). The solution was refluxed for 2 hours 45 minutes and the solvent was removed. The residue was dissolved in CHCl₃, washed with saturated NaHCO₃, and dried over MgSO₄. The solution was filtered through a short column of magnesol, eluting with CHCl₃, and then with 500:500:50 CHCl₃-EtOAc-MeOH. The solvent of filtrate was evaporated. The resulting solid was stirred with EtOAc and collected to yield 850 g (58%) of (2E)-4-(dimethylamino)-N-{7-ethoxy-4-[(4-methoxy-3,6-dioxocyclohexa-1,4-dien-1-yl)amino]quinazolin-6-yl}but-2-enamide as a crystalline orange solid: MS (ESI) m/z 452.2; ¹H NMR (400 MHz, CHLOROFORM-D) δ ppm 1.59 (t, J=8 Hz, 3H) 2.34 (s, 6H) 3.19-3.21 (m, 2H) 3.91 (s, 3H) 4.30-4.35 (m, 2H) 5.99 (d, J=4 Hz, 1H) 6.25 (d, J=16 Hz, 1H) 7.06-7.11 (m, 1H) 7.30 (s, 1H) 8.07 (d, J=4 Hz, 1H) 8.14 (s, 1H) 8.81 (s, 1H) 8.98 (s, 1H) 9.30 (s, 1H).

### EXAMPLES 369-377

(2E)-4-(dimethylamino)-N-{7-ethoxy-4-[(4-chloro-3,6-dioxocyclohexa-1,4-dien-1-I)amino]quinazolin-6-yl}but-2-enamide was dissolved in methylene chloride and treated with sodium phenoxide (trihydrate, 2.0 equivalents) and the appropriate alcohol in a 10-fold excess. The reaction was then agitated with a vortex shaker overnight. The reactions that were determined to be complete by LC-MS were washed with water and saturated sodium carbonate, dried over sodium sulfate and concentrated. The resulting residues were purified by either HPLC or crystallization from acetonitrile. The compounds of the invention that were made using this method are listed in Table 31.

TABLE 31

| Example | Compound Name | MS | HRMS |
|---|---|---|---|
| 369 | (2E)-4-(dimethylamino)-N-[7-ethoxy- | MS (ESI) m/z | HRMS: calcd for |
| | 4-({4-[(3-fluorobenzyl)oxy]-3,6- | 546.2 | C₂₉H₂₈FN₅O₅ + H+, |
| | dioxocyclohexa-1,4-dien-1- | | 546.21472; found (ESI- |
| | yl}amino)quinazolin-6-yl]but-2- | | FTMS, [M + H]¹⁺), |
| | enamide | | 546.21347 |
| 370 | (2E)-4-(dimethylamino)-N-[7-ethoxy- | MS (ESI) m/z | HRMS: calcd for |
| | 4-({4-[2-fluoro-1- | 516.2 | C₂₅H₂₇F₂N₅O₅ + H+, |
| | (fluoromethyl)ethoxy]-3,6- | | 516.20530; found (ESI- |
| | dioxocyclohexa-1,4-dien-1- | | FTMS, [M + H]¹⁺), |
| | yl}amino)quinazolin-6-yl]but-2- | | 516.20519 |
| | enamide | | |
| 371 | (2E)-N-[4-({4-[(3,4- | MS (ESI) m/z | |
| | difluorobenzyl)oxy]-3,6- | 564.2 | |
| | dioxocyclohexa-1,4-dien-1- | | |
| | yl}amino)-7-ethoxyquinazolin-6-yl]-4- | | |
| | (dimethylamino)but-2-enamide | | |
| 372 | (2E)-N-(4-{[4-(benzyloxy)-3,6- | MS (ESI) m/z | HRMS: calcd for |
| | dioxocyclohexa-1,4-dien-1- | 528.2 | C₂₉H₂₉N₅O₅ + H+, |
| | yl]amino}-7-ethoxyquinazolin-6-yl)-4- | | 528.22415; found (ESI- |
| | (dimethylamino)but-2-enamide | | FTMS, [M + H]¹⁺), |
| | | | 528.22382 |
| | | MS (ESI) m/z | |
| | | 285.1 | |
| | | MS (ESI) m/z | |
| | | 264.6 | |
| 373 | (2E)-4-(dimethylamino)-N-(4-{[3,6- | MS (ESI) m/z | HRMS: calcd for |
| | dioxo-4-(pyridin-2- | 529.1 | C₂₈H₂₈N₆O₅ + H+, |
| | ylmethoxy)cyclohexa-1,4-dien-1- | | 529.21940; found (ESI- |
| | yl]amino}-7-ethoxyquinazolin-6- | | FTMS, [M + H]¹⁺), |
| | yl)but-2-enamide | | 529.21897 |
| | | MS (ESI) m/z | |
| | | 265 | |
| 374 | (2E)-N-[4-({4-[(3-chlorobenzyl)oxy]- | MS (ESI) m/z | HRMS: calcd for |
| | 3,6-dioxocyclohexa-1,4-dien-1- | 562 | C₂₉H₂₈ClN₅O₅ + H+, |
| | yl}amino)-7-ethoxyquinazolin-6-yl]-4- | | 562.18517; found (ESI- |
| | (dimethylamino)but-2-enamide | | FTMS, [M + H]¹⁺), |
| | | | 562.18608 |
| | | MS (ESI) m/z | |
| | | 281.5 | |
| 375 | (2E)-4-(dimethylamino)-N-(4-{[3,6- | MS (ESI) m/z | HRMS: calcd for |
| | dioxo-4-(2- | 534; | C₂₇H₂₇N₅O₅S + H+, |
| | thienylmethoxy)cyclohexa-1,4-dien- | | 534.18057; found (ESI- |
| | 1-yl]amino}-7-ethoxyquinazolin-6- | | FTMS, [M + H]¹⁺), |
| | yl)but-2-enamide | | 534.18094 |
| | | MS (ESI) m/z | |
| | | 288 | |
| | | MS (ESI) m/z | |
| | | 267.5 | |
| 376 | (2E)-4-(dimethylamino)-N-[7-ethoxy- | MS (ESI) m/z | HRMS: calcd for |
| | 4-({4-[(3-methoxybenzyl)oxy]-3,6- | 558.1 | C₃₀H₃₁N₅O₆ + H+, |
| | dioxocyclohexa-1,4-dien-1- | | 558.23471; found (ESI- |
| | yl}amino)quinazolin-6-yl]but-2- | | FTMS, [M + H]¹⁺), |
| | enamide | | 558.23403 |
| 377 | (2E)-4-(dimethylamino)-N-[7-ethoxy- | MS (ESI) m/z | HRMS: calcd for |
| | 4-({4-[(2-methylbenzyl)oxy]-3,6- | 542.1 | C₃₀H₃₁N₅O₅ + H+, |
| | dioxocyclohexa-1,4-dien-1- | | 542.23980; found (ESI- |
| | yl}amino)quinazolin-6-yl]but-2- | | FTMS, [M + H]¹⁺), |
| | enamide | | 542.23995 |
| | | MS (ESI) m/z | |
| | | 292 | |
| | | MS (ESI) m/z | |
| | | 271.5 | |

### EXAMPLE 378

### 2-({7-[3-(diethylamino)propoxy]-6-methoxyquinazolin-4-yl}amino)-5-methoxybenzo-1,4-quinone

To a solution of 2-({7-[3-(diethylamino)propoxy]-6-methoxyquinazolin-4-yl}amino)-5-chlorobenzo-1,4-quinone (˜1.9 mmol) in dichloromethane (115 mL), CsCO₃ (1.91 mmol) and the appropriate alcohol (˜1.45 mol) was added. The reaction mixture was stirred at room temperature for 2.5 hours and filtered through a short column of silica gel. The solvent was removed in rotary evaporator. The residue was chromatographed on silica gel, eluting with CHCl₃/EtOAc from 7:3 to 5:5. Product fraction was collected and concentrated in a rotary evaporator. The residue was stirred in small amount of CH₃CN. The resulting solid was filtered to yield 0.2 g (25%) of title compound as a red crystalline solid: MS (ESI+) m/z 441.2; HRMS: calcd for C₂₃H₂₈N₄O₅+H+, 441.21325; found (ESI-FTMS, [M+H]¹⁺), 441.21361.

### EXAMPLE 379

### 2,3,5-tris(ethylthio)-6-{[6-methoxy-7-(2-methoxyethoxy)quinazolin-4-yl]amino}benzo-1,4-quinone

To a degassed stirred solution of acetonitrile:deionized (MilliQ) water (1:1, 1000 mL) of the quinone (˜0.1 mmol, 40 mg) under N₂ added ethanethiol (10 quiv., ˜0.1 mL) was added. The solution was stirred until starting material was consumed shown by TLC or LCMS (1 hour-5 days). At the end of the reaction, 2.9 g of 0.7 mmol/g loading maleimide resin (Silicycle, Si-maleimide) was added to scavenge the ethanethiol. The suspension was stirred overnight then filtered (medium frit), extracted with 3×150 mL EtOAc dried with Na₂SO₃ and concentrated in vacuo (30-40° C.). The crude residue was purified by RP-HPLC (C18 Phenomenex Luna 150×30 mm, 20-80% MeCN:water 0.02% TFA). NaCl was added to the isolated fractions and extracted into DCM, dried with Na₂SO₃ concentrated in vacuo (30-40° C.) giving 3 mg of title compound: MS (ESI) m/z 536.2

What is claimed is:

1. A compound of formula 1 having the structure: 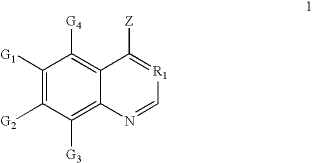
wherein:
R₁ is N, C—CN, C—H, C—F, C—Cl, C—Br, or C—I G₁, G₂, G₃, and G₄ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, alkylamido of 2-7 carbon atoms, halomethyl, alkyl-N-alkylamido of 4-10 carbon atoms, alkanoyloxy of 2-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, trifluoromethoxy, phenylacetyl, cyano, nitro, carboxy, carboalkoxy of 2-7 carbon atoms, carboalkyl of 2-7 carbon atoms, phenoxy, phenyl, thiophenoxy, benzyl, amino, hydroxyamino, alkoxyamino of 1-4 carbon atoms, alkylamino of 1-6 carbon atoms, dialkylamino of 2 to 12 carbon atoms, N-alkylcarbamoyl, N,N-dialkylcarbamoyl, N-alkyl-N-alkenylamino of 4 to 12 carbon atoms, N,N-dialkenylamino of 6-12 carbon atoms, phenylamino, benzylamino, R₂NH, 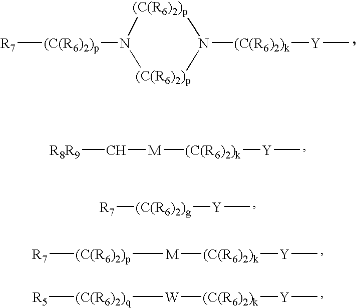
with the proviso that G₃ or G₄ are not R₂NH;
R₂, is selected from the group consisting of 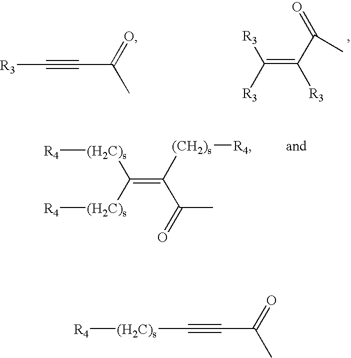
R₃ is, independently, hydrogen, alkyl of 1-6 carbon atoms, carboxy, carboalkoxy of 1-6 carbon atoms, phenyl, carboalkyl of 2-7 carbon atoms, 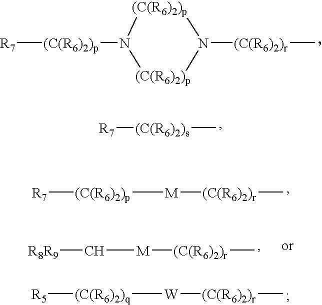
R₄ is Cl, Br, or I;
R₆ is hydrogen, alkyl of 1-6 carbon atoms, alkenyl of 2-6 carbon atoms, alkynyl of 2-6 carbon atoms, cycloalkyl of 1-6 carbon atoms, carboalkyl of 2-7 carbon atoms, carboxyalkyl 2-7 carbon atoms, phenyl, or phenyl optionally substituted with one or more halogen, alkoxy of 1-6 carbon atoms, trifluoromethyl, amino, alkylamino of 1-3 carbon atoms, dialkylamino of 2-6 carbon atoms, nitro, cyano, azido, halomethyl, alkoxymethyl of 2-7 carbon atoms, alkanoyloxymethyl of 2-7 carbon atoms, alkylthio of 1-6 carbon atoms, hydroxy, carboxyl, carboalkoxy of 2-7 carbon atoms, phenoxy, phenyl, thiophenoxy, benzoyl, benzyl, phenylamino, benzylamino, alkanoylamino of 1-6 carbon atoms, or alkyl of 1-6 carbon atoms; with the proviso that the alkenyl or alkynyl moiety is bound to a nitrogen or oxygen atom through a saturated carbon atom;
R₇ is —NR₆R₆, —OR₆, —R₄, —N(R₆)₃⁺ or —NR₆(OR₆);
M is >NR₆, —O—, >N—(C(R₆)₂)ₚNR₆R₆, or >N—(C(R₆)₂)ₚ—OR₆, or a divalent phenyl radical;
W is >NR₆, —O—, a divalent phenyl radical, or is a bond;
R₅ is a phenyl radical or a heterocyclic radical selected from the group consisting of morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane, tetrahydropyran, and 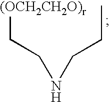
wherein the phenyl radical or the heterocylic radical may be optionally mono- or di-substituted on carbon with R₆, hydroxy, —N(R₆)₂, —OR₆—(C(R₆)₂)ₛOR₆, or —(C(R₆)₂)ₛN(R₆)₂ and
wherein the heterocylic radical may be optionally mono-substituted on nitrogen with R₆ and optionally mono or di-substituted on a saturated carbon with divalent radicals —O— or —O(C(R₆)₂)ₛO—;
R₈ and R₉ are each, independently, —(C(R₆)₂)ᵣNR₆R₆, or —(C(R₆)₂)ᵣOR₆;
Y is a divalent radical selected from the group consisting of 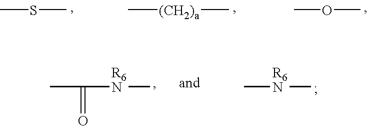
a=0-1;
g=1-6;
k=0-4;
p=2-4;
q=0-4;
r=1-4;
s=1-6;
provided that
when R₆ is alkenyl of 2-7 carbon atoms or alkynyl of 2-7 carbon atoms, such alkenyl or alkynyl moiety is bound to a nitrogen or oxygen atom through a saturated carbon atom;
and provided that
when Y is —NR₆— and R₇ is —NR₆R₆, —N(R₆)₃⁺, or —NR₆(OR₆), then g=2-6;
when M is —O— and R₇ is —OR₆ then p=1-4;
when Y is —NR₆— then k=2-4;
when Y is —O— and M or W is —O— then k=1-4;
when W is not a bond or a divalent phenyl radical with R₅ bonded through a nitrogen atom then q=2-4,
when M is a divalent phenyl radical then p=0-4 and r=0-4,
when W is a divalent phenyl radical then r=0-4,
and when W is a bond with R₅ bonded through a nitrogen atom and Y is —O— or —NR₆— then k=2-4;
Z is a radical selected from the group 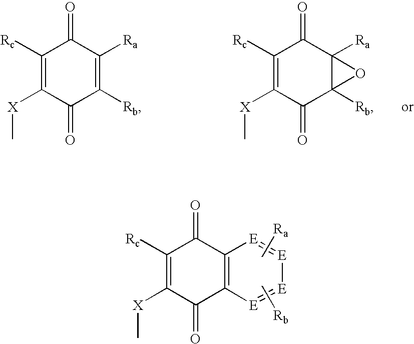
X is a divalent radical selected from the group —NH—, >NR₁₀, —O—, and —S—;
R₁₀ is an hydrogen, an alkyl group from 1-6 carbon atoms, phenyl or benzyl;
Rₐ, R_{b}, R_{c} are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxyalkyl of 1-6 carbon atoms, haloalkyl of 1-6 carbon atoms, alkanoyloxy of 2-6 carbon atoms, alkenoyloxy of 3-8 carbon atoms, alkynoyloxy of 3-8 carbon atoms, alkylamido of 2-7 carbon atoms, alkanoyloxymethyl of 2-7 carbon atoms, alkenoyloxymethyl of 4-9 carbon atoms, alkynoyloxymethyl of 4-9 carbon atoms, alkoxyalkyl of 2-14 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, phenylacetyl, alkenylsulfonamido of 2-6 carbon atoms, alkynylsulfonamido of 2-6 carbon atoms, hydroxy, trifluoromethyl, trifluoromethoxy, cyano, nitro, azido, carboxy, carboalkoxy of 2-7 carbon atoms, carboalkyl of 2-7 carbon atoms, phenoxy, phenyl, thiophenoxy, benzyl, benzyloxy, benzylthio, amino, hydroxyamino, alkoxyamino of 1-4 carbon atoms, alkylamino of 1-6 carbon atoms, dialkylamino of 2 to 12 carbon atoms, N-alkylcarbamoyl of 2 to 6 carbon atoms, N,N-dialkylcarbamoyl of 2 to 12 carbon atoms, N-alkyl-N-alkenylamino of 4 to 12 carbon atoms, N,N-dialkenylamino of 6-12 carbon atoms, phenylamino, benzylamino, 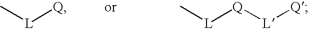
when attached to a double bond at contiguous carbon atoms, Rₐ and R_{b} can be taken together as the divalent radicals —(C(R₁₀)₂)₃—, —C(R₁₀)₂)₄—, —X—(C(R₁₀)₂)₃—, —X—(C(R₁₀)₂)₂—X—, —C(R₁₀)₂—X—(C(R₁₀)₂)₂—, or —C(R₁₀)₂—X—C(R₁₀)₂—;
Q and Q′ are a phenyl mono or divalent radical which may be optionally substituted with 1-5 halogen atoms, or mono- di- or tri-substituted with a substituent selected from the group consisting of hydrogen, alkyl of 1-6 carbon atoms, alkenyl of 2-6 carbon atoms, alkynyl of 2-6 carbon atoms, azido, hydroxyalkyl of 1-6 carbon atoms, alkylamido of 2-7 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, benzoyl, amino, phenylacetyl, alkylamino of 1-6 carbon atoms, dialkylamino of 2 to 12 carbon atoms, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N-alkylcarbamoyl of 2 to 6 carbon atoms, N,N-dialkylcarbamoyl of 2 to 12 carbon atoms, N,N-dialkylaminoalkoxy of 3-10 carbon atoms, mercapto, and benzoylamino, or
Q and Q′ are a mono or divalent radical comprising a 3-8-membered heterocyclic ring where the heterocyclic ring contains 1 to 3 heteroatoms selected from N, O, and S; wherein the heterocyclic ring may be optionally substituted with 1-5 halogen atoms, or mono- or di-substituted with a substituent selected from the group consisting of oxo, thio, alkyl of 1-6 carbon atoms, alkenyl of 2-6 carbon atoms, alkynyl of 2-6 carbon atoms, azido, alkylamido of 2-7 carbon atoms, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, phenylacetyl, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylcarbamoyl of 2 to 6 carbon atoms, N,N-dialkylcarbamoyl of 2 to 12 carbon atoms, N-alkylaminoalkoxy of 2-9 carbon atoms, N,N-dialkylaminoalkoxy of 3-10 carbon atoms, mercapto, and benzoylamino, or
Q and Q′ are a mono or divalent radical comprising a fused or bridged bicyclic or tricyclic carbocyclic ring system or a fused or bridged bicyclic or tricyclic heterocyclic ring system of 6 to 18 atoms, where the bicyclic or tricyclic heterocyclic ring system contains 1 to 4 heteroatoms selected from N, O, and S; wherein the bicyclic or tricyclic carbocyclic ring system or the bicyclic or tricyclic heterocyclic ring system may be optionally substituted with 1-5 halogen atoms, or mono-, di-, tri-, or tetra-substituted with a substituent selected from the group consisting of oxo, thio, alkyl of 1-6 carbon atoms, alkenyl of 2-6 carbon atoms, alkynyl of 2-6 carbon atoms, azido, alkylamido of 2-7 carbon atoms, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenylacetyl, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylcarbamoyl of 2 to 6 carbon atoms, N,N-dialkylcarbamoyl of 2 to 12 carbon atoms, N-alkylaminoalkoxy of 2-9 carbon atoms, N,N-dialkylaminoalkoxy of 3-10 carbon atoms, mercapto, and benzoylamino, or
Q and Q′ are hydrogen or a mono or divalent radical comprising straight or cyclic alkyl groups of 1 to 10 carbon atoms, both of which can optionally be branched, substituted with 1-6 halogen groups, or contain sites of unsaturation, or be;
L and L′ are divalent radicals selected from the group 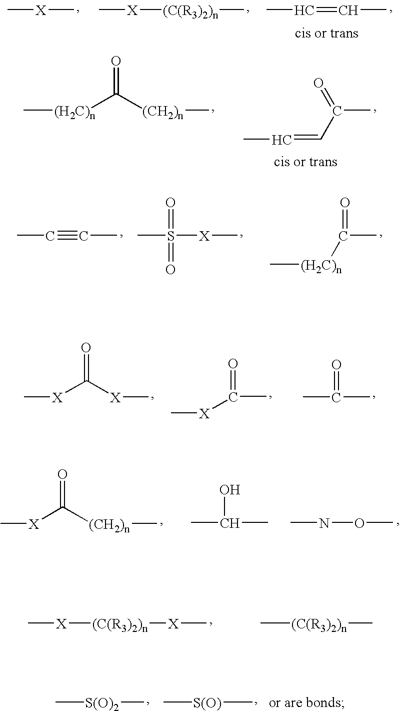
n is an integer from 1 to 4;
E is CH or N with the proviso that there be no more than 2 ring nitrogen atoms;
it is provided that when Z is the moiety 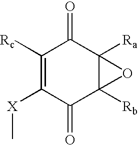
Rₐ and R_{b} are independently hydrogen or are attached to the ring only via carbon atoms;
or a pharmaceutically acceptable salt thereof.

2. The compound of claim 1, wherein R₁ is N, C—H, C—CN, C—F, C—Cl, C—Br, C—I or a pharmaceutically acceptable salt thereof.

3. The compound of claim 2, wherein Z is 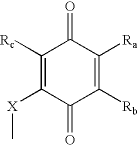
or a pharmaceutically acceptable salt thereof.

4. The compound of claim 2, wherein Z is 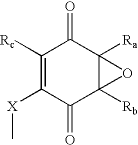
or a pharmaceutically acceptable salt thereof.

5. The compound of claim 2, wherein Z is 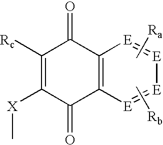
or a pharmaceutically acceptable salt thereof.

6. The compound according to claim 1, selected from the group consisting of:
(a) 2-chloro-5-[(6,7-dimethoxy-4-quinazolinyl)amino]benzo-1,4-quinone;
(b) 2-[(6,7-dimethoxy-4-quinazolinyl)amino]-5-methylbenzo-1,4-quinone;
(c) 4-[(6,7-dimethoxy-4-quinazolinyl)amino]-1-methyl-7-oxabicyclo[4.1.0]hept-3-ene-2,5-dione;
(d) 2-[(6,7-dimethoxy-4-quinazolinyl)amino]-6-methylbenzo-1,4-quinone;
(e) 2-{[6-methoxy-7-(2-methoxyethoxy)-4-quinazolinyl]amino}-5-methylbenzo-1,4-quinone;
(f) 4-{[6-methoxy-7-(2-methoxyethoxy)-4-quinazolinyl]amino}-1-methyl-7-oxabicyclo[4.1.0]hept-3-ene-2,5-dione;
(g) 2-[(6,7-dimethoxy-4-quinazolinyl)amino]-5-ethylbenzo-1,4-quinone;
(h) 4-[(6,7-dimethoxy-4-quinazolinyl)amino]-1-ethyl-7-oxabicyclo[4.1.0]hept-3-ene-2,5-dione;
(i) 2-[(6,7-dimethoxyquinazolin-4-yl)amino]-5-isopropylbenzo-1,4-quinone;
(j) 4-[(6,7-dimethoxyquinazolin-4-yl)amino]-1-isopropyl-7-oxabicyclo[4.1.0]hept-3-ene-2,5-dione;
(k) 2-[(6,7-dimethoxyquinazolin-4-yl)amino]-5-morpholin-4-ylbenzo-1,4-quinone;
(l) 2-[(6,7-dimethoxyquinazolin-4-yl)amino]-5-(methylamino)benzo-1,4-quinone;
(m) 2-[(6,7-dimethoxyquinazolin-4-yl)amino]-5-(dimethylamino)benzo-1,4-quinone;
(n) 2-[(6,7-dimethoxyquinazolin-4-yl)amino]-5-piperidin-1-ylbenzo-1,4-quinone;
(o) 2-[(6,7-dimethoxyquinazolin-4-yl)amino]-5-[methyl(phenyl)amino]benzo-1,4-quinone;
(p) 2-[(6,7-dimethoxyquinazolin-4-yl)amino]-5-phenoxybenzo-1,4-quinone;
(q) 2-(4-chlorophenoxy)-5-[(6,7-dimethoxyquinazolin-4-yl)amino]benzo-1,4-quinone;
(r) 2-[(6,7-dimethoxyquinazolin-4-yl)amino]-5-phenylbenzo-1,4-quinone;
(s) 4-[(6,7-dimethoxyquinazolin-4-yl)amino]-1-phenyl-7-oxabicyclo[4.1.0]hept-3-ene-2,5-dione;
(t) 2-anilino-5-[(6,7-dimethoxyquinazolin-4-yl)amino]benzo-1,4-quinone;
(u) 2-chloro-5-{[6-methoxy-7-(2-methoxyethoxy)quinazolin-4-yl]amino}benzo-1,4-quinone;
(v) 6-methoxy-7-(2-methoxyethoxy)-4-[(4-methyl-3,6-dioxocyclohexa-1,4-dien-1-yl)amino]quinoline-3-carbonitrile;
(w) 1-benzyl-4-{[6-methoxy-7-(2-methoxyethoxy)quinazolin-4-yl]amino}-7-36 oxabicyclo[4.1.0]hept-3-ene-2,5-dione;
(x) 2-(dimethylamino)-5-{[6-methoxy-7-(2-methoxyethoxy)quinazolin-4-yl]amino}benzo-1,4-quinone;
(y) 2-{[6-methoxy-7-(2-methoxyethoxy)quinazolin-4-yl]amino}-5-morpholin-4-ylbenzo-1,4-quinone;
(z) 2-{[6-methoxy-7-(2-methoxyethoxy)quinazolin-4-yl]amino}-5-[methyl(phenyl)amino]benzo-1,4-quinone;
(aa) 2-{[6-methoxy-7-(2-methoxyethoxy)quinazolin-4-yl]amino}-5-[(4-methoxyphenyl)(methyl)amino]benzo-1,4-quinone;
(bb) 2-[cyclohexyl(methyl)amino]-5-{[6-methoxy-7-(2-methoxyethoxy)quinazolin-4-yl]amino}benzo-1,4-quinone;
(cc) 2-[benzyl(methyl)amino]-5-{[6-methoxy-7-(2-methoxyethoxy)quinazolin-4-yl]amino}benzo-1,4-quinone;
(dd) 2-{[6-methoxy-7-(2-methoxyethoxy)quinazolin-4-yl]amino}-5-[(3-methylbenzyl)amino]benzo-1,4-quinone;
(ee) 2-{[6-methoxy-7-(2-methoxyethoxy)quinazolin-4-yl]amino}-5-(4-methylphenoxy)benzo-1,4-quinone;
(ff) 2-{[6-methoxy-7-(2-methoxyethoxy)quinazolin-4-yl]amino}-5-(pyridin-3-yloxy)benzo-1,4-quinone;
(gg) 2-{[6-methoxy-7-(2-methoxyethoxy)quinazolin-4-yl]amino}-5-(2-methylphenoxy)benzo-1,4-quinone;
(hh) 2-{[6-methoxy-7-(2-methoxyethoxy)quinazolin-4-yl]amino}-5-phenoxybenzo-1,4-quinone
(ii) 2-{[6-methoxy-7-(2-methoxyethoxy)quinazolin-4-yl]amino}-5-piperidin-1-yl-benzo-1,4-quinone;
(jj) 2-[(4-fluorophenyl)(methyl)amino]-5-{[6-methoxy-7-(2-methoxyethoxy)quinazolin-4-yl]amino}benzo-1,4-quinone;
(kk) 2-[[4-(dimethylamino)phenyl](methyl)amino]-5-{[6-methoxy-7-(2-methoxyethoxy)quinazolin-4-yl]amino}benzo-1,4-quinone;
(ll) 2-[(3-fluorophenyl)(methyl)amino]-5-{[6-methoxy-7-(2-methoxyethoxy)quinazolin-4-yl]amino}benzo-1,4-quinone;
(mm) 2-[4-(1H-imidazol-1-yl)phenoxy]-5-{[6-methoxy-7-(2-methoxyethoxy)quinazolin-4-yl]amino}benzo-1,4-quinone;
(nn) 2-[(3,4-dimethoxyphenyl)(methyl)amino]-5-{[6-methoxy-7-(2-methoxyethoxy)quinazolin-4-yl]amino}benzo-1,4-quinone;
(oo) 3-[(4-{[6-methoxy-7-(2-methoxyethoxy)quinazolin-4-yl]amino}-3,6-dioxocyclohexa-1,4-dien-1-yl)oxy]benzonitrile;
(pp) 2-{[6-methoxy-7-(2-methoxyethoxy)quinazolin-4-yl]amino}-5-(3-methoxyphenoxy)benzo-1,4-quinone;
(qq) 2-{[6-methoxy-7-(2-methoxyethoxy)quinazolin-4-yl]amino}-5-(4-phenoxyphenoxy)benzo-1,4-quinone;
(rr) 2-(4-fluorophenoxy)-5-{[6-methoxy-7-(2-methoxyethoxy)quinazolin-4-yl]amino}benzo-1,4-quinone;
(ss) 4-[(4-{[6-methoxy-7-(2-methoxyethoxy)quinazolin-4-yl]amino}-3,6-dioxocyclohexa-1,4-dien-1-yl)oxy]benzonitrile;
(tt) 2-{[6-methoxy-7-(2-methoxyethoxy)quinazolin-4-yl]amino}-5-(4-methoxyphenoxy)benzo-1,4-quinone;
(uu) 2-(3-chlorophenoxy)-5-{[6-methoxy-7-(2-methoxyethoxy)quinazolin-4-yl]amino}benzo-1,4-quinone;
(vv) 2-(3-acetylphenoxy)-5-{[6-methoxy-7-(2-methoxyethoxy)quinazolin-4-yl]amino}benzo-1,4-quinone;
(ww) 2-{[6-methoxy-7-(2-methoxyethoxy)quinazolin-4-yl]amino}-5-[4-(methylthio)phenoxy]benzo-1,4-quinone;
(xx) 2-{[6-methoxy-7-(2-methoxyethoxy)quinazolin-4-yl]amino}-5-[4-(trifluoromethyl)phenoxy]benzo-1,4-quinone;
(yy) 2-{[6-methoxy-7-(2-methoxyethoxy)quinazolin-4-yl]amino}-5-(phenylthio)benzo-1,4-quinone;
(zz) 2-(2-methoxyethoxy)-5-{[6-methoxy-7-(2-methoxyethoxy)quinazolin-4-yl]amino}benzo-1,4-quinone;
(aaa) 2-(benzyloxy)-5-{[6-methoxy-7-(2-methoxyethoxy)quinazolin-4-yl]amino}benzo-1,4-quinone;
(bbb) 4-[(4-chloro-3,6-dioxocyclohexa-1,4-dien-1-yl)amino]-6-methoxy-7-(2-methoxyethoxy)quinoline-3-carbonitrile;
(ccc) 4-[(3,6-dioxo-4-phenoxycyclohexa-1,4-dien-1-yl)amino]-6-methoxy-7-(2-methoxyethoxy)quinoline-3-carbonitrile;
(ddd) 4-({4-[4-(1H-imidazol-1-yl)phenoxy]-3,6-dioxocyclohexa-1,4-dien-1-yl}amino)-6-methoxy-7-(2-methoxyethoxy)quinoline-3-carbonitrile;
(eee) 2-methoxy-6-{[6-methoxy-7-(2-methoxyethoxy)quinazolin-4-yl]amino}benzo-1,4-quinone;
(fff) 5-methoxy-3-{[6-methoxy-7-(2-methoxyethoxy)quinazolin-4-yl]amino}-2-(phenylthio)benzo-1,4-quinone;
(ggg) 2-(benzylthio)-5-methoxy-3-{[6-methoxy-7-(2-methoxyethoxy)quinazolin-4-yl]amino}benzo-1,4-quinone;
(hhh) 2,3-dichloro-5-{[6-methoxy-7-(2-methoxyethoxy)quinazolin-4-yl]amino}benzo-1,4-quinone;
(iii) 3-methoxy-5-{[6-methoxy-7-(2-methoxyethoxy)quinazolin-4-yl]amino}-2-(1,3-thiazol-5-ylthio)benzo-1,4-quinone;
(jjj) ethyl {4-[(4-{[6-methoxy-7-(2-methoxyethoxy)quinazolin-4-yl]amino}-3,6-dioxocyclohexa-1,4-dien-1-yl)oxy]phenyl}acetate;
(kkk) 4-[(4-{[6-methoxy-7-(2-methoxyethoxy)quinazolin-4-yl]amino}-3,6-dioxocyclohexa-1,4-dien-1-yl)oxy]benzenesulfonamide;
(lll) 2-(4-benzoylphenoxy)-5-{[6-methoxy-7-(2-methoxyethoxy)quinazolin-4-yl]amino}benzo-1,4-quinone;
(mmm) methyl 3-{4-[(4-{[6-methoxy-7-(2-methoxyethoxy)quinazolin-4-yl]amino}-3,6-dioxocyclohexa-1,4-dien-1-yl)oxy]phenyl}propanoate;
(nnn) 2-(9H-carbazol-2-yloxy)-5-{[6-methoxy-7-(2-methoxyethoxy)quinazolin-4-yl]amino}benzo-1,4-quinone;
(ooo) methyl 4-[(4-{[6-methoxy-7-(2-methoxyethoxy)quinazolin-4-yl]amino}-3,6-dioxocyclohexa-1,4-dien-1-yl)oxy]benzoate;
(ppp) 2-{[6-methoxy-7-(2-methoxyethoxy)quinazolin-4-yl]amino}-5-[3-(trifluoromethyl)phenoxy]benzo-1,4-quinone;
(qqq) 2-(3-fluorophenoxy)-5-{[6-methoxy-7-(2-methoxyethoxy)quinazolin-4-yl]amino}benzo-1,4-quinone;
(rrr) ethyl 5-[(4-{[6-methoxy-7-(2-methoxyethoxy)quinazolin-4-yl]amino}-3,6-dioxocyclohexa-1,4-dien-1-yl)oxy]-2-methyl-1H-indole-3-carboxylate;
(sss) 2-(4-bromophenoxy)-5-{[6-methoxy-7-(2-methoxyethoxy)quinazolin-4-yl]amino}benzo-1,4-quinone;
(ttt) 2-(2-isoxazol-5-yl-4-methylphenoxy)-5-{[6-methoxy-7-(2-methoxyethoxy)quinazolin-4-yl]amino}benzo-1,4-quinone;
(uuu) benzyl 4-[(4-{[6-methoxy-7-(2-methoxyethoxy)quinazolin-4-yl]amino}-3,6-dioxocyclohexa-1,4-dien-1-yl)oxy]benzoate;
(vvv) 2-{[6-methoxy-7-(2-methoxyethoxy)quinazolin-4-yl]amino}-5-[4-(phenylacetyl)phenoxy]benzo-1,4-quinone;
(www) 2-[3-(ethylamino)phenoxy]-5-{[6-methoxy-7-(2-methoxyethoxy)quinazolin-4-yl]amino}benzo-1,4-quinone;
(xxx) 2-[(6-bromo-2-naphthyl)oxy]-5-{[6-methoxy-7-(2-methoxyethoxy)quinazolin-4-yl]amino}benzo-1,4-quinone;
(yyy) 2-[2-(benzyloxy)phenoxy]-5-{[6-methoxy-7-(2-methoxyethoxy)quinazolin-4-yl]amino}benzo-1,4-quinone;
(zzz) 2-(9H-fluoren-2-yloxy)-5-{[6-methoxy-7-(2-methoxyethoxy)quinazolin-4-yl]amino}benzo-1,4-quinone;
(aaaa) 2-[4-(2-aminoethyl)phenoxy]-5-{[6-methoxy-7-(2-methoxyethoxy)quinazolin-4-yl]amino}benzo-1,4-quinone;
(bbbb) 2-{[6-methoxy-7-(2-methoxyethoxy)quinazolin-4-yl]amino}-5-{4-[(2E)-3-phenylprop-2-enoyl]phenoxy}benzo-1,4-quinone;
(cccc) 2-{[6-methoxy-7-(2-methoxyethoxy)quinazolin-4-yl]amino}-5-[4-(1-methyl-1-phenylethyl)phenoxy]benzo-1,4-quinone;
(dddd) 2-chloro-5-methoxy-3-[6-methoxy-7-(2-methoxy-ethoxy)-quinazolin-4-ylamino]benzo-1,4-quinone;
(eeee) 5-methoxy-3-[6-methoxy-7-(2-methoxy-ethoxy)-quinazolin-4-ylamino]-2-(pyridin-2-ylsulfanyl)benzo-1,4-quinone;
(ffff) 2-(2-hydroxy-ethylsulfanyl)-3-[6-methoxy-7-(2-methoxy-ethoxy)-quinazolin-4-ylamino]-[1,4]naphthoquinone;
(gggg) 2-[6-methoxy-7-(2-methoxy-ethoxy)-quinazolin-4-ylamino]-[1,4]naphthoquinone;
(hhhh) 2-chloro-5-({6-methoxy-7-[(1-methylpiperidin-4-yl)methoxy]quinazolin-4-yl}amino)benzo-1,4-quinone;
(iiii) 2-(methoxy)-5-{[6-methoxy-7-(2-methoxyethoxy)quinazolin-4-yl]amino}benzo-1,4-quinone;
(jjjj) 2-[ethyl(methyl)amino]-5-{[6-methoxy-7-(2-methoxyethoxy)quinazolin-4-yl]amino}benzo-1,4-quinone;
(kkkk) 2-(diisobutylamino)-5-{[6-methoxy-7-(2-methoxyethoxy)quinazolin-4-yl]amino}benzo-1,4-quinone;
(llll) 2-(3,5-dimethylpiperidin-1-yl)-5-{[6-methoxy-7-(2-methoxyethoxy)quinazolin-4-yl]amino}benzo-1,4-quinone;
(mmmm) 2-{[6-methoxy-7-(2-methoxyethoxy)quinazolin-4-yl]amino}-5-(3-methylpiperidin-1-yl)benzo-1,4-quinone;
(nnnn) 2-[(2,3-dihydroxypropyl)(methyl)amino]-5-{[6-methoxy-7-(2-methoxyethoxy)quinazolin-4-yl]amino}benzo-1,4-quinone;
(oooo) 2-{[6-methoxy-7-(2-methoxyethoxy)quinazolin-4-yl]amino}-5-(2-methylaziridin-1-yl)benzo-1,4-quinone;
(pppp) 2-[(2R,6S)-2,6-dimethylmorpholin-4-yl]-5-{[6-methoxy-7-(2-methoxyethoxy)quinazolin-4-yl]amino}benzo-1,4-quinone;
(qqqq) 2-(dipropylamino)-5-{[6-methoxy-7-(2-methoxyethoxy)quinazolin-4-yl]amino}benzo-1,4-quinone;
(rrrr) 2-{[6-methoxy-7-(2-methoxyethoxy)quinazolin-4-yl]amino}-5-(2-pyridin-3-ylpiperidin-1-yl)benzo-1,4-quinone;
(ssss) tert-butyl 1-(4-{[6-methoxy-7-(2-methoxyethoxy)quinazolin-4-yl]amino}-3,6-dioxocyclohexa-1,4-dien-1-yl)-L-prolinate;
(tttt) 2-azocan-1-yl-5-{[6-methoxy-7-(2-methoxyethoxy)quinazolin-4-yl]amino}benzo-1,4-quinone;
(uuuu) 2-{[6-methoxy-7-(2-methoxyethoxy)quinazolin-4-yl]amino}-5-[methyl(pentyl)amino]benzo-1,4-quinone;
(vvvv) 2-{4-[4-chloro-3-(trifluoromethyl)phenyl]piperazin-1-yl}-5-{[6-methoxy-7-(2-methoxyethoxy)quinazolin-4-yl]amino}benzo-1,4-quinone;
(wwww) 2-{[6-methoxy-7-(2-methoxyethoxy)quinazolin-4-yl]amino}-5-[(2S)-2-(pyrrolidin-1-ylmethyl)pyrrolidin-1-yl]benzo-1,4-quinone;
(xxxx) 2-[4-(2-fluoro-4-nitrophenyl)piperazin-1-yl]-5-{[6-methoxy-7-(2-methoxyethoxy)quinazolin-4-yl]amino}benzo-1,4-quinone;
(yyyy) 2-[[(3S)-1-benzylpyrrolidin-3-yl](methyl)amino]-5-{[6-methoxy-7-(2-methoxyethoxy)quinazolin-4-yl]amino}benzo-1,4-quinone;
(zzzz) 2-(4-benzylpiperidin-1-yl)-5-{[6-methoxy-7-(2-methoxyethoxy)quinazolin-4-yl]amino}benzo-1,4-quinone;
(aaaaa) 2-[4-(2-hydroxyethyl)piperazin-1-yl]-5-{[6-methoxy-7-(2-methoxyethoxy)quinazolin-4-yl]amino}benzo-1,4-quinone;
(bbbbb) 2-{[6-methoxy-7-(2-methoxyethoxy)quinazolin-4-yl]amino}-5-(4-pyrazin-2-ylpiperazin-1-yl)benzo-1,4-quinone;
(ccccc) 2-[[2-(1H-indol-3-yl)ethyl](methyl)amino]-5-{[6-methoxy-7-(2-methoxyethoxy)quinazolin-4-yl]amino}benzo-1,4-quinone;
(ddddd) ethyl1-(4-{[6-methoxy-7-(2-methoxyethoxy)quinazolin-4-yl]amino}-3,6-dioxocyclohexa-1,4-dien-1-yl)piperidine-4-carboxylate;
(eeeee) 2-{[6-methoxy-7-(2-methoxyethoxy)quinazolin-4-yl]amino}-5-[4-(2-methoxyphenyl)piperidin-1-yl]benzo-1,4-quinone;
(fffff) 2-(4-benzyl-1,4-diazepan-1-yl)-5-{[6-methoxy-7-(2-methoxyethoxy)quinazolin-4-yl]amino}benzo-1,4-quinone;
(ggggg) 2-(1,4′-bipiperidin-1′-yl)-5-{[6-methoxy-7-(2-methoxyethoxy)quinazolin-4-yl]amino}benzo-1,4-quinone;
(hhhhh) tert-butylN-(4-{[6-methoxy-7-(2-methoxyethoxy)quinazolin-4-yl]amino}-3,6-dioxocyclohexa-1,4-dien-1-yl)-N-methylglycinate;
(iiiii) 2-[[2-(3,4-dimethoxyphenyl)ethyl](methyl)amino]-5-{[6-methoxy-7-(2-methoxyethoxy)quinazolin-4-yl]amino}benzo-1,4-quinone;
(jjjjj) 2-{[6-methoxy-7-(2-methoxyethoxy)quinazolin-4-yl]amino}-5-[4-(2-pyrrolidin-1-ylethyl)piperazin-1-yl]benzo-1,4-quinone;
(kkkkk) 2-{[6-methoxy-7-(2-methoxyethoxy)quinazolin-4-yl]amino}-5-[4-(1-methylpiperidin-4-yl)piperazin-1-yl]benzo-1,4-quinone;
(lllll) 2-{[6-methoxy-7-(2-methoxyethoxy)quinazolin-4-yl]amino}-5-[methyl(2-phenylethyl)amino]benzo-1,4-quinone;
(mmmmm) 2-[4-(ethylsulfonyl)piperazin-1-yl]-5-{[6-methoxy-7-(2-methoxyethoxy)quinazolin-4-yl]amino}benzo-1,4-quinone;
(nnnnn) 2-{[6-methoxy-7-(2-methoxyethoxy)quinazolin-4-yl]amino}-5-pyrrolidin-1-ylbenzo-1,4-quinone;
(ooooo) 2-(2,3-dihydro-5H-benzo[f][1,4]oxazepin-4-yl)-5-[6-methoxy-7-(2-methoxy-ethoxy)-quinazolin-4-ylamino]benzo-1,4-quinone;
(ppppp) 2-{4-hydroxy-4-[3-(trifluoromethyl)phenyl]piperidin-1-yl}-5-{[6-methoxy-7-(2-methoxyethoxy)quinazolin-4-yl]amino}benzo-1,4-quinone;
(qqqqq) 2-[(1R,4R)-5-(4-chlorophenyl)-2,5-diazabicyclo[2.2.1]hept-2-yl]-5-{[6-methoxy-7-(2-methoxyethoxy)quinazolin-4-yl]amino}benzo-1,4-quinone;
(rrrrr) 1-{4-[6-methoxy-7-(2-methoxyethoxy)-quinazolin-4-ylamino]-3,6-dioxo-cyclohexa-1,4-dienyl}-piperidine-4-carboxylic acid;
(sssss) 1-(4-{[6-methoxy-7-(2-methoxyethoxy)quinazolin-4-yl]amino}-3,6-dioxocyclohexa-1,4-dien-1-yl)azetidine-3-carboxylic acid;
(ttttt) 2-[[2-(diethylamino)ethyl](methyl)amino]-5-{[6-methoxy-7-(2-methoxyethoxy)quinazolin-4-yl]amino}benzo-1,4-quinone;
(uuuuu) 2-{[6-methoxy-7-(2-methoxyethoxy)quinazolin-4-yl]amino}-5-[2-(trifluoromethyl)pyrrolidin-1-yl]benzo-1,4-quinone;
(vvvvv) N,N-diethyl-1-(4-{[6-methoxy-7-(2-methoxyethoxy)quinazolin-4-yl]amino}-3,6-dioxocyclohexa-1,4-dien-1-yl)piperidine-3-carboxamide;
(wwwww) ethyl 1-(4-{[6-methoxy-7-(2-methoxyethoxy)quinazolin-4-yl]amino}-3,6-dioxocyclohexa-1,4-dien-1-yl)piperidine-3-carboxylate;
(xxxxx) 2-(4-benzylpiperazin-1-yl)-5-{[6-methoxy-7-(2-methoxyethoxy)quinazolin-4-yl]amino}benzo-1,4-quinone;
(yyyyy) 2-[(1,3-dioxolan-2-ylmethyl)(methyl)amino]-5-{[6-methoxy-7-(2-methoxyethoxy)quinazolin-4-yl]amino}benzo-1,4-quinone;
(zzzzz)₂-[[2-(dimethylamino)ethyl](methyl)amino]-5-{[6-methoxy-7-(2-methoxyethoxy)quinazolin-4-yl]amino}benzo-1,4-quinone;
(aaaaaa) 2-[(cyclopropylmethyl)(propyl)amino]-5-{[6-methoxy-7-(2-methoxyethoxy)quinazolin-4-yl]amino}benzo-1,4-quinone;
(bbbbbb) 2-[(2-methoxyethyl)(methyl)amino]-5-{[6-methoxy-7-(2-methoxyethoxy)quinazolin-4-yl]amino}benzo-1,4-quinone;
(cccccc) 2-[6-methoxy-7-(3-methoxy-propyl)-quinazolin-4-ylamino]-5-(3-methylamino-pyrrolidin-1-yl)benzo-1,4-quinone;
(dddddd) 2-[isobutyl(methyl)amino]-5-{[6-methoxy-7-(2-methoxyethoxy)quinazolin-4-yl]amino}benzo-1,4-quinone;
(eeeeee) 2-(4-ethylpiperazin-1-yl)-5-{[6-methoxy-7-(2-methoxyethoxy)quinazolin-4-yl]amino}benzo-1,4-quinone;
(ffffff) 2-[butyl(methyl)amino]-5-{[6-methoxy-7-(2-methoxyethoxy)quinazolin-4-yl]amino}benzo-1,4-quinone;
(gggggg) 2-{[6-methoxy-7-(2-methoxyethoxy)quinazolin-4-yl]amino}-5-[methyl(1-methylpiperidin-4-yl)amino]benzo-1,4-quinone;
(hhhhhh) 2-[3-(hydroxymethyl)piperidin-1-yl]-5-{[6-methoxy-7-(2-methoxyethoxy)quinazolin-4-yl]amino}benzo-1,4-quinone;
(iiiiii) 2-(4-acetylpiperazin-1-yl)-5-{[6-methoxy-7-(2-methoxyethoxy)quinazolin-4-yl]amino}benzo-1,4-quinone;
(jjjjjj) 2-{[6-methoxy-7-(2-methoxyethoxy)quinazolin-4-yl]amino}-5-[methyl(1-methylpyrrolidin-3-yl)amino]benzo-1,4-quinone;
(kkkkkk) 2-[[3-(dimethylamino)propyl](methyl)amino]-5-{[6-methoxy-7-(2-methoxyethoxy)quinazolin-4-yl]amino}benzo-1,4-quinone;
(llllll) 2-(diallylamino)-5-{[6-methoxy-7-(2-methoxyethoxy)quinazolin-4-yl]amino}benzo-1,4-quinone;
(mmmmmm) 2-[(2-furylmethyl)(methyl)amino]-5-{[6-methoxy-7-(2-methoxyethoxy)quinazolin-4-yl]amino}benzo-1,4-quinone;
(nnnnnn) 2-{[6-methoxy-7-(2-methoxyethoxy)quinazolin-4-yl]amino}-5-[(4-morpholin-4-ylphenyl)amino]benzo-1,4-quinone;
(oooooo) 2-[allyl(methyl)amino]-5-{[6-methoxy-7-(2-methoxyethoxy)quinazolin-4-yl]amino}benzo-1,4-quinone;
(pppppp) 2-(2,3-dihydro-1,4-benzodioxin-6-ylamino)-5-{[6-methoxy-7-(2-methoxyethoxy)quinazolin-4-yl]amino}benzo-1,4-quinone;
(qqqqqq) 2-[(4-isopropylphenyl)amino]-5-{[6-methoxy-7-(2-methoxyethoxy)quinazolin-4-yl]amino}benzo-1,4-quinone;
(rrrrrr) 2-[(2-ethylphenyl)amino]-5-{[6-methoxy-7-(2-methoxyethoxy)quinazolin-4-yl]amino}benzo-1,4-quinone;
(ssssss) 2-[(9-ethyl-9H-carbazol-3-yl)amino]-5-{[6-methoxy-7-(2-methoxyethoxy)quinazolin-4-yl]amino}benzo-1,4-quinone;
(tttttt) 2-[ethyl(3-methylphenyl)amino]-5-{[6-methoxy-7-(2-methoxyethoxy)quinazolin-4-yl]amino}benzo-1,4-quinone;
(uuuuuu) 2-[(3,5-di-tert-butylphenyl)amino]-5-{[6-methoxy-7-(2-methoxyethoxy)quinazolin-4-yl]amino}benzo-1,4-quinone;
(vvvvvv) 2-{[4-(4-chlorophenoxy)phenyl]amino}-5-{[6-methoxy-7-(2-methoxyethoxy)quinazolin-4-yl]amino}benzo-1,4-quinone;
(wwwwww) ethyl 5-{4-[(4-{[6-methoxy-7-(2-methoxyethoxy)quinazolin-4-yl]amino}-3,6-dioxocyclohexa-1,4-dien-1-yl)amino]phenyl}-2-methyl-3-furoate;
(xxxxxx) 2-(4-imidazol-1-yl-phenylamino)-5-[6-methoxy-7-(3-methoxypropyl)-quinazolin-4-ylamino]benzo-1,4-quinone;
(yyyyyy) N-(4-{[6-methoxy-7-(2-methoxyethoxy)quinazolin-4-yl]amino}-3,6-dioxocyclohexa-1,4-dien-1-yl)-L-valine;
(zzzzzz) 2-{[6-methoxy-7-(2-methoxyethoxy)quinazolin-4-yl]amino}-5-(pentafluorophenoxy)benzo-1,4-quinone;
(aaaaaaa) 2-{[6-methoxy-7-(2-methoxyethoxy)quinazolin-4-yl]amino}-5-[(2-methoxypropyl)amino]benzo-1,4-quinone;
(bbbbbbb) 2-[(2-hydroxypropyl)amino]-5-{[6-methoxy-7-(2-methoxyethoxy)quinazolin-4-yl]amino}benzo-1,4-quinone;
(ccccccc) 2-{[6-methoxy-7-(2-methoxyethoxy)quinazolin-4-yl]amino}-5-(5-methyl-2-oxo-1,3-oxazolidin-3-yl)benzo-1,4-quinone;
(ddddddd). 3-iodo-2-{[6-methoxy-7-(2-methoxyethoxy)quinazolin-4-yl]amino}-5-methylbenzo-1,4-quinone;
(eeeeeee) 2-Iodo-5-methoxy-3-{[6-methoxy-7-(2-methoxyethoxy)quinazolin-4-yl]amino}benzo-1,4-quinone;
(fffffff) 3-[(2-hydroxyethyl)thio]-2-{[6-methoxy-7-(2-methoxyethoxy)quinazolin-4-yl]amino}-5-methylbenzo-1,4-quinone;
(ggggggg) 2-amino-5-{[6-methoxy-7-(2-methoxyethoxy)quinazolin-4-yl]amino}benzo-1,4-quinone;
(hhhhhhh) 2-chloro-3-methoxy-5-{[6-methoxy-7-(2-methoxyethoxy)quinazolin-4-yl]amino}benzo-1,4-quinone;
(iiiiiii) 2-chloro-3-methoxy-5-{[6-methoxy-7-(2-methoxyethoxy)quinazolin-4-yl]amino}-6-(methylthio)benzo-1,4-quinone;
(jjjjjjj) 5-methoxy-3-{[6-methoxy-7-(2-methoxyethoxy)quinazolin-4-yl]amino}-2-(methylthio)benzo-1,4-quinone;
(kkkkkkk) 2-bromo-6-{[6-methoxy-7-(2-methoxyethoxy)quinazolin-4-yl]amino}benzo-1,4-quinone;
(lllllll) 4-[(4-{[6-methoxy-7-(2-methoxyethoxy)quinazolin-4-yl]amino}-3,6-dioxocyclohexa-1,4-dien-1-yl)oxy]benzamide;
(mmmmmmm) 2-{[6-methoxy-7-(2-methoxyethoxy)quinazolin-4-yl]amino}-5-(3-methylphenoxy)benzo-1,4-quinone;
(nnnnnnn) 2-[4-(benzyloxy)phenoxy]-5-{[6-methoxy-7-(2-methoxyethoxy)quinazolin-4-yl]amino}benzo-1,4-quinone;
(ooooooo) N-{3-[(4-{[6-methoxy-7-(2-methoxyethoxy)quinazolin-4-yl]amino}-3,6-dioxocyclohexa-1,4-dien-1-yl)oxy]phenyl}acetamide;
(ppppppp) 2-(isoquinolin-5-yloxy)-5-{[6-methoxy-7-(2-methoxyethoxy)quinazolin-4-yl]amino}benzo-1,4-quinone;
(qqqqqqq) 2-(2-allylphenoxy)-5-{[6-methoxy-7-(2-methoxyethoxy)quinazolin-4-yl]amino}benzo-1,4-quinone;
(rrrrrrr) 2-{[6-methoxy-7-(2-methoxyethoxy)quinazolin-4-yl]amino}-5-[3-(trifluoromethyl)phenoxy]benzo-1,4-quinone;
(sssssss) 2-(2-benzoylphenoxy)-5-{[6-methoxy-7-(2-methoxyethoxy)quinazolin-4-yl]amino}benzo-1,4-quinone;
(ttttttt) 2-(2-bromophenoxy)-5-{[6-methoxy-7-(2-methoxyethoxy)quinazolin-4-yl]amino}benzo-1,4-quinone;
(uuuuuuu) 2-(2-chlorophenoxy)-5-{[6-methoxy-7-(2-methoxyethoxy)quinazolin-4-yl]amino}benzo-1,4-quinone;
(wvvvvv) 2-[(4-{[6-methoxy-7-(2-methoxyethoxy)quinazolin-4-yl]amino}-3,6-dioxocyclohexa-1,4-dien-1-yl)oxy]benzonitrile;
(wwwwwww) 2-{[6-methoxy-7-(2-methoxyethoxy)quinazolin-4-yl]amino}-5-(quinolin-6-yloxy)benzo-1,4-quinone;
(xxxxxxx) 2-[(1-acetyl-2-naphthyl)oxy]-5-{[6-methoxy-7-(2-methoxyethoxy)quinazolin-4-yl]amino}benzo-1,4-quinone;
(yyyyyyy) 2-[(2-acetyl-1-naphthyl)oxy]-5-{[6-methoxy-7-(2-methoxyethoxy)quinazolin-4-yl]amino}benzo-1,4-quinone;
(zzzzzzz) 2-{[6-methoxy-7-(2-methoxyethoxy)quinazolin-4-yl]amino}-5-[4-(3-oxobutyl)phenoxy]benzo-1,4-quinone;
(aaaaaaaa) 2-(dibenzo[b,d]furan-2-yloxy)-5-{[6-methoxy-7-(2-methoxyethoxy)quinazolin-4-yl]amino}benzo-1,4-quinone;
(bbbbbbbb) 2-{[6-methoxy-7-(2-methoxyethoxy)quinazolin-4-yl]amino}-5-[(2-oxo-1,3-benzoxathiol-6-yl)oxy]benzo-1,4-quinone;
(cccccccc) 2-[(4-chloro-1-naphthyl)oxy]-5-{[6-methoxy-7-(2-methoxyethoxy)quinazolin-4-yl]amino}benzo-1,4-quinone;
(dddddddd) methyl 3-[(4-{[6-methoxy-7-(2-methoxyethoxy)quinazolin-4-yl]amino}-3,6-dioxocyclohexa-1,4-dien-1-yl)oxy]-2-naphthoate;
(eeeeeeee) 2-[2-fluoro-1-(fluoromethyl)ethoxy]-5-{[6-methoxy-7-(2-methoxyethoxy)quinazolin-4-yl]amino}benzo-1,4-quinone;
(ffffffff) 2-(cyclopropylmethoxy)-5-{[6-methoxy-7-(2-methoxyethoxy)quinazolin-4-yl]amino}benzo-1,4-quinone;
(gggggggg) 2-(cyclopentyloxy)-5-{[6-methoxy-7-(2-methoxyethoxy)quinazolin-4-yl]amino}benzo-1,4-quinone;
(hhhhhhhh) 2-(cyclohexylmethoxy)-5-{[6-methoxy-7-(2-methoxyethoxy)quinazolin-4-yl]amino}benzo-1,4-quinone;
(iiiiiiii) 3-[(4-{[6-methoxy-7-(2-methoxyethoxy)quinazolin-4-yl]amino}-3,6-dioxocyclohexa-1,4-dien-1-yl)oxy]propanenitrile;
(jjjjjjjj) 2-{[6-methoxy-7-(2-methoxyethoxy)quinazolin-4-yl]amino}-5-(2-phenoxyethoxy)benzo-1,4-quinone;
(kkkkkkkk) 2-[(3-methoxybenzyl)oxy]-5-{[6-methoxy-7-(2-methoxyethoxy)quinazolin-4-yl]amino}benzo-1,4-quinone;
(llllllll) 2-{[6-methoxy-7-(2-methoxyethoxy)quinazolin-4-yl]amino}-5-(2,2,2-trifluoroethoxy)benzo-1,4-quinone;
(mmmmmmmm) 2-{[6-methoxy-7-(2-methoxyethoxy)quinazolin-4-yl]amino}-5-(tetrahydrofuran-3-yloxy)benzo-1,4-quinone;
(nnnnnnnn) 2-{[6-methoxy-7-(2-methoxyethoxy)quinazolin-4-yl]amino}-5-(pyridin-3-ylmethoxy)benzo-1,4-quinone;
(oooooooo) 2-{[6-methoxy-7-(2-methoxyethoxy)quinazolin-4-yl]amino}-5-{2-[methyl(phenyl)amino]ethoxy}benzo-1,4-quinone;
(pppppppp) 2-{[6-methoxy-7-(2-methoxyethoxy)quinolin-4-yl]amino}-5-[4-(1-methyl-1-phenylethyl)phenoxy]benzo-1,4-quinone;
(qqqqqqqq) 2-(dimethylamino)-5-{[6-methoxy-7-(2-methoxyethoxy)quinolin-4-yl]amino}benzo-1,4-quinone;
(rrrrrrrr) 2-{[6-methoxy-7-(2-methoxyethoxy)quinazolin-4-yl]amino}benzo-1,4-quinone;
(yyyyyyyy) 2-chloro-5-{[6-methoxy-7-(2-methoxyethoxy)quinolin-4yl]amino}benzo-1,4-quinone;
(zzzzzzzz) 2-(2,5-dimethylpyrrolidin-1-yl)-5-{[6-methoxy-7-(2-methoxyethoxy)quinazolin-4-yl]amino}benzo-1,4-quinone;
(aaaaaaaaa) 2-bromo-5-{[6-methoxy-7-(2-methoxyethoxy)quinazolin-4-yl]amino}benzo-1,4-quinone;
(bbbbbbbbb) 2-{[6-methoxy-7-(2-methoxyethoxy)quinazolin-4-yl]amino}-5-[methyl(3-methylphenyl)amino]benzo-1,4-quinone;
(ccccccccc) 2-[benzyl(4-methoxyphenyl)amino]-5-{[6-methoxy-7-(2-methoxyethoxy)quinazolin-4-yl]amino}benzo-1,4-quinone;
(ddddddddd) 2-[ethyl(4-methylphenyl)amino]-5-{[6-methoxy-7-(2-methoxyethoxy)quinazolin-4-yl]amino}benzo-1,4-quinone;
(eeeeeeeee) 2-[butyl(phenyl)amino]-5-{[6-methoxy-7-(2-methoxyethoxy)quinazolin-4-yl]amino}benzo-1,4-quinone;
(fffffffff) 2-[ethyl(phenyl)amino]-5-{[6-methoxy-7-(2-methoxyethoxy)quinazolin-4-yl]amino}benzo-1,4-quinone;
(ggggggggg) 2-{[6-methoxy-7-(2-methoxyethoxy)quinazolin-4-yl]amino}-5-[pentyl(phenyl)amino]benzo-1,4-quinone;
(hhhhhhhhh) 2-(5-bromo-2,3-dihydro-1H-indol-1-yl)-5-{[6-methoxy-7-(2-methoxyethoxy)quinazolin-4-yl]amino}benzo-1,4-quinone;
(iiiiiiiii) 2-(2,3-dihydro-1H-indol-1-yl)-5-{[6-methoxy-7-(2-methoxyethoxy)quinazolin-4-yl]amino}benzo-1,4-quinone;
ojjjjjjj) 2-[(4-chlorophenyl)(methyl)amino]-5-{[6-methoxy-7-(2-methoxyethoxy)quinazolin-4-yl]amino}benzo-1,4-quinone;
(kkkkkkkkk) 2-[1,3-benzodioxol-5-yl(ethyl)amino]-5-{[6-methoxy-7-(2-methoxyethoxy)quinazolin-4-yl]amino}benzo-1,4-quinone;
(lllllllll) 2-[ethyl(1-naphthyl)amino]-5-{[6-methoxy-7-(2-methoxyethoxy)quinazolin-4-yl]amino}benzo-1,4-quinone;
(mmmmmmmmm) 2-[(3-hydroxy-3-phenylpropyl)(methyl)amino]-5-{[6-methoxy-7-(2-methoxyethoxy)quinazolin-4-yl]amino}benzo-1,4-quinone;
(nnnnnnnnn) 2-{[6-methoxy-7-(2-methoxyethoxy)quinazolin-4-yl]amino}-5-[4-(2-naphthylmethyl)piperazin-1-yl]benzo-1,4-quinone;
(ooooooooo) 2-{[6-methoxy-7-(2-methoxyethoxy)quinazolin-4-yl]amino}-5-[4-(1-naphthylmethyl)piperazin-1-yl]benzo-1,4-quinone;
(ppppppppp) 2-[4-(2,4-dimethoxybenzyl)piperazin-1-yl]-5-{[6-methoxy-7-(2-methoxyethoxy)quinazolin-4-yl]amino}benzo-1,4-quinone;
(qqqqqqqqq) 2-[4-(3-chlorobenzyl)piperazin-1-yl]-5-{[6-methoxy-7-(2-methoxyethoxy)quinazolin-4-yl]amino}benzo-1,4-quinone;
(rrrrrrrrr) 2-{[6-methoxy-7-(2-methoxyethoxy)quinazolin-4-yl]amino}-5-[methyl(2-pyridin-2-ylethyl)amino]benzo-1,4-quinone;
(sssssssss) 3-chloro-2-[4-(3-chlorobenzyl)piperazin-1-yl]-5-{[6-methoxy-7-(2-methoxyethoxy)quinazolin-4-yl]amino}benzo-1,4-quinone;
(tttttttt) 4-{[4-(4-{[6-methoxy-7-(2-methoxyethoxy)quinazolin-4-yl]amino}-3,6-dioxocyclohexa-1,4-dien-1-yl)piperazin-1-yl]methyl}benzonitrile;
(uuuuuuuuu) 2-{4-[4-(dimethylamino)benzyl]piperazin-1-yl}-5-{[6-methoxy-7-(2-methoxyethoxy)quinazolin-4-yl]amino}benzo-1,4-quinone;
(vvvvvwvv) 2-{[6-methoxy-7-(2-methoxyethoxy)quinazolin-4-yl]amino}-5-[4-(2-methylbutyl)piperazin-1-yl]benzo-1,4-quinone;
(wwwwwwwww) 2-[4-(1,3-benzodioxol-5-ylmethyl)piperazin-1-yl]-5-{[6-methoxy-7-(2-methoxyethoxy)quinazolin-4-yl]amino}benzo-1,4-quinone;
(xxxxxxxxx) 2-[4-(3-fluorobenzyl)piperazin-1-yl]-5-{[6-methoxy-7-(2-methoxyethoxy)quinazolin-4-yl]amino}benzo-1,4-quinone;
(yyyyyyyyy) 2-{[6-methoxy-7-(2-methoxyethoxy)quinazolin-4-yl]amino}-5-[4-(2-thienylmethyl)piperazin-1-yl]benzo-1,4-quinone;
(zzzzzzzzz) 2-[4-(3,7-dimethyloct-6-en-1-yl)piperazin-1-yl]-5-{[6-methoxy-7-(2-methoxyethoxy)quinazolin-4-yl]amino}benzo-1,4-quinone;
(aaaaaaaaaa) 2-[4-(2-methoxybenzyl)piperidin-1-yl]-5-{[6-methoxy-7-(2-methoxyethoxy)quinazolin-4-yl]amino}benzo-1,4-quinone;
(bbbbbbbbbb) 2-[4-(2-furylmethyl)piperazin-1-yl]-5-{[6-methoxy-7-(2-methoxyethoxy)quinazolin-4-yl]amino}benzo-1,4-quinone;
(cccccccccc) 2-{[6-methoxy-7-(2-methoxyethoxy)quinazolin-4-yl]amino}-5-[4-(pyridin-3-ylmethyl)piperazin-1-yl]benzo-1,4-quinone;
(dddddddddd) 2-[4-(2,4-dimethoxybenzyl)-1,4-diazepan-1-yl]-5-{[6-methoxy-7-(2-methoxyethoxy)quinazolin-4-yl]amino}benzo-1,4-quinone;
(eeeeeeeeee) 2-{[6-methoxy-7-(2-methoxyethoxy)quinazolin-4-yl]amino}-5-[4-(2-methylbutyl)-1,4-diazepan-1-yl]benzo-1,4-quinone;
(ffffffffff) 5-[4-(1,3-benzodioxol-5-ylmethyl)piperazin-1-yl]-3-(ethylthio)-2-{[6-methoxy-7-(2-methoxyethoxy)quinazolin-4-yl]amino}benzo-1,4-quinone;
(gggggggggg) 2-[(2-chlorobenzyl)oxy]-5-{[6-methoxy-7-(2-methoxyethoxy)quinazolin-4-yl]amino}benzo-1,4-quinone;
(hhhhhhhhhh) 2-{[6-methoxy-7-(2-methoxyethoxy)quinazolin-4-yl]amino}-5-(methylthio)benzo-1,4-quinone;
(iiiiiiiiii) 2-isopropoxy-5-{[6-methoxy-7-(2-methoxyethoxy)quinazolin-4-yl]amino}benzo-1,4-quinone;
(jjjjjjjjjj) 2-{[6-methoxy-7-(2-methoxyethoxy)quinazolin-4-yl]amino}-5-(1-methylbutoxy)benzo-1,4-quinone;
(kkkkkkkkkk) 2-(cycloheptyloxy)-5-{[6-methoxy-7-(2-methoxyethoxy)quinazolin-4-yl]amino}benzo-1,4-quinone;
(llllllllll) 2-sec-butoxy-5-{[6-methoxy-7-(2-methoxyethoxy)quinazolin-4-yl]amino}benzo-1,4-quinone;
(mmmmmmmmmm) 2-(1-ethylpropoxy)-5-{[6-methoxy-7-(2-methoxyethoxy)quinazolin-4-yl]amino}benzo-1,4-quinone;
(nnnnnnnnnn) 2-[(1,4-dimethylpentyl)oxy]-5-{[6-methoxy-7-(2-methoxyethoxy)quinazolin-4-yl]amino}benzo-1,4-quinone;
(oooooooooo) 2-{[6-methoxy-7-(2-methoxyethoxy)quinazolin-4-yl]amino}-5-[(1-methylpiperidin-4-yl)oxy]benzo-1,4-quinone;
(pppppppppp) 2-{[6-methoxy-7-(2-methoxyethoxy)quinazolin-4-yl]amino}-5-[(1-methylpiperidin-3-yl)oxy]benzo-1,4-quinone;
(qqqqqqqqqq) 2-[(2-fluorobenzyl)oxy]-5-{[6-methoxy-7-(2-methoxyethoxy)quinazolin-4-yl]amino}benzo-1,4-quinone;
(rrrrrrrrrr) 2-[(3-fluorobenzyl)oxy]-5-{[6-methoxy-7-(2-methoxyethoxy)quinazolin-4-yl]amino}benzo-1,4-quinone;
(ssssssssss) 2-{[6-methoxy-7-(2-methoxyethoxy)quinazolin-4-yl]amino}-5-(tetrahydro-2H-pyran-2-ylmethoxy)benzo-1,4-quinone;
(tttttttttt) 2-[(4-fluorobenzyl)oxy]-5-{[6-methoxy-7-(2-methoxyethoxy)quinazolin-4-yl]amino}benzo-1,4-quinone;
(uuuuuuuuuu) 2-{[6-methoxy-7-(2-methoxyethoxy)quinazolin-4-yl]amino}-5-(tetrahydro-2H-pyran-4-yloxy)benzo-1,4-quinone;
(vvvvvvvvvv) 2-[2-(dimethylamino)-1-methylethoxy]-5-{[6-methoxy-7-(2-methoxyethoxy)quinazolin-4-yl]amino}benzo-1,4-quinone;
(wwwwwwwwww) 2-[(4-methoxybenzyl)oxy]-5-{[6-methoxy-7-(2-methoxyethoxy)quinazolin-4-yl]amino}benzo-1,4-quinone;
(xxxxxxxxxx) 2-(2,3-dihydro-1H-inden-2-yloxy)-5-{[6-methoxy-7-(2-methoxyethoxy)quinazolin-4-yl]amino}benzo-1,4-quinone;
(yyyyyyyyyy) 2-{[6-methoxy-7-(2-methoxyethoxy)quinazolin-4-yl]amino}-5-(3-phenoxypropoxy)benzo-1,4-quinone;
(zzzzzzzzzz) 2-ethoxy-5-{[6-methoxy-7-(2-methoxyethoxy)quinazolin-4-yl]amino}benzo-1,4-quinone;
(aaaaaaaaaaa) 2-{[6-methoxy-7-(2-methoxyethoxy)quinazolin-4-yl]amino}-5-(tetrahydrofuran-3-ylmethoxy)benzo-1,4-quinone;
(bbbbbbbbbbb) 2-{[6-methoxy-7-(2-methoxyethoxy)quinazolin-4-yl]amino}-5-(2,2,2-trifluoro-1-phenylethoxy)benzo-1,4-quinone;
(ccccccccccc) 2-{[6-methoxy-7-(2-methoxyethoxy)quinazolin-4-yl]amino}-5-[(3R)-tetrahydrofuran-3-yloxy]benzo-1,4-quinone;
(ddddddddddd) 2-{[6-methoxy-7-(2-methoxyethoxy)quinazolin-4-yl]amino}-5-[(3S)-tetrahydrofuran-3-yloxy]benzo-1,4-quinone;
(eeeeeeeeeee) 2-{[6-methoxy-7-(2-methoxyethoxy)quinazolin-4-yl]amino}-5-[(3-methyloxetan-3-yl)methoxy]benzo-1,4-quinone;
(fffffffffff) 2-{[1-(4-chlorophenyl)cyclopropyl]methoxy}-5-{[6-methoxy-7-(2-methoxyethoxy)quinazolin-4-yl]amino}benzo-1,4-quinone;
(ggggggggggg) 2-{[6-methoxy-7-(2-methoxyethoxy)quinazolin-4-yl]amino}-5-[(1-methylpyrrolidin-3-yl)oxy]benzo-1,4-quinone;
(hhhhhhhhhhh) 2-{[6-methoxy-7-(2-methoxyethoxy)quinazolin-4-yl]amino}-5-[(pentafluorobenzyl)oxy]benzo-1,4-quinone;
(iiiiiiiiiii) 2-(2,2-difluoroethoxy)-5-{[6-methoxy-7-(2-methoxyethoxy)quinazolin-4-yl]amino}benzo-1,4-quinone;
(jjjjjjjjjjj) 2-[(2,3,3,4,4,5-hexafluorocyclopentyl)oxy]-5-{[6-methoxy-7-(2-methoxyethoxy)quinazolin-4-yl]amino}benzo-1,4-quinone;
(kkkkkkkkkkk) 2-(1,3-benzodioxol-5-ylmethoxy)-5-{[6-methoxy-7-(2-methoxyethoxy)quinazolin-4-yl]amino}benzo-1,4-quinone;
(lllllllllll) 2-{[4-(benzyloxy)-3-methoxybenzyl]oxy}-5-{[6-methoxy-7-(2-methoxyethoxy)quinazolin-4-yl]amino}benzo-1,4-quinone;
(mmmmmmmmmmm) 2-{[4-(benzyloxy)benzyl]oxy}-5-{[6-methoxy-7-(2-methoxyethoxy)quinazolin-4-yl]amino}benzo-1,4-quinone;
(nnnnnnnnnnn) 2-{[6-methoxy-7-(2-methoxyethoxy)quinazolin-4-yl]amino}-5-[(3-phenylprop-2-yn-1-yl)oxy]benzo-1,4-quinone;
(ooooooooooo) 2-{[6-methoxy-7-(2-methoxyethoxy)quinazolin-4-yl]amino}-5-[(3-phenoxybenzyl)oxy]benzo-1,4-quinone;
(ppppppppppp) 2-[(2-hydroxyethyl)amino]-5-{[6-methoxy-7-(3-pyrrolidin-1-ylpropoxy)quinazolin-4-yl]amino}benzo-1,4-quinone;
(qqqqqqqqqq) 2-(2-furylmethoxy)-5-{[6-methoxy-7-(3-pyrrolidin-1-ylpropoxy)quinazolin-4-yl]amino}benzo-1,4-quinone;
(rrrrrrrrrrr) 2-{[6-methoxy-7-(3-pyrrolidin-1-ylpropoxy)quinazolin-4-yl]amino}-5-[(1-methylprop-2-yn-1-yl)oxy]benzo-1,4-quinone;
(sssssssssss) 2-(allyloxy)-5-{[6-methoxy-7-(3-pyrrolidin-1-ylpropoxy)quinazolin-4-yl]amino}benzo-1,4-quinone;
(ttttttttttt) 2-{[6-methoxy-7-(3-pyrrolidin-1-ylpropoxy)quinazolin-4-yl]amino}-5-(prop-2-yn-1-yloxy)benzo-1,4-quinone;
(uuuuuuuuuuu) 2-{[6-methoxy-7-(3-pyrrolidin-1-ylpropoxy)quinazolin-4-yl]amino}-5-[(1-phenylprop-2-yn-1-yl)oxy]benzo-1,4-quinone;
(vvvvvvvvvvv) 2-{[6-methoxy-7-(3-pyrrolidin-1-ylpropoxy)quinazolin-4-yl]amino}-5-(tetrahydrofuran-3-yloxy)benzo-1,4-quinone;
(wwwwwwwwwww) 2-{[6-methoxy-7-(3-pyrrolidin-1-ylpropoxy)quinazolin-4-yl]amino}-5-[(2-methylbenzyl)oxy]benzo-1,4-quinone;
(xxxxxxxxxxx) 2-{[6-methoxy-7-(3-pyrrolidin-1-ylpropoxy)quinazolin-4-yl]amino}-5-{[4-(methylsulfonyl)benzyl]oxy}benzo-1,4-quinone;
(yyyyyyyyyyy) 2-{[6-methoxy-7-(3-pyrrolidin-1-ylpropoxy)quinazolin-4-yl]amino}-5-[(pentafluorobenzyl)oxy]benzo-1,4-quinone;
(zzzzzzzzzzz) 2-({4-[(4-fluorobenzyl)oxy]benzyl}oxy)-5-{[6-methoxy-7-(3-pyrrolidin-1-ylpropoxy)quinazolin-4-yl]amino}benzo-1,4-quinone;
(aaaaaaaaaaa) 2-{[6-methoxy-7-(3-pyrrolidin-1-ylpropoxy)quinazolin-4-yl]amino}-5-{2-[methyl(phenyl)amino]ethoxy}benzo-1,4-quinone;
(bbbbbbbbbbbb) 2-(benzyloxy)-5-{[6-methoxy-7-(3-pyrrolidin-1-ylpropoxy)quinazolin-4-yl]amino}benzo-1,4-quinone;
(cccccccccccc) 2-[(4-chlorobenzyl)oxy]-5-{[6-methoxy-7-(3-pyrrolidin-1-ylpropoxy)quinazolin-4-yl]amino}benzo-1,4-quinone;
(dddddddddddd) 2-{[6-methoxy-7-(3-pyrrolidin-1-ylpropoxy)quinazolin-4-yl]amino}-5-(pyridin-3-ylmethoxy)benzo-1,4-quinone;
(eeeeeeeeeeee) 2-{[6-methoxy-7-(3-pyrrolidin-1-ylpropoxy)quinazolin-4-yl]amino}-5-(pyridin-2-ylmethoxy)benzo-1,4-quinone;
(ffffffffffff) 3-{[(4-{[6-methoxy-7-(3-pyrrolidin-1-ylpropoxy)quinazolin-4-yl]amino}-3,6-dioxocyclohexa-1,4-dien-1-yl)oxy]methyl}benzonitrile;
(gggggggggggg) 2-[2-chloro-1-(fluoromethyl)ethoxy]-5-{[6-methoxy-7-(3-pyrrolidin-1-ylpropoxy)quinazolin-4-yl]amino}benzo-1,4-quinone;
(hhhhhhhhhhhh) 2-{[6-methoxy-7-(3-pyrrolidin-1-ylpropoxy)quinazolin-4-yl]amino}-5-[(3-phenylprop-2-yn-1-yl)oxy]benzo-1,4-quinone;
(iiiiiiiiiiii) 2-[(3-fluorobenzyl)oxy]-5-{[6-methoxy-7-(3-pyrrolidin-1-ylpropoxy)quinazolin-4-yl]amino}benzo-1,4-quinone;
(jjjjjjjjjjjj) 2-(2,2-difluoroethoxy)-5-{[6-methoxy-7-(3-pyrrolidin-1-ylpropoxy)quinazolin-4-yl]amino}benzo-1,4-quinone;
(kkkkkkkkkkkk) 2-[2-fluoro-1-(fluoromethyl)ethoxy]-5-{[6-methoxy-7-(3-pyrrolidin-1-ylpropoxy)quinazolin-4-yl]amino}benzo-1,4-quinone;
(lllllllllll) 2-{[6-methoxy-7-(3-pyrrolidin-1-ylpropoxy)quinazolin-4-yl]amino}-5-(3-phenoxypropoxy)benzo-1,4-quinone;
(mmmmmmmmmmmm) 2-{[6-methoxy-7-(3-pyrrolidin-1-ylpropoxy)quinazolin-4-yl]amino}-5-{[(2E)-3-phenylprop-2-en-1-yl]oxy}benzo-1,4-quinone;
(nnnnnnnnnnnn) 2-methoxy-5-{[6-methoxy-7-(3-pyrrolidin-1-ylpropoxy)quinazolin-4-yl]amino}benzo-1,4-quinone;
(oooooooooooo) 2-(4-benzylpiperazin-1-yl)-5-({6-methoxy-7-[(1-methylpiperidin-4-yl)methoxy]quinazolin-4-yl}amino)benzo-1,4-quinone;
(pppppppppppp) 2-[4-(2-methoxybenzyl)piperidin-1-yl]-5-({6-methoxy-7-[(1-methylpiperidin-4-yl)methoxy]quinazolin-4-yl}amino)benzo-1,4-quinone;
(qqqqqqqqqqq) 2-[2-fluoro-1-(fluoromethyl)ethoxy]-5-({6-methoxy-7-[(1-methylpiperidin-4-yl)methoxy]quinazolin-4-yl}amino)benzo-1,4-quinone;
(rrrrrrrrrrrr) 2-({6-methoxy-7-[(1-methylpiperidin-4-yl)methoxy]quinazolin-4-yl}amino)-5-(2-phenoxyethoxy)benzo-1,4-quinone;
(ssssssssssss) 2-(benzyloxy)-5-({6-methoxy-7-[(1-methylpiperidin-4-yl)methoxy]quinazolin-4-yl}amino)benzo-1,4-quinone;
(tttttttttttt) 2-({6-methoxy-7-[(1-methylpiperidin-4-yl)methoxy]quinazolin-4-yl}amino)-5-{2-[methyl(phenyl)amino]ethoxy}benzo-1,4-quinone;
(uuuuuuuuuuuu) 2-ethoxy-5-({6-methoxy-7-[(1-methylpiperidin-4-yl)methoxy]quinazolin-4-yl}amino)benzo-1,4-quinone;
(vvvvvvvvvvvv) 2-methoxy-5-({6-methoxy-7-[(1-methylpiperidin-4-yl)methoxy]quinazolin-4-yl}amino)benzo-1,4-quinone;
(wwwwwwwwwwww) 2-methoxy-5-{[6-methoxy-7-(3-pyridin-4-ylpropoxy)quinazolin-4-yl]amino}benzo-1,4-quinone;
(xxxxxxxxxxxx) 2-chloro-5-{[6-methoxy-7-(3-pyridin-4-ylpropoxy)quinazolin-4-yl]amino}benzo-1,4-quinone;
(yyyyyyyyyyyy) 2-{[6,7-bis(2-methoxyethoxy)quinazolin-4-yl]amino}benzo-1,4-quinone;
(zzzzzzzzzzzz) 2-{[6,7-bis(2-methoxyethoxy)quinazolin-4-yl]amino}-5-(pyridin-3-ylmethoxy)benzo-1,4-quinone;
(aaaaaaaaaaaa) 2-{[6,7-bis(2-methoxyethoxy)quinazolin-4-yl]amino}-5-[2-fluoro-1-(fluoromethyl)ethoxy]benzo-1,4-quinone;
(bbbbbbbbbbbbb) 2-{[6,7-bis(2-methoxyethoxy)quinazolin-4-yl]amino}-5-methoxybenzo-1,4-quinone;
(ccccccccccccc) 2-{[6,7-bis(2-methoxyethoxy)quinazolin-4-yl]amino}-5-[4-(1H-imidazol-1-yl)phenoxy]benzo-1,4-quinone;
(dddddddddddd) 2-chloro-5-{[6-methoxy-7-(tetrahydro-2H-pyran-2-ylmethoxy)quinazolin-4-yl]amino}benzo-1,4-quinone;
(eeeeeeeeeeeee) 2-methoxy-5-{[6-methoxy-7-(tetrahydro-2H-pyran-2-ylmethoxy)quinazolin-4-yl]amino}benzo-1,4-quinone;
(fffffffffffff) 2-[2-fluoro-1-(fluoromethyl)ethoxy]-5-{[6-methoxy-7-(tetrahydro-2H-pyran-2-ylmethoxy)quinazolin-4-yl]amino}benzo-1,4-quinone;
(ggggggggggggg) 2-chloro-3-methoxy-5-{[6-methoxy-7-(2-methoxyethoxy)quinazolin-4-yl]amino}benzo-1,4-quinone;
(hhhhhhhhhhhhh) 2-chloro-3-isopropoxy-5-{[6-methoxy-7-(2-methoxyethoxy)quinazolin-4-yl]amino}benzo-1,4-quinone;
(iiiiiiiiiiiii) 2-chloro-3-(cyclopropylmethoxy)-5-{[6-methoxy-7-(2-methoxyethoxy)quinazolin-4-yl]amino}benzo-1,4-quinone;
(jjjjjjjjjjjjj) 3-chloro-2-methoxy-5-{[6-methoxy-7-(2-methoxyethoxy)quinazolin-4-yl]amino}benzo-1,4-quinone;
(kkkkkkkkkkkkk) 3-chloro-2-[2-fluoro-1-(fluoromethyl)ethoxy]-5-{[6-methoxy-7-(2-methoxyethoxy)quinazolin-4-yl]amino}benzo-1,4-quinone;
(lllllllllllll) 3-chloro-2-[(3-fluorobenzyl)oxy]-5-{[6-methoxy-7-(2-methoxyethoxy)quinazolin-4-yl]amino}benzo-1,4-quinone;
(mmmmmmmmmmmmm) 3-chloro-2-ethoxy-5-{[6-methoxy-7-(2-methoxyethoxy)quinazolin-4-yl]amino}benzo-1,4-quinone;
(nnnnnnnnnnnnn) 3-chloro-5-{[6-methoxy-7-(2-methoxyethoxy)quinazolin-4-yl]amino}-2-(tetrahydrofuran-3-yloxy)benzo-1,4-quinone;
(ooooooooooooo) 2-({7-[3-(diethylamino)propoxy]-6-methoxyquinazolin-4-yl}amino)-5-methoxybenzo-1,4-quinone;
(ppppppppppppp) 2,3,5-tris(ethylthio)-6-{[6-methoxy-7-(2-methoxyethoxy)quinazolin-4-yl]amino}benzo-1,4-quinone;
(qqqqqqqqqqqq) 3-(ethylthio)-5-methoxy-2-{[6-methoxy-7-(2-methoxyethoxy)quinazolin-4-yl]amino}benzo-1,4-quinone-3-(ethylthio)-5-methoxy-2-{[6-methoxy-7-(2-methoxyethoxy)quinazolin-4-yl]amino}benzene-1,4-diol (1:1);
(rrrrrrrrrrrrr) 2-ethoxy-5-{[6-methoxy-7-(3-pyrrolidin-1-ylpropoxy)quinazolin-4-yl]amino}benzo-1,4-quinone;
(sssssssssssss) 2-{[6-methoxy-7-(3-pyrrolidin-1-ylpropoxy)quinazolin-4-yl]amino}-5-(2-phenoxyethoxy)benzo-1,4-quinone;
(ttttttttttttt) 2-{[6,7-bis(2-methoxyethoxy)quinazolin-4-yl]amino}-5-chlorobenzo-1,4-quinone;
(uuuuuuuuuuuuu) 2-(4-benzylpiperazin-1-yl)-3-chloro-5-{[6-methoxy-7-(2-methoxyethoxy)quinazolin-4-yl]amino}benzo-1,4-quinone;
(vvvvvvvvvvvvv) 3-chloro-2-(3,5-dimethylpiperidin-1-yl)-5-{[6-methoxy-7-(2-methoxyethoxy)quinazolin-4-yl]amino}benzo-1,4-quinone;
(wwwwwwwwwww) 3-chloro-2-(dimethylamino)-5-{[6-methoxy-7-(2-methoxyethoxy)quinazolin-4-yl]amino}benzo-1,4-quinone;
(xxxxxxxxxxxxx) 2,3-dimethoxy-5-{[6-methoxy-7-(2-methoxyethoxy)quinazolin-4-yl]amino}benzo-1,4-quinone;
(yyyyyyyyyyyyy) 2-[2-fluoro-1-(fluoromethyl)ethoxy]-3-methoxy-5-{[6-methoxy-7-(2-methoxyethoxy)quinazolin-4-yl]amino}benzo-1,4-quinone;
(zzzzzzzzzzzzz) (2E)-4-(dimethylamino)-N-{7-ethoxy-4-[(4-methoxy-3,6-dioxocyclohexa-1,4-dien-1-yl)amino]quinazolin-6-yl}but-2-enamide;
(aaaaaaaaaaaaa) (2E)-4-(dimethylamino)-N-[7-ethoxy-4-({4-[(3-fluorobenzyl)oxy]-3,6-dioxocyclohexa-1,4-dien-1-yl}amino)quinazolin-6-yl]but-2-enamide;
(bbbbbbbbbbbbbb) (2E)-4-(dimethylamino)-N-[7-ethoxy-4-({4-[2-fluoro-1-(fluoromethyl)ethoxy]-3,6-dioxocyclohexa-1,4-dien-1-yl}amino)quinazolin-6-yl]but-2-enamide;
(ccccccccccccc) (2E)-N-[4-({4-[(3,4-difluorobenzyl)oxy]-3,6-dioxocyclohexa-1,4-dien-1-yl}amino)-7-ethoxyquinazolin-6-yl]-4-(dimethylamino)but-2-enamide;
(ddddddddddddd) (2E)-N-(4-{[4-(benzyloxy)-3,6-dioxocyclohexa-1,4-dien-1-yl]amino}-7-ethoxyquinazolin-6-yl)-4-(dimethylamino)but-2-enamide;
(eeeeeeeeeeeeee) (2E)-4-(dimethylamino)-N-(4-{[3,6-dioxo-4-(pyridin-2-ylmethoxy)cyclohexa-1,4-dien-1-yl]amino}-7-ethoxyquinazolin-6-yl)but-2-enamide;
(fffffffffffff) (2E)-N-[4-({4-[(3-chlorobenzyl)oxy]-3,6-dioxocyclohexa-1,4-dien-1-yl}amino)-7-ethoxyquinazolin-6-yl]-4-(dimethylamino)but-2-enamide;
(gggggggggggggg) (2E)-4-(dimethylamino)-N-(4-{[3,6-dioxo-4-(2-thienylmethoxy)cyclohexa-1,4-dien-1-yl]amino}-7-ethoxyquinazolin-6-yl)but-2-enamide;
(hhhhhhhhhhhhhh) (2E)-4-(dimethylamino)-N-[7-ethoxy-4-({4-[(3-methoxybenzyl)oxy]-3,6-dioxocyclohexa-1,4-dien-1-yl}amino)quinazolin-6-yl]but-2-enamide;
(iiiiiiiiiiiiii) (2E)-4-(dimethylamino)-N-[7-ethoxy-4-({4-[(2-methylbenzyl)oxy]-3,6-dioxocyclohexa-1,4-dien-1-yl}amino)quinazolin-6-yl]but-2-enamide;
and pharmaceutically acceptable salts thereof.

7. A method of treating a disease characterized, in part, by excessive, abnormal, or inappropriate angiogenesis in a mammal in need thereof which comprises administering to said mammal an effective amount of the compound of claim 1.

8. The method of claim 7, wherein the mammal is human.

9. The method of claim 7, wherein the disease is cancer.

10. The method of claim 7, wherein the disease is diabetic retinopathy.

11. The method of claim 7, wherein the disease is macular degeneration.

12. The method of claim 7, wherein the disease is rheumatoid arthritis.

13. The method according to claim 9, wherein the cancer is selected from the group consisting of breast, kidney, bladder, mouth, larynx, esophagus, stomach, prostate, colon, ovary, and lung.

14. A method of inhibiting a tyrosine kinase enzyme consisting of contacting said enzyme with the compound of claim 1, wherein said compound binds irreversibly to said enzyme.

15. The compound of claim 15, wherein the tyrosine kinase enzyme is kinase domain receptor (KDR).

16. A pharmaceutical composition comprising the compound of claim 1 and a pharmaceutically acceptable carrier.
